(19) United States
(12) Patent Application Publication
(10) Pub. No.: US 2024/0000972 A1
(43) Pub. Date: Jan. 4, 2024

(54) RNA-TARGETING COMPOSITIONS AND METHODS FOR TREATING CAG REPEAT DISEASES

(71) Applicant: Locanabio, Inc., San Diego, CA (US)

(72) Inventors: David A. NELLES, San Diego, CA (US); Ranjan BATRA, San Diego, CA (US); Daniela ROTH, San Diego, CA (US); Dimitrios ZISOULIS, San Diego, CA (US); Angeline TA, San Diego, CA (US)

(21) Appl. No.: 18/039,813

(22) PCT Filed: Dec. 1, 2021
(86) PCT No.: PCT/US2021/061482

§ 371 Date: Jun. 1, 2023

Related U.S. Application Data
(60) Provisional application No. 63/130,060, filed on Dec. 23, 2020.
(60) Provisional application No. 63/119,977, filed on Dec. 1, 2020.

(51) Int. Cl. A61K 48/00; C12N 15/86; A61P 25/28; C12N 15/11; C12N 9/22

(52) U.S. Cl. CPC A61K 48/0058; C12N 15/86; A61P 25/28; C12N 15/11; C12N 9/22

(57) ABSTRACT

Disclosed are RNA-targeting gene therapy compositions and methods for destroying or blocking toxic target CAG repeat RNA and treating CAG repeat disorders such as Huntington's Disease (HD) and Spinocerebellar Ataxia Type 1 (SCA1).

### RELATED APPLICATIONS

This application claims benefit of, and priority to, U.S. Ser. No. 63/119,977 filed on Dec. 1, 2020 and U.S. Ser. No. 63/130,060 filed on Dec. 23, 2020; the contents of each are hereby incorporated by reference in their entireties.

### FIELD OF THE DISCLOSURE

The disclosure is directed to molecular biology, gene therapy, and compositions and methods for modifying expression and activity of RNA molecules.

### INCORPORATION BY REFERENCE OF SEQUENCE LISTING

The contents of the text file named “LOCN_008_001WO_SeqList_ST25”, which was created on Dec. 1, 2021 and is 140 KB in size, are hereby incorporated by reference in their entirety.

### BACKGROUND

There are long-felt but unmet needs in the art for providing effective gene therapies, particularly gene therapies which target the underlying pathogenic RNA causing a disease.

Over 20 unstable microsatellite repeat expansion (MRE) have been identified as the cause of neurological disease in humans. (Rohilla and Gagnon, Acta Neuropahtologica Communications, (2017) 5:63.) Pathogenic RNAs expressed from these repetitive MRE tracts in microsatellite repeat expansion causes a range of debilitating and often devastating diseases and disorders. These repeat RNAs, their location within the genes, the ranges of normal and disease-causing repeat length and the clinical outcomes differ. Unstable repeats can be located in the coding or non-coding region of a gene. Available treatments address symptoms of these MRE diseases but do not target their underlying etiology.

The most common trinucleotide repeat causing disease by altering protein physiology is the CAG MRE. The translation of the CAG MRE results in a polyQ tract. Many different disorders share a CAG repeat in the coding region of a gene. Although expansion sizes, structures, cellular localization and functions of the resulting proteins differ, all CAG MRE-induced diseases are neurodegenerative and/or neuromuscular diseases or disorders.

HD is a fatal disorder caused by CAG repeat expansion in the Huntingtin (HTT) gene. The disease leads to degeneration of striatal neurons leading to uncontrolled movements, emotional problems, and dementia. There are currently more than 40,000 patients, and 200,000 at risk patients, in the US.

Expansion CAG repeats also cause a group of Spinocerebellar Ataxias (SCAs), of which there are nine SCAs described to date, and of which a subset of SCAs is caused by the presence of CAG MREs. SCA1 is caused by the presence of CAG trinucleotide repeats in the ATXN1 gene. SCA type 1 (SCA1) is a rare autodominant disorder characterized by progressive issues with movement. SCA1 symptoms include coordination and balance (ataxia), speech and swallowing difficulties, muscle stiffness (spasticity), and weakness in eye muscles which control eye movements (nystagmus), and cognitive impairment associated with processing, learning and memory. SCA1 affects 1 to 2 per 100,000 worldwide.

To overcome the absence of disease-modifying therapies for these CAG MRE diseases and disorders, therapeutics need to be delineated and developed for providing effective, sustained, and scalable treatment. RNA-targeting gene therapy systems are ideal for targeting pathogenic trinucleotide repeats such as CAG MREs which are the responsible for the underlying pathology of the disease and disorders.

Accordingly, the disclosure provides gene therapy compositions and methods for specifically targeting and destroying toxic RNAs expressed from repetitive tracts in microsatellite repeat expansion (MRE) diseases known as trinucleotide CAG repeat disorders such as Huntington's Disease (HD) and Spinocerebellar Ataxias (SCAs). RNA-targeting gene therapy compositions and systems capable of eliminating toxic CAG repeats, and methods using the same for treating CAG MRE-causing diseases and disorders, are provided herein.

### SUMMARY

The disclosure provides compositions and methods for CAG-repeat disorders. The compositions and methods disclosed herein result in dose-dependent reduction in CAGexP (CAG-repeat expansion) RNA via either destruction or blocking.

The disclosure provides compositions and methods for treating CAG MRE-causing diseases and disorders.

Disclosed herein is a method of treating Huntington's Disease (HD) in a mammal comprising administering a composition to a toxic target CAG microsatellite repeat expansion (MRE) molecule in tissues of the mammal, wherein the composition comprises a nucleic acid sequence encoding a non-guided RNA-binding fusion protein comprising a) a PUF RNA-binding sequence or Cas13d RNA-binding protein capable of binding a toxic target CAG RNA repeat sequence, and b) an endonuclease capable of cleaving the toxic target CAG RNA repeat sequence, whereby the level of expression of the toxic target RNA is reduced.

Disclosed herein is a method of treating Spinocerebellar Ataxia Type 1 (SCA1), in a mammal comprising administering a composition to a toxic target CAG microsatellite repeat expansion (MRE) molecule in tissues of the mammal, wherein the composition comprises a nucleic acid sequence encoding a non-guided RNA-binding fusion protein comprising a) a PUF RNA-binding sequence or Cas13d RNA-binding protein capable of binding a toxic target CAG RNA repeat sequence, and b) an endonuclease capable of cleaving the toxic target CAG RNA repeat sequence, whereby the level of expression of the toxic target RNA is reduced.

The disclosure provides a composition comprising a nucleic acid sequence encoding an RNA-binding polypeptide comprising a non-guided RNA binding polypeptide or a guided RNA-binding polypeptide capable of binding a toxic target CAG repeat RNA sequence.

In some embodiments, the RNA-binding polypeptide is a fusion protein. In some embodiments, the fusion protein comprises the RNA binding polypeptide fused to an endonuclease capable of cleaving the toxic CAG repeat RNA sequence.

In some embodiments, the non-guided RNA binding polypeptide is a PUF or PUMBY protein. In some embodiments, the guided RNA-binding polypeptide is a Cas13d protein. In some embodiments, the cas13d protein is catalytically dead.

In some embodiments, the casl3d protein comprises an amino acid sequence set forth in any one of SEQ ID NOs 587 or 590-594.

In some embodiments, the endonuclease is a nuclease domain of a ZC3H12A zinc-finger endonuclease.

In some embodiments, the PUF RNA binding protein comprises an amino acid sequence set forth in any one of SEQ ID NOs 444-451, 461, 480-488, 549-557, or 656. In some embodiments, the PUF RNA binding protein comprises an amino acid sequence set forth in SEQ ID NO: 549 or 480.

In some embodiments, the toxic target CAG RNA repeat sequence comprises any one of the nucleic acid sequences set forth in SEQ ID NOs 453-456 or 472-479. In some embodiments, the toxic target CAG RNA repeat sequence comprises the nucleic acid sequence set forth in any one of SEQ ID NO: 453 or 472.

In some embodiments, the CAG-targeting PUF protein is encoded by a nucleic acid sequence as set forth in SEQ ID NO: 577, 581, 614, 619, 621, or 622.

In some embodiments, wherein the PUF or PUMBY protein is a human PUF or PUMBY protein. In some embodiments, the PUF or PUMBY protein is linked to the ZC3H12A endonuclease by a linker sequence.

In some embodiments, the linker comprises the amino acid sequence set forth in SEQ ID NO: 411.

In some embodiments, the fusion protein comprises one or more signal sequences selected from the group consisting of a nuclear localization sequence (NLS), and a nuclear export sequence (NES).

In some embodiments, the ZC3H12A zinc finger nuclease comprises the amino acid sequence set forth in SEQ ID NO: 358 or SEQ ID NO: 359.

In some embodiments, the fusion protein comprises the amino acid sequence set forth in any one of SEQ ID NO: 460. In some embodiments, the fusion protein is encoded by a nucleic acid sequence comprising SEQ ID NO: 574-582.

In some embodiments, the nucleic acid molecule encoding the fusion protein comprises a promoter. In some embodiments, the promoter is a tCAG promoter, EFS/UBB promoter, or synapsin promoter.

A vector comprising the composition of any embodiment of the disclosure.

In some embodiments, the vector is selected from the group consisting of: adeno-associated virus (AAV), retrovirus, lentivirus, adenovirus, nanoparticle, micelle, liposome, lipoplex, polymersome, polyplex, and dendrimer. In some embodiments, is an AAV vector.

In some embodiments, the AAV vector comprises: a first AAV ITR sequence; a first promoter sequence; a polynucleotide sequence encoding for at least one CAG-repeat RNA binding polypeptide; and a second AAV ITR sequence.

In some embodiments, the CAG-repeat RNA binding polypeptide comprises a PUF or PUMBY protein. The AAV vector of any embodiment of the disclosure, wherein the polynucleotide sequence encoding the PUF or PUMBY sequence comprises a nucleic acid sequence set forth in SEQ ID NO: 577, 581, 614, 619, 621, or 622.

In some embodiments, the CAG-repeat RNA binding polypeptide comprises a Cas13d protein. In some embodiments, the polynucleotide sequence encoding the Cas13d sequence comprises a nucleic acid sequence set forth in SEQ ID NO: 587 or 590-594.

In some embodiments, the first promoter sequence comprises a nucleic acid sequence set forth in SEQ ID NO: 389, 627, or 613.

In some embodiments, the first AAV ITR sequence comprises a nucleic acid sequence set forth in SEQ ID NO: 597 or 598. In some embodiments, the second AAV ITR sequence comprises a nucleic acid sequence set forth in SEQ ID NO: 597 or 598.

In some embodiments, the vector further comprises a second promoter sequence.

In some embodiments, wherein the second promoter controls expression of a guide RNA (gRNA) wherein the gRNA comprises (i) a DR sequence and (ii) a spacer sequence. In some embodiments, the second promoter comprises a nucleic acid sequence set forth in SEQ ID NO: 519.

In some embodiments, the vector further comprises a polyA sequence. In some embodiments, the vector comprises at least one linker sequence.

In some embodiments, the vector comprises at least one nuclear localization sequence. In some embodiments, the vector is encoded be a nucleic set forth in any of one of SEQ ID NO: 588, 589, 624, or 625.

The disclosure provides a pharmaceutical composition comprising: a) the AAV viral vector of any embodiment of the disclosure; and b) at least one pharmaceutically acceptable excipient and/or additive.

The disclosure provides an AAV viral vector comprising: a) an AAV vector of any embodiment of the disclosure; and b) an AAV capsid protein.

In some embodiments, the AAV capsid protein is an AAV1 capsid protein, an AAV2 capsid protein, an AAV4 capsid protein, an AAV5 capsid protein, an AAV6 capsid protein, an AAV7 capsid protein, an AAV8 capsid protein, an AAV9 capsid protein, an AAV10 capsid protein, an AAV11 capsid protein, an AAV12 capsid protein, an AAV13 capsid protein, an AAVPHP.B capsid protein, an AAVrh74 capsid protein or an AAVrh.10 capsid protein. In some embodiments, the AAV capsid protein is an AAV9 or AAVrh10 capsid protein

The disclosure provides a cell comprising the vector of any embodiment of the disclosure.

The disclosure provides a method of treating a CAG repeat disease in a mammal comprising administering a composition or AAV vector according to any composition of the disclosure to a toxic target CAG microsatellite repeat expansion (MRE) RNA sequence in tissues of the mammal whereby the level of expression of the toxic target RNA is reduced.

In some embodiments, the composition or AAV vector is administered to the subject intravenously, intrathecally, intracerebrally, intraventricularly, intranasally, intratracheally, intra-aurally, intra-ocularly, or peri-ocularly, orally, rectally, transmucosally, inhalationally, transdermally, parenterally, subcutaneously, intradermally, intramuscularly, intracistemally, intranervally, intrapleurally, topically, intralymphatically, intracisternally or intranerve.

In some embodiments, the composition or AAV vector is administered to the subject intravenously. In some embodiments, the CAG repeat disorder is Huntington's Disease (HD) or Spinocerebellar Ataxia Type 1 (SCA1)

In some embodiments, the reduced level of expression of the toxic target RNA thereby ameliorates symptoms of HD or SCA1 in the mammal.

In some embodiments, the level of expression of the toxic target RNA is reduced compared to the reduction in the level of expression of untreated toxic target CAG RNA.

In some embodiments, the toxic CAG repeat is a CAG³⁶ or more. In some embodiments, the toxic CAG repeat is a CAG⁸⁰ repeat. In some embodiments, the level of reduction is between 1-fold and 20-fold.

Disclosed herein is a composition comprising a nucleic acid sequence encoding a non-guided RNA-binding fusion protein comprising a) a PUF or PUMBY protein capable of binding a toxic target CAG repeat RNA sequence and b) an endonuclease capable of cleaving the toxic target RNA sequence, wherein the endonuclease is a nuclease domain of a ZC3H12A zinc-finger endonuclease.

In some embodiments, the PUF RNA binding protein comprises any one of SEQ ID NOs 444-451, 461, 480-488, or 549-557.

In some embodiments, the PUF RNA binding protein comprises SEQ ID NO: 549 or 480.

In some embodiments, the toxic target CAG RNA repeat sequence comprises any one of SEQ ID NOs 453-456 or 472-479.

In some embodiments, the toxic target CAG RNA repeat sequence comprises SEQ ID NO: 453 or 472.

In some embodiments, the CAG-targeting PUF protein is encoded by a nucleic acid sequence comprising any one of SEQ ID NOs 577 or 581.

In some embodiments, the PUF or PUMBY protein is a human PUF or PUMBY protein.

In some embodiments, the PUF or PUMBY protein is linked to the ZC3H12A by a VDTANGS (SEQ ID NO: 411) linker.

In some embodiments, the fusion protein comprises one or more signal sequence selected from the group consisting of a nuclear localization sequence (NLS), and a nuclear export sequence (NES).

In some embodiments, the ZC3H12A zinc finger nuclease comprises SEQ ID NO: 358 or SEQ ID NO: 359.

In some embodiments, the fusion protein is encoded by a nucleic acid sequence comprising any one of SEQ ID NOs 574-582.

In some embodiments, the nucleic acid molecule encoding the fusion protein comprises a promoter.

In some embodiments, the promoter is a tCAG promoter.

Disclosed herein is a vector comprising any of the preceding compositions.

In some embodiments, the vector is selected from the group consisting of: adeno-associated virus (AAV), retrovirus, lentivirus, adenovirus, nanoparticle, micelle, liposome, lipoplex, polymersome, polyplex, and dendrimer.

In some embodiments, is an AAV vector.

In some embodiments, the AAV vector is AAV9, AAVrh10, or AAVrh.74.

Disclosed herein is a cell comprising the vector of any preceding embodiment.

Disclosed herein is a method of treating CAG repeat disease in a mammal comprising administering a composition to a toxic target CAG microsatellite repeat expansion (MRE) RNA sequence in tissues of the mammal, wherein the composition comprises a nucleic acid sequence encoding a non-guided RNA-binding fusion protein comprising a) a PUF RNA-binding protein capable of binding a toxic target CAG RNA repeat sequence, and b) an endonuclease capable of cleaving the toxic target CAG RNA repeat sequence, whereby the level of expression of the toxic target RNA is reduced.

In some embodiments, the PUF RNA binding protein comprises any one of SEQ ID NOs 444-451, 461, 480-488, or 549-557.

In some embodiments, the PUF RNA binding protein comprises SEQ ID NO: 549 or 480.

In some embodiments, the toxic target CAG RNA repeat sequence comprises any one of SEQ ID NOs 453-456 or 472-479.

In some embodiments, the toxic target CAG RNA repeat sequence comprises SEQ ID NO: 453 or 472.

In some embodiments, the composition is administered to the tissue of the mammal by intrastriatal administration.

In some embodiments, the reduced level of expression of the toxic target RNA thereby ameliorates symptoms of the CAG repeat disorder in the mammal.

In some embodiments, the level of expression of the toxic target RNA is reduced compared to the reduction in the level of expression of untreated toxic target CAG RNA.

In some embodiments, the level of reduction is between 1-fold and 20-fold.

In some embodiments, the endonuclease is a domain of a ZC3H12A zinc-finger endonuclease.

In some embodiments, the domain of the ZC3H12A zinc finger nuclease comprises SEQ ID NO: 358 or SEQ ID NO: 359.

In some embodiments, the nucleic acid sequence encoding the fusion protein comprises a promoter.

In some embodiments, the promoter is a tCAG promoter.

In some embodiments, the promoter is a neuron-specific promoter.

In some embodiments, the neuron-specific promoter is a synapsin promoter.

In some embodiments, the fusion protein is encoded by a nucleic acid sequence comprising any one of SEQ ID NOs 574-582.

A composition comprising a nucleic acid sequence encoding a non-naturally occurring or engineered clustered regularly interspaced short palindromic repeats (CRISPR)-associated (Cas) system comprising: (a) at least one RNA-guided RNse Cas protein; and b) at least one cognate CRISPR-Cas system guide RNA (gRNA) capable of forming a complex with one of the at least one Cas proteins, wherein the gRNA comprises (i) a DR sequence and (ii) a spacer sequence, wherein the spacer sequence hybridizes with the target CAG MRE molecule, and wherein the spacer sequence comprises a spacer sequence selected from the group consisting of: tgctgctgctgctgctgctgctgctg (guide 1, SEQ ID NO: 457), gctgctgctgctgctgctgctgctgc (guide 2, SEQ ID NO: 458), and ctgctgctgctgctgctgctgctgct (guide 3, SEQ ID NO: 458) or a portion thereof, wherein the CRISPR-Cas system is capable of binding and cleaving the target CAG MRE, wherein the CRISPR-Cas system is catalytically inactive, and wherein the CRISPR-Cas is capable of binding but not cleaving the target CAG MRE.

In some embodiments, the Cas protein is Cas13a, Cas13b, Cas13c, or Cas13d. In some embodiments, the Cas protein is Cas13d.

In some embodiments, the RNA-guided RNase Cas protein or the non-guided RNA-binding polypeptide is a first RNA-binding polypeptide which is fused with a second RNA-binding polypeptide. In one embodiment, the second RNA-binding polypeptide is capable of binding RNA in a manner in which it associates with RNA. In some embodiments, the second RNA-binding polypeptide is capable of associating with RNA in a manner in which it cleaves RNA. In one embodiment, the second RNA-binding polypeptide is a nuclease domain of a ZC3H12A zinc-finger endonuclease.

In some embodiments, nucleic acid encoding the Cas or dCas system comprises a promoter. In some embodiments, the promoter is an EFS promoter. In some embodiments, the promoter is a neuron-specific promoter. In some embodiments, the neuron-specific promoter is a synapsin promoter.

In some embodiments, the CAG repeat disorder is HD or SCA1.

In some embodiments, the toxic CAG repeat is a CAG³⁶ or more.

In some embodiments, the toxic CAG repeat is a CAG⁸⁰ repeat.

In another embodiment of the method, the composition is administered to the tissue of the mammal by intracerebellar or intrastriatal administration.

In another embodiment, the reduced level of expression of the toxic target RNA thereby ameliorates symptoms of the disease in the mammal.

In another embodiment, the level of expression of the toxic target RNA is reduced compared to the reduction in the level of expression of untreated toxic target CAG RNA.

In another embodiment, the level of reduction is between 1-fold and 20-fold or elimination of the toxic CAG repeats is between about 20%-100%.

In another embodiment, the endonuclease is a nuclease domain of a ZC3H12A zinc-finger endonuclease.

In another embodiment, the nucleic acid sequence comprises a promoter.

In another embodiment, the promoter is a tCAG promoter.

In another embodiment, the fusion protein comprises one or more signal sequences selected from the group consisting of NLS, and NES.

In one embodiment the NLS or NES is a human NLS or human NES. In another embodiment, the human NLS is human pRB-NLS: KRSAEGSNPPKPLKKLR (SEQ ID NO: 442) or human RB-NLS (extended version): DRVLKRSAEGSNPPKPLKKLR (SEQ ID NO: 543).

In another embodiment, the nucleic acid molecule encoding the fusion protein comprises a promoter.

In another embodiment, the promoter is a tCAG promoter.

Disclosed herein is a method of treating CAG repeat disorder HD or SCA1 in a mammal comprising administering a composition to a toxic target CAG microsatellite repeat expansion (MRE) molecule in tissues of the mammal, wherein the composition comprises a nucleic acid sequence encoding a non-naturally occurring or engineered clustered regularly interspaced short palindromic repeats (CRISPR)-associated (Cas) system comprising: (a) at least one RNA-guided RNase Cas protein; and (b) at least one cognate CRISPR-Cas system guide RNA (gRNA) capable of forming a complex with one of the at least one Cas proteins, wherein the gRNA comprises (i) a DR sequence and (ii) a spacer sequence, wherein the spacer sequence hybridizes with the target CAG MRE molecule, and whereby the complex formed by the composition directly targets and destroys the target CAG MRE molecule thereby treating the disease in the mammal.

In another embodiment of the preceding method, the spacer sequence comprises a spacer sequence selected from the group consisting of: tgctgctgctgctgctgctgctgctg (guide 1, SEQ ID NO: 457), gctgctgctgctgctgctgctgctgc (guide 2, SEQ ID NO: 458), and ctgctgctgctgctgctgctgctgct (guide 3, SEQ ID NO: 459).

In another embodiment, the composition is administered to the tissue of the mammal by intrastriatal or intracerebellar administration.

In another embodiment, the RNA-guided RNase Cas protein is selected from the group consisting of Cas13a, Cas13b, Cas13c, Cas13d, and an RNA-binding portion thereof.

In another embodiment, the RNA-guided RNase Cas protein is Cas13d or an RNA-binding portion thereof.

In another embodiment, the RNA-guided RNase Cas protein which is catalytically deactivated (dCas).

In another embodiment, the dCas protein is linked to an endonuclease.

In another embodiment, the endonuclease is a nuclease domain of a ZC3H12A zinc-finger endonuclease

In another embodiment, the nucleic acid molecule comprises a promoter capable of driving expression of the RNA-guided Cas protein.

In another embodiment, the promoter is an EFS promoter.

Disclosed herein is a composition comprising a nucleic acid sequence encoding a non-naturally occurring or engineered clustered regularly interspaced short palindromic repeats (CRISPR)-associated (Cas) system comprising: (a) at least one RNA-guided RNase Cas protein; and b) at least one cognate CRISPR-Cas system guide RNA (gRNA) capable of forming a complex with one of the at least one Cas proteins, wherein the gRNA comprises (i) a DR sequence and (ii) a spacer sequence, wherein the spacer sequence hybridizes with the target CAG MRE molecule, and wherein the spacer sequence comprises a spacer sequence selected from the group consisting of tgctgctgctgctgctgctgctgctg (guide 1, SEQ ID NO: 457), gctgctgctgctgctgctgctgctgc (guide 2, SEQ ID NO: 458), and ctgctgctgctgctgctgctgctgct (guide 3, SEQ ID NO: 458).

Disclosed herein is a vector comprising any of the preceding compositions.

In another embodiment, the vector is selected from the group consisting of: adeno-associated virus (AAV), retrovirus, lentivirus, adenovirus, nanoparticle, micelle, liposome, lipoplex, polymersome, polyplex, and dendrimer.

In another embodiment, the vector is an AAV vector.

In another embodiment, the AAV vector is AA9, AAVrh10, or AAVrh.74.

Disclosed herein is a cell comprising the vector.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows results of a CAG⁸⁰ qPCR assay which demonstrate exemplary embodiments of the CAG-targeting Cas13d compositions and PUF compositions disclosed herein destroy toxic CAG repeats. Reduction of the toxic repeats in a Cas13d-based system (labeled Cas13d-L1) is shown using three different guides CAG-g1, CAG-g2, and CAG-g3. Reduction of the toxic repeats in a PUF-based system is shown using an exemplary nucleic acid molecule encoding a 8PUF(CAG)-E17 fusion protein (labeled CAG-f1 targeting frame 1: CAGCAGCA, and a CAG-f2 targeting frame 2: GCAGCAC). E17 is a domain of the ZC3H12A nuclease. Results are normalized to non-targeting controls and shown as mean+/−s.d. of biological replicates (n=2).

FIG. 2 shows the results of an RNA Fluorescence In Situ Hybridization (FISH) assay with the exemplary CAG-targeting Cas13d and PUF compositions disclosed herein as compared to non-targeting controls. CosM6 cells were co-transfected with the CAG-80 reporter gene and either non-targeting (left) or CAG-targeting Cas13d (right). Cells were fixed with 4% PFA 48 hours post transfection and RNA FISH was performed with (CAG)10 antisense DNA probe labeled with Alexa-546 (red) followed by Immunofluorescence with anti-polyQ primary antibody and anti-mouse secondary antibody labeled with Alexa-488 (green).

FIG. 3A-C shows exemplary vector configurations of the CAG-repeat gene therapy compositions disclosed herein. FIG. 3A illustrates a CAG-repeat gene therapy construct configuration comprising CAG-targeting PUF-E17 operably linked to truncated CAG promoter (tCAG). FIG. 3B illustrates a CAG-repeat gene therapy construct configuration comprising a CAG-targeting catalytically deactivated Cas13d fused to E17 and corresponding guide operably linked to EFS promoter. FIG. 3C illustrates a CAG-repeat gene therapy construct configuration comprising a CAG-targeting Cas13d and corresponding guide operably linked to EFS promoter.

FIG. 4 depicts an alignment of a CAG-targeting PUF with human PUM1 with mismatches highlighted.

FIG. 5 depicts allele preferential CAG targeting with the compositions disclosed herein. CAG expansions (CAG^{exp}) in HD prevents Exon1-2 splicing leading to overproduction of CAG^{exp} containing HTT Exon1 isoforms. In some aspects, CAG^{exp} containing HTT Exon1 isoforms are referred to as mutant HTT (mHTT).

FIG. 6A is a graph depicting percent change in body weight in mice treated with either an AAVrh10-1684 vector or AAVrh10-1589 vector at a mid-dose relative to a sham control.

FIG. 6B is a table depicting the vector composition of the AAVrh10-1684 vector and the AAVrh10-1589 vector. AAVrh10-1684 comprises an EFS/UBB promoter controlling expression of a CAG-targeted PUF protein lacking an endonuclease fusion. AAVrh10-1589 comprises an EFS/UBB promoter controlling expression of an E17 endonuclease lacking a CAG-targeting RNA binding protein.

FIG. 7 is a series of images depicting expression of AAVrh10-1383 (LBIO-210; CAG-targeting PUF) in non-human primates before (FIG. 7A) and after (FIG. 7B) delivery optimization.

FIG. 8A is a schematic detailing the reduction in mutant HTT protein levels via CAG repeat targeting fusion proteins comprising a CAG-repeat RNA binding protein and an endonuclease wherein the fusion protein binds the mutant HTT mRNA which is cleaved by the endonuclease.

FIG. 8B is a schematic detailing the reduction in mutant HTT protein levels via CAG repeat targeting proteins wherein the CAG repeat targeting protein binds the mutant HTT and blocks translation. In some aspects, the CAG repeat targeting protein comprises an endonuclease fusion. In some aspects, the CAG repeat targeting protein does not comprise an endonuclease fusion.

FIG. 9A is a table depicting vector constructs used in FIGS. 9B and 9C. Study HD08 group 1 is divided into two halves (hemispheres): hemi 1 utilized AAV9-rCas9-PIN and a non-targeting (NT) guide RNA (AAV9-1475) while the other hemi (hemi 2) utilized AAV9-rCas9-PIN with a CAG repeat-targeting guide RNA (AAV9-1347). Study HD08b was divided into group 2 AV9-RCas9-PIN+CAG guide (AAV9-1347) and group 3 AAV9-RCas9-PIN+NT guide (AAV9-1475).

FIG. 9B is a series of graphs depicting relative mutant HTT (mHTT) RNA levels* and protein (soluble mHTT) levels in mice following treatment with RCas9+NT or RCas9+CAG (Study HD08). *mHTT RNA levels Normalized to Atp5b and Eif4a2.

FIG. 9C is a series of graphs depicting relative mutant HTT (mHTT) RNA levels in mice following treatment with AAV9-rCas9-PIN+AAV-1475 (NT guide)) or AAV9-rCas9-PIN+AAV9-1347 (CAG guide) and relative Darpp32 levels and relative Pdel0a levels*. (Study HD08b). *Normalized to Atp5b and Eif4a2.

FIG. 10A is a series of fluorescent images of zQ175 P1 cortical neuron cultures immunohistochemically stained for NeuN or GFAP. Cultures are shown to contain both neurons and astrocytes.

FIG. 10B is a fluorescent image depicting expression of green fluorescent protein (GFP) following transduction with an AAVrh.10-GFP vector demonstrating that the zQ175 P1 cortical neuron cultures are readily transduced by AAVrh10.

FIG. 10C is a graph depicting mutant HTT RNA levels in zQ175 P1 cortical neuron cultures following transduction with control (UTC), Syn Clover, or A01380 (PUF(CAG)-E17) at 1E4, 1E5, or 1E6 MOI doses.

FIG. 11A is a series of images of Huntington Disease patient-derived fibroblasts.

FIG. 11B is an image of a gel depicting both wild-type and mutated HTT.

FIG. 12 is a graph depicting lack of mHTT expression in P1 neuronal cultures derived from untreated wild-type (WT) and HET (heterozygous) pups as measured by qRT-PCR. HET-specific expression of mHTT is demonstrated using raw Cts (cycle thresholds).

FIG. 13A is a graph depicting mHTT expression normalized as a percentage of UTC expression in P1 neurons derived from heterozygous zQ175 mouse pups transduced with CAG-targeting PUF and Seq212 vector constructs at 1E5 and 1E6 MOI for 7 days. Samples include untreated control (UTC), A01383_1E5 (1×10⁵ vg), A01477_1E5, A01477_1E6, A01479_1E5, A01479_1E6, A01553_1E5, A01553_1E6, and AA09sh.

FIG. 13B is a graph depicting wt HTT expression normalized as a percentage of UTC expression in P1 neurons derived from heterozygous zQ175 mouse pups transduced with CAG-targeting PUF and Seq212 vector constructs at 1E5 and 1E6 MOI for 7 days. Samples include untreated control (UTC), A01383_1E5 (1×10⁵ vg), A01477_1E5, A01477_1E6, A01479_1E5, A01479_1E6, A01553_1E5, A01553_1E6, and AA09sh.

FIG. 14A is a graph depicting mHTT expression measured by Meso Scale Discovery Immunoassay (MSD) in P1 neurons derived from heterozygous zQ175 mouse pups transduced with CAG-targeting PUF and CAG-targeting cas13d vectors at 1E5 or 1E6 MOI for 7 days. Samples include untreated control (UTC), A01383, A01479, A01922, and wt. Data is presented for two mice pups.

FIG. 14B is a graph depicting mHTT expression normalized as a percentage of UTC expression in P1 neurons derived from heterozygous zQ175 mouse pups transduced with CAG-targeting PUF and CAG-targeting casl3d vectors at 1E5 or 1E6 MOI for 7 days. Samples include untreated control (UTC), A01383, A01479, A01922, and wt. Data is presented for two mice pups.

FIG. 15A is a graph depicting casl3d Seq212 expression in P1 neurons derived from heterozygous zQ175 mouse pups transduced with CAG-targeting cas13d Seq212 constructs at 1E5 and 1E6 MOI for 7 days. Cas13d expression is normalized to ATP5b. Vectors assessed include A01477, A01479, and A01553.

FIG. 15B is a graph depicting casl3d guide RNA expression in P1 neurons derived from heterozygous zQ175 mouse pups transduced with CAG-targeting cas13d Seq212 constructs at 1E5 and 1E6 MOI for 7 days. Vectors assessed include A01477, A01479, and A01553.

FIG. 16A is a series of graphs depicting expression of neuronal and microglial activation biomarkers AIF1, PDE10A, PPPIR1B, and RBFOX3 in P1 neurons transduced with CAG-targeting PUF A01383 at 1E5 MOI for 7 days relative to UTC cells.

FIG. 16B is a series of graphs depicting expression of neuronal and microglial activation biomarkers PDE10A, PPPIR1B, and RBFOX3 in P1 neurons transduced with CAG-targeting PUF A01383 at 1E5 MOI for 7 days relative to UTC cells.

FIG. 17 is graph depicting fold change differences in cytotoxicity relative to UTC in P1 neurons transduced with CAG-targeting constructs at 1E5 MOI for 7 days. Samples include, wt, heterozygous (het), A01383 vector, A01684 vector, A01479 vector, or A01922 vector.

FIG. 18A is a schematic depicting a CAG-targeting PUF protein suitable for binding CAG-repeat RNA and blocking the RNA resulting in destruction of bound RNA and/or inhibition of translation of the bound RNA.

FIG. 18B is a schematic depicting a CAG-targeting dCas13d protein suitable for binding CAG-repeat RNA and blocking the RNA resulting in destruction of bound RNA and/or inhibition of translation of the bound RNA.

FIG. 19 is a table listing exemplary AAV vector comprising CAG-targeting compositions of the disclosure.

### DETAILED DESCRIPTION

The disclosure provides RNA-targeting gene therapy compositions and methods for treating CAG trinucleotide repeat- or CAG MRE-causing diseases and/or disorders such as HD and SCA1.

HD and SCA1 are fatal, progressive autosomal dominant diseases caused by expanded CAG repeats in HTT and ATXN1 genes, respectively. These repeats code for polyglutamine tracts, the size of which correlates with onset and progression of the diseases.

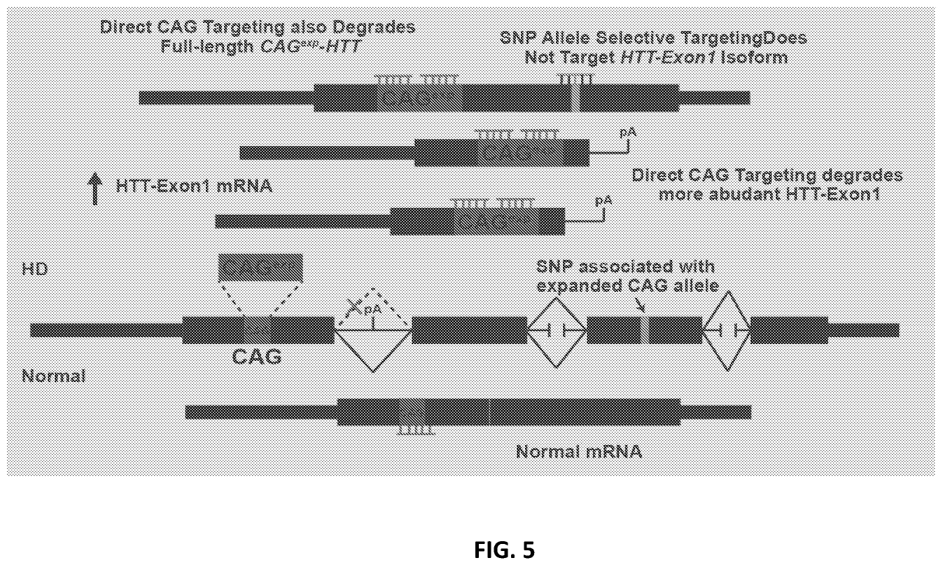

The human Huntingtin (HTT) gene has 67 exons. CAG repeat expansions in Exon1 lead to polyQ protein aggregation and HD. HD disease onset is inversely correlated with the number of CAG repeats. All single nucleotide polymorphisms (SNPs) are linked with the expanded CAG allele downstream of Exon 1. Targeting HTT in an allele specific manner utilizing SNPs linked with expansion will target the highly pathogenic short CAG containing HTTexon1 isoform. Targeting Exon 1 outside the CAG repeats will not lead to allele specific knockdown. The gene therapy compositions and methods disclosed here for treating HD target CAG repeats in an allele preferential manner and allows for expression of normal HTT protein (FIG. 5).

In HD, the CAG segment is repeated 36 to 120 times within the mutant HTT gene compared to what is considered the normal CAG repeat of 10 to 35 times within the HTT gene. An increase in the size of the CAG segment leads to the production of an abnormally long version of the huntingtin protein, which is cut into smaller, toxic fragments that bind together and accumulate in neurons, disrupting the normal functions of these cells. This disfunction and eventual death of neurons in certain areas of the brain underlie the signs and symptoms of HD.

In SCA1, the CAG segment is repeated 40 to more than 80 times within the mutant ATXN1 gene compared to what is considered the normal CAG repeat of 4 to 39 times in the ATXN1 gene. This increase in the CAG segment leads to the production of an abnormally long version of the ataxin-1 protein which folds into the wrong 3-dimensional shape. This abnormality in protein folding causes the protein to cluster with other proteins to form clumps (aggregates) within the nucleus of the cells and leads to cell damage and ultimate cell death. Targeting and eliminating (or blocking) CAG repeats is a therapeutic strategy for HD and SCA1.

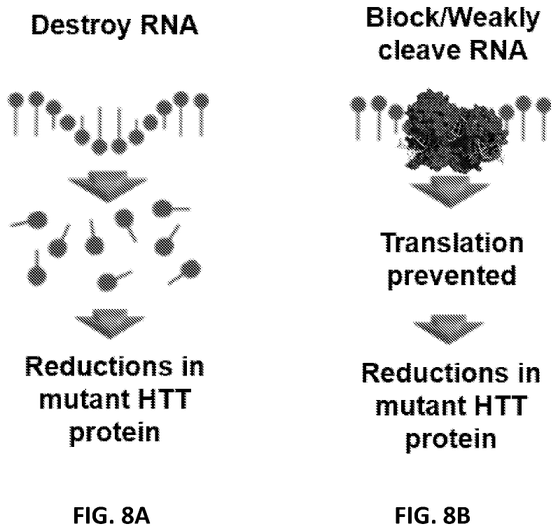

The gene therapy compositions disclosed herein provide improved cleavage of toxic CAG repeats in methods of treating CAG-repeat diseases and/or disorders (FIG. 8A). In other embodiments of the disclosure, gene therapy compositions disclosed herein block the expression of toxic CAG-repeat containing mRNA transcripts (FIG. 8B). These gene therapy compositions are capable of specifically targeting toxic CAG repeat RNA and providing long-term repair of the disease phenotypes associated with diseases such as HD and SCA1. These gene therapy compositions also provide efficient cleavage or blocking of toxic CAG repeat RNA. Such gene therapy compositions for targeting CAG MREs are important for scaling of therapeutic systems in manufacturing because the components of the compositions are a small enough size to rely on a unitary (single) vector. The gene therapy compositions disclosed herein are capable of achieving more effective knockdown or blocking of the toxic CAG repeats compared to non-treatment.

Disclosed herein are compositions comprising nucleic acid molecules, and vectors comprising the same, encoding guided or non-guided RNA-binding systems capable of binding toxic CAG repeat RNA for treating CAG-repeat diseases such as HD and SCA1. Such compositions are capable of targeting and binding for either knockdown/destruction or blocking the toxic CAG repeats. In some aspects, compositions suitable for blocking CAG-repeat RNA bind a CAG-repeat containing RNA and prevent translation of the CAG-repeat RNA. In some aspects, this prevented translation results in reduced protein expression from CAG-repeat containing RNA sequences. These systems comprise either RNA-guided RNase Cas, such as Cas13d, or non-guided PUF, PUMBY or PPR protein configurations.

In any of the preceding or subsequent RNA-targeting compositions for treating HD or SCA1, any particular construct element (e.g., linker, promoter, signal sequence, etc.,) described in the context of a specific RNA-targeting composition, can be substituted for another of the same element type (e.g., linker, promoter, signal sequence, etc.). In some embodiments, any particular construct element can be omitted or removed (such as a tag sequence). In other words, the exemplary combinations of elements in any particular gene therapy composition described herein is not intended to be limiting.

### Exemplary Blocking RNA-targeting Compositions

Expanded CAG (CAG^{exp}) repeats in HTT or ATXN1 mRNA lead to protein aggregation of HTT or ataxin-1 causing loss of their function. PUF(CAG) or dCas13d(CAG) will bind CAGexP RNA directly and block the CAG^{exp} RNA leading to sequestration of blocked/inhibited translation ultimately resulting in reduced levels of mutated protein such as mHTTT or mATXN1.

Exemplary blocking CAG-targeting PUF protein compositions include:

PUFs targeting CAG frame 2 (blocking) w/ myc tag

| | Protein | | Target | |
|---|---|---|---|---|
| Construct | Type | Elements | Sequence | Amino Acid Sequence of PUF |
| A01684 | 8PUF | N-terminal PUF; | GCAGCAGC | GRSRLLEDFRNNRYPNLQLREIAG |
| | | linker between | (SEQ ID NO: | HIMEFSQDQHGSRFIRLKLERATP |
| | | PUF and myc | 476) | AERQLVFNEILQAAYQLMVDVFG |
| | | tag (GGS); | | SYVIEKFFEFGSLEQKLALAERIRG |
| | | C-terminal myc | | HVLSLALQMYGCRVIQKALEFIPS |
| | | tag | | DQQNEMVRELDGHVLKCVKDQN |
| | | | | GSYVVRKCIECVQPQSLQFIIDAFK |
| | | | | GQVFALSTHPYGSRVIERILEHCLP |
| | | | | DQTLPILEELHQHTEQLVQDQYGC |
| | | | | YVIQHVLEHGRPEDKSKIVAEIRG |
| | | | | NVLVLSQHKFASYVVRKCVTHAS |
| | | | | RTERAVLIDEVCTMNDGPHSALY |
| | | | | TMMKDQYASYVVEKMIDVAEPG |
| | | | | QRKIVMHKIRPHIATLRKYTYGKH |
| | | | | ILAKLEKYYMKNGVDLG (SEQ ID |
| | | | | NO: 549) |
| A01683 | 8PUF | PUF | GCAGCAGC | GRSRLLEDFRNNRYPNLQLREIAG |
| | | | (SEQ ID NO: | HIMEFSQDQHGSRFIRLKLERATP |
| | | | 476) | AERQLVFNEILQAAYQLMVDVFG |
| | | | | SYVIEKFFEFGSLEQKLALAERIRG |
| | | | | HVLSLALQMYGCRVIQKALEFIPS |
| | | | | DQQNEMVRELDGHVLKCVKDQN |
| | | | | GSYVVRKCIECVQPQSLQFIIDAFK |
| | | | | GQVFALSTHPYGSRVIERILEHCLP |
| | | | | DQTLPILEELHQHTEQLVQDQYGC |
| | | | | YVIQHVLEHGRPEDKSKIVAEIRG |
| | | | | NVLVLSQHKFASYVVRKCVTHAS |
| | | | | RTERAVLIDEVCTMNDGPHSALY |
| | | | | TMMKDQYASYVVEKMIDVAEPG |
| | | | | QRKIVMHKIRPHIATLRKYTYGKH |
| | | | | ILAKLEKYYMKNGVDLG (SEQ ID |
| | | | | NO: 549) |

### RNA-Guided CAG-Repeat RNA Binding Systems

In some embodiments, the RNA-guided RNA-binding system is an RNase Cas-based RNA-guided RNA-binding polypeptide. In some embodiments, a nucleic acid sequence encodes an RNA-guided RNA-binding polypeptide which is an RNase Cas protein (or a deactivated RNase Cas protein). In one embodiment, the nucleic acid sequence further comprises a gRNA sequence comprising a spacer sequence which binds to a toxic target CAG repeat RNA and a direct repeat (DR) sequence which binds to the RNase Cas protein.

In one embodiment, a Cas13d(CAG) system is catalytically active, in which case, the Cas13d nucleoprotein complex cleaves and destroys toxic RNA CAG repeats. In another embodiment, a Cas13d(CAG) system is catalytically inactive, in which case, the Cas13d nucleoprotein complex binds and blocks (but does not cleave) the RNA CAG repeats. In yet another embodiment, a Cas13d(CAG) comprises a catalytically inactive Cas13d(CAG) fused to an endonuclease which is capable of cleaving the toxic RNA CAG repeats. In such an embodiment, the endonuclease is an active RNase. Exemplary endonucleases with RNase activity can be found herein, and these include, for example, a domain from a ZC3H12A zinc-finger (also referred herein as E17) or a PIN endonuclease.

TABLE 1
Exemplary spacer sequences used in sgRNAs for CAG
targeting with RNase Cas systems for treating CAG-
repeat disease:

| Spacer | Spacer Sequences |
|---|---|
| 1 | tgctgctgctgctgctgctgctgctg (SEQ ID NO: 457) |
| 2 | gctgctgctgctgctgctgctgctgc (SEQ ID NO: 458) |
| 3 | ctgctgctgctgctgctgctgctgct (SEQ ID NO: 459) |

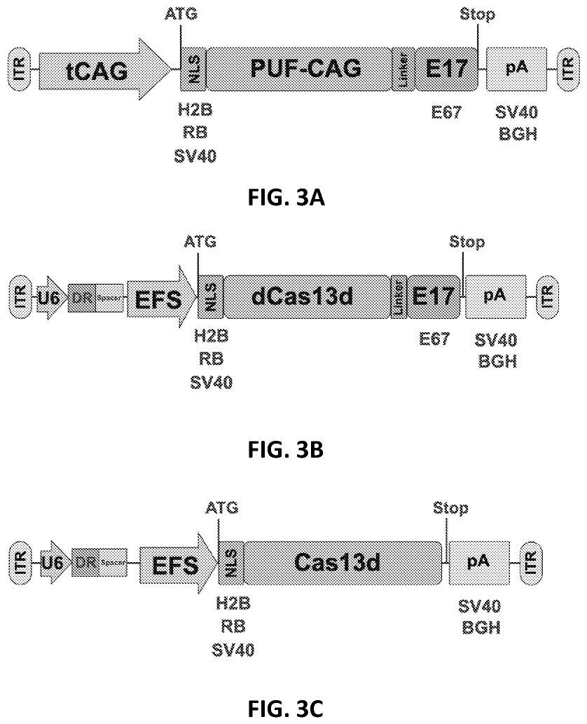

In one embodiment, the RNase Cas protein is a Cas13 protein. In another embodiment, the Cas13 protein is a Cas13d protein. In another embodiment, the Cas13d protein is a deactivated RNase Cas13d protein (dCas13d). In another embodiment, the dCas13d protein is a fusion protein comprising 1) dCas13d and 2) a polypeptide encoding a protein or fragment thereof having nuclease activity. In another embodiment, the dCas13d protein is a fusion protein comprising 1) dCas13d and 2) a nuclease domain of ZC3H12A, a zinc-finger endonuclease, (referred to as E17 herein). In some embodiments, the Cas configuration comprises a signal sequence(s) such as NLS(s) and/or NES(s). In some embodiments, the dCas13d is linked to E17 via a linker sequence. In one embodiment, the linker sequence is VDTANGS (SEQ ID NO: 411). In some embodiments, the nucleic acid sequence encoding the Cas13d or dCas13d fusion proteins are operably linked to at least one promoter sequence. In some embodiments, the promoter sequence comprises an enhancer and/or an intron. In some embodiments, the promoter sequence is an EFS promoter sequence, tCAG promoter sequence, EFS/UBB promoter sequence, EFS promoter sequence, or synapsin sequence (FIG. 3B, FIG. 3C, FIG. 20A, and FIG. 20B).

In some embodiments, the nucleic acid sequence comprises a first promoter sequence that controls expression of a Cas13d protein or Cas13d fusion protein and a second promoter sequences that controls expression of the at least one guide RNA sequence. In some embodiments, the Cas13d or dCas13d system targets expanded CAG repeats, wherein the CAG repeats are CAG³⁶ or more. In some embodiments, the CAG repeats are CAG⁸⁰. In some aspects, CAG³⁶ or CAG⁸⁰ refers to 36 CAG repeats or 80 CAG repeats in the HTT or ATXN1 gene. Any other number of CAG repeats are possible, including at least 1, 2, 3, 4, 5, 6, 7, 8, 9, 10, 15, 20, 25, 30, 35, 36, 37, 38, 39, 40, 41, 42, 43, 44, 45, 46, 47, 48, 49, 50, 55, 60, 65, 70, 75, 76, 77, 78, 79, 80, 81, 82, 83, 84, 85, 90, 95, 100, 105, 110, 115, 120 CAG repeats, or any other number of CAG repeats in between.

In some embodiments, a CAG-repeat targeting dCas13d protein of the disclosure comprises from N-terminal to C-terminal: dCas13d (dSeq212), a linker, an SV-40 NLS, a linker, and an HA tag. In some embodiments, a dCas13d protein of the disclosure comprises from N-terminal to C-terminal: dCas13d (dSeq212), a linker, an SV-40 NLS, and a linker. In some aspects, the CAG-repeat targeting dCas13d protein of the disclosure is set forth in Table A. In some aspects, the CAG-repeat targeting dCas13d protein is used for methods of blocking CAG-repeat RNA sequence expression.

TABLE A
CAG-repeat targeting dCas 13d protein

| | |
|---|---|
| Dead Seq212 | KKKHQSAAEKRQVKKLKNQEKAQKYASEPSPLQSDTAGVECSQKKTVVS |
| | HIASSKTLAKAMGLKSTLVMGDKLVITSFAASKAVGGAGYKSANIEKITDL |
| | QGRVIEEHERMFSADVGEKNIELSKNDCHTNVNNPVVTNIGKDYIGLKSRL |
| | EQEFFGKTFENDNLHVQLAYNILDIKKILGTYVNNIIYIFYNLNRAGTGRDE |
| | RMYDDLIGTLYAYKPMEAQQTYLLKGDKDMRRFEEVKQLLQNTSAYYVY |
| | YGTLFEKVKAKSKKEQRAKEAEIDACTAHNYDVLRLLSLMRQLCMHSVA |
| | GTAFKLAESALFNIEDVLSADLKEILDEAFSGAVNKLNDGFVQHSGNNLYV |
| | LQQLYPNETIERIAEKYYRLTVRKEDLNMGVNIKKLRELIVGQYFPEVLDK |
| | EYDLSKNGDSVVTYRSKIYTVMNYILLYYLEDHDSSRESMVEALRQNREG |
| | DEGKEEIYRQFAKKVWNGVSGLFGVCLNLFKTEKRNKFRSKVALPDVSGA |
| | AYMLSSENIDYFVKMLFFVCKFLDGKEINELLCALINKFDNIADILDAAAQC |
| | GSSVWFVDSYRFFERSRRISAQIRIVKNIASKDFKKSKKDSDESYPEQLYLD |
| | ALALLGDVISKYKQNRDGSVVIDDQGNAVLTEQYKRFRYEFFEEIKRDESG |
| | GIKYKKSGKPEYNHQRRNFILNNVLKSKWFFYVVKYNRPSSCRELMKNKE |
| | ILRFVLRDIPDSQVRRYFKAVQGEEAYASAEAMRTRLVDALSQFSVTACLD |
| | EVGGMTDKEFASQRAVDSKEKLRAIIRLYLTVAYLITKSMVKVNTRFSIAF |
| | SVLERDYYLLIDGKKKSSDYTGEDMLALTRKFVGEDAGLYREWKEKNAE |
| | AKDKYFDKAERKKVLRQNDKMIRKMHFTPHSLNYVQKNLESVQSNGLAA |
| | VIKEYRNAVAALNIINRLDEYIGSARADSYYSLYCYCLQMYLSKNFSVGYL |
| | INVQKQLEEHHTYMKDLMWLLNIPFAYNLARYKNLSNEKLFYDEEAAAE |
| | KADKAENERGE (SEQ ID NO: 587) |
| Linker | GS |
| SV-40 NLS | PKKKRKV (SEQ ID NO: 437) |
| Linker | ED |
| HA Tag | YPYDVPDYA (SEQ ID NO: 586) |

In some embodiments, a CAG-repeat targeting casl3d or dCas13d protein of the disclosure comprises from N-terminal to C-terminal: dCas13d (dSeq212), a linker, an SV-40 NLS, a linker, and an HA tag. In some embodiments, a dCas13d protein of the disclosure comprises from N-terminal to C-terminal: dCas13d (dSeq212), a linker, and an SV-40 NLS. In some aspects, the CAG-repeat targeting dCas13d protein of the disclosure is set forth in Table B. In some aspects, the CAG-repeat targeting dCas13d protein is used for methods of blocking CAG-repeat RNA sequence expression.

TABLE B
CAG-repeat targeting dCas13d protein

| Plasmid Element | Amino Acid Sequences |
|---|---|
| Dead Seq212 | KKKHQSAAEKRQVKKLKNQEKAQKYASEPSPLQSDTAGVECSQKKTVVS |
| | HIASSKTLAKAMGLKSTLVMGDKLVITSFAASKAVGGAGYKSANIEKITDL |
| | QGRVIEEHERMFSADVGEKNIELSKNDCHTNVNNPVVTNIGKDYIGLKSRL |
| | EQEFFGKTFENDNLHVQLAYNILDIKKILGTYVNNIIYIFYNLNRAGTGRDE |
| | RMYDDLIGTLYAYKPMEAQQTYLLKGDKDMRRFEEVKQLLQNTSAYYVY |
| | YGTLFEKVKAKSKKEQRAKEAEIDACTAHNYDVLRLLSLMAQLCMASVA |
| | GTAFKLAESALFNIEDVLSADLKEILDEAFSGAVNKLNDGFVQHSGNNLYV |
| | LQQLYPNETIERIAEKYYRLTVRKEDLNMGVNIKKLRELIVGQYFPEVLDK |
| | EYDLSKNGDSVVTYRSKIYTVMNYILLYYLEDHDSSRESMVEALRQNREG |
| | DEGKEEIYRQFAKKVWNGVSGLFGVCLNLFKTEKRNKFRSKVALPDVSGA |
| | AYMLSSENIDYFVKMLFFVCKFLDGKEINELLCALINKFDNIADILDAAAQC |
| | GSSVWFVDSYRFFERSRRISAQIRIVKNIASKDFKKSKKDSDESYPEQLYLD |
| | ALALLGDVISKYKQNRDGSVVIDDQGNAVLTEQYKRFRYEFFEEIKRDESG |
| | GIKYKKSGKPEYNHQRRNFILNNVLKSKWFFYVVKYNRPSSCRELMKNKE |
| | ILRFVLRDIPDSQVRRYFKAVQGEEAYASAEAMRTRLVDALSQFSVTACLD |
| | EVGGMTDKEFASQRAVDSKEKLRAIIRLYLTVAYLITKSMVKVNTRFSIAF |
| | SVLERDYYLLIDGKKKSSDYTGEDMLALTRKFVGEDAGLYREWKEKNAE |
| | AKDKYFDKAERKKVLRQNDKMIRKMHFTPHSLNYVQKNLESVQSNGLAA |
| | VIKEYANAVAALNIINRLDEYIGSARADSYYSLYCYCLQMYLSKNFSVGYL |
| | INVQKQLEEHHTYMKDLMWLLNIPFAYNLARYKNLSNEKLFYDEEAAAE |
| | KADKAENERGE (SEQ ID NO: 590) |
| Linker | GS |
| SV-40 NLS | PKKKRKV (SEQ ID NO: 437) |
| Linker | ED |
| HA Tag | YPYDVPDYA (SEQ ID NO: 586) |

In some embodiments, a CAG-repeat targeting dCas13d protein of the disclosure comprises from N-terminal to C-terminal: dCas13d (dSeq212), a linker, an SV-40 NLS, a linker, and an HA tag. In some embodiments, a dCas13d protein of the disclosure comprises from N-terminal to C-terminal: dCas13d (dSeq212), a linker, an SV-40 NLS, and a linker. In some aspects, the CAG-repeat targeting dCas13d protein of the disclosure is set forth in Table C. In some aspects, the CAG-repeat targeting dCas13d protein is used for methods of blocking CAG-repeat RNA sequence expression.

TABLE C
CAG-repeat targeting dCas13d protein

| Plasmid Element | Amino Acid Sequences |
|---|---|
| Dead Seq212 | KKKHQSAAEKRQVKKLKNQEKAQKYASEPSPLQSDTAGVECSQKKTVVS |
| | HIASSKTLAKAMGLKSTLVMGDKLVITSFAASKAVGGAGYKSANIEKITDL |
| | QGRVIEEHERMFSADVGEKNIELSKNDCHTNVNNPVVTNIGKDYIGLKSRL |
| | EQEFFGKTFENDNLHVQLAYNILDIKKILGTYVNNIIYIFYNLNRAGTGRDE |
| | RMYDDLIGTLYAYKPMEAQQTYLLKGDKDMRRFEEVKQLLQNTSAYYVY |
| | YGTLFEKVKAKSKKEQRAKEAEIDACTAHNYDVLRLLSLMRQLCMHSVA |
| | GTAFKLAESALFNIEDVLSADLKEILDEAFSGAVNKLNDGFVQHSGNNLYV |
| | LQQLYPNETIERIAEKYYRLTVRKEDLNMGVNIKKLRELIVGQYFPEVLDK |
| | EYDLSKNGDSVVTYRSKIYTVMNYILLYYLEDHDSSRESMVEALRQNREG |
| | DEGKEEIYRQFAKKVWNGVSGLFGVCLNLFKTEKRNKFRSKVALPDVSGA |
| | AYMLSSENIDYFVKMLFFVCKFLDGKEINELLCALINKFDNIADILDAAAQC |
| | GSSVWFVDSYRFFERSRRISAQIRIVKNIASKDFKKSKKDSDESYPEQLYLD |
| | ALALLGDVISKYKQNRDGSVVIDDQGNAVLTEQYKRFRYEFFEEIKRDESG |
| | GIKYKKSGKPEYNHQRRNFILNNVLKSKWFFYVVKYNRPSSCRELMKNKE |
| | ILRFVLRDIPDSQVRRYFKAVQGEEAYASAEAMRTRLVDALSQFSVTACLD |
| | EVGGMTDKEFASQRAVDSKEKLRAIIRLYLTVAYLITKSMVKVNTRFSIAF |
| | SVLERDYYLLIDGKKKSSDYTGEDMLALTRKFVGEDAGLYREWKEKNAE |
| | AKDKYFDKAERKKVLRQNDKMIRKMHFTPHSLNYVQKNLESVQSNGLAA |
| | VIKEYANAVAHLNIINRLDEYIGSARADSYYSLYCYCLQMYLSKNFSVGYL |
| | INVQKQLEEHHTYMKDLMWLLNIPFAYNLARYKNLSNEKLFYDEEAAAE |
| | KADKAENERGE (SEQ ID NO: 590) |
| Linker | GS |
| SV-40 NLS | PKKKRKV (SEQ ID NO: 437) |
| Linker | ED |
| HA Tag | YPYDVPDYA (SEQ ID NO: 586). |

CAG-repeat targeting dCas 13d protein

| Plasmid Element | Amino Acid Sequences |
|---|---|
| Dead Seq212 | KKKHQSAAEKRQVKKLKNQEKAQKYASEPSPLQSDTAGVECSQKKTVVS |
| | HIASSKTLAKAMGLKSTLVMGDKLVITSFAASKAVGGAGYKSANIEKITDL |
| | QGRVIEEHERMFSADVGEKNIELSKNDCHTNVNNPVVTNIGKDYIGLKSRL |
| | EQEFFGKTFENDNLHVQLAYNILDIKKILGTYVNNIIYIFYNLNRAGTGRDE |
| | RMYDDLIGTLYAYKPMEAQQTYLLKGDKDMRRFEEVKQLLQNTSAYYVY |
| | YGTLFEKVKAKSKKEQRAKEAEIDACTAHNYDVLRLLSLMRQLCMHSVA |
| | GTAFKLAESALFNIEDVLSADLKEILDEAFSGAVNKLNDGFVQHSGNNLYV |
| | LQQLYPNETIERIAEKYYRLTVRKEDLNMGVNIKKLRELIVGQYFPEVLDK |
| | EYDLSKNGDSVVTYRSKIYTVMNYILLYYLEDHDSSRESMVEALRQNREG |
| | DEGKEEIYRQFAKKVWNGVSGLFGVCLNLFKTEKRNKFRSKVALPDVSGA |
| | AYMLSSENIDYFVKMLFFVCKFLDGKEINELLCALINKFDNIADILDAAAQC |
| | GSSVWFVDSYRFFERSRRISAQIRIVKNIASKDFKKSKKDSDESYPEQLYLD |
| | ALALLGDVISKYKQNRDGSVVIDDQGNAVLTEQYKRFRYEFFEEIKRDESG |
| | GIKYKKSGKPEYNHQRRNFILNNVLKSKWFFYVVKYNRPSSCRELMKNKE |
| | ILRFVLRDIPDSQVRRYFKAVQGEEAYASAEAMRTRLVDALSQFSVTACLD |
| | EVGGMTDKEFASQRAVDSKEKLRAIIRLYLTVAYLITKSMVKVNTRFSIAF |
| | SVLERDYYLLIDGKKKSSDYTGEDMLALTRKFVGEDAGLYREWKEKNAE |
| | AKDKYFDKAERKKVLRQNDKMIRKMHFTPHSLNYVQKNLESVQSNGLAA |
| | VIKEYANAVAHLNIINRLDEYIGSARADSYYSLYCYCLQMYLSKNFSVGYL |
| | INVQKQLEEHHTYMKDLMWLLNIPFAYNLARYKNLSNEKLFYDEEAAAE |
| | KADKAENERGE (SEQ ID NO: 591) |
| Linker | GS |
| SV-40 NLS | PKKKRKV (SEQ ID NO: 437) |
| Linker | ED |
| HA Tag | YPYDVPDYA (SEQ ID NO: 586) |

CAG-repeat targeting dCas13d protein

| Plasmid Element | Amino Acid Sequences |
|---|---|
| Dead Seq212 | KKKHQSAAEKRQVKKLKNQEKAQKYASEPSPLQSDTAGVECSQKKTVVS |
| | HIASSKTLAKAMGLKSTLVMGDKLVITSFAASKAVGGAGYKSANIEKITDL |
| | QGRVIEEHERMFSADVGEKNIELSKNDCHTNVNNPVVTNIGKDYIGLKSRL |
| | EQEFFGKTFENDNLHVQLAYNILDIKKILGTYVNNIIYIFYNLNRAGTGRDE |
| | RMYDDLIGTLYAYKPMEAQQTYLLKGDKDMRRFEEVKQLLQNTSAYYVY |
| | YGTLFEKVKAKSKKEQRAKEAEIDACTAHNYDVLRLLSLMRQLCMASVA |
| | GTAFKLAESALFNIEDVLSADLKEILDEAFSGAVNKLNDGFVQHSGNNLYV |
| | LQQLYPNETIERIAEKYYRLTVRKEDLNMGVNIKKLRELIVGQYFPEVLDK |
| | EYDLSKNGDSVVTYRSKIYTVMNYILLYYLEDHDSSRESMVEALRQNREG |
| | DEGKEEIYRQFAKKVWNGVSGLFGVCLNLFKTEKRNKFRSKVALPDVSGA |
| | AYMLSSENIDYFVKMLFFVCKFLDGKEINELLCALINKFDNIADILDAAAQC |
| | GSSVWFVDSYRFFERSRRISAQIRIVKNIASKDFKKSKKDSDESYPEQLYLD |
| | ALALLGDVISKYKQNRDGSVVIDDQGNAVLTEQYKRFRYEFFEEIKRDESG |
| | GIKYKKSGKPEYNHQRRNFILNNVLKSKWFFYVVKYNRPSSCRELMKNKE |
| | ILRFVLRDIPDSQVRRYFKAVQGEEAYASAEAMRTRLVDALSQFSVTACLD |
| | EVGGMTDKEFASQRAVDSKEKLRAIIRLYLTVAYLITKSMVKVNTRFSIAF |
| | SVLERDYYLLIDGKKKSSDYTGEDMLALTRKFVGEDAGLYREWKEKNAE |
| | AKDKYFDKAERKKVLRQNDKMIRKMHFTPHSLNYVQKNLESVQSNGLAA |
| | VIKEYRNAVAHLNIINRLDEYIGSARADSYYSLYCYCLQMYLSKNFSVGYL |
| | INVQKQLEEHHTYMKDLMWLLNIPFAYNLARYKNLSNEKLFYDEEAAAE |
| | KADKAENERGE (SEQ ID NO: 592) |
| Linker | GS |
| SV-40 NLS | PKKKRKV (SEQ ID NO: 437) |
| Linker | ED |
| HA Tag | YPYDVPDYA (SEQ ID NO: 586) |

CAG-repeat targeting dCas13d protein

| Plasmid Element | Amino Acid Sequences |
|---|---|
| Dead Seq212 | KKKHQSAAEKRQVKKLKNQEKAQKYASEPSPLQSDTAGVECSQKKTVVS |
| | HIASSKTLAKAMGLKSTLVMGDKLVITSFAASKAVGGAGYKSANIEKITDL |
| | QGRVIEEHERMFSADVGEKNIELSKNDCHTNVNNPVVTNIGKDYIGLKSRL |
| | EQEFFGKTFENDNLHVQLAYNILDIKKILGTYVNNIIYIFYNLNRAGTGRDE |
| | RMYDDLIGTLYAYKPMEAQQTYLLKGDKDMRRFEEVKQLLQNTSAYYVY |
| | YGTLFEKVKAKSKKEQRAKEAEIDACTAHNYDVLRLLSLMAQLCMHSVA |
| | GTAFKLAESALFNIEDVLSADLKEILDEAFSGAVNKLNDGFVQHSGNNLYV |
| | LQQLYPNETIERIAEKYYRLTVRKEDLNMGVNIKKLRELIVGQYFPEVLDK |
| | EYDLSKNGDSVVTYRSKIYTVMNYILLYYLEDHDSSRESMVEALRQNREG |
| | DEGKEEIYRQFAKKVWNGVSGLFGVCLNLFKTEKRNKFRSKVALPDVSGA |
| | AYMLSSENIDYFVKMLFFVCKFLDGKEINELLCALINKFDNIADILDAAAQC |
| | GSSVWFVDSYRFFERSRRISAQIRIVKNIASKDFKKSKKDSDESYPEQLYLD |
| | ALALLGDVISKYKQNRDGSVVIDDQGNAVLTEQYKRFRYEFFEEIKRDESG |
| | GIKYKKSGKPEYNHQRRNFILNNVLKSKWFFYVVKYNRPSSCRELMKNKE |
| | ILRFVLRDIPDSQVRRYFKAVQGEEAYASAEAMRTRLVDALSQFSVTACLD |
| | EVGGMTDKEFASQRAVDSKEKLRAIIRLYLTVAYLITKSMVKVNTRFSIAF |
| | SVLERDYYLLIDGKKKSSDYTGEDMLALTRKFVGEDAGLYREWKEKNAE |
| | AKDKYFDKAERKKVLRQNDKMIRKMHFTPHSLNYVQKNLESVQSNGLAA |
| | VIKEYRNAVAHLNIINRLDEYIGSARADSYYSLYCYCLQMYLSKNFSVGYL |
| | INVQKQLEEHHTYMKDLMWLLNIPFAYNLARYKNLSNEKLFYDEEAAAE |
| | KADKAENERGE (SEQ ID NO: 593) |
| Linker | GS |
| SV-40 NLS | PKKKRKV (SEQ ID NO: 437) |
| Linker | ED |
| HA Tag | YPYDVPDYA (SEQ ID NO: 586) |

CAG-repeat targeting dCas13d protein

| Plasmid Element | Amino Acid Sequences |
|---|---|
| Dead Seq212 | KKKHQSAAEKRQVKKLKNQEKAQKYASEPSPLQSDTAGVECSQKKTVVS |
| | HIASSKTLAKAMGLKSTLVMGDKLVITSFAASKAVGGAGYKSANIEKITDL |
| | QGRVIEEHERMFSADVGEKNIELSKNDCHTNVNNPVVTNIGKDYIGLKSRL |
| | EQEFFGKTFENDNLHVQLAYNILDIKKILGTYVNNIIYIFYNLNRAGTGRDE |
| | RMYDDLIGTLYAYKPMEAQQTYLLKGDKDMRRFEEVKQLLQNTSAYYVY |
| | YGTLFEKVKAKSKKEQRAKEAEIDACTAHNYDVLRLLSLMRQLCMHSVA |
| | GTAFKLAESALFNIEDVLSADLKEILDEAFSGAVNKLNDGFVQHSGNNLYV |
| | LQQLYPNETIERIAEKYYRLTVRKEDLNMGVNIKKLRELIVGQYFPEVLDK |
| | EYDLSKNGDSVVTYRSKIYTVMNYILLYYLEDHDSSRESMVEALRQNREG |
| | DEGKEEIYRQFAKKVWNGVSGLFGVCLNLFKTEKRNKFRSKVALPDVSGA |
| | AYMLSSENIDYFVKMLFFVCKFLDGKEINELLCALINKFDNIADILDAAAQC |
| | GSSVWFVDSYRFFERSRRISAQIRIVKNIASKDFKKSKKDSDESYPEQLYLD |
| | ALALLGDVISKYKQNRDGSVVIDDQGNAVLTEQYKRFRYEFFEEIKRDESG |
| | GIKYKKSGKPEYNHQRRNFILNNVLKSKWFFYVVKYNRPSSCRELMKNKE |
| | ILRFVLRDIPDSQVRRYFKAVQGEEAYASAEAMRTRLVDALSQFSVTACLD |
| | EVGGMTDKEFASQRAVDSKEKLRAIIRLYLTVAYLITKSMVKVNTRFSIAF |
| | SVLERDYYLLIDGKKKSSDYTGEDMLALTRKFVGEDAGLYREWKEKNAE |
| | AKDKYFDKAERKKVLRQNDKMIRKMHFTPHSLNYVQKNLESVQSNGLAA |
| | VIKEYRNAVAHLNIINRLDEYIGSARADSYYSLYCYCLQMYLSKNFSVGYL |
| | INVQKQLEEHHTYMKDLMWLLNIPFAYNLARYANLSNEKLFYDEEAAAE |
| | KADKAENERGE (SEQ ID NO: 594) |
| Linker | GS |
| SV-40 NLS | PKKKRKV (SEQ ID NO: 437) |
| Linker | ED |
| HA Tag | YPYDVPDYA (SEQ ID NO: 586) |

In some embodiments, a CAG-repeat targeting dCas13d fusion protein of the disclosure comprises from N-terminal to C-terminal: an SV-40 NLS sequence, dCas13d (dSeq212) sequence, a linker sequence, an SV-40 NLS, a ZC3HT2A endonuclease (E17), a linker sequence, and a myc tag. In some embodiments, a CAG-repeat targeting dCas13d fusion protein of the disclosure comprises from N-terminal to C-terminal: an SV-40 NLS sequence, dCas13d (dSeq212) sequence, a linker sequence, an SV-40 NLS, and a ZC3H12A endonuclease (E17). In some aspects, the CAG-repeat targeting dCas13d protein of the disclosure is set forth in Table D. In some aspects, the CAG-repeat targeting dCas13d protein is used for methods of binding and cleaving CAG-repeat RNA sequences.

TABLE D
CAG-repeat targeting dCas13d protein

| Plasmid Element | Amino Acid Sequences |
|---|---|
| SV-40 NLS | PKKKRKV (SEQ ID NO: 437) |
| Linker | GGS |
| Dead Seq212 | KKKHQSAAEKRQVKKLKNQEKAQKYASEPSPLQSDTAGVECSQKKTVVS |
| | HIASSKTLAKAMGLKSTLVMGDKLVITSFAASKAVGGAGYKSANIEKITDL |
| | QGRVIEEHERMFSADVGEKNIELSKNDCHTNVNNPVVTNIGKDYIGLKSRL |
| | EQEFFGKTFENDNLHVQLAYNILDIKKILGTYVNNIIYIFYNLNRAGTGRDE |
| | RMYDDLIGTLYAYKPMEAQQTYLLKGDKDMRRFEEVKQLLQNTSAYYVY |
| | YGTLFEKVKAKSKKEQRAKEAEIDACTAHNYDVLRLLSLMRQLCMHSVA |
| | GTAFKLAESALFNIEDVLSADLKEILDEAFSGAVNKLNDGFVQHSGNNLYV |
| | LQQLYPNETIERIAEKYYRLTVRKEDLNMGVNIKKLRELIVGQYFPEVLDK |
| | EYDLSKNGDSVVTYRSKIYTVMNYILLYYLEDHDSSRESMVEALRQNREG |
| | DEGKEEIYRQFAKKVWNGVSGLFGVCLNLFKTEKRNKFRSKVALPDVSGA |
| | AYMLSSENIDYFVKMLFFVCKFLDGKEINELLCALINKFDNIADILDAAAQC |
| | GSSVWFVDSYRFFERSRRISAQIRIVKNIASKDFKKSKKDSDESYPEQLYLD |
| | ALALLGDVISKYKQNRDGSVVIDDQGNAVLTEQYKRFRYEFFEEIKRDESG |
| | GIKYKKSGKPEYNHQRRNFILNNVLKSKWFFYVVKYNRPSSCRELMKNKE |
| | ILRFVLRDIPDSQVRRYFKAVQGEEAYASAEAMRTRLVDALSQFSVTACLD |
| | EVGGMTDKEFASQRAVDSKEKLRAIIRLYLTVAYLITKSMVKVNTRFSIAF |
| | SVLERDYYLLIDGKKKSSDYTGEDMLALTRKFVGEDAGLYREWKEKNAE |
| | AKDKYFDKAERKKVLRQNDKMIRKMHFTPHSLNYVQKNLESVQSNGLAA |
| | VIKEYRNAVAALNIINRLDEYIGSARADSYYSLYCYCLQMYLSKNFSVGYL |
| | INVQKQLEEHHTYMKDLMWLLNIPFAYNLARYKNLSNEKLFYDEEAAAE |
| | KADKAENERGE (SEQ ID NO: 587) |
| Linker | GGGGSGGGGSGGGGS (SEQ ID NO: 415) |
| E17 | GGGTPKAPNLEPPLPEEEKEGSDLRPVVIDGSNVAMSHGNKEVFSCRGILL |
| | AVNWFLERGHTDITVFVPSWRKEQPRPDVPITDQHILRELEKKKILVFTPSR |
| | RVGGKRVVCYDDRFIVKLAYESDGIVVSNDTYRDLQGERQEWKRFIEERL |
| | LMYSFVNDKFMPPDDPLGRHGPSLDNFLRKKPLTLE (SEQ ID NO: 358) |
| Linker | GGS |
| Myc Tag | EQKLISEEDL (SEQ ID NO: 595) |

In some embodiments, a CAG-repeat targeting dCas13d fusion protein of the disclosure comprises from N-terminal to C-terminal: an SV-40 NLS sequence, a linker sequence, a dCas13d (dSeq212) sequence, a linker sequence, a ZC3H12A endonuclease (E17), a linker sequence, and a myc tag. In some embodiments, a CAG-repeat targeting dCas13d fusion protein of the disclosure comprises from N-terminal to C-terminal: an SV-40 NLS sequence, a linker sequence, a dCas13d (dSeq212) sequence, a linker sequence, and a ZC3H12A endonuclease (E17). In some aspects, the CAG-repeat targeting dCas13d protein of the disclosure is set forth in Table E. In some aspects, the CAG-repeat targeting dCas13d protein is used for methods of binding and cleaving CAG-repeat RNA sequences.

TABLE 3
CAG-repeat targeting dCas13d protein

| Plasmid Element | Amino Acid Sequences |
|---|---|
| SV-40 NLS | PKKKRKV |
| Linker | GGS |
| Dead Seq212 | KKKHQSAAEKRQVKKLKNQEKAQKYASEPSPLQSDTAGVECSQKKTVVS |
| | HIASSKTLAKAMGLKSTLVMGDKLVITSFAASKAVGGAGYKSANIEKITDL |
| | QGRVIEEHERMFSADVGEKNIELSKNDCHTNVNNPVVTNIGKDYIGLKSRL |
| | EQEFFGKTFENDNLHVQLAYNILDIKKILGTYVNNIIYIFYNLNRAGTGRDE |
| | RMYDDLIGTLYAYKPMEAQQTYLLKGDKDMRRFEEVKQLLQNTSAYYVY |
| | YGTLFEKVKAKSKKEQRAKEAEIDACTAHNYDVLRLLSLMAQLCMASVA |
| | GTAFKLAESALFNIEDVLSADLKEILDEAFSGAVNKLNDGFVQHSGNNLYV |
| | LQQLYPNETIERIAEKYYRLTVRKEDLNMGVNIKKLRELIVGQYFPEVLDK |
| | EYDLSKNGDSVVTYRSKIYTVMNYILLYYLEDHDSSRESMVEALRQNREG |
| | DEGKEEIYRQFAKKVWNGVSGLFGVCLNLFKTEKRNKFRSKVALPDVSGA |
| | AYMLSSENIDYFVKMLFFVCKFLDGKEINELLCALINKFDNIADILDAAAQC |
| | GSSVWFVDSYRFFERSRRISAQIRIVKNIASKDFKKSKKDSDESYPEQLYLD |
| | ALALLGDVISKYKQNRDGSVVIDDQGNAVLTEQYKRFRYEFFEEIKRDESG |
| | GIKYKKSGKPEYNHQRRNFILNNVLKSKWFFYVVKYNRPSSCRELMKNKE |
| | ILRFVLRDIPDSQVRRYFKAVQGEEAYASAEAMRTRLVDALSQFSVTACLD |
| | EVGGMTDKEFASQRAVDSKEKLRAIIRLYLTVAYLITKSMVKVNTRFSIAF |
| | SVLERDYYLLIDGKKKSSDYTGEDMLALTRKFVGEDAGLYREWKEKNAE |
| | AKDKYFDKAERKKVLRQNDKMIRKMHFTPHSLNYVQKNLESVQSNGLAA |
| | VIKEYANAVAALNIINRLDEYIGSARADSYYSLYCYCLQMYLSKNFSVGYL |
| | INVQKQLEEHHTYMKDLMWLLNIPFAYNLARYKNLSNEKLFYDEEAAAE |
| | KADKAENERGE (SEQ ID NO: 590) |
| Linker | GGGGSGGGGSGGGGS (SEQ ID NO: 415) |
| E17 | GGGTPKAPNLEPPLPEEEKEGSDLRPVVIDGSNVAMSHGNKEVFSCRGILL |
| | AVNWFLERGHTDITVFVPSWRKEQPRPDVPITDQHILRELEKKKILVFTPSR |
| | RVGGKRVVCYDDRFIVKLAYESDGIVVSNDTYRDLQGERQEWKRFIEERL |
| | LMYSFVNDKFMPPDDPLGRHGPSLDNFLRKKPLTLE (SEQ ID NO: 358) |
| Linker | GGS |
| Myc Tag | EQKLISEEDL (SEQ ID NO: 595) |

In some embodiments, a CAG-repeat targeting dCas13d fusion protein of the disclosure comprises from N-terminal to C-terminal: a ZC3H12A endonuclease (E17), a linker sequence, a dCas13d (dSeq212) sequence, a linker sequence, an SV-40 NLS, a linker sequence, and an HA tag. In some embodiments, a CAG-repeat targeting dCas13d fusion protein of the disclosure comprises from N-terminal to C-terminal: a ZC3H12A endonuclease (E17), a linker sequence, a dCas13d (dSeq212) sequence, a linker sequence, and an SV-40 NLS. In some aspects, the CAG-repeat targeting dCas13d protein of the disclosure is set forth in Table F. In some aspects, the CAG-repeat targeting dCas13d protein is used for methods of binding and cleaving CAG-repeat RNA sequences.

TABLE F
CAG-repeat targeting dCas13d protein

| Plasmid Element | Amino Acid Sequences |
|---|---|
| E17 | GGGTPKAPNLEPPLPEEEKEGSDLRPVVIDGSNVAMSHGNKEVFSCRGILL |
| | AVNWFLERGHTDITVFVPSWRKEQPRPDVPITDQHILRELEKKKILVFTPSR |
| | RVGGKRVVCYDDRFIVKLAYESDGIVVSNDTYRDLQGERQEWKRFIEERL |
| | LMYSFVNDKFMPPDDPLGRHGPSLDNFLRKKPLTLE (SEQ ID NO: 358) |
| Linker | GGGGSGGGGSGGGGS (SEQ ID NO: 415) |
| Dead Seq212 | KKKHQSAAEKRQVKKLKNQEKAQKYASEPSPLQSDTAGVECSQKKTVVS |
| | HIASSKTLAKAMGLKSTLVMGDKLVITSFAASKAVGGAGYKSANIEKITDL |
| | QGRVIEEHERMFSADVGEKNIELSKNDCHTNVNNPVVTNIGKDYIGLKSRL |
| | EQEFFGKTFENDNLHVQLAYNILDIKKILGTYVNNIIYIFYNLNRAGTGRDE |
| | RMYDDLIGTLYAYKPMEAQQTYLLKGDKDMRRFEEVKQLLQNTSAYYVY |
| | YGTLFEKVKAKSKKEQRAKEAEIDACTAHNYDVLRLLSLMAQLCMASVA |
| | GTAFKLAESALFNIEDVLSADLKEILDEAFSGAVNKLNDGFVQHSGNNLYV |
| | LQQLYPNETIERIAEKYYRLTVRKEDLNMGVNIKKLRELIVGQYFPEVLDK |
| | EYDLSKNGDSVVTYRSKIYTVMNYILLYYLEDHDSSRESMVEALRQNREG |
| | DEGKEEIYRQFAKKVWNGVSGLFGVCLNLFKTEKRNKFRSKVALPDVSGA |
| | AYMLSSENIDYFVKMLFFVCKFLDGKEINELLCALINKFDNIADILDAAAQC |
| | GSSVWFVDSYRFFERSRRISAQIRIVKNIASKDFKKSKKDSDESYPEQLYLD |
| | ALALLGDVISKYKQNRDGSVVIDDQGNAVLTEQYKRFRYEFFEEIKRDESG |
| | GIKYKKSGKPEYNHQRRNFILNNVLKSKWFFYVVKYNRPSSCRELMKNKE |
| | ILRFVLRDIPDSQVRRYFKAVQGEEAYASAEAMRTRLVDALSQFSVTACLD |
| | EVGGMTDKEFASQRAVDSKEKLRAIIRLYLTVAYLITKSMVKVNTRFSIAF |
| | SVLERDYYLLIDGKKKSSDYTGEDMLALTRKFVGEDAGLYREWKEKNAE |
| | AKDKYFDKAERKKVLRQNDKMIRKMHFTPHSLNYVQKNLESVQSNGLAA |
| | VIKEYANAVAALNIINRLDEYIGSARADSYYSLYCYCLQMYLSKNFSVGYL |
| | INVQKQLEEHHTYMKDLMWLLNIPFAYNLARYKNLSNEKLFYDEEAAAE |
| | KADKAENERGE (SEQ ID NO: 590) |
| Linker | GS |
| SV40 NLS | PKKKRKV (SEQ ID NO: 437) |
| Linker | ED |
| HA Tag | YPYDVPDYA (SEQ ID NO: 586) |

### Non-Guided CAG-Repeat RNA Binding Systems

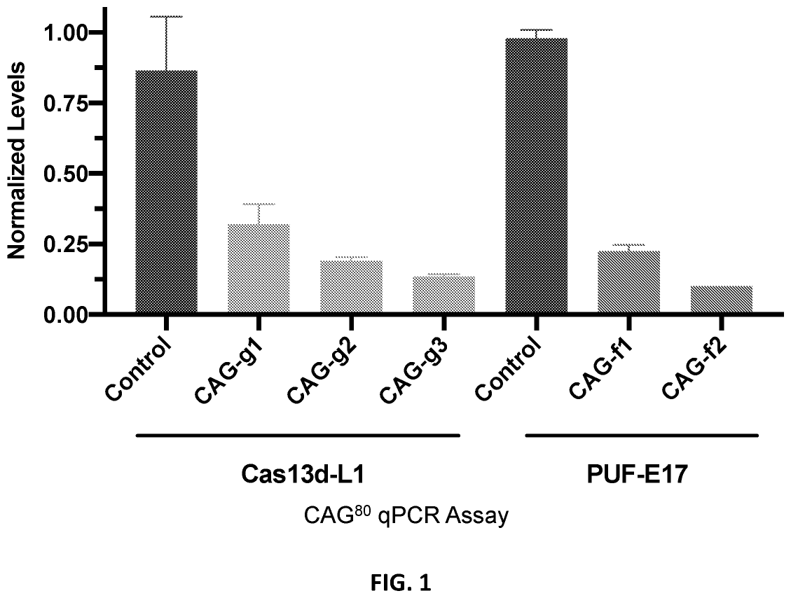

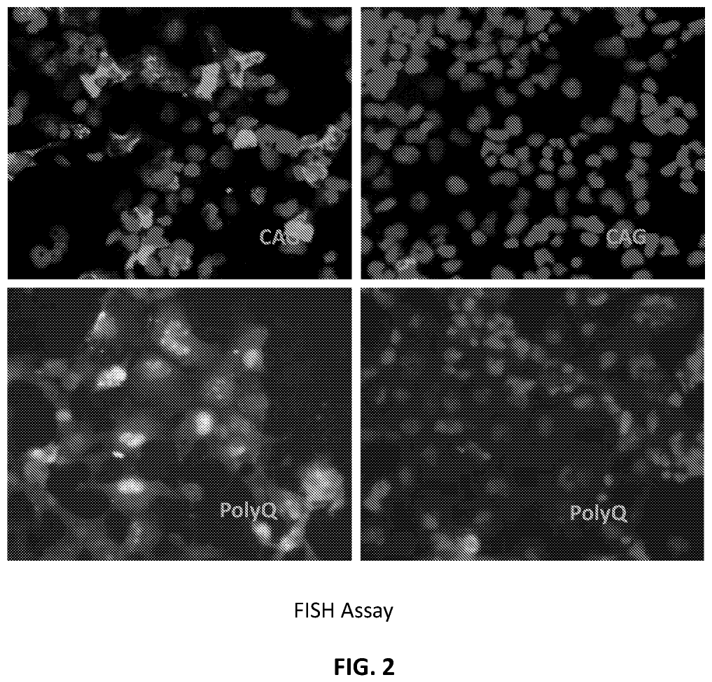

In some embodiments, the RNA-binding system for targeting CAG toxic repeats does not comprise an RNA-guided RNA-binding polypeptide. In some embodiments, the RNA-binding system is comprised of a non-RNA-guided RNA-binding polypeptide. In some embodiments, the RNA-binding system is comprised of a non-RNA-guided RNA-binding polypeptide such as a PUF protein or a PUMBY protein, or RNA-binding portion thereof. In one embodiment, a non-guided RNA-binding fusion protein disclosed herein comprises a) a PUF or PUMBY RNA-binding sequence capable of binding a toxic target CAG repeat RNA sequence comprising CAGCAGCA (SEQ ID NO: 453) or GCAGCAGC (SEQ ID NO: 476) and b) an endonuclease capable of cleaving the toxic target CAG repeat sequence. The target CAG repeat frame 1 (CAG-f1 in FIG. 1) is CAGCAGCA (SEQ ID NO: 453) and the target CAG repeat frame 2 (CAG-f2 in FIG. 1) is GCAGCAGC (SEQ ID NO: 476). In another embodiment, the target CAG repeat frame is CAG repeat frame 3 which is AGCAGCAG (SEQ ID NO: 472).

In another embodiment, the toxic target RNA sequence comprises a target RNA sequence selected from the group consisting of CAGCAGCAGCAGCA (SEQ ID NO: 454), CAGCAGCAGCAGCAG (SEQ ID NO: 455), CAGCAGCAGCAGCAGC (SEQ ID NO: 456), GCAGCAGCAGCAGC (SEQ ID NO: 477), GCAGCAGCAGCAGCA (SEQ ID NO: 478), GCAGCAGCAGCAGCAG (SEQ ID NO: 479), AGCAGCAGCAGCAG (SEQ ID NO: 473), AGCAGCAGCAGCAGC (SEQ ID NO: 474), and AGCAGCAGCAGCAGCA (SEQ ID NO: 475).

In one embodiment, the PUF or PUMBY RNA-binding fusion protein comprises a) PUF or PUMBY CAG-targeting protein and b) a nuclease domain of ZC3H12A, a zinc-finger endonuclease, (referred to as E17 herein). In some embodiments, the CAG-targeting PUF or PUMBY fusion protein is configured with the N-terminal to C-terminal orientation as follows:
PUF(CAG)-E17, wherein PUF(CAG) is a CAG targeting PUF;
E17-PUF(CAG);
PUMBY(CAG)-E17, wherein PUMBY(CAG) is a CAG targeting PUMBY; or
E17-PUMBY(CAG).

In some embodiments, the PUF or PUMBY fusion configurations include a linker between the PUF(CAG) or PUMBY(CAG) and the E17 nuclease domain. In one embodiment, the linker sequence is VDTANGS (SEQ ID NO: 411).

In some embodiments, the CAG-targeting PUF or PUMBY fusion protein comprising a linker is configured N-terminal to C-terminal as follows:
PUF(CAG)-linker-E17
E17-linker-PUF(CAG)
PUMBY(CAG)-linker-E17; or
E17-linker-PUMBY(CAG).

In one embodiment, the CAG-targeting PUF or PUMBY fusion protein configuration from N-terminal to C-terminal is the orientation PUF(CAG)-VDTANGS-E17 or PUMBY(CAG)-VDTANGS-E17. In another embodiment, the CAG-targeting PUF or PUMBY fusion protein configuration from N-terminal to C-terminal is the orientation E17-VDTANGS-PUF(CAG) or E17-VDTANGS-PUMBY(CAG).

In some embodiments, the PUF or PUMBY configurations include one or more signal sequences and/or tags such as FLAG, NLS, NES or a combination thereof. In one embodiment, the FLAG tag sequence is DYKDDDDK (SEQ ID NO: 436). In one embodiment, the NLS is a human NLS. In another embodiment, the human NLS is human pRB-NLS: KRSAEGSNPPKPLKKLR (SEQ ID NO: 442) or human RB-NLS (extended version): DRVLKRSAEGSNPPKPLKKLR (SEQ ID NO: 543).

In one embodiment, the configuration comprises two different tags and/or signal sequences. In another embodiment, the configuration comprises two or more signal sequences. In some embodiments, the signal(s) is located at the N-terminal. In some embodiments, the signal(s) is located at the C-terminal. In some embodiments, a signal(s) is located at the N-terminal and a signal(s) is located at the C-terminal. In one embodiment, the CAG-targeting PUF or PUMBY fusion protein comprising one or more signals and/or tags is configured N-terminal to C-terminal as follows:
FLAG-NLS-PUF(CAG)-linker-E17;
FLAG-NLS-PUMBY(CAG)-linker-E17;
NLS-PUF(CAG)-linker-E17; or
NLS-PUMBY(CAG)-linker-E17.

In one embodiment, the CAG-targeting PUF or PUMBY fusion protein comprising one or more tags is configured N-terminal to C-terminal as follows:
FLAG-NLS-PUF(CAG)-VDTANGS-E17;
FLAG-NLS-PUMBY(CAG)-VDTANGS-E17;
NLS-PUF(CAG)-VDTANGS-E17; or
NLS-PUMBY(CAG)-VDTANGS-E17
NLS-PUF(CAG)-VDTANGS-E17-NES.

TABLE 2
Exemplary 8PUF configuration for targeting CAG
MRE

| Protein | Target | |
|---|---|---|
| Type | Sequence | Amino Acid Sequence of PUF |
| 8PUF | CAGCAGCA | GRSRLLEDFRNNRYPNLQLREIAGHI |
| | (SEQ ID NO: | MEFSQDQHGSRFIQLKLERATPAERQ |
| | 453) - Frame | LVFNEILQAAYQLMVDVFGSYVIRKF |
| | 1 | FEFGSLEQKLALAERIRGHVLSLALQ |
| | | MYGSRVIEKALEFIPSDQQNEMVREL |
| | | DGHVLKCVKDQNGCYVVQKCIECV |
| | | QPQSLQFIIDAFKGQVFALSTHPYGSR |
| | | VIRRILEHCLPDQTLPILEELHQHTEQ |
| | | LVQDQYGSYVIEHVLEHGRPEDKSKI |
| | | VAEIRGNVLVLSQHKFACNVVQKCV |
| | | THASRTERAVLIDEVCTMNDGPHSA |
| | | LYTMMKDQYASYVVRKMIDVAEPG |
| | | QRKIVMHKIRPHIATLRKYTYGKHIL |
| | | AKLEKYYMKNGVDLG |
| | | (SEQ ID NO: 480) |
| 8PUF | GCAGCAGC | GRSRLLEDFRNNRYPNLQLREIAGHI |
| | (SEQ ID NO: | MEFSQDQHGSRFIRLKLERATPAERQ |
| | 476) - Frame | LVFNEILQAAYQLMVDVFGSYVIEKF |
| | 2 | FEFGSLEQKLALAERIRGHVLSLALQ |
| | | MYGCRVIQKALEFIPSDQQNEMVRE |
| | | LDGHVLKCVKDQNGSYVVRKCIECV |
| | | QPQSLQFIIDAFKGQVFALSTHPYGSR |
| | | VIERILEHCLPDQTLPILEELHQHTEQ |
| | | LVQDQYGCYVIQHVLEHGRPEDKSK |
| | | IVAEIRGNVLVLSQHKFASYVVRKCV |
| | | THASRTERAVLIDEVCTMNDGPHSA |
| | | LYTMMKDQYASYVVEKMIDVAEPG |
| | | QRKIVMHKIRPHIATLRKYTYGKHIL |
| | | AKLEKYYMKNGVDLG |
| | | (SEQ ID NO: 549) |
| 8PUF | GCAGCAGC | GRSRLLEDFRNNRYPNLQLREIAGHI |
| | (SEQ ID NO: | MEFSQDQHGSRFIRLKLERATPAERQ |
| | 476) - Frame | LVFNEILQAAYQLMVDVFGSYVIEKF |
| | 2 - R4 amino | FEFGSLEQKLALAERIRGHVLSLALQ |
| | acid 13 H | MYGCRVIQKALEFIPSDQQNEMVRE |
| | | LDGHVLKCVKDQNGSHVVRKCIECV |
| | | QPQSLQFIIDAFKGQVFALSTHPYGSR |
| | | VIERILEHCLPDQTLPILEELHQHTEQ |
| | | LVQDQYGCYVIQHVLEHGRPEDKSK |
| | | IVAEIRGNVLVLSQHKFASYVVRKCV |
| | | THASRTERAVLIDEVCTMNDGPHSA |
| | | LYTMMKDQYASYVVEKMIDVAEPG |
| | | QRKIVMHKIRPHIATLRKYTYGKHIL |
| | | AKLEKYYMKNGVDLG (SEQ ID NO: |
| | | 568) |
| 8PUF | AGCAGCAG | GRSRLLEDFRNNRYPNLQLREIAGHI |
| | (SEQ ID NO: | MEFSQDQHGSRFIELKLERATPAERQ |
| | 472) - Frame | LVFNEILQAAYQLMVDVFGCYVIQK |
| | 3 | FFEFGSLEQKLALAERIRGHVLSLAL |
| | | QMYGSYVIRKALEFIPSDQQNEMVR |
| | | ELDGHVLKCVKDQNGSYVVEKCIEC |
| | | VQPQSLQFIIDAFKGQVFALSTHPYG |
| | | CRVIQRILEHCLPDQTLPILEELHQHT |
| | | EQLVQDQYGSYVIRHVLEHGRPEDK |
| | | SKIVAEIRGNVLVLSQHKFASNVVEK |
| | | CVTHASRTERAVLIDEVCTMNDGPH |
| | | SALYTMMKDQYACYVVQKMIDVAE |
| | | PGQRKIVMHKIRPHIATLRKYTYGKH |
| | | ILAKLEKYYMKNGVDLG (SEQ ID |
| | | NO: 444) |

In one embodiment, the PUF(CAG) or PUMBY(CAG) fusion construct targets expanded CAG repeats, wherein the CAG repeats are CAG³⁶ or more. In another embodiment, the CAG repeats are CAG⁸⁰. In some aspects, CAG³⁶ or CAG⁸⁰ refers to 36 CAG repeats or 80 CAG repeats in the HTT or SCA1 gene. Any other number of CAG repeats are possible, including at least 1, 2, 3, 4, 5, 6, 7, 8, 9, 10, 15, 20, 25, 30, 35, 36, 37, 38, 39, 40, 41, 42, 43, 44, 45, 46, 47, 48, 49, 50, 55, 60, 65, 70, 75, 76, 77, 78, 79, 80, 81, 82, 83, 84, 85, 90, 95, 100, 105, 110, 115, 120 CAG repeats, or any other number of CAG repeats in between.

In one embodiment, the nucleic acid sequence encoding the PUF(CAG) or PUMBY(CAG) protein or fusion construct is operably linked to a promoter sequence for expression in a cell. In one embodiment, the promoter sequence is a truncated CAG (tCAG) promoter (FIG. 3A). In some embodiments, the promoter sequence comprises an enhancer sequence and/or an intron sequence. In one embodiment, the promoter is a EFS/UBB promoter. In some embodiments, the promoter sequence is a neuron-specific promoter.

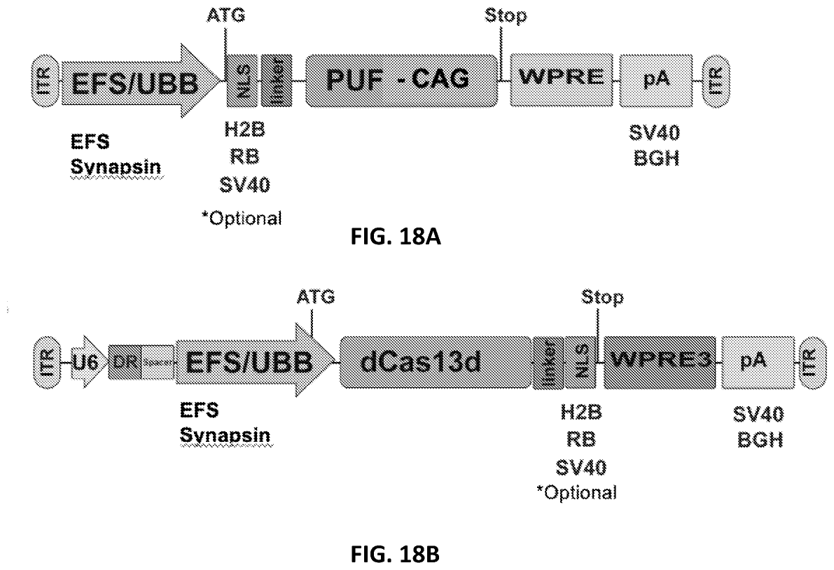

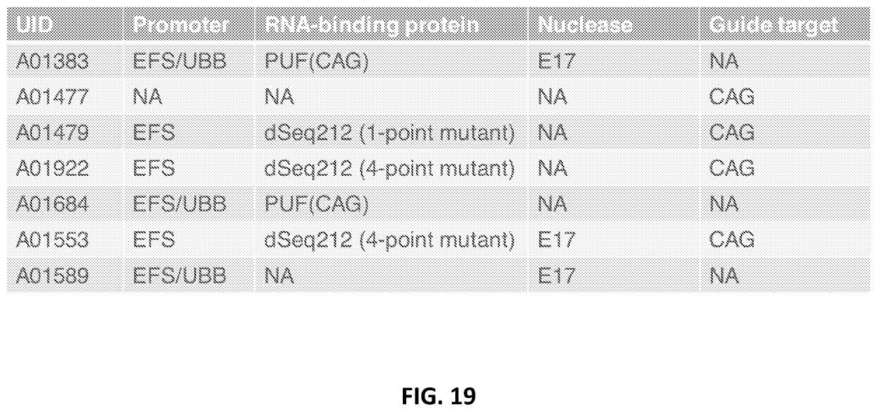

In one embodiment, the nucleic acid encoding the Cas13d(CAG) or dCas13d(CAG) (dCas13d(CAG) with or without an endonuclease) is operably linked to a promoter sequence for expression in a cell (FIG. 3A-3C and FIG. 18A-18B). In one embodiment, the promoter sequence is an EFS promoter (FIG. 3C or FIG. 18A-18B). In one embodiment, the promoter is a EFS/UBB promoter (FIG. 18A-18B). In one embodiment, the promoter is a synapsin promoter (FIG. 18A-18B). In some embodiments, the promoter sequence comprises an enhancer sequence and/or an intron sequence. In some embodiments, the promoter sequence is a neuron-specific promoter.

In another embodiment, the PUF(CAG) or PUMBY(CAG) or Cas13d(CAG) or dCas13d(CAG) configurations are packaged in an AAV vector. In one embodiment, the AAV vector is an AAV9 vector. In another embodiment, the AAV vector is an AAVrh74 vector.

In another embodiment, the PUF(CAG) or PUMBY(CAG) configurations are packaged in an AAV vector. In one embodiment, the AAV vector is an AAV9 or AAVrh10 vector.

### Guide RNAs for RNA-Guided RNA-Binding Proteins

The terms guide RNA (gRNA) and single guide RNA (sgRNA) are used interchangeably throughout the disclosure.

Guide RNAs (gRNAs) of the disclosure may comprise of a spacer sequence and a “direct repeat” (DR) sequence. In some embodiments, a guide RNA is a single guide RNA (sgRNA) comprising a contiguous spacer sequence and DR sequence. In some embodiments, the spacer sequence and the DR sequence are not contiguous. In some embodiments, the gRNA comprises a DR sequence. DR sequences refer to the repetitive sequences in the CRISPR locus (naturally-occurring in a bacterial genome or plasmid) that are interspersed with the spacer sequences. It is well known that one would be able to infer the DR sequence of a corresponding (or cognate) Cas protein if the sequence of the associated CRISPR locus is known. In some embodiments, a guide RNA comprises a direct repeat (DR) sequence and a spacer sequence. In some embodiments, a sequence encoding a guide RNA or single guide RNA of the disclosure comprises or consists of a spacer sequence and a DR sequence, that are separated by a linker sequence. In some embodiments, the linker sequence may comprise or consist of 1, 2, 3, 4, 5, 6, 7, 8, 9, 10, 15, 20, 25, 30, 35, 40, 45, 50 or any number of nucleotides (nt) in between. In some embodiments, the linker sequence may comprise at least 1, 2, 3, 4, 5, 6, 7, 8, 9, 10, 15, 20, 25, 30, 35, 40, 45, 50 or any number of nucleotides in between. In some embodiments, the DR sequence is a Cas13d DR sequence.

In one embodiment, the gRNA that hybridizes with the one or more target RNA molecules in a Cas 13d-mediated manner includes one or more direct repeat (DR) sequences, one or more spacer sequences, such as, e.g., one or more sequences comprising an array of DR-spacer-DR-spacer. In one embodiment, a plurality of gRNAs are generated from a single array, wherein each gRNA can be different, for example target different RNAs or target multiple regions of a single RNA, or combinations thereof. In some embodiments, an isolated gRNA includes one or more direct repeat sequences, such as an unprocessed (e.g., about 36 nt) or processed DR (e.g., about 30 nt). In some embodiments, a gRNA can further include one or more spacer sequences specific for (e.g., is complementary to) the target RNA. In certain such embodiments, multiple polIII promoters can be used to drive multiple gRNAs, spacers and/or DRs. In one embodiment, a guide array comprises a DR (about 36 nt)-spacer (about 30 nt)-DR (about 36 nt)-spacer (about 30 nt).

Guide RNAs (gRNAs) of the disclosure may comprise non-naturally occurring nucleotides. In some embodiments, a guide RNA of the disclosure or a sequence encoding the guide RNA comprises or consists of modified or synthetic RNA nucleotides. Exemplary modified RNA nucleotides include, but are not limited to, pseudouridine (Ψ), dihydrouridine (D), inosine (I), and 7-methylguanosine (m7G), hypoxanthine, xanthine, xanthosine, 7-methylguanine, 5, 6-Dihydrouracil, 5-methylcytosine, 5-methylcytidine, 5-hydropxymethylcytosine, isoguanine, and isocytosine.

Guide RNAs (gRNAs) of the disclosure may bind modified RNA within a target sequence. Within a target sequence, guide RNAs (gRNAs) of the disclosure may bind modified or mutated (e.g., pathogenic) RNA. Exemplary epigenetically or post-transcriptionally modified RNA include, but are not limited to, 2′-O-Methylation (2′-OMe) (2′-O-methylation occurs on the oxygen of the free 2′-OH of the ribose moiety), N6-methyladenosine (m6A), and 5-methylcytosine (m5C).

In some embodiments of the compositions of the disclosure, a guide RNA of the disclosure comprises at least one sequence encoding a non-coding C/D box small nucleolar RNA (snoRNA) sequence. In some embodiments, the snoRNA sequence comprises at least one sequence that is complementary to the target RNA, wherein the target sequence of the RNA molecule comprises at least one 2′-OMe. In some embodiments, the snoRNA sequence comprises at least one sequence that is complementary to the target RNA, wherein the at least one sequence that is complementary to the target RNA comprises a box C motif (RUGAUGA) and a box D motif (CUGA).

Spacer sequences of the disclosure bind to the target sequence of an RNA molecule. In some embodiments, spacer sequences of the disclosure bind to pathogenic target RNA.

In some embodiments of the compositions of the disclosure, the sequence comprising the gRNA further comprises a spacer sequence that specifically binds to the target RNA sequence. In some embodiments, the spacer sequence has at least 50%, 55%, 60%, 65%, 70%, 75%, 80%, 87%, 90%, 95%, 97%, 99% or any percentage in between of complementarity to the target RNA sequence. In some embodiments, the spacer sequence has 100% complementarity to the target RNA sequence. In some embodiments, the spacer sequence comprises or consists of 20 nucleotides. In some embodiments, the spacer sequence comprises or consists of 21 nucleotides, 22 nucleotides, 23 nucleotides, 24 nucleotides, 25 nucleotides, 26 nucleotides, 27 nucleotides, 28 nucleotides, or 29 nucleotides. In some embodiments, the spacer sequence comprises or consists of 26 nucleotides. In some embodiments, the spacer sequence is non-processed and comprises or consists of 30 nucleotides. In some embodiments the non-processed spacer sequence comprises or consists of 30-36 nucleotides.

DR sequences of the disclosure bind the Cas polypeptide of the disclosure. Upon binding of the spacer sequence of the gRNA to the target RNA sequence, the Cas protein bound to the DR sequence of the gRNA is positioned at the target RNA sequence. A DR sequence having sufficient complementarity to its cognate Cas protein, or nucleic acid thereof, binds selectively to the target nucleic acid sequence of the Cas protein and has at least 50%, 55%, 60%, 65%, 70%, 75%, 80%, 85%, 90%, 95%, 96, 97%, 98%, 99%, or any percentage identity in between to the sequence. In some embodiments, a sequence having sufficient complementarity has 100% identity. In some embodiments, DR sequences of the disclosure comprise a secondary structure or a tertiary structure. Exemplary secondary structures include, but are not limited to, a helix, a stem loop, a bulge, a tetraloop and a pseudoknot. Exemplary tertiary structures include, but are not limited to, an A-form of a helix, a B-form of a helix, and a Z-form of a helix. Exemplary tertiary structures include, but are not limited to, a twisted or helicized stem loop. Exemplary tertiary structures include, but are not limited to, a twisted or helicized pseudoknot. In some embodiments, DR sequences of the disclosure comprise at least one secondary structure or at least one tertiary structure. In some embodiments, DR sequences of the disclosure comprise one or more secondary structure(s) or one or more tertiary structure(s).

In some embodiments of the compositions of the disclosure, a guide RNA or a portion thereof selectively binds to a tetraloop motif in an RNA molecule of the disclosure. In some embodiments, a target sequence of an RNA molecule comprises a tetraloop motif. In some embodiments, the tetraloop motif is a “GRNA” motif comprising or consisting of one or more of the sequences of GAAA, GUGA, GCAA or GAGA.

In some embodiments of the compositions of the disclosure, a guide RNA or a portion thereof that binds to a target sequence of an RNA molecule hybridizes to the target sequence of the RNA molecule. In some embodiments, a guide RNA or a portion thereof that binds to a first RNA binding protein or to a second RNA binding protein covalently binds to the first RNA binding protein or to the second RNA binding protein. In some embodiments, a guide RNA or a portion thereof that binds to a first RNA binding protein or to a second RNA binding protein non-covalently binds to the first RNA binding protein or to the second RNA binding protein.

In some embodiments of the compositions of the disclosure, a guide RNA or a portion thereof comprises or consists of between 10 and 100 nucleotides, inclusive of the endpoints. In some embodiments, a spacer sequence of the disclosure comprises or consists of between 10 and 30 nucleotides, inclusive of the endpoints. In some embodiments, a spacer sequence of the disclosure comprises or consists of 15, 16, 17, 18, 19, 20, 21, 22, 23, 24, 25, 26, 27, 28, 29 or 30 nucleotides. In some embodiments, the spacer sequence of the disclosure comprises or consists of 20 nucleotides. In some embodiments, the spacer sequence of the disclosure comprises or consists of 21 nucleotides. In some embodiments, the spacer sequence of the disclosure comprises or consists of 26 nucleotides.

Guide molecules generally exist in various states of processing. In one example, an unprocessed guide RNA is 36 nt of DR followed by 30-32 nt of spacer. The guide RNA is processed (truncated/modified) by Cas 13d itself or other RNases into the shorter “mature” form. In some embodiments, an unprocessed guide sequence is about, or at least about 30, 35, 40, 45, 50, 55, 60, 61, 62, 63, 64, 65, 66, 67, 68, 69, 70, 71, 72, 73, 74, 75, or more nucleotides (nt) in length. In some embodiments, a processed guide sequence is about 44 to 60 nt (such as 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 nt). In some embodiments, an unprocessed spacer is about 28-32 nt long (such as 25, 26, 27, 28, 29, 30, 31, 32, 33, 34, or 35 nt) while the mature (processed) spacer can be about 10 to 30 nt, 10 to 25 nt, 14 to 25 nt, 20 to 22 nt, or 14-30 nt (such as 10, 11, 12, 13, 14, 15, 16, 17, 18, 19, 20, 21, 22, 23, 24, 25, 26, 27, 28, 29, 30, 31, 32, 33, 34, or 35 nt). In some embodiments, an unprocessed DR is about 36 nt (such as 30, 31, 32, 33, 34, 35, 36, 37, 38, 39, 40 or 41 nt), while the processed DR is about 30 nt (such as 25, 26, 27, 28, 29, 30, 31, 32, 33, 34, or 35 nt). In some embodiments, a DR sequence is truncated by 1-10 nucleotides (such as 1, 2, 3, 4, 5, 6, 7, 8, 9, to 10 nucleotides at e.g., the 5′ end in order to be expressed as mature pre-processed guide RNAs.

In some embodiments of the compositions of the disclosure, a guide RNA or a portion thereof does not comprise a nuclear localization sequence (NLS).

In some embodiments of the compositions of the disclosure, a guide RNA or a portion thereof comprises a sequence complementary to a protospacer flanking sequence (PFS). In some embodiments, including those wherein a guide RNA or a portion thereof comprises a sequence complementary to a PFS, the first RNA binding protein may comprise a sequence isolated or derived from a Cas13 protein. In some embodiments, including those wherein a guide RNA or a portion thereof comprises a sequence complementary to a PFS, the first RNA binding protein may comprise a sequence encoding a Cas13 protein or an RNA-binding portion thereof. In some embodiments, the guide RNA or a portion thereof does not comprise a sequence complementary to a PFS.

In some embodiments of the compositions of the disclosure, vectors comprising guide RNA sequences of the disclosure comprises a promoter sequence to drive expression of the guide RNA. In some embodiments, a vector comprising a guide RNA sequence of the disclosure comprises a promoter sequence to drive expression of the guide RNA. In some embodiments, the promoter to drive expression of the guide RNA is a constitutive promoter. In some embodiments, the promoter sequence is an inducible promoter. In some embodiments, the promoter is a sequence is a tissue-specific and/or cell-type specific promoter. In some embodiments, the promoter is a hybrid or a recombinant promoter. In some embodiments, the promoter is a promoter capable of expressing the guide RNA in a mammalian cell. In some embodiments, the promoter is a promoter capable of expressing the guide RNA in a human cell. In some embodiments, the promoter is a promoter capable of expressing the guide RNA and restricting the guide RNA to the nucleus of the cell. In some embodiments, the promoter is a human RNA polymerase promoter or a sequence isolated or derived from a sequence encoding a human RNA polymerase promoter. In some embodiments, the promoter is a U6 promoter or a sequence isolated or derived from a sequence encoding a U6 promoter. In some embodiments, the U6 promoter is a human U6 promoter. In some embodiments, the promoter is a human tRNA promoter or a sequence isolated or derived from a sequence encoding a human tRNA promoter. In some embodiments, the promoter is a human valine tRNA promoter or a sequence isolated or derived from a sequence encoding a human valine tRNA promoter.

In some embodiments of the compositions of the disclosure, a promoter to drive expression of the guide RNA further comprises a regulatory element. In some embodiments, a vector comprising a promoter sequence to drive expression of the guide RNA further comprises a regulatory element. In some embodiments, a regulatory element enhances expression of the guide RNA. Exemplary regulatory elements include, but are not limited to, an enhancer element, an intron, an exon, or a combination thereof.

In some embodiments of the compositions of the disclosure, a vector of the disclosure comprises one or more of a sequence encoding a guide RNA, a promoter sequence to drive expression of the guide RNA and a sequence encoding a regulatory element. In some embodiments of the compositions of the disclosure, the vector further comprises a sequence encoding a fusion protein of the disclosure.

### RNA-Guided RNA-Binding Proteins

In some embodiments of the compositions of the disclosure, gRNAs correspond to target RNA molecules and an RNA-guided RNA binding protein. In some embodiments, the gRNAs correspond to an RNA-guided RNA binding fusion protein, wherein the fusion protein comprises first and second RNA binding proteins. In some embodiments, the first RNA-binding protein in the fusion protein is a deactivated RNA-binding protein, e.g., a deactivated Cas or catalytic dead Cas protein. In some embodiments, along a sequence encoding the RNA-binding fusion protein, the sequence encoding the first RNA binding protein is positioned 5′ of the sequence encoding the second RNA binding protein. In some embodiments, along a sequence encoding the fusion protein, the sequence encoding the first RNA binding protein is positioned 3′ of the sequence encoding the second RNA binding protein.

In some embodiments of the compositions of the disclosure, the sequence encoding the first RNA binding protein comprises a sequence isolated or derived from a protein capable of binding an RNA molecule. In some embodiments, the sequence encoding the first RNA binding protein comprises a sequence isolated or derived from a protein capable of selectively binding an RNA molecule and not binding a DNA molecule, a mammalian DNA molecule or any DNA molecule. In some embodiments, the sequence encoding the first RNA binding protein comprises a sequence isolated or derived from a protein capable of binding an RNA molecule and inducing a break in the RNA molecule. In some embodiments, the sequence encoding the first RNA binding protein comprises a sequence isolated or derived from a protein capable of binding an RNA molecule, inducing a break in the RNA molecule, and not binding a DNA molecule, a mammalian DNA molecule or any DNA molecule. In some embodiments, the sequence encoding the first RNA binding protein comprises a sequence isolated or derived from a protein capable of binding an RNA molecule, inducing a break in the RNA molecule, and neither binding nor inducing a break in a DNA molecule, a mammalian DNA molecule or any DNA molecule.

In some embodiments of the compositions of the disclosure, the sequence encoding the first RNA-guided RNA binding protein comprises a sequence isolated or derived from a protein with no DNA nuclease activity.

In some embodiments of the compositions of the disclosure, the sequence encoding the RNA-guided RNA binding protein disclosed herein comprises a sequence isolated or derived from a CRISPR Cas protein. In some embodiments, the CRISPR Cas protein is not a Type II CRISPR Cas protein. In some embodiments, the CRISPR Cas protein is not a Cas9 protein.

In some embodiments of the compositions of the disclosure, the sequence encoding the RNA-guided RNA binding protein comprises a Type VI CRISPR Cas protein or portion thereof. In some embodiments, the Type VI CRISPR Cas protein comprises a Cas13 protein or portion thereof. Exemplary Cas13 proteins of the disclosure may be isolated or derived from any species, including, but not limited to, bacteria or archaea. Exemplary Cas13 proteins of the disclosure may be isolated or derived from any species, including, but not limited to, Leptotrichia wadei, Listeria seeligeri serovar 1/2b (strain ATCC 35967/DSM 20751/CIP 100100/SLCC 3954), Lachnospiraceae bacterium, Clostridium aminophilum DSM 10710, Carnobacterium gallinarum DSM 4847, Paludibacter propionicigenes WB4, Listeria weihenstephanensis FSL R9-0317, Listeria weihenstephanensis FSL R9-0317, bacterium FSL M6-0635 (Listeria newyorkensis), Leptotrichia wadei F0279, Rhodobacter capsulatus SB 1003, Rhodobacter capsulatus R121, Rhodobacter capsulatus DE442 and Corynebacterium ulcerans. Exemplary Cas13 proteins of the disclosure may be DNA nuclease inactivated. Exemplary Cas13 proteins of the disclosure include, but are not limited to, Cas13a, Cas13b, Cas13c, Cas13d and orthologs thereof. Exemplary Cas13b proteins of the disclosure include, but are not limited to, subtypes 1 and 2 referred to herein as Csx27 and Csx28, respectively.

Exemplary Cas13a proteins include, but are not limited to:

| Cas13a | Cas13a | | | |
|---|---|---|---|---|
| number | abbreviation | Organism name | Accession number | Direct Repeat sequence |
| Cas13a1 | LshCas13a | Leptotrichia | WP_018451595.1 | CCACCCCAATATCGAAGGGGACTAA |
| | | shahii | | AAC (SEQ ID NO: 393) |
| Cas13a2 | LwaCas13a | Leptotrichia | WP_021746774.1 | GATTTAGACTACCCCAAAAACGAAG |
| | | wadei | | GGGACTAAAAC (SEQ ID NO: |
| | | | | 394) |
| Cas13a3 | LseCas13a | Listeria seeligeri | WP_012985477.1 | GTAAGAGACTACCTCTATATGAAAG |
| | | | | AGGACTAAAAC (SEQ ID NO: |
| | | | | 395) |
| Cas13a4 | LbmCas13a | Lachnospiraceae | WP_044921188.1 | GTATTGAGAAAAGCCAGATATAGTT |
| | | bacterium | | GGCAATAGAC (SEQ ID NO: 396) |
| | | MA2020 | | |
| Cas13a5 | LbnCas13a | Lachnospiraceae | WP_022785443.1 | GTTGATGAGAAGAGCCCAAGATAG |
| | | bacterium | | AGGGCAATAAC (SEQ ID NO: |
| | | NK4A179 | | 397) |
| Cas13a6 | CamCas13a | [Clostridium] | WP_031473346.1 | GTCTATTGCCCTCTATATCGGGCTGT |
| | | aminophilum | | TCTCCAAAC (SEQ ID NO: 398) |
| | | DSM 10710 | | |
| Cas13a7 | CgaCas13a | Carnobacterium | WP_034560163.1 | ATTAAAGACTACCTCTAAATGTAAG |
| | | gallinarum DSM | | AGGACTATAAC (SEQ ID NO: |
| | | 4847 | | 399) |
| Cas13a8 | Cga2Cas13a | Carnobacterium | WP_034563842.1 | AATATAAACTACCTCTAAATGTAAG |
| | | gallinarum DSM | | AGGACTATAAC (SEQ ID NO: |
| | | 4847 | | 400) |
| Cas13a9 | Pprcas 13a | Paludibacter | WP_013443710.1 | CTTGTGGATTATCCCAAAATTGAAG |
| | | propionicigenes | | GGAACTACAAC (SEQ ID NO: |
| | | WB4 | | 401) |
| Cas13a10 | LweCas13a | Listeria | WP_036059185.1 | GATTTAGAGTACCTCAAAATAGAAG |
| | | weihenstephanensis | | AGGTCTAAAAC (SEQ ID NO: |
| | | FSL R9-0317 | | 402) |
| Cas13a11 | LbfCas13a | Listeriaceae | WP_036091002.1 | GATTTAGAGTACCTCAAAACAAAAG |
| | | bacterium FSL | | AGGACTAAAAC (SEQ ID NO: |
| | | M6-0635 | | 403) |
| | | (Listeria | | |
| | | newyorkensis) | | |
| Cas13a12 | Lwa2cas13a | Leptotrichia | WP_021746774.1 | GATATAGATAACCCCAAAAACGAA |
| | | wadei F0279 | | GGGATCTAAAAC (SEQ ID NO: |
| | | | | 404) |
| Cas13a13 | RcsCas13a | Rhodobacter | WP_013067728.1 | GCCTCACATCACCGCCAAGACGACG |
| | | capsulatus SB | | GCGGACTGAAC (SEQ ID NO: 405) |
| | | 1003 | | |
| Cas13a14 | RcrCas13a | Rhodobacter | WP_023911507.1 | GCCTCACATCACCGCCAAGACGACG |
| | | capsulatus R121 | | GCGGACTGAAC (SEQ ID NO: |
| | | | | 406) |
| Cas13a15 | RcdCas13a | Rhodobacter | WP_023911507.1 | GCCTCACATCACCGCCAAGACGACG |
| | | capsulatus | | GCGGACTGAAC (SEQ ID NO: |
| | | DE442 | | 407) |

Exemplary wild type Cas13a proteins of the disclosure may comprise or consist of the amino acid sequence of SEQ ID NO: 408.

Exemplary Cas13b proteins include, but are not limited to:

| | Cas13b | Cas13b |
|---|---|---|
| Species | Accession | Size (aa) |
| Paludibacter propionicigenes WB4 | WP_013446107.1 | 1155 |
| Prevotella sp. P5-60 | WP_044074780.1 | 1091 |
| Prevotella sp. P4-76 | WP_044072147.1 | 1091 |
| Prevotella sp. P5-125 | WP_044065294.1 | 1091 |
| Prevotella sp. P5-119 | WP_042518169.1 | 1091 |
| Capnocytophaga canimorsus Cc5 | WP_013997271.1 | 1200 |
| Phaeodactylibacter xiamenensis | WP_044218239.1 | 1132 |
| Porphyromonas gingivalis W83 | WP_005873511.1 | 1136 |
| Porphyromonas gingivalis F0570 | WP_021665475.1 | 1136 |
| Porphyromonas gingivalis ATCC 33277 | WP_012458151.1 | 1136 |
| Porphyromonas gingivalis F0185 | ERJ81987.1 | 1136 |
| Porphyromonas gingivalis F0185 | WP_021677657.1 | 1136 |
| Porphyromonas gingivalis SJD2 | WP_023846767.1 | 1136 |
| Porphyromonas gingivalis F0568 | ERJ65637.1 | 1136 |
| Porphyromonas gingivalis W4087 | ERJ87335.1 | 1136 |
| Porphyromonas gingivalis W4087 | WP_021680012.1 | 1136 |
| Porphyromonas gingivalis F0568 | WP_021663197.1 | 1136 |
| Porphyromonas gingivalis | WP_061156637.1 | 1136 |
| Porphyromonas gulae | WP_039445055.1 | 1136 |
| Bacteroides pyogenes F0041 | ERI81700.1 | 1116 |
| Bacteroides pyogenes JCM 10003 | WP_034542281.1 | 1116 |
| Alistipes sp. ZOR0009 | WP_047447901.1 | 954 |
| Flavobacterium branchiophilum FL-15 | WP_014084666.1 | 1151 |
| Prevotella sp. MA2016 | WP_036929175.1 | 1323 |
| Myroides odoratimimus CCUG 10230 | EHO06562.1 | 1160 |
| Myroides odoratimimus CCUG 3837 | EKB06014.1 | 1158 |
| Myroides odoratimimus CCUG 3837 | WP_006265509.1 | 1158 |
| Myroides odoratimimus CCUG 12901 | WP_006261414.1 | 1158 |
| Myroides odoratimimus CCUG 12901 | EHO08761.1 | 1158 |
| Myroides odoratimimus (NZ_CP013690.1) | WP_058700060.1 | 1160 |
| Bergeyella zoohelcum ATCC 43767 | EKB54193.1 | 1225 |
| Capnocytophaga cynodegmi | WP_041989581.1 | 1219 |
| Bergeyella zoohelcum ATCC 43767 | WP_002664492.1 | 1225 |
| Flavobacterium sp. 316 | WP_045968377.1 | 1156 |
| Psychroflexus torquis ATCC 700755 | WP_015024765.1 | 1146 |
| Flavobacterium columnare ATCC 49512 | WP_014165541.1 | 1180 |
| Flavobacterium columnare | WP_060381855.1 | 1214 |
| Flavobacterium columnare | WP_063744070.1 | 1214 |
| Flavobacterium columnare | WP_065213424.1 | 1215 |
| Chryseobacterium sp. YR477 | WP_047431796.1 | 1146 |
| Riemerella anatipestifer ATCC | WP_004919755.1 | 1096 |
| 11845 = DSM 15868 | | |
| Riemerella anatipestifer RA-CH-2 | WP_015345620.1 | 949 |
| Riemerella anatipestifer | WP_049354263.1 | 949 |
| Riemerella anatipestifer | WP_061710138.1 | 951 |
| Riemerella anatipestifer | WP_064970887.1 | 1096 |
| Prevotella saccharolytica F0055 | EKY00089.1 | 1151 |
| Prevotella saccharolytica JCM 17484 | WP_051522484.1 | 1152 |
| Prevotella buccae ATCC 33574 | EFU31981.1 | 1128 |
| Prevotella buccae ATCC 33574 | WP_004343973.1 | 1128 |
| Prevotella buccae D17 | WP_004343581.1 | 1128 |
| Prevotella sp. MSX73 | WP_007412163.1 | 1128 |
| Prevotella pallens ATCC 700821 | EGQ18444.1 | 1126 |
| Prevotella pallens ATCC 700821 | WP_006044833.1 | 1126 |
| Prevotella intermedia | WP_036860899.1 | 1127 |
| ATCC 25611 = DSM 20706 | | |
| Prevotella intermedia | WP_061868553.1 | 1121 |
| Prevotella intermedia 17 | AFJ07523.1 | 1135 |
| Prevotella intermedia | WP_050955369.1 | 1133 |
| Prevotella intermedia | BAU18623.1 | 1134 |
| Prevotella intermedia ZT | KJJ86756.1 | 1126 |
| Prevotella aurantiaca JCM 15754 | WP_025000926.1 | 1125 |
| Prevotella pleuritidis F0068 | WP_021584635.1 | 1140 |
| Prevotella pleuritidis JCM 14110 | WP_036931485.1 | 1117 |
| Prevotella falsenii DSM | WP_036884929.1 | 1134 |
| 22864 = JCM 15124 | | |
| Porphyromonas gulae | WP_039418912.1 | 1176 |
| Porphyromonas sp. COT-052 OH4946 | WP_039428968.1 | 1176 |
| Porphyromonas gulae | WP_039442171.1 | 1175 |
| Porphyromonas gulae | WP_039431778.1 | 1176 |
| Porphyromonas gulae | WP_046201018.1 | 1176 |
| Porphyromonas gulae | WP_039434803.1 | 1176 |
| Porphyromonas gulae | WP_039419792.1 | 1120 |
| Porphyromonas gulae | WP_039426176.1 | 1120 |
| Porphyromonas gulae | WP_039437199.1 | 1120 |
| Porphyromonas gingivalis TDC60 | WP_013816155.1 | 1120 |
| Porphyromonas gingivalis ATCC 33277 | WP_012458414.1 | 1120 |
| Porphyromonas gingivalis A7A1-28 | WP_058019250.1 | 1176 |
| Porphyromonas gingivalis JCVI SC001 | EOA10535.1 | 1176 |
| Porphyromonas gingivalis W50 | WP_005874195.1 | 1176 |
| Porphyromonas gingivalis | WP_052912312.1 | 1176 |
| Porphyromonas gingivalis AJW4 | WP_053444417.1 | 1120 |
| Porphyromonas gingivalis | WP_039417390.1 | 1120 |
| Porphyromonas gingivalis | WP_061156470.1 | 1120 |

Exemplary wild type Bergeyella zoohelcum ATCC 43767 Cas13b (BzCas13b) proteins of the disclosure may comprise or consist of the amino acid sequence of SEQ ID NO: 409.

In some embodiments of the compositions of the disclosure, the sequence encoding the RNA binding protein comprises a sequence isolated or derived from a Cas13d protein. Cas13d is an effector of the type VI-D CRISPR-Cas systems. In some embodiments, the Cas13d protein is an RNA-guided RNA endonuclease enzyme that can cut or bind RNA. In some embodiments, the Cas13d protein can include one or more higher eukaryotes and prokaryotes nucleotide-binding (HEPN) domains. In some embodiments, the Cas13d protein can include either a wild-type or mutated HEPN domain. In some embodiments, the Cas13d protein includes a mutated HEPN domain that cannot cut RNA but can process guide RNA. In some embodiments, the Cas13d protein does not require a protospacer flanking sequence. Also see WO Publication No. WO2019/040664 & US2019/0062724, which is incorporated herein by reference in its entirety, for further examples and sequences of Cas13d protein, without limitation.

In some embodiments, Cas13d sequences of the disclosure include without limitation SEQ ID NOS: 1-296 of WO 2019/040664, so numbered herein and included herewith.

SEQ ID NO: 1 is an exemplary Cas13d sequence from Eubacterium siraeum containing a HEPN site.

SEQ ID NO: 2 is an exemplary Cas13d sequence from Eubacterium siraeum containing a mutated HEPN site.

SEQ ID NO: 3 is an exemplary Cas13d sequence from uncultured Ruminococcus sp. containing a HEPN site.

SEQ ID NO: 4 is an exemplary Cas13d sequence from uncultured Ruminococcus sp. containing a mutated HEPN site.

SEQ ID NO: 5 is an exemplary Cas13d sequence from Gut_metagenome_contig2791000549.

SEQ ID NO: 6 is an exemplary Cas13d sequence from Gut_metagenome_contig855000317

SEQ ID NO: 7 is an exemplary Cas13d sequence from Gut_metagenome_contig3389000027.

SEQ ID NO: 8 is an exemplary Cas13d sequence from Gut_metagenome_contig8061000170.

SEQ ID NO: 9 is an exemplary Cas13d sequence from Gut_metagenome_contigl509000299.

SEQ ID NO: 10 is an exemplary Cas13d sequence from Gut_metagenome_contig9549000591.

SEQ ID NO: 11 is an exemplary Cas13d sequence from Gut_metagenome_contig71000500.

SEQ ID NO: 12 is an exemplary Cas13d sequence from human gut metagenome.

SEQ ID NO: 13 is an exemplary Cas13d sequence from Gut_metagenome_contig3915000357.

SEQ ID NO: 14 is an exemplary Cas13d sequence from Gut_metagenome_contig4719000173.

SEQ ID NO: 15 is an exemplary Cas13d sequence from Gut_metagenome_contig6929000468.

SEQ ID NO: 16 is an exemplary Cas13d sequence from Gut_metagenome_contig7367000486.

SEQ ID NO: 17 is an exemplary Cas13d sequence from Gut_metagenome_contig7930000403.

SEQ ID NO: 18 is an exemplary Cas13d sequence from Gut_metagenome_contig993000527.

SEQ ID NO: 19 is an exemplary Cas13d sequence from Gut_metagenome_contig6552000639.

SEQ ID NO: 20 is an exemplary Cas13d sequence from Gut_metagenome_contigl1932000246.

SEQ ID NO: 21 is an exemplary Cas13d sequence from Gut_metagenome_contigl2963000286.

SEQ ID NO: 22 is an exemplary Cas13d sequence from Gut_metagenome_contig2952000470.

SEQ ID NO: 23 is an exemplary Cas13d sequence from Gut_metagenome_contig451000394.

SEQ ID NO: 24 is an exemplary Cas13d sequence from Eubacterium_siraeum_DSM_15702.

SEQ ID NO: 25 is an exemplary Cas13d sequence from gut_metagenome_P19E0k2120140920,_c369000003.

SEQ ID NO: 26 is an exemplary Cas13d sequence from Gut_metagenome_contig7593000362.

SEQ ID NO: 27 is an exemplary Cas13d sequence from Gut_metagenome_contigl2619000055.

SEQ ID NO: 28 is an exemplary Cas13d sequence from Gut_metagenome_contigl405000151.

SEQ ID NO: 29 is an exemplary Cas13d sequence from Chicken_gut_metagenome_c298474.

SEQ ID NO: 30 is an exemplary Cas13d sequence from Gut_metagenome_contigl516000227.

SEQ ID NO: 31 is an exemplary Cas13d sequence from Gut_metagenome_contigl838000319.

SEQ ID NO: 32 is an exemplary Cas13d sequence from Gut_metagenome_contig13123000268.

SEQ ID NO: 33 is an exemplary Cas13d sequence from Gut_metagenome_contig5294000434.

SEQ ID NO: 34 is an exemplary Cas13d sequence from Gut_metagenome_contig6415000192.

SEQ ID NO: 35 is an exemplary Cas13d sequence from Gut_metagenome_contig6144000300.

SEQ ID NO: 36 is an exemplary Cas13d sequence from Gut_metagenome_contig9118000041.

SEQ ID NO: 37 is an exemplary Cas13d sequence from Activated_sludge_metagenome_transcript_124486.

SEQ ID NO: 38 is an exemplary Cas13d sequence from Gut_metagenome_contig1322000437.

SEQ ID NO: 39 is an exemplary Cas13d sequence from Gut_metagenome_contig4582000531.

SEQ ID NO: 40 is an exemplary Cas13d sequence from Gut_metagenome_contig9190000283.

SEQ ID NO: 41 is an exemplary Cas13d sequence from Gut_metagenome_contigl709000510.

SEQ ID NO: 42 is an exemplary Cas13d sequence from M24_(LSQX01212483_Anaerobic_digester_metagenome) with a HEPN domain.

SEQ ID NO: 43 is an exemplary Cas13d sequence from Gut_metagenome_contig3833000494.

SEQ ID NO: 44 is an exemplary Cas13d sequence from Activated_sludge_metagenome_transcript_117355.

SEQ ID NO: 45 is an exemplary Cas13d sequence from Gut_metagenome_contigl1061000330.

SEQ ID NO: 46 is an exemplary Cas13d sequence from Gut_metagenome_contig338000322 from sheep gut metagenome.

SEQ ID NO: 47 is an exemplary Cas13d sequence from human gut metagenome.

SEQ ID NO: 48 is an exemplary Cas13d sequence from Gut_metagenome_contig9530000097.

SEQ ID NO: 49 is an exemplary Cas13d sequence from Gut_metagenome_contigl750000258.

SEQ ID NO: 50 is an exemplary Cas13d sequence from Gut_metagenome_contig5377000274.

SEQ ID NO: 51 is an exemplary Cas13d sequence from gut_metagenome_P19E0k2120140920_c248000089.

SEQ ID NO: 52 is an exemplary Cas13d sequence from Gut_metagenome_contigll400000031.

SEQ ID NO: 53 is an exemplary Cas13d sequence from Gut_metagenome_contig7940000191.

SEQ ID NO: 54 is an exemplary Cas13d sequence from Gut_metagenome_contig6049000251.

SEQ ID NO: 55 is an exemplary Cas13d sequence from Gut_metagenome_contigl137000500.

SEQ ID NO: 56 is an exemplary Cas13d sequence from Gut_metagenome_contig9368000105.

SEQ ID NO: 57 is an exemplary Cas13d sequence from Gut_metagenome_contig546000275.

SEQ ID NO: 58 is an exemplary Cas13d sequence from Gut_metagenome_contig7216000573.

SEQ ID NO: 59 is an exemplary Cas13d sequence from Gut_metagenome_contig4806000409.

SEQ ID NO: 60 is an exemplary Cas13d sequence from Gut_metagenome_contig10762000480.

SEQ ID NO: 61 is an exemplary Cas13d sequence from Gut_metagenome_contig4114000374.

SEQ ID NO: 62 is an exemplary Cas13d sequence from Ruminococcus_flavefaciens_FD1.

SEQ ID NO: 63 is an exemplary Cas13d sequence from Gut_metagenome_contig7093000170.

SEQ ID NO: 64 is an exemplary Cas13d sequence from Gut_metagenome_contigl1113000384.

SEQ ID NO: 65 is an exemplary Cas13d sequence from Gut_metagenome_contig6403000259.

SEQ ID NO: 66 is an exemplary Cas13d sequence from Gut_metagenome_contig6193000124.

SEQ ID NO: 67 is an exemplary Cas13d sequence from Gut_metagenome_contig721000619.

SEQ ID NO: 68 is an exemplary Cas13d sequence from Gut_metagenome_contigl666000270.

SEQ ID NO: 69 is an exemplary Cas13d sequence from Gut_metagenome_contig2002000411.

SEQ ID NO: 70 is an exemplary Cas13d sequence from Ruminococcus_albus.

SEQ ID NO: 71 is an exemplary Cas13d sequence from Gut_metagenome_contig13552000311.

SEQ ID NO: 72 is an exemplary Cas13d sequence from Gut_metagenome_contig10037000527.

SEQ ID NO: 73 is an exemplary Cas13d sequence from Gut_metagenome_contig238000329.

SEQ ID NO: 74 is an exemplary Cas13d sequence from Gut_metagenome_contig2643000492.

SEQ ID NO: 75 is an exemplary Cas13d sequence from Gut_metagenome_contig874000057.

SEQ ID NO: 76 is an exemplary Cas13d sequence from Gut_metagenome_contig4781000489.

SEQ ID NO: 77 is an exemplary Cas13d sequence from Gut_metagenome_contigl2144000352.

SEQ ID NO: 78 is an exemplary Cas13d sequence from Gut_metagenome_contig5590000448.

SEQ ID NO: 79 is an exemplary Cas13d sequence from Gut_metagenome_contig9269000031.

SEQ ID NO: 80 is an exemplary Cas13d sequence from Gut_metagenome_contig8537000520.

SEQ ID NO: 81 is an exemplary Cas13d sequence from Gut_metagenome_contigl845000130.

SEQ ID NO: 82 is an exemplary Cas13d sequence from gut_metagenome_P13E0k2120140920_c3000072.

SEQ ID NO: 83 is an exemplary Cas13d sequence from gut_metagenome_P1 E0k2120140920_cI000078.

SEQ ID NO: 84 is an exemplary Cas13d sequence from Gut_metagenome_contigl2990000099.

SEQ ID NO: 85 is an exemplary Cas13d sequence from Gut_metagenome_contig525000349.

SEQ ID NO: 86 is an exemplary Cas13d sequence from Gut_metagenome_contig7229000302.

SEQ ID NO: 87 is an exemplary Cas13d sequence from Gut_metagenome_contig3227000343.

SEQ ID NO: 88 is an exemplary Cas13d sequence from Gut_metagenome_contig7030000469.

SEQ ID NO: 89 is an exemplary Cas13d sequence from Gut_metagenome_contig5149000068.

SEQ ID NO: 90 is an exemplary Cas13d sequence from Gut_metagenome_contig400200045.

SEQ ID NO: 91 is an exemplary Cas13d sequence from Gut_metagenome_contig10420000446.

SEQ ID NO: 92 is an exemplary Cas13d sequence from new_flavefaciens_strain_XPD3002 (CasRx).

SEQ ID NO: 93 is an exemplary Cas13d sequence from M26_Gut_metagenome_contig698000307.

SEQ ID NO: 94 is an exemplary Cas13d sequence from M36_Uncultured_Eubacterium_sp_TS28_c40956.

SEQ ID NO: 95 is an exemplary Cas13d sequence from M12_gut_metagenome_P25C0k2120140920_c134000066.

SEQ ID NO: 96 is an exemplary Cas13d sequence from human gut metagenome.

SEQ ID NO: 97 is an exemplary Cas13d sequence from MlO_gut_metagenome P25C90k2120 1 40920_c2800004 1.

SEQ ID NO: 98 is an exemplary Cas13d sequence from 30 Ml I_gut_metagenome_P25C7k2120140920_c4078000105.

SEQ ID NO: 99 is an exemplary Cas13d sequence from gut_metagenome_P25C0k2120140920_c32000045.

SEQ ID NO: 100 is an exemplary Cas13d sequence from M13_gut_metagenome P23C7k2120140920_c3000067.

SEQ ID NO: 101 is an exemplary Cas13d sequence from M5_gut_metagenome_Pl8E90k2120140920.

SEQ ID NO: 102 is an exemplary Cas13d sequence from M21_gut_metagenome_Pl8EMk2120140920.

SEQ ID NO: 103 is an exemplary Cas13d sequence from M7_gut_metagenome P38C7k2120 1 40920_c484 1 000003.

SEQ ID NO: 104 is an exemplary Cas13d sequence from Ruminococcus_bicirculans.

SEQ ID NO: 105 is an exemplary Cas13d sequence.

SEQ ID NO: 106 is an exemplary Cas13d consensus sequence.

SEQ ID NO: 107 is an exemplary Cas13d sequence from M18_gut_metagenome_P22EOk2120140920_c3395000078.

SEQ ID NO: 108 is an exemplary Cas13d sequence from M17_gut_metagenome_P22E90k2120140920_c 114.

SEQ ID NO: 109 is an exemplary Cas13d sequence from Ruminococcus_sp_CAG57.

SEQ ID NO: 110 is an exemplary Cas13d sequence from gut_metagenome_Pl 1E90k2120140920_c43000123.

SEQ ID NO: 111 is an exemplary Cas13d sequence from M6_gut_metagenome_P13E90k2120 1 40920_c7000009.

SEQ ID NO: 112 is an exemplary Cas13d sequence from M19_gut_metagenome_Pl 7E90k2120140920.

SEQ ID NO: 113 is an exemplary Cas13d sequence from gut_metagenome_P17E0k2120140920,_c87000043.

SEQ ID NO: 114 is an exemplary human codon optimized Eubacterium siraeum Cas13d nucleic acid sequence.

SEQ ID NO: 115 is an exemplary human codon optimized Eubacterium siraeum Cas13d nucleic acid sequence with a mutant HEPN domain.

SEQ ID NO: 116 is an exemplary human codon-optimized Eubacterium siraeum Cas13d nucleic acid sequence with N-terminal NLS.

SEQ ID NO: 117 is an exemplary human codon-optimized Eubacterium siraeum Cas13d nucleic acid sequence with N- and C-terminal NLS tags.

SEQ ID NO: 118 is an exemplary human codon-optimized uncultured Ruminococcus sp. Cas13d 30 nucleic acid sequence.

SEQ ID NO: 119 is an exemplary human codon-optimized uncultured Ruminococcus sp. Cas13d nucleic acid sequence with a mutant HEPN domain.

SEQ ID NO: 120 is an exemplary human codon-optimized uncultured Ruminococcus sp. Cas13d nucleic acid sequence with N-terminal NLS.

SEQ ID NO: 121 is an exemplary human codon-optimized uncultured Ruminococcus sp. Cas13d nucleic acid sequence with N- and C-terminal NLS tags.

SEQ ID NO: 122 is an exemplary human codon-optimized uncultured Ruminococcus flavefaciens FDl Cas13d nucleic acid sequence.

SEQ ID NO: 123 is an exemplary human codon-optimized uncultured Ruminococcus favefaciens FDl Cas13d nucleic acid sequence with mutated HEPN domain.

SEQ ID NO: 124 is an exemplary Cas13d nucleic acid sequence from Ruminococcus bicirculans.

SEQ ID NO: 125 is an exemplary Cas13d nucleic acid sequence from Eubacterium siraeum.

SEQ ID NO: 126 is an exemplary Cas13d nucleic acid sequence from Ruminococcus flavefaciens FD1.

SEQ ID NO: 127 is an exemplary Cas13d nucleic acid sequence from Ruminococcus albus.

SEQ ID NO: 128 is an exemplary Cas13d nucleic acid sequence from Ruminococcus flavefaciens XPD.

SEQ ID NO: 129 is an exemplary consensus DR nucleic acid sequence for E. siraeum Cas13d.

SEQ ID NO: 130 is an exemplary consensus DR nucleic acid sequence for Rum. Sp. Cas13d.

SEQ ID NO: 131 is an exemplary consensus DR nucleic acid sequence for Rum. Flavefaciens strain XPD3002 Cas13d (CasRx).

SEQ ID NOS: 132-137 are exemplary consensus DR nucleic acid sequences.

SEQ ID NO: 138 is an exemplary 50% consensus sequence for seven full-length Cas13d orthologues.

SEQ ID NO: 139 is an exemplary Cas13d nucleic acid sequence from Gut metagenome PlEO.

SEQ ID NO: 140 is an exemplary Cas13d nucleic acid sequence from Anaerobic digester.

SEQ ID NO: 141 is an exemplary Cas13d nucleic acid sequence from Ruminococcus sp. CAG:57.

SEQ ID NO: 142 is an exemplary human codon-optimized uncultured Gut metagenome PlEO Cas13d nucleic acid sequence.

SEQ ID NO: 143 is an exemplary human codon-optimized Anaerobic Digester Cas13d nucleic acid sequence.

SEQ ID NO: 144 is an exemplary human codon-optimized Ruminococcus flavefaciens XPD Cas13d nucleic acid sequence.

SEQ ID NO: 145 is an exemplary human codon-optimized Ruminococcus albus Cas13d nucleic acid sequence.

SEQ ID NO: 146 is an exemplary processing of the Ruminococcus sp. CAG:57 CRISPR array.

SEQ ID NO: 147 is an exemplary Cas13d protein sequence from contig emb |OBVH01003037.1, human gut metagenome sequence (also found in WGS contigs emb |OBXZ01000094.1| and emb |OBJFO1000033.1.

SEQ ID NO: 148 is an exemplary consensus DR nucleic acid sequence (goes with SEQ ID NO: 147).

SEQ ID NO: 149 is an exemplary Cas13d protein sequence from contig tpg |DBYI01000091.1| (Uncultivated Ruminococcus flavefaciens UBA1190 assembled from bovine gut metagenome).

SEQ ID NOS: 150-152 are exemplary consensus DR nucleic acid sequences (goes with SEQ ID NO: 149).

SEQ ID NO: 153 is an exemplary Cas13d protein sequence from contig tpg |DJXD01000002.1| (uncultivated Ruminococcus assembly, UBA7013, from sheep gutmetagenome).

SEQ ID NO: 154 is an exemplary consensus DR nucleic acid sequence (goes with SEQ ID NO: 153).

SEQ ID NO: 155 is an exemplary Cas13d protein sequence from contig OGZC01000639.1 (human gut metagenome assembly).

SEQ ID NOS: 156-177 are exemplary consensus DR nucleic acid sequences (goes with SEQ ID NO: 155).

SEQ ID NO: 158 is an exemplary Cas13d protein sequence from contig emb |OHBM01000764.1 (human gut metagenome assembly).

SEQ ID NO: 159 is an exemplary consensus DR nucleic acid sequence (goes with SEQ ID NO: 158).

SEQ ID NO: 160 is an exemplary Cas13d protein sequence from contig emb |0HCP01000044.1 (human gut metagenome assembly).

SEQ ID NO: 161 is an exemplary consensus DR nucleic acid sequence (goes with SEQ ID NO: 160).

SEQ ID NO: 162 is an exemplary Cas13d protein sequence from contig embl0GDF01008514.1 (human gut metagenome assembly).

SEQ ID NO: 163 is an exemplary consensus DR nucleic acid sequence (goes with SEQ ID NO: 162).

SEQ ID NO: 164 is an exemplary Cas13d protein sequence from contig emb |0GPN01002610.1 (human gut metagenome assembly).

SEQ ID NO: 165 is an exemplary consensus DR nucleic acid sequence (goes with SEQ ID NO: 164).

SEQ ID NO: 166 is an exemplary Cas13d protein sequence from contig NFIR01000008. 1 (Eubacterium sp. An3, from chicken gut metagenome).

SEQ ID NO: 167 is an exemplary consensus DR nucleic acid sequence (goes with SEQ ID NO: 166).

SEQ ID NO: 168 is an exemplary Cas13d protein sequence from contig NFLV01000009.1 (Eubacterium sp. An11 from chicken gut metagenome).

SEQ ID NO: 169 is an exemplary consensus DR nucleic acid sequence (goes with SEQ ID NO: 168).

SEQ ID NOS: 171-174 are an exemplary Cas13d motif sequences.

SEQ ID NO: 175 is an exemplary Cas13d protein sequence from contig OJMM01002900 human gut metagenome sequence.

SEQ ID NO: 176 is an exemplary consensus DR nucleic acid sequence (goes with SEQ ID NO: 175).

SEQ ID NO: 177 is an exemplary Cas13d protein sequence from contig ODAI011611274.1 gut metagenome sequence.

SEQ ID NO: 178 is an exemplary consensus DR nucleic acid sequence (goes with SEQ ID NO: 177).

SEQ ID NO: 179 is an exemplary Cas13d protein sequence from contig OIZX01000427.1.

SEQ ID NO: 180 is an exemplary consensus DR nucleic acid sequence (goes with SEQ ID NO: 179).

SEQ ID NO: 181 is an exemplary Cas13d protein sequence from contig emb |OCVV012889144.1|.

SEQ ID NO: 182 is an exemplary consensus DR nucleic acid sequence (goes with SEQ ID NO: 181).

SEQ ID NO: 183 is an exemplary Cas13d protein sequence from contig OCTW011587266.1

SEQ ID NO: 184 is an exemplary consensus DR nucleic acid sequence (goes with SEQ ID NO: 183).

SEQ ID NO: 185 is an exemplary Cas13d protein sequence from contig emb |OGNFO 1009141.1.

SEQ ID NO: 186 is an exemplary consensus DR nucleic acid sequence (goes with SEQ ID NO: 185).

SEQ ID NO: 187 is an exemplary Cas13d protein sequence from contig emb |OIEN01002196.1.

SEQ ID NO: 188 is an exemplary consensus DR nucleic acid sequence (goes with SEQ ID NO: 187).

SEQ ID NO: 189 is an exemplary Cas13d protein sequence from contig e-k87_11092736.

SEQ ID NOS: 190-193 are exemplary consensus DR nucleic acid sequences (goes with SEQ ID NO: 189).

SEQ ID NO: 194 is an exemplary Cas13d sequence from Gut_metagenome_contig6893000291.

SEQ ID NOS: 195-197 are exemplary Cas13d motif sequences.

SEQ ID NO: 198 is an exemplary Cas13d protein sequence from Ga0224415_10007274.

SEQ ID NO: 199 is an exemplary consensus DR nucleic acid sequence (goes with SEQ ID NO: 198).

SEQ ID NO: 200 is an exemplary Cas13d protein sequence from EMG_10003641.

SEQ ID NO: 202 is an exemplary Cas13d protein sequence from Ga0129306_1000735.

SEQ ID NO: 201 is an exemplary consensus DR nucleic acid sequence (goes with SEQ ID NO: 200).

SEQ ID NO: 202 is an exemplary Cas13d protein sequence from Ga0129306_1000735.

SEQ ID NO: 203 is an exemplary consensus DR nucleic acid sequence (goes with SEQ ID NO: 203

SEQ ID NO: 204 is an exemplary Cas13d protein sequence from GaO129317_1 008067.

SEQ ID NO: 205 is an exemplary consensus DR nucleic acid sequence (goes with SEQ ID NO: 204).

SEQ ID NO: 206 is an exemplary Cas13d protein sequence from Ga0224415_10048792.

SEQ ID NO: 207 is an exemplary consensus DR nucleic acid sequence (goes with SEQ ID NO: 206).

SEQ ID NO: 208 is an exemplary Cas13d protein sequence from 160582958_gene49834.

SEQ ID NO: 209 is an exemplary consensus DR nucleic acid sequence (goes with SEQ ID NO: 208).

SEQ ID NO: 210 is an exemplary Cas13d protein sequence from 250twins_35838_GLOI10300.

SEQ ID NO: 211 is an exemplary consensus DR nucleic acid sequence (goes with SEQ ID NO: 210).

SEQ ID NO: 212 is an exemplary Cas13d protein sequence from 250twins_36050_GLOI58985.

SEQ ID NO: 213 is an exemplary consensus DR nucleic acid sequence (goes with SEQ ID NO: 212).

SEQ ID NO: 214 is an exemplary Cas13d protein sequence from 31009_GL0034153.

SEQ ID NO: 215 is an exemplary consensus DR nucleic acid sequence (goes with SEQ ID NO: 214).

SEQ ID NO: 216 is an exemplary Cas13d protein sequence from 530373_GL0023589.

SEQ ID NO: 217 is an exemplary consensus DR nucleic acid sequence (goes with SEQ ID NO: 216).

SEQ ID NO: 218 is an exemplary Cas13d protein sequence from BMZ-l 1B_GL0037771.

SEQ ID NO: 219 is an exemplary consensus DR nucleic acid sequence (goes with SEQ ID NO: 218).

SEQ ID NO: 220 is an exemplary Cas13d protein sequence from BMZ-l 1B_GL0037915.

SEQ ID NO: 221 is an exemplary consensus DR nucleic acid sequence (goes with SEQ ID NO: 220).

SEQ ID NO: 222 is an exemplary Cas13d protein sequence from BMZ-l 1B_GL00696 1 7.

SEQ ID NO: 223 is an exemplary consensus DR nucleic acid sequence (goes with SEQ ID NO: 222).

SEQ ID NO: 224 is an exemplary Cas13d protein sequence from DLF014_GL0011914.

SEQ ID NO: 225 is an exemplary consensus DR nucleic acid sequence (goes with SEQ ID NO: 224).

SEQ ID NO: 226 is an exemplary Cas13d protein sequence from EYZ-362B_GL0088915.

SEQ ID NO: 227-228 are exemplary consensus DR nucleic acid sequences (goes with SEQ ID NO: 226).

SEQ ID NO: 229 is an exemplary Cas13d protein sequence from Ga0099364 10024192.

SEQ ID NO: 230 is an exemplary consensus DR nucleic acid sequence (goes with SEQ ID NO: 229).

SEQ ID NO: 231 is an exemplary Cas13d protein sequence from Ga0187910_10006931.

SEQ ID NO: 232 is an exemplary consensus DR nucleic acid sequence (goes with SEQ ID NO: 231).

SEQ ID NO: 233 is an exemplary Cas13d protein sequence from Ga0187910_10015336.

SEQ ID NO: 234 is an exemplary consensus DR nucleic acid sequence (goes with SEQ ID NO: 233).

SEQ ID NO: 235 is an exemplary Cas13d protein sequence from Ga0187910_10040531.

SEQ ID NO: 236 is an exemplary consensus DR nucleic acid sequence (goes with SEQ ID NO: 23).

SEQ ID NO: 237 is an exemplary Cas13d protein sequence from Ga0187911_10069260.

SEQ ID NO: 238 is an exemplary consensus DR nucleic acid sequence (goes with SEQ ID NO: 237).

SEQ ID NO: 239 is an exemplary Cas13d protein sequence from MH0288_GL0082219.

SEQ ID NO: 240 is an exemplary consensus DR nucleic acid sequence (goes with SEQ ID NO: 239).

SEQ ID NO: 241 is an exemplary Cas13d protein sequence from O2.UC29-0_GL0096317.

SEQ ID NO: 242 is an exemplary consensus DR nucleic acid sequence (goes with SEQ ID NO: 241).

SEQ ID NO: 243 is an exemplary Cas13d protein sequence from PIG-014_GL0226364.

SEQ ID NO: 244 is an exemplary consensus DR nucleic acid sequence (goes with SEQ ID NO: 243).

SEQ ID NO: 245 is an exemplary Cas13d protein sequence from PIG-018_GL0023397.

SEQ ID NO: 246 is an exemplary consensus DR nucleic acid sequence (goes with SEQ ID NO: 245).

SEQ ID NO: 247 is an exemplary Cas13d protein sequence from PIG-025_GL0099734.

SEQ ID NO: 248 is an exemplary consensus DR nucleic acid sequence (goes with SEQ ID NO: 247).

SEQ ID NO: 249 is an exemplary Cas13d protein sequence from PIG-028_GL0185479.

SEQ ID NO: 250 is an exemplary consensus DR nucleic acid sequence (goes with SEQ ID NO: 249).

SEQ ID NO: 251 is an exemplary Cas13d protein sequence from −Ga0224422_10645759.

SEQ ID NO: 252 is an exemplary consensus DR nucleic acid sequence (goes with SEQ ID NO: 251).

SEQ ID NO: 253 is an exemplary Cas13d protein sequence from ODAI chimera.

SEQ ID NO: 254 is an exemplary consensus DR nucleic acid sequence (goes with SEQ ID NO: 253).

SEQ ID NO: 255 is an HEPN motif.

SEQ ID NOs: 256 and 257 are exemplary Cas13d nuclear localization signal amino acid and nucleic acid sequences, respectively.

SEQ ID NOs: 258 and 260 are exemplary SV40 large T antigen nuclear localization signal amino acid and nucleic acid sequences, respectively.

SEQ ID NO: 259 is a dCas9 target sequence.

SEQ ID NO: 261 is an artificial Eubacterium siraeum nCasl array targeting ccdB.

SEQ ID NO: 262 is a full 36 nt direct repeat.

SEQ ID NOs: 263-266 are spacer sequences.

SEQ ID NO: 267 is an artificial uncultured Ruminoccus sp. nCasl array targeting ccdB.

SEQ ID NO: 268 is a full 36 nt direct repeat.

SEQ ID NOs: 269-272 are spacer sequences.

SEQ ID NO: 273 is a ccdB target RNA sequence.

SEQ ID NOs: 274-277 are spacer sequences.

SEQ ID NO: 278 is a mutated Cas13d sequence, NLS-Ga_0531(trunc)-NLS-HA. This mutant has a deletion of the non-conserved N-terminus.

SEQ ID NO: 279 is a mutated Cas13d sequence, NES-Ga_0531(trunc)-NES-HA. This mutant has a deletion of the non-conserved N-terminus.

SEQ ID NO: 280 is a full-length Cas13d sequence, NLS-RfxCas13d-NLS-HA.

SEQ ID NO: 281 is a mutated Cas13d sequence, NLS-RfxCas13d(del5)-NLS-HA. This mutant has a deletion of amino acids 558-587.

SEQ ID NO: 282 is a mutated Cas13d sequence, NLS-RfxCas13d(del5.12)-NLS-HA. This mutant has a deletion of amino acids 558-587 and 953-966.

SEQ ID NO: 283 is a mutated Cas13d sequence, NLS-RfxCas13d(del5.13)-NLS-HA. This mutant has a deletion of amino acids 376-392 and 558-587.

SEQ ID NO: 284 is a mutated Cas13d sequence, NLS-RfxCas13d(del5.12+5. 13)-NLS-HA. This mutant has a deletion of amino acids 376-392, 558-587, and 953-966.

SEQ ID NO: 285 is a mutated Cas13d sequence, NLS-RfxCas13d(dell3)-NLS-HA. This mutant has a deletion of amino acids 376-392.

SEQ ID NO: 286 is an effector sequence used to edit expression of ADAR2. Amino acids 1 to 969 are dRfxCas13, aa 970 to 991 are an NLS sequence, and amino acids 992 to 1378 are ADAR2DD.

SEQ ID NO: 287 is an exemplary HIV NES protein sequence.

SEQ ID NOS: 288-291 are exemplary Cas13d motif sequences.

SEQ ID NO: 292 is Cas13d ortholog sequence MH_4866.

SEQ ID NO: 293 is an exemplary Cas13d protein sequence from 037_-_emblOIZA01000315.11

SEQ ID NO: 294 is an exemplary Cas13d protein sequence from PIG-022 GL002635 1.

SEQ ID NO: 295 is an exemplary Cas13d protein sequence from PIG-046_GL0077813.

SEQ ID NO: 296 is an exemplary Cas13d protein sequence from pig chimera.

SEQ ID NO: 297 is an exemplary nuclease-inactive or dead Cas13d (dCas13d) protein sequence from Ruminococcus flavefaciens XPD3002 (CasRx)

SEQ ID NO: 298 is an exemplary Cas13d protein sequence.

SEQ ID NO: 299 is an exemplary Cas13d protein sequence from (contig tpg|DJXD01000002.1|; uncultivated Ruminococcus assembly, UBA7013, from sheep gut metagenome).

SEQ ID NO: 300 is an exemplary Cas13d direct repeat nucleotide sequence from Cas13d (contig tpg|DJXD01000002.1|; uncultivated Ruminococcus assembly, UBA7013, from sheep gut metagenome (goes with SEQ ID NO: 299).

SEQ ID NO: 301 is an exemplary Cas13d protein contig emb|OBLI01020244.

Yan et al. (2018) Mol Cell. 70(2):327-339 (doi: 10.1016/j.molcel.2018.02.2018) and Konermann et al. (2018) Cell 173(3):665-676 (doi: 10.1016/j.cell/2018.02.033) have described Cas13d proteins and both of which are incorporated by reference herein in their entireties. Also see WO Publication Nos. WO2018/183403 (CasM, which is Cas13d) and WO2019/006471 (Cas13d), which are incorporated herein by reference in their entirety.

SEQ ID NO: 587 is an exemplary casl3d with no catalytic activity, referred to as deactivatedCas13d or dCas13d.

SEQ ID NO: 590 is an exemplary cas13d with no catalytic activity, referred to as deactivatedCas13d or dCas13d.

SEQ ID NO: 591 is an exemplary cas13d with no catalytic activity, referred to as deactivatedCas13d or dCas13d.

SEQ ID NO: 592 is an exemplary cas13d with no catalytic activity, referred to as deactivatedCas13d or dCas13d.

SEQ ID NO: 593 is an exemplary cas13d with no catalytic activity, referred to as deactivatedCas13d or dCas13d.

SEQ ID NO: 594 is an exemplary cas13d with no catalytic activity, referred to as deactivatedCas13d or dCas13d.

SEQ ID NO: 303 is an exemplary CasM protein from Eubacterium siraeum.

SEQ ID NO: 304 is an exemplary CasM protein from Ruminococcus sp., isolate 2789STDY5834971.

SEQ ID NO: 305 is an exemplary CasM protein from Ruminococcus bicirculans.

SEQ ID NO: 306 is an exemplary CasM protein from Ruminococcus sp., isolate 2789STDY5608892.

SEQ ID NO: 307 is an exemplary CasM protein from Ruminococcus sp. CAG:57.

SEQ ID NO: 308 is an exemplary CasM protein from Ruminococcus flavefaciens FD-1.

SEQ ID NO: 309 is an exemplary CasM protein from Ruminococcus albus strain KH2T6.

SEQ ID NO: 310 is an exemplary CasM protein from Ruminococcus flavefaciens strain XPD3002.

SEQ ID NO: 311 is an exemplary CasM protein from Ruminococcus sp., isolate 2789STDY5834894.

SEQ ID NO: 312 is an exemplary RtcB homolog.

SEQ ID NO: 313 is an exemplary WYL from Eubacterium siraeum+C-terminal NLS.

SEQ ID NO: 314 is an exemplary WYL from Ruminococcus sp. isolate 2789STDY5834971+C-term NLS.

SEQ ID NO: 315 is an exemplary WYL from Ruminococcus bicirculans+C-term NLS.

SEQ ID NO: 316 is an exemplary WYL from Ruminococcus sp. isolate 2789STDY5608892+C-term NLS.

SEQ ID NO: 317 is an exemplary WYL from Ruminococcus sp. CAG:57+C-term NLS.

SEQ ID NO: 318 is an exemplary WYL from Ruminococcus flavefaciens FD-1+C-term NLS.

SEQ ID NO: 319 is an exemplary WYL from Ruminococcus albus strain KH2T6+C-term NLS.

SEQ ID NO: 320 is an exemplary WYL from Ruminococcus flavefaciens strain XPD3002+C-term NLS.

SEQ ID NO: 321 is an exemplary RtcB from Eubacterium siraeum+C-term NLS.

SEQ ID NO: 322 is an exemplary direct repeat sequence of Ruminococcus flavefaciens XPD3002 Cas13d (CasRx).

Exemplary wild type Cas13d proteins of the disclosure may comprise or consist of the amino acid sequence SEQ ID NO: 92 or SEQ ID NO: 298 (Cas13d protein also known as CasRx).

An exemplary direct repeat sequence of Ruminococcus flavefaciens XPD3002 Cas13d (CasRx) comprises the nucleic acid sequence:

(SEQ ID NO: 302)
AACCCCTACCAACTGGTCGGGGTTTGAAAC.

gRNA Target Sequences

The compositions of the disclosure bind and destroy a target sequence of an RNA molecule comprising a pathogenic repeat sequence. In one embodiment, the target RNA comprises a sequence motif corresponding to a spacer sequence of the guide RNA corresponding to the RNA-guided RNA-binding protein. In some embodiments, one or more spacer sequences are used to target one or more target sequences. In some embodiments, multiple spacers are used to target multiple target RNAs. Such target RNAs can be different target sites within the same RNA molecule or can be different target sites within different RNA molecules. Spacer sequences can also target non-coding RNA. In some embodiments, multiple promoters, e.g., Pol III promoters) can be used to drive multiple spacers in a gRNA for targeting multiple target RNAs. In one embodiment, the destruction of the target RNA(s) or target sequence motif(s) reduces expression of pathogenic CAG repeat RNA thereby treating CAG repeat disease such as HD or SCA1 and/or ameliorating one or more symptoms associated with CAG repeat diseases such as HD or SCA1.

In some embodiments of the compositions and methods of the disclosure, the sequence motif of the target RNA is a signature of a disease or disorder.

A sequence motif of the disclosure may be isolated or derived from a sequence of foreign or exogenous sequence found in a genomic sequence, and therefore translated into an mRNA molecule of the disclosure or a sequence of foreign or exogenous sequence found in an RNA sequence of the disclosure.

A target sequence motif of the disclosure may comprise, consist of, be situated by, or be associated with a mutation in an endogenous sequence that causes a disease or disorder. The mutation may comprise or consist of a sequence substitution, inversion, deletion, insertion, transposition, or any combination thereof.

A target sequence motif of the disclosure may comprise or consist of a repeated sequence. In some embodiments, the repeated sequence may be associated with a microsatellite instability (MSI). MSI at one or more loci results from impaired DNA mismatch repair mechanisms of a cell of the disclosure. A hypervariable sequence of DNA may be transcribed into an mRNA of the disclosure comprising a target sequence comprising or consisting of the hypervariable sequence.

A target sequence motif of the disclosure may comprise or consist of a biomarker. The biomarker may indicate a risk of developing a disease or disorder. The biomarker may indicate a healthy gene (low or no determinable risk of developing a disease or disorder. The biomarker may indicate an edited gene. Exemplary biomarkers include, but are not limited to, single nucleotide polymorphisms (SNPs), sequence variations or mutations, epigenetic marks, splice acceptor sites, exogenous sequences, heterologous sequences, and any combination thereof.

A target sequence motif of the disclosure may comprise or consist of a secondary, tertiary or quaternary structure. The secondary, tertiary or quaternary structure may be endogenous or naturally occurring. The secondary, tertiary or quaternary structure may be induced or non-naturally occurring. The secondary, tertiary or quaternary structure may be encoded by an endogenous, exogenous, or heterologous sequence.

In some embodiments of the compositions and methods of the disclosure, a target sequence of an RNA molecule comprises or consists of between 2 and 100 nucleotides or nucleic acid bases, inclusive of the endpoints. In some embodiments, the target sequence of an RNA molecule comprises or consists of between 2 and 50 nucleotides or nucleic acid bases, inclusive of the endpoints. In some embodiments, the target sequence of an RNA molecule comprises or consists of between 2 and 20 nucleotides or nucleic acid bases, inclusive of the endpoints. In some embodiments, the target sequence of an RNA molecule comprises or consists of between 20-30 nucleotides or nucleic acid bases, inclusive of the endpoints. In some embodiments, the target sequence of an RNA molecule comprises or consists of about 26 nucleotides or nucleic acid bases, inclusive of the endpoints.

In some embodiments of the compositions and methods of the disclosure, a target sequence of an RNA molecule is continuous. In some embodiments, the target sequence of an RNA molecule is discontinuous. For example, the target sequence of an RNA molecule may comprise or consist of one or more nucleotides or nucleic acid bases that are not contiguous because one or more intermittent nucleotides are positioned in between the nucleotides of the target sequence.

In some embodiments of the compositions and methods of the disclosure, a target sequence of an RNA molecule is naturally occurring. In some embodiments, the target sequence of an RNA molecule is non-naturally occurring. Exemplary non-naturally occurring target sequences may comprise or consist of sequence variations or mutations, chimeric sequences, exogenous sequences, heterologous sequences, chimeric sequences, recombinant sequences, sequences comprising a modified or synthetic nucleotide or any combination thereof.

In some embodiments of the compositions and methods of the disclosure, a target sequence of an RNA molecule binds to a guide RNA of the disclosure. In some embodiments of the compositions and methods of the disclosure, one or more target sequences of an RNA molecule binds to one or more guide RNA spacer sequences of the disclosure.

In some embodiments of the compositions and methods of the disclosure, a target sequence of an RNA molecule binds to a first RNA binding protein of the disclosure.

In some embodiments of the compositions and methods of the disclosure, a target sequence of an RNA molecule binds to a second RNA binding protein of the disclosure.

Compositions of the disclosure comprise a gRNA comprising a spacer sequence that specifically binds to a target toxic CAG RNA repeat sequence. In some embodiments, the spacer which binds the target CAG RNA repeat sequence comprises or consists of about 20-30 nucleotides. In some embodiments, a gRNA comprises one or more spacer sequences.

Exemplary gRNA spacer sequences of the disclosure that specifically bind to a target CAG sequence of an RNA molecule are SEQ ID NOs 457-459.

### Endonucleases

In some embodiments, the compositions of the disclosure comprise a second RNA binding protein which comprises or consists of a nuclease or endonuclease domain. In some embodiments, the second RNA-binding protein is an effector protein. In some embodiments, the second RNA binding protein binds RNA in a manner in which it associates with RNA. In some embodiments, the second RNA binding protein associates with RNA in a manner in which it cleaves RNA. In some embodiments, the second RNA-binding protein is fused to a first RNA-binding protein which is a PUF, PUMBY, or PPR-based protein. In one embodiment, the second RNA-binding protein is fused to a first RNA-binding protein which is a catalytically deactivated Cas-based (dCas-based) protein.

In some embodiments of the compositions of the disclosure, the second RNA binding protein comprises or consists of an RNase.

In some embodiments, the second RNA binding protein comprises or consists of an RNase1. In some embodiments, the RNase1 protein comprises or consists of SEQ ID NO: 325.

In some embodiments, the second RNA binding protein comprises or consists of an RNase4. In some embodiments, the RNase4 protein comprises or consists of SEQ ID NO: 326.

In some embodiments, the second RNA binding protein comprises or consists of an RNase6. In some embodiments, the RNase6 protein comprises or consists of SEQ ID NO: 327.

In some embodiments, the second RNA binding protein comprises or consists of an RNase7. In some embodiments, the RNase7 protein comprises or consists of SEQ ID NO: 328.

In some embodiments, the second RNA binding protein comprises or consists of an RNase8. In some embodiments, the RNase8 protein comprises or consists of SEQ ID NO: 329.

In some embodiments, the second RNA binding protein comprises or consists of an RNase2. In some embodiments, the RNase2 protein comprises or consists of SEQ ID NO: 330.

In some embodiments, the second RNA binding protein comprises or consists of an RNase6PL. In some embodiments, the RNase6PL protein comprises or consists of SEQ ID NO: 331.

In some embodiments, the second RNA binding protein comprises or consists of an RNaseL. In some embodiments, the RNaseL protein comprises or consists of SEQ ID NO: 332.

In some embodiments, the second RNA binding protein comprises or consists of an RNaseT2. In some embodiments, the RNaseT2 protein comprises or consists of SEQ ID NO: 333.

In some embodiments, the second RNA binding protein comprises or consists of an RNase1 1. In some embodiments, the RNase1 1 protein comprises or consists of SEQ ID NO: 334.

In some embodiments, the second RNA binding protein comprises or consists of an RNaseT2-like. In some embodiments, the RNaseT2-like protein comprises or consists of SEQ ID NO: 335.

In some embodiments of the compositions of the disclosure, the second RNA binding protein comprises or consists of a mutated RNase.

In some embodiments, the second RNA binding protein comprises or consists of a mutated RNase1 (RNase1(K41R)) polypeptide. In some embodiments, the RNase1(K41R) polypeptide comprises or consists of SEQ ID NO: 336.

In some embodiments, the second RNA binding protein comprises or consists of a mutated RNase1 (RNase1(K41R, D121E)) polypeptide. In some embodiments, the RNase1 (RNase1(K41R, D121E)) polypeptide comprises or consists of SEQ ID NO: 337.

In some embodiments, the second RNA binding protein comprises or consists of a mutated RNase1 (RNase1(K41R, D121E, H119N)) polypeptide. In some embodiments, the RNase1 (RNase1(K41R, D121E, H119N)) polypeptide comprises or consists of SEQ ID NO: 338.

In some embodiments, the second RNA binding protein comprises or consists of a mutated RNase1. In some embodiments, the second RNA binding protein comprises or consists of a mutated RNase1 (RNase1(H119N)) polypeptide. In some embodiments, the RNase1 (RNase1(H119N)) polypeptide comprises or consists of SEQ ID NO: 339.

In some embodiments, the second RNA binding protein comprises or consists of a mutated RNase1 (RNase1(R39D, N67D, N88A, G89D, R91D, H119N)) polypeptide.

In some embodiments, the RNase1 (RNase1(R39D, N67D, N88A, G89D, R91D, H119N)) polypeptide comprises or consists of SEQ ID NO: 340.

In some embodiments, the second RNA binding protein comprises or consists of a mutated RNase1 (RNase1(R39D, N67D, N88A, G89D, R91D, H, 119N)) polypeptide. In some embodiments, the RNase1 (RNase1(R39D, N67D, N88A, G89D, R91D, H119N, K41R, D121E)) polypeptide comprises or consists of SEQ ID NO: 341.

In some embodiments, the second RNA binding protein comprises or consists of a mutated RNase1 (RNase1(R39D, N67D, N88A, G89D, R91D, H119N)) polypeptide. In some embodiments, the RNase1 (RNase1(R39D, N67D, N88A, G89D, R91D)) polypeptide comprises or consists of SEQ ID NO: 342.

In some embodiments, the second RNA binding protein comprises or consists of a mutated RNase1 (RNase1 (R39D, N67D, N88A, G89D, R91D, H119N, K41R, D121E)) polypeptide that comprises or consists of SEQ ID NO: 343.

In some embodiments of the compositions of the disclosure, the second RNA binding protein comprises or consists of a NOB1 polypeptide. In some embodiments, the NOB1 polypeptide comprises or consists of SEQ ID NO: 344.

In some embodiments of the compositions of the disclosure, the second RNA binding protein comprises or consists of an endonuclease. In some embodiments, the second RNA binding protein comprises or consists of an endonuclease V (ENDOV). In some embodiments, the ENDOV protein comprises or consists of SEQ ID NO: 345.

In some embodiments, the second RNA binding protein comprises or consists of an endonuclease G (ENDOG). In some embodiments, the ENDOG protein comprises or consists of SEQ ID NO: 346.

In some embodiments, the second RNA binding protein comprises or consists of an endonuclease D1 (ENDOD1). In some embodiments, the ENDOD1 protein comprises or consists of SEQ ID NO: 347.

In some embodiments, the second RNA binding protein comprises or consists of a Human flap endonuclease-1 (hFEN1). In some embodiments, the hFEN1 polypeptide comprises or consists of SEQ ID NO: 348.

In some embodiments, the second RNA binding protein comprises or consists of a DNA repair endonuclease XPF (ERCC4) polypeptide. In some embodiments, the ERCC4 polypeptide comprises or consists of SEQ ID NO: 349.

In some embodiments of the compositions of the disclosure, the second RNA binding protein comprises or consists of an Endonuclease III-like protein 1 (NTHL) polypeptide. In some embodiments, the NTHL polypeptide comprises or consists of SEQ ID NO: 340.

In some embodiments of the compositions of the disclosure, the second RNA binding protein comprises or consists of a human Schlafen 14 (hSLFN14) polypeptide. In some embodiments, the hSLFN14 polypeptide comprises or consists of SEQ ID NO: 351.

In some embodiments of the compositions of the disclosure, the second RNA binding protein comprises or consists of a human beta-lactamase-like protein 2 (hLACTB2) polypeptide. In some embodiments, the hLACTB2 polypeptide comprises or consists of SEQ ID NO: 352.

In some embodiments of the compositions of the disclosure, the second RNA binding protein comprises or consists of an apurinic/apyrimidinic (AP) endodeoxyribonuclease (APEX) polypeptide. In some embodiments, the second RNA binding protein comprises or consists of an apurinic/apyrimidinic (AP) endodeoxyribonuclease (APEX2) polypeptide. In some embodiments, the APEX2 polypeptide comprises or consists of SEQ ID NO: 353.

In some embodiments, the APEX2 polypeptide comprises or consists of SEQ ID NO: 354.

In some embodiments, the second RNA binding protein comprises or consists of an apurinic or apyrimidinic site lyase (APEX1) polypeptide. In some embodiments, the APEX1 polypeptide comprises or consists of SEQ ID NO: 355.

In some embodiments of the compositions of the disclosure, the second RNA binding protein comprises or consists of an angiogenin (ANG) polypeptide. In some embodiments, the ANG polypeptide comprises or consists of SEQ ID NO: 356.

In some embodiments of the compositions of the disclosure, the second RNA binding protein comprises or consists of a heat responsive protein 12 (HRSP12) polypeptide. In some embodiments, the HRSP12 polypeptide comprises or consists of SEQ ID NO: 357.

In some embodiments of the compositions of the disclosure, the second RNA binding protein comprises or consists of a Zinc Finger CCCH-Type Containing 12A (ZC3H12A) polypeptide. In some embodiments, the ZC3H12A polypeptide is an endonuclease domain of the ZC3H12A polypeptide which comprises or consists of SEQ ID NO: 358, also referred to as E17 herein. In some embodiments, the ZC3H12A polypeptide comprises or consists of SEQ ID NO: 359.

In some embodiments of the compositions of the disclosure, the second RNA binding protein comprises or consists of a Reactive Intermediate Imine Deaminase A (RIDA) polypeptide. In some embodiments, the RIDA polypeptide comprises or consists of SEQ ID NO: 360.

In some embodiments of the compositions of the disclosure, the second RNA binding protein comprises or consists of a Phospholipase D Family Member 6 (PDL6) polypeptide. In some embodiments, the PDL6 polypeptide comprises or consists of SEQ ID NO: 361.

In some embodiments of the compositions of the disclosure, the second RNA binding protein comprises or consists of a mitochondrial ribonuclease P catalytic subunit (KIAA0391) polypeptide. In some embodiments, the KIAA0391 polypeptide comprises or consists of SEQ ID NO: 362.

In some embodiments of the compositions of the disclosure, the second RNA binding protein comprises or consists of an argonaute 2 (AGO2) polypeptide. In some embodiments of the compositions of the disclosure, the AGO2 polypeptide comprises or consists of SEQ ID NO: 363.

In some embodiments of the compositions of the disclosure, the second RNA binding protein comprises or consists of a mitochondrial nuclease EXOG (EXOG) polypeptide. In some embodiments, the EXOG polypeptide comprises or consists of SEQ ID NO: 364.

In some embodiments of the compositions of the disclosure, the second RNA binding protein comprises or consists of a Zinc Finger CCCH-Type Containing 12D (ZC3H12D) polypeptide. In some embodiments, the ZC3H12D polypeptide comprises or consists of SEQ ID NO: 365.

In some embodiments of the compositions of the disclosure, the second RNA binding protein comprises or consists of an endoplasmic reticulum to nucleus signaling 2 (ERN2) polypeptide. In some embodiments, the ERN2 polypeptide comprises or consists of SEQ ID NO: 366.

In some embodiments of the compositions of the disclosure, the second RNA binding protein comprises or consists of a pelota mRNA surveillance and ribosome rescue factor (PELO) polypeptide. In some embodiments, the PELO polypeptide comprises or consists of SEQ ID NO: 367.

In some embodiments of the compositions of the disclosure, the second RNA binding protein comprises or consists of a YBEY metallopeptidase (YBEY) polypeptide. In some embodiments, the YBEY polypeptide comprises or consists of SEQ ID NO: 368.

In some embodiments of the compositions of the disclosure, the second RNA binding protein comprises or consists of a cleavage and polyadenylation specific factor 4 like (CPSF4L) polypeptide. In some embodiments, the CPSF4L polypeptide comprises or consists of SEQ ID NO: 369.

In some embodiments of the compositions of the disclosure, the second RNA binding protein comprises or consists of an hCG_2002731 polypeptide. In some embodiments, the hCG_2002731 polypeptide comprises or consists of SEQ ID NO: 370.

In some embodiments, the hCG_2002731 polypeptide comprises or consists of SEQ ID NO: 371.

In some embodiments of the compositions of the disclosure, the second RNA binding protein comprises or consists of an Excision Repair Cross-Complementation Group 1 (ERCC1) polypeptide. In some embodiments, the ERCC1 polypeptide comprises or consists of SEQ ID NO: 372.

In some embodiments of the compositions of the disclosure, the second RNA binding protein comprises or consists of a ras-related C3 botulinum toxin substrate 1 isoform (RAC1) polypeptide. In some embodiments, the RAC1 polypeptide comprises or consists of SEQ ID NO: 373.

In some embodiments of the compositions of the disclosure, the second RNA binding protein comprises or consists of a Ribonuclease A A1 (RAA1) polypeptide. In some embodiments, the RAA1 polypeptide comprises or consists of SEQ ID NO: 374.

In some embodiments of the compositions of the disclosure, the second RNA binding protein comprises or consists of a Ras Related Protein (RAB1) polypeptide. In some embodiments, the RAB1 polypeptide comprises or consists of SEQ ID NO: 375.

In some embodiments of the compositions of the disclosure, the second RNA binding protein comprises or consists of a DNA Replication Helicase/Nuclease 2 (DNA2) polypeptide. In some embodiments, the DNA2 polypeptide comprises or consists of SEQ ID NO: 376.

In some embodiments of the compositions of the disclosure, the second RNA binding protein comprises or consists of a FLJ35220 polypeptide. In some embodiments, the FLJ35220 polypeptide comprises or consists of SEQ ID NO: 377.

In some embodiments of the compositions of the disclosure, the second RNA binding protein comprises or consists of a FLJ13173 polypeptide. In some embodiments, the FLJ13173 polypeptide comprises or consists of SEQ ID NO: 378.

In some embodiments of the compositions of the disclosure, the second RNA binding protein comprises or consists of Teneurin Transmembrane Protein (TENM) polypeptide. In some embodiments, the second RNA binding protein comprises or consists of Teneurin Transmembrane Protein 1 (TENM1) polypeptide. In some embodiments, the TENM1 polypeptide comprises or consists of SEQ ID NO: 379.

In some embodiments, the second RNA binding protein comprises or consists of Teneurin Transmembrane Protein 2 (TENM2) polypeptide. In some embodiments, the TENM2 polypeptide comprises or consists of SEQ ID NO: 380.

In some embodiments of the compositions of the disclosure, the second RNA binding protein comprises or consists of a Ribonuclease Kappa (RNaseK) polypeptide. In some embodiments, the RNaseK polypeptide comprises or consists of SEQ ID NO: 381.

In some embodiments of the compositions of the disclosure, the second RNA binding protein comprises or consists of a transcription activator-like effector nuclease (TALEN) polypeptide or a nuclease domain thereof. In some embodiments, the TALEN polypeptide comprises or consists of SEQ ID NO: 382. In some embodiments, the TALEN polypeptide comprises or consists of SEQ ID NO: 383.

In some embodiments of the compositions of the disclosure, the second RNA binding protein comprises or consists a zinc finger nuclease polypeptide or a nuclease domain thereof. In some embodiments, the second RNA binding protein comprises or consists of a ZNF638 polypeptide or a nuclease domain thereof. In some embodiments, the ZNF638 polypeptide comprises or consists of SEQ ID NO: 384.

In some embodiments of the compositions of the disclosure, the second RNA binding protein comprises or consists of a PIN domain derived from the human SMG6 protein, also commonly known as telomerase-binding protein EST1A isoform 3, NCBI Reference Sequence: NP_001243756.1. In some embodiments, the PIN from hSMG6 is used herein in the form of a Cas fusion protein and as an internal control, for example, and without limitation. In some embodiments, the PIN polypeptide comprises or consists of SEQ ID NO: 626.

In some embodiments of the compositions of the disclosure, the composition further comprises (a) a sequence comprising a gRNA that specifically binds within an RNA molecule and (b) a sequence encoding a nuclease. In some embodiments, a nuclease comprises a sequence isolated or derived from a CRISPR/Cas protein. In some embodiments, a nuclease comprises a sequence isolated or derived from a TALEN or a nuclease domain thereof. In some embodiments, a nuclease comprises a sequence isolated or derived from a zinc finger nuclease or a nuclease domain thereof.

### AAV Vectors

An “AAV vector” as used herein refers to a vector comprising, consisting essentially of, or consisting of one or more nucleic acid molecules and one or more AAV inverted terminal repeat sequences (ITRs). In some aspects, the nucleic acid molecule encodes for a CAG-repeat targeting protein and/or composition of the disclosure. Such AAV vectors can be replicated and packaged into infectious viral particles when present in a host cell that provides the functionality of rep and cap gene products, for example, by transfection of the host cell. In some aspects, AAV vectors contain a promoter, at least one nucleic acid that may encode at least one protein or RNA, and/or an enhancer and/or a terminator within the flanking ITRs that is packaged into the infectious AAV particle. The encapsidated nucleic acid portion may be referred to as the AAV vector genome. Plasmids containing AAV vectors may also contain elements for manufacturing purposes, e.g., antibiotic resistance genes, origin of replication sequences etc., but these are not encapsidated and thus do not form part of the AAV particle.

In some aspects, an AAV vector can comprise at least one nucleic acid molecule encoding a CAG-repeat targeting composition of the disclosure. In some aspects, an AAV vector can comprise at least one regulatory sequence. In some aspects, an AAV vector can comprise at least one AAV inverted terminal (ITR) sequence. In some aspects, an AAV vector can comprise a first ITR sequence and a second ITR sequence. In some aspects, an AAV vector can comprise at least one promoter sequence. In some aspects, an AAV vector can comprise at least one enhancer sequence. In some aspects, an AAV vector can comprise at least one polyA sequence. In some aspects, an AAV vector can comprise at least one linker sequence. In some aspects, an AAV vector of the disclosure can comprise at least on nuclear localization signals. In some aspects, an AAV vector of the disclosure can comprise a CAG-repeat targeting PUF or PUMBY protein, peptide, or fragment thereof. In some aspects, an AAV vector of the disclosure can comprise a Cas protein, peptide, or fragment thereof. In some aspects, an AAV vector of the disclosure can comprise an endonuclease protein, peptide, or fragment thereof. In some aspects, an AAV vector of the disclosure can comprise a guide RNA, in some cases a CAG-repeat targeting guide RNA. In some aspects, AAV vectors of the disclosure can comprise a fusion protein comprising one or more elements of the disclosure, including, but not limited to, a CAG-repeat targeting protein (such as a Cas, PUF, or PUMBY) and an endonuclease. Optionally, fusion proteins of the AAV vector can further comprise a linker amino acid sequence between the one or more elements of the disclosure.

In some aspects, a AAV vector can comprise a first AAV ITR sequence, a promoter sequence, a CAG-repeat targeting composition nucleic acid molecule, a regulatory sequence and a second AAV ITR sequence. In some aspects, an AAV vector can comprise, in the 5′ to 3′ direction, a first AAV ITR sequence, a promoter sequence, a transgene nucleic acid molecule, and a second AAV ITR sequence.

### CAG-Targeting Cas13d Vectors

In some embodiments of the compositions of the disclosure, CAG-targeting Cas13d compositions are packaged as AAV vectors. In some embodiments, CAG-targeting Cas13d compositions packaged as AAV vectors are set forth in SEQ ID NOs 518, 528, 534, 536, and 539.

In some embodiments, an AAV vector comprising a CAG-targeting Cas13d composition comprises from 5′ to 3′: a human U6 promoter, a cas13d gRNA, wherein the gRNA comprises a direct repeat sequence and a CAG targeting spacer sequence, an EFS promoter, a kozak sequence, a SV40 NLS sequence, a linker sequence, a sequence encoding Cas13d, a linker sequence, a SV40 NLS sequence, a linker sequence, an HA tag sequence, and a BGH poly a sequence.

In some embodiments, a nucleic acid encoding a CAG-targeting Cas13d composition is set forth in SEQ ID NO: 518. In some embodiments, the CAG-targeting Cas13d composition is arranged as depicted in Table 3.

TABLE 3
CAG-targeting Cas13d composition for packaging in AAV unitary
vectors

| Plasmid | |
|---|---|
| Element | Nucleic Acid Sequences |
| Human U6 | gagggcctatttcccatgattccttcatatttgcatatacgatacaaggctgttagagagataattggaattaatttgactgtaaa |
| promoter | cacaaagatattagtacaaaatacgtgacgtagaaagtaataatttcttgggtagtttgcagttttaaaattatgttttaaaatgg |
| | actatcatatgcttaccgtaacttgaaagtatttcgatttcttggctttatatatcttGTGGAAAGGACGAAACACC (SEQ ID |
| | NO: 519) |
| CasRx direct | AACCCCTACCAACTGGTCGGGGTTTGAAAC (SEQ ID NO: 302) |
| repeat (DR) | |
| Spacer (CTG | ctgctgctgctgctgctgctgctgct (SEQ ID NO: 459) |
| guide 3) | |
| EFS promoter | TAGGTCTTGAAAGGAGTGGGAATTGGCTCCGGTGCCCGTCAGTGGGCAGAGCGCA |
| | CATCGCCCACAGTCCCCGAGAAGTTGGGGGGAGGGGTCGGCAATTGATCCGGTGC |
| | CTAGAGAAGGTGGCGCGGGGTAAACTGGGAAAGTGATGTCGTGTACTGGCTCCGC |
| | CTTTTTCCCGAGGGTGGGGGAGAACCGTATATAAGTGCAGTAGTCGCCGTGAACGT |
| | TCTTTTTCGCAACGGGTTTGCCGCCAGAACACAGG (SEQ ID NO: 520) |
| Kozak | AGAACCATG (SEQ ID NO: 546) |
| Sequence | |
| SV-40 NLS | CCCAAGAAgAAGAGAAAGGTG (SEQ ID NO: 524) |
| Linker | GAGGCCAGC (SEQ ID NO: 521) |
| CasRx | ATCGAAAAAAAAAAGTCCTTCGCCAAGGGCATGGGCGTGAAGTCCACACTCGTGT |
| | CCGGCTCCAAAGTGTACATGACAACCTTCGCCGAAGGCAGCGACGCCAGGCTGGA |
| | AAAGATCGTGGAGGGCGACAGCATCAGGAGCGTGAATGAGGGCGAGGCCTTCAG |
| | CGCTGAAATGGCCGATAAAAACGCCGGCTATAAGATCGGCAACGCCAAATTCAGC |
| | CATCCTAAGGGCTACGCCGTGGTGGCTAACAACCCTCTGTATACAGGACCCGTCCA |
| | GCAGGATATGCTCGGCCTGAAGGAAACTCTGGAAAAGAGGTACTTCGGCGAGAGC |
| | GCTGATGGCAATGACAATATTTGTATCCAGGTGATCCATAACATCCTGGACATTGA |
| | AAAAATCCTCGCCGAATACATTACCAACGCCGCCTACGCCGTCAACAATATCTCCG |
| | GCCTGGATAAGGACATTATTGGATTCGGCAAGTTCTCCACAGTGTATACCTACGAC |
| | GAATTCAAAGACCCCGAGCACCATAGGGCCGCTTTCAACAATAACGATAAGCTCA |
| | TCAACGCCATCAAGGCCCAGTATGACGAGTTCGACAACTTCCTCGATAACCCCAGA |
| | CTCGGCTATTTCGGCCAGGCCTTTTTCAGCAAGGAGGGCAGAAATTACATCATCAA |
| | TTACGGCAACGAATGCTATGACATTCTGGCCCTCCTGAGCGGACTGAGGCACTGGG |
| | TGGTCCATAACAACGAAGAAGAGTCCAGGATCTCCAGGACCTGGCTCTACAACCT |
| | CGATAAGAACCTCGACAACGAATACATCTCCACCCTCAACTACCTCTACGACAGG |
| | ATCACCAATGAGCTGACCAACTCCTTCTCCAAGAACTCCGCCGCCAACGTGAACTA |
| | TATTGCCGAAACTCTGGGAATCAACCCTGCCGAATTCGCCGAACAATATTTCAGAT |
| | TCAGCATTATGAAAGAGCAGAAAAACCTCGGATTCAATATCACCAAGCTCAGGGA |
| | AGTGATGCTGGACAGGAAGGATATGTCCGAGATCAGGAAAAATCATAAGGTGTTC |
| | GACTCCATCAGGACCAAGGTCTACACCATGATGGACTTTGTGATTTATAGGTATTA |
| | CATCGAAGAGGATGCCAAGGTGGCTGCCGCCAATAAGTCCCTCCCCGATAATGAG |
| | AAGTCCCTGAGCGAGAAGGATATCTTTGTGATTAACCTGAGGGGCTCCTTCAACGA |
| | CGACCAGAAGGATGCCCTCTACTACGATGAAGCTAATAGAATTTGGAGAAAGCTC |
| | GAAAATATCATGCACAACATCAAGGAATTTAGGGGAAACAAGACAAGAGAGTAT |
| | AAGAAGAAGGACGCCCCTAGACTGCCCAGAATCCTGCCCGCTGGCCGTGATGTTT |
| | CCGCCTTCAGCAAACTCATGTATGCCCTGACCATGTTCCTGGATGGCAAGGAGATC |
| | AACGACCTCCTGACCACCCTGATTAATAAATTCGATAACATCCAGAGCTTCCTGAA |
| | GGTGATGCCTCTCATCGGAGTCAACGCTAAGTTCGTGGAGGAATACGCCTTTTTCA |
| | AAGACTCCGCCAAGATCGCCGATGAGCTGAGGCTGATCAAGTCCTTCGCTAGAAT |
| | GGGAGAACCTATTGCCGATGCCAGGAGGGCCATGTATATCGACGCCATCCGTATTT |
| | TAGGAACCAACCTGTCCTATGATGAGCTCAAGGCCCTCGCCGACACCTTTTCCCTG |
| | GACGAGAACGGAAACAAGCTCAAGAAAGGCAAGCACGGCATGAGAAATTTCATT |
| | ATTAATAACGTGATCAGCAATAAAAGGTTCCACTACCTGATCAGATACGGTGATCC |
| | TGCCCACCTCCATGAGATCGCCAAAAACGAGGCCGTGGTGAAGTTCGTGCTCGGC |
| | AGGATCGCTGACATCCAGAAAAAACAGGGCCAGAACGGCAAGAACCAGATCGAC |
| | AGGTACTACGAAACTTGTATCGGAAAGGATAAGGGCAAGAGCGTGAGCGAAAAG |
| | GTGGACGCTCTCACAAAGATCATCACCGGAATGAACTACGACCAATTCGACAAGA |
| | AAAGGAGCGTCATTGAGGACACCGGCAGGGAAAACGCCGAGAGGGAGAAGTTTA |
| | AAAAGATCATCAGCCTGTACCTCACCGTGATCTACCACATCCTCAAGAATATTGTC |
| | AATATCAACGCCAGGTACGTCATCGGATTCCATTGCGTCGAGCGTGATGCTCAACT |
| | GTACAAGGAGAAAGGCTACGACATCAATCTCAAGAAACTGGAAGAGAAGGGATT |
| | CAGCTCCGTCACCAAGCTCTGCGCTGGCATTGATGAAACTGCCCCCGATAAGAGA |
| | AAGGACGTGGAAAAGGAGATGGCTGAAAGAGCCAAGGAGAGCATTGACAGCCTC |
| | GAGAGCGCCAACCCCAAGCTGTATGCCAATTACATCAAATACAGCGACGAGAAGA |
| | AAGCCGAGGAGTTCACCAGGCAGATTAACAGGGAGAAGGCCAAAACCGCCCTGA |
| | ACGCCTACCTGAGGAACACCAAGTGGAATGTGATCATCAGGGAGGACCTCCTGAG |
| | AATTGACAACAAGACATGTACCCTGTTCAGAAACAAGGCCGTCCACCTGGAAGTG |
| | GCCAGGTATGTCCACGCCTATATCAACGACATTGCCGAGGTCAATTCCTACTTCCA |
| | ACTGTACCATTACATCATGCAGAGAATTATCATGAATGAGAGGTACGAGAAAAGC |
| | AGCGGAAAGGTGTCCGAGTACTTCGACGCTGTGAATGACGAGAAGAAGTACAACG |
| | ATAGGCTCCTGAAACTGCTGTGTGTGCCTTTCGGCTACTGTATCCCCAGGTTTAAG |
| | AACCTGAGCATCGAGGCCCTGTTCGATAGGAACGAGGCCGCCAAGTTCGACAAGG |
| | AGAAAAAGAAGGTGTCCGGCAATTCC (SEQ ID NO: 144) |
| SV-40 poly A | AACTTGTTTATTGCAGCTTATAATGGTTACAAATAAAGCAATAGCATCACAAATTT |
| | CACAAATAAAGCATTTTTTTCACTGCATTCTAGTTGTGGTTTGTCCAAACTCATCAA |
| | TGTATCTTA (SEQ ID NO: 533) |

In some embodiments, a CAG-targeting Cas13d composition comprises from N- to C-terminus: a human U6 promoter, a cas3d gRNA, wherein the gRNA comprises a direct repeat sequence and a CAG targeting spacer sequence, an EFS promoter, a kozak sequence, a sequence encoding Cas13d, a linker sequence, a SV40 NLS sequence, and a SV40 poly a sequence. In some embodiments, a nucleic acid encoding a CAG-targeting Cas13d composition is set forth in SEQ ID NO: 528. In some embodiments, the CAG-targeting Cas13d composition is arranged as depicted in Table 4.

TABLE 4
CAG-targeting Cas13d composition for packaging in AAV unitary
vectors

| Plasmid Element | Nucleic Acid Sequences |
|---|---|
| Human U6 promoter | GAGGGCCTATTTCCCATGATTCCTTCATATTTGCATATACGATACAAGGCTGTTAG |
| | AGAGATAATTAGAATTAATTTGACTGTAAACACAAAGATATTAGTACAAAATACG |
| | TGACGTAGAAAGTAATAATTTCTTGGGTAGTTTGCAGTTTTAAAATTATGTTTTAA |
| | AATGGACTATCATATGCTTACCGTAACTTGAAAGTATTTCGATTTCTTGGCTTTATA |
| | TATCTTGTGGAAAGGACGAAACACC (SEQ ID NO: 519) |
| Seq198 direct repeat | Caagtaaacccctaccaagtggtcggggtttgaaac (SEQ ID NO: 199) |
| (DR) | |
| Spacer (CTG guide 3) | ctgctgctgctgctgctgctgctgct (SEQ ID NO: 459) |
| EFS promoter | TAGGTCTTGAAAGGAGTGGGAATTGGCTCCGGTGCCCGTCAGTGGGCAGAGCGCA |
| | CATCGCCCACAGTCCCCGAGAAGTTGGGGGGAGGGGTCGGCAATTGATCCGGTGC |
| | CTAGAGAAGGTGGCGCGGGGTAAACTGGGAAAGTGATGTCGTGTACTGGCTCCGC |
| | CTTTTTCCCGAGGGTGGGGGAGAACCGTATATAAGTGCAGTAGTCGCCGTGAACGT |
| | TCTTTTTCGCAACGGGTTTGCCGCCAGAACACAGG (SEQ ID NO: 520) |
| Kozak Sequence | gccgccaccATG (SEQ ID NO: 529) |
| Cas13d Seq198 | ATCGAAAAGAAGAAGAGCTACGCTAAAGGAATGGGCCTGAAAAGCACCATCGTGT |
| | CCGGCTCTAAAGTGTACATGACAACCTTCGGCGATGGCAGCGAGGCCAGACTGGA |
| | GAAGGTGGTTGAGAACGATAGCATCAAGACCCTGCACGAGGGCGAGGCCTTCAGC |
| | GCTGAGATGACCGACAACAACGCCGGCTATAAGATCGGAAACGTGAAGTTCTCCC |
| | ACCCTAACGGCTACGACGTGGTCGCCAACAACCCTTTCTACACCGGCCCTGTGCAG |
| | CAGGACATGCTGGGCCTGAAAGAAATCCTGGAAAGACGGTACTTTGGATCTAGCA |
| | CAGACGGTAACAATACCATCTGCATCCAAATCATCCACAATATCCTGGATATCGAA |
| | AAAATTCTGGCAGAGTACATCACAAACGCAGTGTACGCCACCAACAACATCATCG |
| | ATCCTGATAACGACGTGATCGGCGGCAAGAAGTTCACCAGCATTAAAACCTTCGC |
| | CCAGTTCTCCGCCAGCGACAGCAGCAACGATTTCGAGCAGTTCCTGAAAAATCCCA |
| | GACTCGGCTACCTGGGCAAAGCCTTTTTCTACAAGGACGGCAAGCGGAACAACAG |
| | ACAGAAGGATCCTATCGAGTGTTACCACCTGCTGGCCCTGCTGTGCGGCCTGCGTA |
| | ATTGGGTTGTGCACAACAACGAGGAAAAGGACCTGATCAAGTACACCTGGTTGTA |
| | TAACCTGGACAAGTACCTGGATGCCGAGTACATCACCACCCTGAACTACATGTACA |
| | ACGACATCGGCGACGAGTTGACGGACTCTTTCTCCAAGAACAGCGCCGCCAATAT |
| | CAACTACATCGCCGAGACCCTGGGAATCGACCCCAAGACCTTCGCCGAGCAATAC |
| | TTCCGGTTCTCTATCATGAAGGAACAGAAAAACCTGGGATTCAACCTGACCAAGCT |
| | GAGAGAGGTGATGCTGGACCGGAAGGACATGAGCGAGATCAGAGAGAACCACAA |
| | CGACTTCGATTCTATCAGAGCCAAGGTGTACACAATGATGGACTTCGTGATCTATC |
| | GGTACTACATCGAAGAGGCCGCTAAGGTGAACGCCGCCAACAAGAGCCTGCCCGA |
| | CAACGAGAAGAGCCTGAGCGAGAAAGACATCTTCGTGATTTCACTCAGAGGCAGC |
| | TTCAACGAAGATCAGAAGGATCGGCTGTACTACGACGAGGCGCAAAGACTGTGGT |
| | CCAAGGTGGGCAAACTGATGCTGAAAATCAAGAAGTTCCGGGGCAAGGACACCAG |
| | AAAGTACAAGAATATGGGCACACCTAGAATCCGGAGGCTGATCCCTGAGGGCAGA |
| | GATATCAGCACCTTCTCCAAGCTGATGTACGCTCTGACTATGTTCCTGGACGGCAA |
| | GGAGATCAATGACCTGCTGACCACACTGATCAACAAATTCGACAACATCCAGAGC |
| | TTCTTAAAGGTGATGCCTCTGATCGGCGTGAACGCCAAATTTGCCGAAGAATATAG |
| | TTTCTTCAACAACTCTGAAAAAATCGCCGACGAACTGCGGCTGATCAAGAGCTTTG |
| | CTAGAATGGGAGAACCCGTGGCTGACGCCAGAAGAGCCATGTATATCGACGCAAT |
| | TCGCATCCTGGGCACCGATCTCTCCGACGACGAGCTGAAGGCCCTGGCTGATTCTT |
| | TTAGCCTGGACGAGAACGGCAATAAGCTGGGGAAGGGCAAGCACGGCATGAGAA |
| | ACTTCATCATTAACAACGTGATAACAAATAAGAGATTCCACTACCTGATCCGGTAC |
| | GGCAACCCAGTCCACCTGCATGAGATCGCCAAGAATGAAGCCGTGGTCAAGTTTG |
| | TGCTGGGAAGAATCGCCGATATCCAGAAGAAACAGGGCCAGAACGGCAAGAACC |
| | AGATCGATAGATACTACGAGACATGCATCGGCAAGGACTCTTCTAAAAGCGTGGC |
| | CGAGAAGGTGAACGCCCTGACCAAGATCATCACAGGCATGAACTACGACCAGTTC |
| | GACAGCAGACGGAACGTGATCGAAAACACCGGCGCCGGCAACGCCGAGAGAGAA |
| | AAGTACAAGAAGATCATCAGCCTGTACCTGACAGTGATCTACCACATCCTGAAGA |
| | ACATTGTTAATATCAACTCAAGATACGTGATCGGATTTCACTGCGTGGAGAGAGAT |
| | GCCCAGCTGTATAAGGAAAAGGGCTACGACATTAATCTGAAAAAGCTGAAAGACA |
| | AGGGATTCACAAGCGTGACCAAGCTGTGCGCCGGAATCGACGAGGAATGCAAGGA |
| | CGTCGAAAAGGAAATGACCGAGCGGGCCAAGGCCTCTTTCGCTGCCCTGGAAACC |
| | GCCAACCCCAAGCTGTACGCCACATACATCAACTACTCTGATGAAGAGAAGAATG |
| | CCGAACTGAGAAAGCAGATCAATAGAGAGAAGGCCAAAACCGCCCTGAACGCTC |
| | ATCTGCGCAACACCAAGTGGAACGTGATCATCCGGGAAGATCTTCTGAGAAGAGA |
| | TAACAAGGCTTGTAAAATCTTCAGAAATAAGGTCGCCCACCTGGAGGCCATCCGA |
| | TACGCTCACCTGTACATCAACGACATCGCTGAGGTGAATAGCTATTTTCAGTTTTA |
| | CCACTACATCATGCAGCGGAGGATCATGGCCGAACGGTACGACAAGAGCAGCGGC |
| | AAGGTTAGAGAATACTTCGACGCCGTGAACAATGAGAAAAAATACAACGATAGAC |
| | TGCTGAAGCTCCTCTGTGTGCCATTCGGCTACTGCATCCCTAGATTCAAGAATCTG |
| | AGCATCGAGGCCCTGTTCGACATGAACGAGGCCGTGAAGTTTGATAAGGAAAAGA |
| | AG (SEQ ID NO: 530) |
| Linker | GGATCC (SEQ ID NO: 531) |
| SV40 NLS | CCCAAAAAAAAAAGGAAGGTG (SEQ ID NO: 532) |
| SV-40 poly A | AACTTGTTTATTGCAGCTTATAATGGTTACAAATAAAGCAATAGCATCACAAATTT |
| | CACAAATAAAGCATTTTTTTCACTGCATTCTAGTTGTGGTTTGTCCAAACTCATCAA |
| | TGTATCTTA (SEQ ID NO: 533) |

In some embodiments, an AAV vector comprising a CAG-targeting Cas13d composition comprises from 5′ to 3′: a human U6 promoter, a cas13d gRNA, wherein the gRNA comprises a direct repeat sequence and a CAG targeting spacer sequence, an EFS promoter, a kozak sequence, a sequence encoding Cas13d, a linker sequence, a SV40 NLS sequence, and an SV40 poly a sequence. In some embodiments, a nucleic acid encoding a CAG-targeting Cas13d composition is set forth in SEQ ID NO: 534. In some embodiments, the CAG-targeting Cas13d composition is arranged as depicted in Table 5.

TABLE 5
CAG-targeting Cas13d composition for packaging in AAV unitary
vectors

| Plasmid Element | Nucleic Acid Sequences |
|---|---|
| Human U6 promoter | GAGGGCCTATTTCCCATGATTCCTTCATATTTGCATATACGATACAAGGCTGTTAG |
| | AGAGATAATTAGAATTAATTTGACTGTAAACACAAAGATATTAGTACAAAATACG |
| | TGACGTAGAAAGTAATAATTTCTTGGGTAGTTTGCAGTTTTAAAATTATGTTTTAA |
| | AATGGACTATCATATGCTTACCGTAACTTGAAAGTATTTCGATTTCTTGGCTTTATA |
| | TATCTTGTGGAAAGGACGAAACACC (SEQ ID NO: 519) |
| Seq179 direct repeat | Actatagccctgccggaaatgacagggttctacaac (SEQ ID NO: 180) |
| (DR) | |
| Spacer (CTG guide 3) | ctgctgctgctgctgctgctgctgct (SEQ ID NO: 459) |
| EFS promoter | TAGGTCTTGAAAGGAGTGGGAATTGGCTCCGGTGCCCGTCAGTGGGCAGAGCGCA |
| | CATCGCCCACAGTCCCCGAGAAGTTGGGGGGAGGGGTCGGCAATTGATCCGGTGC |
| | CTAGAGAAGGTGGCGCGGGGTAAACTGGGAAAGTGATGTCGTGTACTGGCTCCGC |
| | CTTTTTCCCGAGGGTGGGGGAGAACCGTATATAAGTGCAGTAGTCGCCGTGAACGT |
| | TCTTTTTCGCAACGGGTTTGCCGCCAGAACACAGG (SEQ ID NO: 520) |
| Kozak Sequence | gccgccaccATG (SEQ ID NO: 529) |
| Cas 13d Seq179 | GCCAAAAAGAAGAAAACCGCTCGCCAACTGAGAGAAGAAATGCAACAACAGCGG |
| | AAACAGGCCATTCAGAAGCAACAAGAACAGAGACAAGAGAAAGCCGCCGCCGCT |
| | CGCGAGACAGCCGCCCCCGAACAGCCTGCTGCCGCTCCTGTGCCAAAGCGGCAAA |
| | GAAAATCTCTGGCCAAAGCCGCCGGACTGAAGTCCAACTTCATCTTGGACCCACA |
| | GAGAAGAACAACAGTGATGACAGCTTTTGGCCAGGGCAGCACCGCCATCCTGGAG |
| | AAGCAGATCGTGGACAGAGCCATCAGCGACCTGCAGCCGGTTCAGCAGTTCCAAG |
| | TGGAACCTGCCAGTGCCGCCAAGTACAGGCTGAAGAATAGCCGGGTGAGATTCCC |
| | CAACGTGACAGCTGACGATCCTCTGTATAGACGGAAGGATGGCGGCTTCGTGCCT |
| | GGCATGGACGCCCTCAGAAGAAAGAACGTACTGGAACAGAGATTCTTCGGCAAGT |
| | CTTTCGCCGATAACATCCACATCCAGATGATCTACAGCATCCTGGACATCCACAAG |
| | ATCCTCGCTGCCGCGAGCGGCCACATCGTGCACCTGCTCAATATCGTGAATGGCTC |
| | AAAAGATAGAGACTTCATCGGCATGCTGGCCGCCCACGTGCTGTACAATGAGCTG |
| | AACGAGGAGGCCAAGCGGAGCATCGCCGACTTTTGCAAGAGTCCCAGACTGATCT |
| | ACTACTCTGCTGCTTTCTACGAGACATTGGACAACGGCAAGAGCGAGCGACGGTCT |
| | AACGAGGACATCTTCAACATCCTGGCCCTGATGACCTGTCTGAGAAATTTCAGCAG |
| | CCACCACAGCATCGCCATCAAGGTGAAGGACTACAGCGCCGCTGGCCTGTACAAC |
| | CTGCGGAGACTGGGACCTGACATGAAGAAAATGCTGGACACCTTCTACACCGAGG |
| | CCTTCATCCAGCTTAACCAGAGCTTCCAGGACCACAACACCACAAACCTGACATGT |
| | CTGTTCGATATCCTGAACATCTCTGATAGCGCCAGACAGAAGCAGCTGGCTGAGG |
| | AATTTTATAGATACGTGGTGTTCAAGGAACAAAAGAACTTGGGATTCTCCGTGCGG |
| | AAGCTGAGAGAGGAAATGCTGCTGCTGCCAGACGCTGCCGTGATCGCCGATAAGC |
| | GGTACGACACCTGCAGATCCAAGCTGTACAACCTGATGGACTTCCTGATCCTGAGA |
| | GTGTACAGAACCGGCAGAGCCGACAGATGCGACAAGCTGCCTGAGGCCCTGCGGG |
| | CCGCCCTGACCGACGAGGAAAAGGCCGTGGTGTACCACAAAGAAGCCCTGAGCCT |
| | GTGGAACGAGATGAGAACCCTGATCCTCGACGGCCTGCTGCCTCAGATGACACCT |
| | GAGAACCTGAGCAGACTGTCCGGTCAGAAAAGAAAGGGCGAACTGTCTCTGGATG |
| | ACGCCATGCTGAAAGAGTGCCTGTACGAGCCCGGACCTGTGCCCGAGGATGCTGC |
| | CCCTGAGGAAGCCAACGCCGAGTACTTCTGCCGGATGATCTACCTGGCCACCCTGT |
| | TTATGGATGGCAAGGAGATCAACACCCTGCTGACCACCCTGATTAGCAAATTCGA |
| | GAACATCGCCGCCTTCCTGCAGACCATGGAACAGCTGAACATCGAGGCCGAGCTG |
| | GGCCCTGAATACGCCATGTTTACCAGAAGCAGAGCCGTAGCCGAGCAGCTGAGAG |
| | TGATCAACAGCTTCGCCCTGATGAAGAAGCCTCAGGTGAATGCCAAGCAGCAGCT |
| | GTACAGAGCCGCTGTCACCCTGCTGGGAACAGAGGACCCTGACGGCGTGACCGAT |
| | GAGATGCTGTGCATCGACCCCGTGACCGGCAAGATGCTGCCTCCTAACCAGAGGC |
| | ATCATGGCGACACCGGCTTACGGAACTTCATCGCAAACAACGTGGTGGAAAGCCG |
| | GAGATTCCAGTACTTAATCCGGTACAGCGATCCTGCTCAGCTGCACCAGCTCGCCA |
| | GCAACAAGAAGCTGGTCAGATTCGTGCTGAGCAGCATCCCCGACACACAGATCAA |
| | CAGATACTATGAAACCTGTGGCCAGACCAGACTGGCCGGCAGAGCCGCCAAGGTG |
| | GAATTCCTGACAGACATGATTGCCGCCATCAGATTCGACCAGTTTCGGGATGTCAA |
| | TCAGAAAGAGCGCGGCGCCAATACTCAGAAAGAAAGATATAAGGCCATGCTTGGC |
| | CTGTACCAGACCGTGCTGTACCTGGCTGTTAAAAATCTGGTGAACATTAACGCCAG |
| | ATACGTGATGGCCTTCCACTGCGTGGAGCGGGATATGTTTCTGTATGACGGCGAGC |
| | TGACAGATCCCAAGGGCGAGAGCGTGTCTGCTTTCCTGGCTGTGAATGGAAAGAA |
| | GGGCGTGCAGCCTCAGTACCTGCTGCTGACCCAGCTGTTTATCCGGCGGGATTACC |
| | TTAAGCGGAGTGCATGCGAGCAGATCCAGCACAACATGGAAAACATCTCCGACCG |
| | GCTGCTGCGGGAATACCGGAACGCCGTCGCCCACCTGAATGTGATAGCCCATCTG |
| | GCTGACTACTCTGCCGACATGAGAGAAATCACCAGCTACTACGGCTTGTATCACTA |
| | CCTGATGCAGAGACACCTCTTCAAAAGACACGCCTGGCAGATCAGACAGCCTGAA |
| | AGGCCAACTGAGGAGGAACAGAAGCTCATCGAGCAGGAGCAGAAGCAGCTGGCC |
| | TGGGAGAAGGCCCTGTTTGACAAGACCCTGCAGTACCACAGCTACAACAAGGACC |
| | TGGTGAAGGCTCTTAACGCCCCCTTCGGATACAACCTGGCAAGATACAAGAACCT |
| | GTCTATCGAGCCTCTGTTCAGCAAAGAAGCCGCTCCTGCCGCCGAGATCAAGGCCA |
| | CACACGCC (SEQ ID NO: 535) |
| Linker | GGATCC (SEQ ID NO: 531) |
| SV40 NLS | CCCAAAAAAAAAAGGAAGGTG (SEQ ID NO: 532) |
| SV-40 poly A | AACTTGTTTATTGCAGCTTATAATGGTTACAAATAAAGCAATAGCATCACAAATTT |
| | CACAAATAAAGCATTTTTTTCACTGCATTCTAGTTGTGGTTTGTCCAAACTCATCAA |
| | TGTATCTTA (SEQ ID NO: 533) |

In some embodiments, an AAV vector comprising a CAG-targeting Cas13d composition comprises from 5′ to 3′: a human U6 promoter, a cas13d gRNA, wherein the gRNA comprises a direct repeat sequence and a CAG targeting spacer sequence, an EFS promoter, a kozak sequence, a sequence encoding Cas13d, a linker sequence, an SV40 NLS sequence, and anSV40 poly a sequence. In some embodiments, a nucleic acid encoding a CAG-targeting Cas13d composition is set forth in SEQ ID NO: 536. In some embodiments, the CAG-targeting Cas13d composition is arranged as depicted in Table 6.

TABLE 6
CAG-targeting Cas13d composition for packaging in AAV unitary
vectors

| Plasmid Element | Nucleic Acid Sequences |
|---|---|
| Human U6 promoter | GAGGGCCTATTTCCCATGATTCCTTCATATTTGCATATACGATACAAGGCTGTTAG |
| | AGAGATAATTAGAATTAATTTGACTGTAAACACAAAGATATTAGTACAAAATACG |
| | TGACGTAGAAAGTAATAATTTCTTGGGTAGTTTGCAGTTTTAAAATTATGTTTTAA |
| | AATGGACTATCATATGCTTACCGTAACTTGAAAGTATTTCGATTTCTTGGCTTTATA |
| | TATCTTGTGGAAAGGACGAAACACC (SEQ ID NO: 519) |
| Seq42 direct repeat | GACCAACACCTCTGCAAAACTGCAGGGGTCTAAAAC (SEQ ID NO: 537) |
| (DR) | |
| Spacer (CTG guide 3) | ctgctgctgctgctgctgctgctgct (SEQ ID NO: 459) |
| EFS promoter | TAGGTCTTGAAAGGAGTGGGAATTGGCTCCGGTGCCCGTCAGTGGGCAGAGCGCA |
| | CATCGCCCACAGTCCCCGAGAAGTTGGGGGGAGGGGTCGGCAATTGATCCGGTGC |
| | CTAGAGAAGGTGGCGCGGGGTAAACTGGGAAAGTGATGTCGTGTACTGGCTCCGC |
| | CTTTTTCCCGAGGGTGGGGGAGAACCGTATATAAGTGCAGTAGTCGCCGTGAACGT |
| | TCTTTTTCGCAACGGGTTTGCCGCCAGAACACAGG (SEQ ID NO: 520) |
| Kozak Sequence | gccgccaccATG (SEQ ID NO: 529) |
| Cas13d Seq42 | AACAATAAGAGAAAGACAAAGGCCAAGGCCGCTGGACTGAAGAGCGTCTTTTTTG |
| | ATCAGAAGCAAGCCGTGCTGACCACATTCGCCAAGGGCAACAACTCCCAGATCGA |
| | GAAGAAAGTGGTCAACAGCGAGGTCAAAGATCTGAGACAGCCTCCCGCCTTTGAT |
| | CTGGAACTGAAGGAGAAGACCTTCTATATCTCCGGCAAGAACAACATTAACACAT |
| | CTAGGGAGAACCCTCTGGCTAGCGCTTCTCTGCCTCTCTCCAAGAGGCAAAGGATT |
| | AGAGCCGAGAGGATCAAGAGAGCTAGAGAAGAAAATAGACCCTACCATAATGTC |
| | AAGAGGGTGGGAGAGGACGATCTGAGAGCCAAGGCTGACCTCGAGAAACACTAC |
| | TTCGGCAAGGAGTACAGCGATAATCTGAAAATTCAGATTATTTATAATATCCTCGA |
| | CATCAACAAAATCATCAGCCCCTATATCAATGACATCGTCTACTCCATGAACAATC |
| | TGGCTAGAAACGACGAGTATATCGATGGAAAAATCGACGTGATCGGCTCCCTCTC |
| | CTCCACCACAGACTACTCCTCCTTCATGAGCCCCAACAAGGATCTGGAAAAGGAA |
| | AAAAAGTTTTCCTTCCATAGAGAAAACTACAAAAAATTCGTCGAGGCCAGCAAGC |
| | CCTACATGAGGTACTATGGAAAGGTGTTTATTAGAGACGTGAAGAAAAGCAAGCT |
| | CTCCACCGGAAAGGGCGAGAAGATTGAGGTGATGTATAGATCCGACGAGGAAATT |
| | TTCACCATTTTTCAAATTCTGAGCTATGTGAGACAATCCATCATGCACAACGACAT |
| | CGGAAACAAGAGCAGCATTCTGGCCATCGAAAAGTACCCCGCCAGATTCGTCGGC |
| | TTTCTGAGCGACCTCCTCAAAACCAAGACAAACGATGTCAATAGAATGTTCATTGA |
| | CAATAACAGCCAAACAAACTTCTGGGTGCTCTTCAGCATCTTCGGACTGCAAGATC |
| | ACACCAGCGGAGCCGACAAGATCTGTAGAAATTTCTACGACTTCGTGATCAAGGC |
| | CGACAGCAAAAACCTCGGATTCTCCCTCAAGAAGATCAGAGAGCTGATGCTCGAT |
| | CTGCCTAACGCCAACATGCTGAGAGATCACCAATTCGATACCGTGAGGAGCAAGT |
| | TTTATACCCTCCTCGACTTCATTATCTATCAACACTATCTCGAGGAGAAGTCCAGA |
| | ATCGACAACATGGTGGAGAAGCTGAGGATGACCCTCAAGGAAGAGGAAAAGGAA |
| | GTGCTCTACGCTGCCGAGGCCAAGATTGTGTGGAATGCCATCGGAGCCAAGGTCA |
| | TCAACAAGCTCGTGCCCATGATGAATGGCGATGCTCTGAAGGAGATCAAGAGAAA |
| | AAATAGAGATAGAAAGCTCCCTCAGAGCGTGATCGCCACAGTGCAAGTGAATTCC |
| | GACGCCAATGTGTTCTCCGGACTGATCTACTTTCTGACACTGTTTCTCGACGGCAA |
| | GGAGATCAACGAGATGGTGAGCAACCTCATCACCAAGTTCGAGAACATTGACTCT |
| | CTGCTGCATGTCGATAGAGAAATCTACAAGTCCGACGAGAAGGATCTGGATCTCG |
| | AGATCGAGAAGCTGGCCCTCTTTTTCAAGGGCGTGGTGAGGCCTAATGCCAAGAC |
| | AGATACCGGCGCCGGAGAGATCTCCAAGAGCTTCTCCATCTTCCAGAGCGCCGAA |
| | AGGATTATCGAGGAACTGAAGTTCATTAAGAACGTCACAAGAATGGATAACGAGA |
| | TCTTCCCTAGCGAGGGCGTGTTCCTCGATGCCGCTAACGTGCTCGGCGTCAGAGGC |
| | GATGACTTTGACTTTAGCAATGAGTTTGTCGGAGACGATCTGCACAGCGACGCTAA |
| | TAAGAAGATTATTAACAAGATCAATGGCACCAAGGAGGACAGAAATCTGAGGAAC |
| | TTTATTATTAATAACGTCGTGAAGAGCAGAAGGTTTCAGTATATCGCTAGACACAT |
| | GAATACACACTACGTCAAGCAGCTCGCCAATAACGAGACACTGAATAGATTCGTG |
| | CTGAACAAGATGGGAGACGCCAAGATCATCAATAGGTACTACGAGTCCATCTCCG |
| | GCAATACCCCCAATATTGAGGTCAGAAGCCAAATCGACTACCTCGTCAAGAGACT |
| | GAGGAGCTTCAGCTTCGAAGACCTCAACGACGTCAAGCAAAAGGTGAGACCCGGC |
| | ACCAATGAGAGCATCGAGAAGGAGAAGAAAAAGGCCCTCGTCGGACTGTGCCTCA |
| | CAATTCAGTACCTCGTGTATAAAAATCTGGTGAATATCAACGCTAGGTACACCACC |
| | GCTTTCTACTGTCTGGAGAGGGACTCCAAACTGAAAGGCTTTGGCGTGGACGTGTG |
| | GAGAGATTTCGAATCCTACACCGCTCTGACCAATCACTTTATCAAAGAAGGCTATC |
| | TGCCCGTGAGAAAGGCTGAAATTCTGAGGGCCAATCTGAAGCATCTGGACTGTGA |
| | AGACGGCTTCAAATATTACAGAAACCAAGTGACCCACCTCAACGCCATTAGAGTC |
| | GCCTATAAATATATCAACGAGATTAAATCCGTGCACAGCTACTTCGCCCTCTACCA |
| | CTACATCATGCAGAGACATCTGTACGACAGCCTCCAAGCCAAAGCTAAGGACTCC |
| | TCCGGCTTCGTGATCGACGCTCTGAAGAAATCCTTCGAGCACAAGATCTACAGCAA |
| | AGATCTGCTCCACGTGCTGCACTCCCCCTTCGGCTATAATACCGCTAGATATAAAA |
| | ATCTGAGCATCGAGGCCCTCTTCGACAAGAACGAATCCAGACCCGAGGTGAATCC |
| | CCTCTCCACCAATGAT (SEQ ID NO: 538) |
| Linker | GGATCC (SEQ ID NO: 531) |
| SV40 NLS | CCCAAAAAAAAAAGGAAGGTG (SEQ ID NO: 532) |
| SV-40 poly A | AACTTGTTTATTGCAGCTTATAATGGTTACAAATAAAGCAATAGCATCACAAATTT |
| | CACAAATAAAGCATTTTTTTCACTGCATTCTAGTTGTGGTTTGTCCAAACTCATCAA |
| | TGTATCTTA (SEQ ID NO: 533) |

In some embodiments, an AAV vector comprising a CAG-targeting Cas13d composition comprises from 5′ to 3′: a human U6 promoter, a cas13d gRNA, wherein the gRNA comprises a direct repeat sequence and a CAG targeting spacer sequence, an EFS promoter, a kozak sequence, a sequence encoding Cas13d, a linker sequence, an SV40 NLS sequence, and anSV40 poly a sequence. In some embodiments, a nucleic acid encoding a CAG-targeting Cas13d composition is set forth in SEQ ID NO: 539. In some embodiments, the CAG-targeting Cas13d composition is arranged as depicted in Table 7.

TABLE 7
CAG-targeting Cas13d composition for packaging in AAV unitary
vectors

| Plasmid Element | Nucleic Acid Sequences |
|---|---|
| Human U6 promoter | GAGGGCCTATTTCCCATGATTCCTTCATATTTGCATATACGATACAAGGCTGTTAG |
| | AGAGATAATTAGAATTAATTTGACTGTAAACACAAAGATATTAGTACAAAATACG |
| | TGACGTAGAAAGTAATAATTTCTTGGGTAGTTTGCAGTTTTAAAATTATGTTTTAA |
| | AATGGACTATCATATGCTTACCGTAACTTGAAAGTATTTCGATTTCTTGGCTTTATA |
| | TATCTTGTGGAAAGGACGAAACACC (SEQ ID NO: 519) |
| Seq212 direct repeat | gtacaatagccctgcagtaaggcagggttctaAGAC (SEQ ID NO: 213) |
| Spacer (CTG guide 3) | ctgctgctgctgctgctgctgctgct (SEQ ID NO: 459) |
| EFS promoter | TAGGTCTTGAAAGGAGTGGGAATTGGCTCCGGTGCCCGTCAGTGGGCAGAGCGCA |
| | CATCGCCCACAGTCCCCGAGAAGTTGGGGGGAGGGGTCGGCAATTGATCCGGTGC |
| | CTAGAGAAGGTGGCGCGGGGTAAACTGGGAAAGTGATGTCGTGTACTGGCTCCGC |
| | CTTTTTCCCGAGGGTGGGGGAGAACCGTATATAAGTGCAGTAGTCGCCGTGAACGT |
| | TCTTTTTCGCAACGGGTTTGCCGCCAGAACACAGG (SEQ ID NO: 520) |
| Kozak Sequence | gccgccaccATG (SEQ ID NO: 529) |
| Cas13d Seq212 | AAGAAGAAGCACCAGAGCGCCGCCGAGAAGAGGCAAGTGAAGAAGCTCAAGAAT |
| | CAAGAGAAGGCCCAGAAGTACGCTAGCGAGCCTTCCCCCCTCCAGAGCGATACAG |
| | CTGGCGTGGAATGCTCCCAGAAAAAGACAGTCGTCAGCCACATTGCCAGCTCCAA |
| | GACACTGGCCAAGGCTATGGGACTCAAATCCACACTGGTCATGGGCGACAAGCTG |
| | GTCATCACCAGCTTTGCTGCTAGCAAGGCTGTCGGAGGCGCTGGCTACAAAAGCG |
| | CTAACATTGAAAAAATCACAGATCTGCAAGGAAGGGTCATTGAGGAGCACGAAAG |
| | GATGTTTAGCGCCGATGTCGGAGAGAAAAATATCGAACTGAGCAAGAATGACTGC |
| | CACACCAACGTCAACAACCCCGTGGTGACCAACATCGGAAAGGATTACATCGGAC |
| | TGAAATCTAGGCTGGAGCAAGAGTTTTTCGGCAAGACATTCGAGAATGACAATCT |
| | GCATGTGCAGCTGGCCTACAATATCCTCGACATCAAGAAAATTCTGGGAACCTATG |
| | TGAACAATATCATTTATATCTTCTACAATCTGAATAGGGCTGGCACCGGCAGAGAT |
| | GAGAGGATGTATGACGACCTCATCGGCACACTGTACGCTTACAAACCCATGGAGG |
| | CTCAACAGACCTATCTGCTCAAAGGCGACAAGGATATGAGGAGGTTTGAGGAGGT |
| | GAAACAGCTGCTGCAAAACACCTCCGCTTACTATGTGTATTACGGCACACTGTTCG |
| | AGAAGGTGAAGGCTAAGAGCAAGAAGGAACAGAGGGCTAAGGAGGCCGAAATCG |
| | ACGCTTGTACCGCCCATAACTACGATGTGCTGAGACTGCTGTCCCTCATGAGGCAG |
| | CTGTGCATGCACTCCGTCGCTGGAACAGCCTTTAAGCTGGCTGAGTCCGCTCTGTT |
| | CAACATTGAGGATGTGCTCAGCGCCGATCTGAAGGAAATCCTCGATGAAGCCTTCT |
| | CCGGCGCCGTGAACAAGCTCAATGACGGATTCGTGCAGCACTCCGGCAACAATCT |
| | GTACGTGCTCCAGCAGCTGTACCCTAATGAGACCATCGAGAGAATCGCCGAGAAG |
| | TACTACAGACTCACCGTGAGGAAGGAGGATCTGAACATGGGAGTCAACATTAAAA |
| | AGCTGAGGGAGCTGATCGTGGGCCAATACTTTCCCGAGGTCCTCGACAAAGAATA |
| | CGACCTCTCCAAGAATGGAGACAGCGTGGTGACATACAGAAGCAAGATTTATACC |
| | GTGATGAATTACATTCTGCTGTATTACCTCGAGGACCACGACTCCAGCAGAGAAAG |
| | CATGGTCGAAGCTCTGAGACAAAACAGAGAGGGCGATGAAGGCAAGGAGGAGAT |
| | CTATAGACAGTTTGCCAAGAAGGTGTGGAACGGCGTGTCCGGACTGTTTGGCGTGT |
| | GTCTGAACCTCTTCAAGACCGAAAAGAGAAACAAGTTTAGGAGCAAAGTCGCCCT |
| | CCCCGATGTGTCCGGCGCTGCCTATATGCTCTCCTCCGAGAACATCGACTACTTTGT |
| | CAAGATGCTCTTCTTTGTGTGTAAGTTTCTGGATGGCAAAGAAATCAACGAGCTGC |
| | TGTGCGCTCTGATCAACAAATTTGATAATATTGCCGATATTCTGGATGCTGCCGCT |
| | CAATGTGGCTCCTCCGTCTGGTTCGTGGACAGCTATAGGTTCTTCGAGAGATCTAG |
| | GAGGATTAGCGCCCAGATTAGAATCGTGAAGAACATCGCTTCCAAGGATTTTAAG |
| | AAATCCAAGAAGGATTCCGATGAGAGCTACCCCGAGCAGCTGTATCTGGATGCTC |
| | TGGCTCTGCTCGGAGACGTCATCTCCAAGTACAAGCAGAATAGAGATGGCAGCGT |
| | CGTCATCGATGACCAAGGCAATGCCGTGCTGACAGAGCAATACAAGAGGTTTAGA |
| | TATGAATTTTTCGAGGAGATCAAGAGGGACGAAAGCGGCGGCATCAAGTACAAGA |
| | AGTCCGGAAAACCCGAGTACAACCATCAGAGAAGGAATTTTATTCTGAATAATGT |
| | GCTGAAAAGCAAATGGTTTTTCTATGTGGTGAAGTACAATAGGCCCAGCAGCTGC |
| | AGAGAACTGATGAAGAATAAGGAAATTCTGAGGTTCGTGCTGAGAGACATCCCCG |
| | ACTCCCAAGTGAGAAGATACTTTAAGGCCGTCCAAGGAGAGGAAGCTTACGCTAG |
| | CGCCGAAGCTATGAGGACAAGACTGGTCGACGCTCTGTCCCAATTTAGCGTCACA |
| | GCTTGTCTGGATGAAGTGGGCGGCATGACAGACAAGGAATTCGCCTCCCAGAGGG |
| | CCGTCGATAGCAAAGAAAAACTGAGAGCCATCATCAGACTGTATCTGACAGTCGC |
| | CTATCTGATTACCAAGAGCATGGTGAAGGTGAATACAAGGTTTAGCATTGCCTTTA |
| | GCGTGCTGGAGAGGGACTACTATCTGCTCATTGACGGCAAGAAGAAATCCAGCGA |
| | CTACACCGGAGAGGATATGCTGGCTCTGACCAGAAAATTTGTGGGCGAAGATGCT |
| | GGACTGTATAGAGAGTGGAAAGAGAAGAACGCTGAAGCCAAGGACAAATATTTTG |
| | ACAAGGCCGAAAGGAAGAAGGTGCTGAGACAGAACGATAAGATGATCAGAAAGA |
| | TGCACTTCACACCCCACTCCCTCAATTACGTCCAAAAGAATCTCGAAAGCGTCCAG |
| | AGCAACGGACTGGCCGCCGTCATCAAGGAATATAGAAATGCCGTCGCTCACCTCA |
| | ATATCATCAATAGACTGGACGAGTACATTGGCTCCGCTAGGGCTGATAGCTACTAC |
| | TCTCTGTACTGTTACTGCCTCCAAATGTATCTGAGCAAGAACTTCAGCGTGGGCTA |
| | CCTCATCAACGTGCAAAAGCAGCTGGAGGAGCACCACACCTACATGAAGGATCTC |
| | ATGTGGCTGCTCAACATCCCCTTCGCTTACAACCTCGCCAGATACAAAAATCTGTC |
| | CAACGAAAAACTCTTTTACGACGAGGAAGCCGCCGCCGAAAAGGCTGACAAGGCT |
| | GAGAACGAGAGAGGCGAA (SEQ ID NO: 540) |
| Linker | GGAAGC (SEQ ID NO: 531) |
| SV40 NLS | CCCAAGAAGAAAAGGAAGGTC (SEQ ID NO: 532) |
| SV-40 poly A | AACTTGTTTATTGCAGCTTATAATGGTTACAAATAAAGCAATAGCATCACAAATTT |
| | CACAAATAAAGCATTTTTTTCACTGCATTCTAGTTGTGGTTTGTCCAAACTCATCAA |
| | TGTATCTTA (SEQ ID NO: 533) |

In some embodiments, an AAV vector comprising a nucleic acid encoding a CAG-targeting Cas13d composition comprises from 5′ to 3′: a sequence encoding a 5′ ITR (a first ITR), a sequence encoding an human U6 promoter, a dCas13d seq212 direct repeat, a sequence encoding a CAG guide 3 spacer sequence, a sequence encoding an EFS promoter, a sequence encoding a kozak sequence, a sequence encoding a dCas13d seq212 protein, a sequence encoding a linker sequence, a sequence encoding an SV-40 NLS, a sequence encoding a linker sequence, a sequence encoding an HA tag, a sequence encoding a WPRE, a sequence encoding an SV-40 polyA, and a 3′ ITR (a second ITR). In some embodiments, the CAG-targeting Cas13d composition is arranged as depicted in Table G. In some embodiments, vector A01479 is suitable for blocking. In some aspects, A01479 is encoded by a nucleic acid sequence comprising SEQ ID NO: 588.

In some embodiments, the vector set forth in Table G is referred to as A01479.

TABLE G1
Vector A01479 encoding a CAG-repeat targeting dCas13d protein for blocking

| Plasmid | |
|---|---|
| Element | Nucleic Acid Sequences |
| 5' ITR | Cctgcaggcagctgcgcgctcgctcgctcactgaggccgcccgggcgtcgggcgacctttggtcgcccggcctcag |
| | tgagcgagcgagcgcgcagagagggagtggccaactccatcactaggggttcct (SEQ ID NO: 597) |
| Human U6 | Gagggcctatttcccatgattccttcatatttgcatatacgatacaaggctgttagagagataattggaattaatttgactgt |
| promoter | aaacacaaagatattagtacaaaatacgtgacgtagaaagtaataatttcttgggtagtttgcagttttaaaattatgttttaa |
| | aatggactatcatatgcttaccgtaacttgaaagtatttcgatttcttggctttatatatcttgtggaaaggacgaaacacc |
| | (SEQ ID NO: 519) |
| Seq212 direct | Tagccctgcagtaaggcagggttctaagac (SEQ ID NO: 596) |
| repeat (DR) | |
| Spacer (CAG | Ctgctgctgctgctgctgctgctgct (SEQ ID NO: 459) |
| guide 3) | |
| EFS promoter | Taggtcttgaaaggagtgggaattggctccggtgcccgtcagtgggcagagcgcacatcgcccacagtccccgaga |
| | agttggggggaggggtcggcaattgatccggtgcctagagaaggtggcgcggggtaaactgggaaagtgatgtcgt |
| | gtactggctccgcctttttcccgaggggggggagaaccgtatataagtgcagtagtcgccgtgaacgttctttttcgcaa |
| | cgggtttgccgccagaacacagg (SEQ ID NO: 520) |
| Kozak Sequence | GCCGCCACCATG (SEQ ID NO: 529) |
| Dead Seq212 | AAGAAGAAGCACCAGAGCGCCGCCGAGAAGAGGCAAGTGAAGAAGCT |
| | CAAGAATCAAGAGAAGGCCCAGAAGTACGCTAGCGAGCCTTCCCCCCT |
| | CCAGAGCGATACAGCTGGCGTGGAATGCTCCCAGAAAAAGACAGTCGT |
| | CAGCCACATTGCCAGCTCCAAGACACTGGCCAAGGCTATGGGACTCAA |
| | ATCCACACTGGTCATGGGCGACAAGCTGGTCATCACCAGCTTTGCTGCT |
| | AGCAAGGCTGTCGGAGGCGCTGGCTACAAAAGCGCTAACATTGAAAAA |
| | ATCACAGATCTGCAAGGAAGGGTCATTGAGGAGCACGAAAGGATGTTT |
| | AGCGCCGATGTCGGAGAGAAAAATATCGAACTGAGCAAGAATGACTGC |
| | CACACCAACGTCAACAACCCCGTGGTGACCAACATCGGAAAGGATTAC |
| | ATCGGACTGAAATCTAGGCTGGAGCAAGAGTTTTTCGGCAAGACATTC |
| | GAGAATGACAATCTGCATGTGCAGCTGGCCTACAATATCCTCGACATCA |
| | AGAAAATTCTGGGAACCTATGTGAACAATATCATTTATATCTTCTACAA |
| | TCTGAATAGGGCTGGCACCGGCAGAGATGAGAGGATGTATGACGACCT |
| | CATCGGCACACTGTACGCTTACAAACCCATGGAGGCTCAACAGACCTAT |
| | CTGCTCAAAGGCGACAAGGATATGAGGAGGTTTGAGGAGGTGAAACAG |
| | CTGCTGCAAAACACCTCCGCTTACTATGTGTATTACGGCACACTGTTCG |
| | AGAAGGTGAAGGCTAAGAGCAAGAAGGAACAGAGGGCTAAGGAGGCC |
| | GAAATCGACGCTTGTACCGCCCATAACTACGATGTGCTGAGACTGCTGT |
| | CCCTCATGAGGCAGCTGTGCATGCACTCCGTCGCTGGAACAGCCTTTAA |
| | GCTGGCTGAGTCCGCTCTGTTCAACATTGAGGATGTGCTCAGCGCCGAT |
| | CTGAAGGAAATCCTCGATGAAGCCTTCTCCGGCGCCGTGAACAAGCTC |
| | AATGACGGATTCGTGCAGCACTCCGGCAACAATCTGTACGTGCTCCAGC |
| | AGCTGTACCCTAATGAGACCATCGAGAGAATCGCCGAGAAGTACTACA |
| | GACTCACCGTGAGGAAGGAGGATCTGAACATGGGAGTCAACATTAAAA |
| | AGCTGAGGGAGCTGATCGTGGGCCAATACTTTCCCGAGGTCCTCGACA |
| | AAGAATACGACCTCTCCAAGAATGGAGACAGCGTGGTGACATACAGAA |
| | GCAAGATTTATACCGTGATGAATTACATTCTGCTGTATTACCTCGAGGA |
| | CCACGACTCCAGCAGAGAAAGCATGGTCGAAGCTCTGAGACAAAACAG |
| | AGAGGGCGATGAAGGCAAGGAGGAGATCTATAGACAGTTTGCCAAGA |
| | AGGTGTGGAACGGCGTGTCCGGACTGTTTGGCGTGTGTCTGAACCTCTT |
| | CAAGACCGAAAAGAGAAACAAGTTTAGGAGCAAAGTCGCCCTCCCCGA |
| | TGTGTCCGGCGCTGCCTATATGCTCTCCTCCGAGAACATCGACTACTTT |
| | GTCAAGATGCTCTTCTTTGTGTGTAAGTTTCTGGATGGCAAAGAAATCA |
| | ACGAGCTGCTGTGCGCTCTGATCAACAAATTTGATAATATTGCCGATAT |
| | TCTGGATGCTGCCGCTCAATGTGGCTCCTCCGTCTGGTTCGTGGACAGC |
| | TATAGGTTCTTCGAGAGATCTAGGAGGATTAGCGCCCAGATTAGAATCG |
| | TGAAGAACATCGCTTCCAAGGATTTTAAGAAATCCAAGAAGGATTCCG |
| | ATGAGAGCTACCCCGAGCAGCTGTATCTGGATGCTCTGGCTCTGCTCGG |
| | AGACGTCATCTCCAAGTACAAGCAGAATAGAGATGGCAGCGTCGTCAT |
| | CGATGACCAAGGCAATGCCGTGCTGACAGAGCAATACAAGAGGTTTAG |
| | ATATGAATTTTTCGAGGAGATCAAGAGGGACGAAAGCGGCGGCATCAA |
| | GTACAAGAAGTCCGGAAAACCCGAGTACAACCATCAGAGAAGGAATTT |
| | TATTCTGAATAATGTGCTGAAAAGCAAATGGTTTTTCTATGTGGTGAAG |
| | TACAATAGGCCCAGCAGCTGCAGAGAACTGATGAAGAATAAGGAAATT |
| | CTGAGGTTCGTGCTGAGAGACATCCCCGACTCCCAAGTGAGAAGATAC |
| | TTTAAGGCCGTCCAAGGAGAGGAAGCTTACGCTAGCGCCGAAGCTATG |
| | AGGACAAGACTGGTCGACGCTCTGTCCCAATTTAGCGTCACAGCTTGTC |
| | TGGATGAAGTGGGCGGCATGACAGACAAGGAATTCGCCTCCCAGAGGG |
| | CCGTCGATAGCAAAGAAAAACTGAGAGCCATCATCAGACTGTATCTGA |
| | CAGTCGCCTATCTGATTACCAAGAGCATGGTGAAGGTGAATACAAGGT |
| | TTAGCATTGCCTTTAGCGTGCTGGAGAGGGACTACTATCTGCTCATTGA |
| | CGGCAAGAAGAAATCCAGCGACTACACCGGAGAGGATATGCTGGCTCT |
| | GACCAGAAAATTTGTGGGCGAAGATGCTGGACTGTATAGAGAGTGGAA |
| | AGAGAAGAACGCTGAAGCCAAGGACAAATATTTTGACAAGGCCGAAA |
| | GGAAGAAGGTGCTGAGACAGAACGATAAGATGATCAGAAAGATGCAC |
| | TTCACACCCCACTCCCTCAATTACGTCCAAAAGAATCTCGAAAGCGTCC |
| | AGAGCAACGGACTGGCCGCCGTCATCAAGGAATATAGAAATGCCGTCG |
| | CTgcCCTCAATATCATCAATAGACTGGACGAGTACATTGGCTCCGCTAG |
| | GGCTGATAGCTACTACTCTCTGTACTGTTACTGCCTCCAAATGTATCTGA |
| | GCAAGAACTTCAGCGTGGGCTACCTCATCAACGTGCAAAAGCAGCTGG |
| | AGGAGCACCACACCTACATGAAGGATCTCATGTGGCTGCTCAACATCCC |
| | CTTCGCTTACAACCTCGCCAGATACAAAAATCTGTCCAACGAAAAACTC |
| | TTTTACGACGAGGAAGCCGCCGCCGAAAAGGCTGACAAGGCTGAGAAC |
| | GAGAGAGGCGAA (SEQ ID NO: 599) |
| Linker | GGAAGC |
| SV-40 NLS | CCCAAGAAGAAAAGGAAGGTC (SEQ ID NO: 532) |
| Linker | GAGGAC |
| HA Tag | TACCCCTACGATGTGCCCGACTACGCC (SEQ ID NO: 608) |
| WPRE3 | GATAATCAACCTCTGGATTACAAAATTTGTGAAAGATTGACTGGTATTC |
| | TTAACTATGTTGCTCCTTTTACGCTATGTGGATACGCTGCTTTAATGCCT |
| | TTGTATCATGCTATTGCTTCCCGTATGGCTTTCATTTTCTCCTCCTTGTAT |
| | AAATCCTGGTTAGTTCTTGCCACGGCGGAACTCATCGCCGCCTGCCTTG |
| | CCCGCTGCTGGACAGGGGCTCGGCTGTTGGGCACTGACAATTCCGTGG |
| | (SEQ ID NO: 609) |
| SV-40 poly A | AACTTGTTTATTGCAGCTTATAATGGTTACAAATAAAGCAATAGCATCA |
| | CAAATTTCACAAATAAAGCATTTTTTTCACTGCATTCTAGTTGTGGTTTG |
| | TCCAAACTCATCAATGTATCTTA (SEQ ID NO: 533) |
| 3'ITR | Aggaacccctagtgatggagttggccactccctctctgcgcgctcgctcgctcactgaggccgggcgaccaaaggtc |
| | gcccgacgcccgggctttgcccgggcggcctcagtgagcgagcgagcgcgcagctgcctgcagg (SEQ ID |
| | NO: 598) |

In some embodiments, an AAV vector comprising a nucleic acid encoding a CAG-targeting Cas13d composition comprises from 5′ to 3′: a sequence encoding a 5′ ITR (a first ITR), a sequence encoding an human U6 promoter, a dCas13d seq212 direct repeat, a sequence encoding a CAG guide 3 spacer sequence, a sequence encoding an EFS promoter, a sequence encoding a kozak sequence, a sequence encoding a dCas13d seq212 protein, a sequence encoding a linker sequence, a sequence encoding an SV-40 NLS, a sequence encoding a linker sequence, a sequence encoding an HA tag, a sequence encoding a WPRE, a sequence encoding an SV-40 polyA, and a 3′ ITR (a second ITR). In some embodiments, a nucleic acid encoding the vector is set forth in in SEQ ID NO: 589. In some embodiments, the CAG-targeting Cas13d composition is arranged as depicted in Table H. In some embodiments, vector A01922 is suitable for blocking. In some aspects, vector A01922 is encoded by a nucleic acid sequence comprising SEQ ID NO: 589.

In some embodiments, the vector set forth in Table H is referred to as A01922.

TABLE H
Vector A01922 encoding a CAG-repeat targeting dCas13d fusion for blocking

| Plasmid | |
|---|---|
| Element | Nucleic Acid Sequences |
| 5' ITR | Cctgcaggcagctgcgcgctcgctcgctcactgaggccgcccgggcgtcgggcgacctttggtcgcccggcctcag |
| | tgagcgagcgagcgcgcagagagggagtggccaactccatcactaggggttcct (SEQ ID NO: 597) |
| Human U6 | Gagggcctatttcccatgattccttcatatttgcatatacgatacaaggctgttagagagataattggaattaatttgactgt |
| promoter | aaacacaaagatattagtacaaaatacgtgacgtagaaagtaataatttcttgggtagtttgcagttttaaaattatgttttaa |
| | aatggactatcatatgcttaccgtaacttgaaagtatttcgatttcttggctttatatatcttgtggaaaggacgaaacacc |
| | (SEQ ID NO: 519) |
| Seq212 direct | Tagccctgcagtaaggcagggttctaagac (SEQ ID NO: 596) |
| repeat (DR) | |
| Spacer (CAG | Ctgctgctgctgctgctgctgctgct (SEQ ID NO: 459) |
| guide 3) | |
| EFS promoter | Taggtcttgaaaggagtgggaattggctccggtgcccgtcagtgggcagagcgcacatcgcccacagtccccgaga |
| | agttggggggaggggtcggcaattgatccggtgcctagagaaggtggcgcggggtaaactgggaaagtgatgtcgt |
| | gtactggctccgcctttttcccgagggtgggggagaaccgtatataagtgcagtagtcgccgtgaacgttctttttcgcaa |
| | cgggtttgccgccagaacacagg (SEQ ID NO: 520) |
| Kozak Sequence | GCCGCCACCATG (SEQ ID NO: 529) |
| Dead Seq212 | AAGAAGAAGCACCAGAGCGCCGCCGAGAAGAGGCAAGTGAAGAAGCT |
| | CAAGAATCAAGAGAAGGCCCAGAAGTACGCTAGCGAGCCTTCCCCCCT |
| | CCAGAGCGATACAGCTGGCGTGGAATGCTCCCAGAAAAAGACAGTCGT |
| | CAGCCACATTGCCAGCTCCAAGACACTGGCCAAGGCTATGGGACTCAA |
| | ATCCACACTGGTCATGGGCGACAAGCTGGTCATCACCAGCTTTGCTGCT |
| | AGCAAGGCTGTCGGAGGCGCTGGCTACAAAAGCGCTAACATTGAAAAA |
| | ATCACAGATCTGCAAGGAAGGGTCATTGAGGAGCACGAAAGGATGTTT |
| | AGCGCCGATGTCGGAGAGAAAAATATCGAACTGAGCAAGAATGACTGC |
| | CACACCAACGTCAACAACCCCGTGGTGACCAACATCGGAAAGGATTAC |
| | ATCGGACTGAAATCTAGGCTGGAGCAAGAGTTTTTCGGCAAGACATTC |
| | GAGAATGACAATCTGCATGTGCAGCTGGCCTACAATATCCTCGACATCA |
| | AGAAAATTCTGGGAACCTATGTGAACAATATCATTTATATCTTCTACAA |
| | TCTGAATAGGGCTGGCACCGGCAGAGATGAGAGGATGTATGACGACCT |
| | CATCGGCACACTGTACGCTTACAAACCCATGGAGGCTCAACAGACCTAT |
| | CTGCTCAAAGGCGACAAGGATATGAGGAGGTTTGAGGAGGTGAAACAG |
| | CTGCTGCAAAACACCTCCGCTTACTATGTGTATTACGGCACACTGTTCG |
| | AGAAGGTGAAGGCTAAGAGCAAGAAGGAACAGAGGGCTAAGGAGGCC |
| | GAAATCGACGCTTGTACCGCCCATAACTACGATGTGCTGAGACTGCTGT |
| | CCCTCATGgcGCAGCTGTGCATGgcCTCCGTCGCTGGAACAGCCTTTAAG |
| | CTGGCTGAGTCCGCTCTGTTCAACATTGAGGATGTGCTCAGCGCCGATC |
| | TGAAGGAAATCCTCGATGAAGCCTTCTCCGGCGCCGTGAACAAGCTCA |
| | ATGACGGATTCGTGCAGCACTCCGGCAACAATCTGTACGTGCTCCAGCA |
| | GCTGTACCCTAATGAGACCATCGAGAGAATCGCCGAGAAGTACTACAG |
| | ACTCACCGTGAGGAAGGAGGATCTGAACATGGGAGTCAACATTAAAAA |
| | GCTGAGGGAGCTGATCGTGGGCCAATACTTTCCCGAGGTCCTCGACAA |
| | AGAATACGACCTCTCCAAGAATGGAGACAGCGTGGTGACATACAGAAG |
| | CAAGATTTATACCGTGATGAATTACATTCTGCTGTATTACCTCGAGGAC |
| | CACGACTCCAGCAGAGAAAGCATGGTCGAAGCTCTGAGACAAAACAGA |
| | GAGGGCGATGAAGGCAAGGAGGAGATCTATAGACAGTTTGCCAAGAA |
| | GGTGTGGAACGGCGTGTCCGGACTGTTTGGCGTGTGTCTGAACCTCTTC |
| | AAGACCGAAAAGAGAAACAAGTTTAGGAGCAAAGTCGCCCTCCCCGAT |
| | GTGTCCGGCGCTGCCTATATGCTCTCCTCCGAGAACATCGACTACTTTG |
| | TCAAGATGCTCTTCTTTGTGTGTAAGTTTCTGGATGGCAAAGAAATCAA |
| | CGAGCTGCTGTGCGCTCTGATCAACAAATTTGATAATATTGCCGATATT |
| | CTGGATGCTGCCGCTCAATGTGGCTCCTCCGTCTGGTTCGTGGACAGCT |
| | ATAGGTTCTTCGAGAGATCTAGGAGGATTAGCGCCCAGATTAGAATCGT |
| | GAAGAACATCGCTTCCAAGGATTTTAAGAAATCCAAGAAGGATTCCGA |
| | TGAGAGCTACCCCGAGCAGCTGTATCTGGATGCTCTGGCTCTGCTCGGA |
| | GACGTCATCTCCAAGTACAAGCAGAATAGAGATGGCAGCGTCGTCATC |
| | GATGACCAAGGCAATGCCGTGCTGACAGAGCAATACAAGAGGTTTAGA |
| | TATGAATTTTTCGAGGAGATCAAGAGGGACGAAAGCGGCGGCATCAAG |
| | TACAAGAAGTCCGGAAAACCCGAGTACAACCATCAGAGAAGGAATTTT |
| | ATTCTGAATAATGTGCTGAAAAGCAAATGGTTTTTCTATGTGGTGAAGT |
| | ACAATAGGCCCAGCAGCTGCAGAGAACTGATGAAGAATAAGGAAATTC |
| | TGAGGTTCGTGCTGAGAGACATCCCCGACTCCCAAGTGAGAAGATACTT |
| | TAAGGCCGTCCAAGGAGAGGAAGCTTACGCTAGCGCCGAAGCTATGAG |
| | GACAAGACTGGTCGACGCTCTGTCCCAATTTAGCGTCACAGCTTGTCTG |
| | GATGAAGTGGGCGGCATGACAGACAAGGAATTCGCCTCCCAGAGGGCC |
| | GTCGATAGCAAAGAAAAACTGAGAGCCATCATCAGACTGTATCTGACA |
| | GTCGCCTATCTGATTACCAAGAGCATGGTGAAGGTGAATACAAGGTTTA |
| | GCATTGCCTTTAGCGTGCTGGAGAGGGACTACTATCTGCTCATTGACGG |
| | CAAGAAGAAATCCAGCGACTACACCGGAGAGGATATGCTGGCTCTGAC |
| | CAGAAAATTTGTGGGCGAAGATGCTGGACTGTATAGAGAGTGGAAAGA |
| | GAAGAACGCTGAAGCCAAGGACAAATATTTTGACAAGGCCGAAAGGA |
| | AGAAGGTGCTGAGACAGAACGATAAGATGATCAGAAAGATGCACTTCA |
| | CACCCCACTCCCTCAATTACGTCCAAAAGAATCTCGAAAGCGTCCAGAG |
| | CAACGGACTGGCCGCCGTCATCAAGGAATATgcAAATGCCGTCGCTgcCC |
| | TCAATATCATCAATAGACTGGACGAGTACATTGGCTCCGCTAGGGCTGA |
| | TAGCTACTACTCTCTGTACTGTTACTGCCTCCAAATGTATCTGAGCAAG |
| | AACTTCAGCGTGGGCTACCTCATCAACGTGCAAAAGCAGCTGGAGGAG |
| | CACCACACCTACATGAAGGATCTCATGTGGCTGCTCAACATCCCCTTCG |
| | CTTACAACCTCGCCAGATACAAAAATCTGTCCAACGAAAAACTCTTTTA |
| | CGACGAGGAAGCCGCCGCCGAAAAGGCTGACAAGGCTGAGAACGAGA |
| | GAGGCGAA (SEQ ID NO: 600) |
| Linker | GGAAGC |
| SV-40 NLS | CCCAAGAAGAAAAGGAAGGTC (SEQ ID NO: 532) |
| Linker | GAGGAC |
| HA Tag | TACCCCTACGATGTGCCCGACTACGCC (SEQ ID NO: 608) |
| WPRE3 | GATAATCAACCTCTGGATTACAAAATTTGTGAAAGATTGACTGGTATTC |
| | TTAACTATGTTGCTCCTTTTACGCTATGTGGATACGCTGCTTTAATGCCT |
| | TTGTATCATGCTATTGCTTCCCGTATGGCTTTCATTTTCTCCTCCTTGTAT |
| | AAATCCTGGTTAGTTCTTGCCACGGCGGAACTCATCGCCGCCTGCCTTG |
| | CCCGCTGCTGGACAGGGGCTCGGCTGTTGGGCACTGACAATTCCGTGG |
| | (SEQ ID NO: 609) |
| SV-40 poly A | AACTTGTTTATTGCAGCTTATAATGGTTACAAATAAAGCAATAGCATCA |
| | CAAATTTCACAAATAAAGCATTTTTTTCACTGCATTCTAGTTGTGGTTTG |
| | TCCAAACTCATCAATGTATCTTA (SEQ ID NO: 533) |
| 3'ITR | Aggaacccctagtgatggagttggccactccctctctgcgcgctcgctcgctcactgaggccgggcgaccaaaggtc |
| | gcccgacgcccgggctttgcccgggcggcctcagtgagcgagcgagcgcgcagctgcctgcagg (SEQ ID |
| | NO: 598) |

In some embodiments, an AAV vector comprising a nucleic acid encoding a CAG-targeting Cas13d composition comprises from 5′ to 3′: a sequence encoding a 5′ ITR (a first ITR), a sequence encoding an human U6 promoter, a dCas13d seq212 direct repeat, a sequence encoding a CAG guide 3 spacer sequence, a sequence encoding an EFS promoter, a sequence encoding a kozak sequence, a sequence encoding a dCas13d seq212 protein, a sequence encoding a linker sequence, a sequence encoding an SV-40 NLS, a sequence encoding a linker sequence, a sequence encoding an HA tag, a sequence encoding a WPRE, a sequence encoding an SV-40 polyA, and a 3′ ITR (a second ITR). In some embodiments, the CAG-targeting Cas13d composition is arranged as depicted in Table I.

TABLE I
Vector encoding a CAG-repeat targeting dCas 13d fusion

| Plasmid | |
|---|---|
| Element | Nucleic Acid Sequences |
| 5' ITR | Cctgcaggcagctgcgcgctcgctcgctcactgaggccgcccgggcgtcgggcgacctttggtcgcccggcctcag |
| | tgagcgagcgagcgcgcagagagggagtggccaactccatcactaggggttcct (SEQ ID NO: 597) |
| Human U6 | Gagggcctatttcccatgattccttcatatttgcatatacgatacaaggctgttagagagataattggaattaatttgactgt |
| promoter | aaacacaaagatattagtacaaaatacgtgacgtagaaagtaataatttcttgggtagtttgcagttttaaaattatgttttaa |
| | aatggactatcatatgcttaccgtaacttgaaagtatttcgatttcttggctttatatatcttgtggaaaggacgaaacacc |
| | (SEQ ID NO: 519) |
| Seq212 direct | Tagccctgcagtaaggcagggttctaagac (SEQ ID NO: 596) |
| repeat (DR) | |
| Spacer (CAG | Ctgctgctgctgctgctgctgctgct (SEQ ID NO: 459) |
| guide 3) | |
| EFS promoter | Taggtcttgaaaggagtgggaattggctccggtgcccgtcagtgggcagagcgcacatcgcccacagtccccgaga |
| | agttggggggaggggtcggcaattgatccggtgcctagagaaggtggcgcggggtaaactgggaaagtgatgtcgt |
| | gtactggctccgcctttttcccgaggggggggagaaccgtatataagtgcagtagtcgccgtgaacgttctttttcgcaa |
| | cgggtttgccgccagaacacagg (SEQ ID NO: 520) |
| Kozak Sequence | GCCGCCACCATG (SEQ ID NO: 529) |
| Dead Seq212 | AAGAAGAAGCACCAGAGCGCCGCCGAGAAGAGGCAAGTGAAGAAGCT |
| | CAAGAATCAAGAGAAGGCCCAGAAGTACGCTAGCGAGCCTTCCCCCCT |
| | CCAGAGCGATACAGCTGGCGTGGAATGCTCCCAGAAAAAGACAGTCGT |
| | CAGCCACATTGCCAGCTCCAAGACACTGGCCAAGGCTATGGGACTCAA |
| | ATCCACACTGGTCATGGGCGACAAGCTGGTCATCACCAGCTTTGCTGCT |
| | AGCAAGGCTGTCGGAGGCGCTGGCTACAAAAGCGCTAACATTGAAAAA |
| | ATCACAGATCTGCAAGGAAGGGTCATTGAGGAGCACGAAAGGATGTTT |
| | AGCGCCGATGTCGGAGAGAAAAATATCGAACTGAGCAAGAATGACTGC |
| | CACACCAACGTCAACAACCCCGTGGTGACCAACATCGGAAAGGATTAC |
| | ATCGGACTGAAATCTAGGCTGGAGCAAGAGTTTTTCGGCAAGACATTC |
| | GAGAATGACAATCTGCATGTGCAGCTGGCCTACAATATCCTCGACATCA |
| | AGAAAATTCTGGGAACCTATGTGAACAATATCATTTATATCTTCTACAA |
| | TCTGAATAGGGCTGGCACCGGCAGAGATGAGAGGATGTATGACGACCT |
| | CATCGGCACACTGTACGCTTACAAACCCATGGAGGCTCAACAGACCTAT |
| | CTGCTCAAAGGCGACAAGGATATGAGGAGGTTTGAGGAGGTGAAACAG |
| | CTGCTGCAAAACACCTCCGCTTACTATGTGTATTACGGCACACTGTTCG |
| | AGAAGGTGAAGGCTAAGAGCAAGAAGGAACAGAGGGCTAAGGAGGCC |
| | GAAATCGACGCTTGTACCGCCCATAACTACGATGTGCTGAGACTGCTGT |
| | CCCTCATGAGGCAGCTGTGCATGCACTCCGTCGCTGGAACAGCCTTTAA |
| | GCTGGCTGAGTCCGCTCTGTTCAACATTGAGGATGTGCTCAGCGCCGAT |
| | CTGAAGGAAATCCTCGATGAAGCCTTCTCCGGCGCCGTGAACAAGCTC |
| | AATGACGGATTCGTGCAGCACTCCGGCAACAATCTGTACGTGCTCCAGC |
| | AGCTGTACCCTAATGAGACCATCGAGAGAATCGCCGAGAAGTACTACA |
| | GACTCACCGTGAGGAAGGAGGATCTGAACATGGGAGTCAACATTAAAA |
| | AGCTGAGGGAGCTGATCGTGGGCCAATACTTTCCCGAGGTCCTCGACA |
| | AAGAATACGACCTCTCCAAGAATGGAGACAGCGTGGTGACATACAGAA |
| | GCAAGATTTATACCGTGATGAATTACATTCTGCTGTATTACCTCGAGGA |
| | CCACGACTCCAGCAGAGAAAGCATGGTCGAAGCTCTGAGACAAAACAG |
| | AGAGGGCGATGAAGGCAAGGAGGAGATCTATAGACAGTTTGCCAAGA |
| | AGGTGTGGAACGGCGTGTCCGGACTGTTTGGCGTGTGTCTGAACCTCTT |
| | CAAGACCGAAAAGAGAAACAAGTTTAGGAGCAAAGTCGCCCTCCCCGA |
| | TGTGTCCGGCGCTGCCTATATGCTCTCCTCCGAGAACATCGACTACTTT |
| | GTCAAGATGCTCTTCTTTGTGTGTAAGTTTCTGGATGGCAAAGAAATCA |
| | ACGAGCTGCTGTGCGCTCTGATCAACAAATTTGATAATATTGCCGATAT |
| | TCTGGATGCTGCCGCTCAATGTGGCTCCTCCGTCTGGTTCGTGGACAGC |
| | TATAGGTTCTTCGAGAGATCTAGGAGGATTAGCGCCCAGATTAGAATCG |
| | TGAAGAACATCGCTTCCAAGGATTTTAAGAAATCCAAGAAGGATTCCG |
| | ATGAGAGCTACCCCGAGCAGCTGTATCTGGATGCTCTGGCTCTGCTCGG |
| | AGACGTCATCTCCAAGTACAAGCAGAATAGAGATGGCAGCGTCGTCAT |
| | CGATGACCAAGGCAATGCCGTGCTGACAGAGCAATACAAGAGGTTTAG |
| | ATATGAATTTTTCGAGGAGATCAAGAGGGACGAAAGCGGCGGCATCAA |
| | GTACAAGAAGTCCGGAAAACCCGAGTACAACCATCAGAGAAGGAATTT |
| | TATTCTGAATAATGTGCTGAAAAGCAAATGGTTTTTCTATGTGGTGAAG |
| | TACAATAGGCCCAGCAGCTGCAGAGAACTGATGAAGAATAAGGAAATT |
| | CTGAGGTTCGTGCTGAGAGACATCCCCGACTCCCAAGTGAGAAGATAC |
| | TTTAAGGCCGTCCAAGGAGAGGAAGCTTACGCTAGCGCCGAAGCTATG |
| | AGGACAAGACTGGTCGACGCTCTGTCCCAATTTAGCGTCACAGCTTGTC |
| | TGGATGAAGTGGGCGGCATGACAGACAAGGAATTCGCCTCCCAGAGGG |
| | CCGTCGATAGCAAAGAAAAACTGAGAGCCATCATCAGACTGTATCTGA |
| | CAGTCGCCTATCTGATTACCAAGAGCATGGTGAAGGTGAATACAAGGT |
| | TTAGCATTGCCTTTAGCGTGCTGGAGAGGGACTACTATCTGCTCATTGA |
| | CGGCAAGAAGAAATCCAGCGACTACACCGGAGAGGATATGCTGGCTCT |
| | GACCAGAAAATTTGTGGGCGAAGATGCTGGACTGTATAGAGAGTGGAA |
| | AGAGAAGAACGCTGAAGCCAAGGACAAATATTTTGACAAGGCCGAAA |
| | GGAAGAAGGTGCTGAGACAGAACGATAAGATGATCAGAAAGATGCAC |
| | TTCACACCCCACTCCCTCAATTACGTCCAAAAGAATCTCGAAAGCGTCC |
| | AGAGCAACGGACTGGCCGCCGTCATCAAGGAATATAGAAATGCCGTCG |
| | CTgcCCTCAATATCATCAATAGACTGGACGAGTACATTGGCTCCGCTAG |
| | GGCTGATAGCTACTACTCTCTGTACTGTTACTGCCTCCAAATGTATCTGA |
| | GCAAGAACTTCAGCGTGGGCTACCTCATCAACGTGCAAAAGCAGCTGG |
| | AGGAGCACCACACCTACATGAAGGATCTCATGTGGCTGCTCAACATCCC |
| | CTTCGCTTACAACCTCGCCAGATACAAAAATCTGTCCAACGAAAAACTC |
| | TTTTACGACGAGGAAGCCGCCGCCGAAAAGGCTGACAAGGCTGAGAAC |
| | GAGAGAGGCGAA (SEQ ID NO: 601) |
| Linker | GGAAGC |
| SV-40 NLS | CCCAAGAAGAAAAGGAAGGTC (SEQ ID NO: 532) |
| Linker | GAGGAC |
| HA Tag | TACCCCTACGATGTGCCCGACTACGCC (SEQ ID NO: 608) |
| WPRE3 | GATAATCAACCTCTGGATTACAAAATTTGTGAAAGATTGACTGGTATTC |
| | TTAACTATGTTGCTCCTTTTACGCTATGTGGATACGCTGCTTTAATGCCT |
| | TTGTATCATGCTATTGCTTCCCGTATGGCTTTCATTTTCTCCTCCTTGTAT |
| | AAATCCTGGTTAGTTCTTGCCACGGCGGAACTCATCGCCGCCTGCCTTG |
| | CCCGCTGCTGGACAGGGGCTCGGCTGTTGGGCACTGACAATTCCGTGG |
| | (SEQ ID NO: 609) |
| SV-40 poly A | AACTTGTTTATTGCAGCTTATAATGGTTACAAATAAAGCAATAGCATCA |
| | CAAATTTCACAAATAAAGCATTTTTTTCACTGCATTCTAGTTGTGGTTTG |
| | TCCAAACTCATCAATGTATCTTA (SEQ ID NO: 533) |
| 3'ITR | Aggaacccctagtgatggagttggccactccctctctgcgcgctcgctcgctcactgaggccgggcgaccaaaggtc |
| | gcccgacgcccgggctttgcccgggggcctcagtgagcgagcgagcgcgcagctgcctgcagg (SEQ ID |
| | NO: 598) |

In some embodiments, an AAV vector comprising a nucleic acid encoding a CAG-targeting Cas13d composition comprises from 5′ to 3′: a sequence encoding a 5′ ITR (a first ITR), a sequence encoding an human U6 promoter, a dCas13d seq212 direct repeat, a sequence encoding a CAG guide 3 spacer sequence, a sequence encoding an EFS promoter, a sequence encoding a kozak sequence, a sequence encoding a dCas13d seq212 protein, a sequence encoding a linker sequence, a sequence encoding an SV-40 NLS, a sequence encoding a linker sequence, a sequence encoding an HA tag, a sequence encoding a WPRE, a sequence encoding an SV-40 polyA, and a 3′ ITR (a second ITR). In some embodiments, the CAG-targeting Cas13d composition is arranged as depicted in Table J.

TABLE J
Vector encoding a CAG-repeat targeting dCas13d fusion

| Plasmid | |
|---|---|
| Element | Nucleic Acid Sequences |
| 5′ ITR | Cctgcaggcagctgcgcgctcgctcgctcactgaggccgcccgggcgtcgggcgacctttggtcgcccggcctcag |
| | tgagcgagcgagcgcgcagagagggagtggccaactccatcactaggggttcct (SEQ ID NO: 597) |
| Human U6 | Gagggcctatttcccatgattccttcatatttgcatatacgatacaaggctgttagagagataattggaattaatttgactgt |
| promoter | aaacacaaagatattagtacaaaatacgtgacgtagaaagtaataatttcttgggtagtttgcagttttaaaattatgttttaa |
| | aatggactatcatatgcttaccgtaacttgaaagtatttcgatttcttggctttatatatcttgtggaaaggacgaaacacc |
| | (SEQ ID NO: 519) |
| Seq212 direct | Tagccctgcagtaaggcagggttctaagac (SEQ ID NO: 596) |
| repeat (DR) | |
| Spacer (CAG | Ctgctgctgctgctgctgctgctgct (SEQ ID NO: 459) |
| guide 3) | |
| EFS promoter | Taggtcttgaaaggagtgggaattggctccggtgcccgtcagtgggcagagcgcacatcgcccacagtccccgaga |
| | agttggggggaggggtcggcaattgatccggtgcctagagaaggtggcgcggggtaaactgggaaagtgatgtcgt |
| | gtactggctccgcctttttcccgaggggggggagaaccgtatataagtgcagtagtcgccgtgaacgttctttttcgcaa |
| | cgggtttgccgccagaacacagg (SEQ ID NO: 520) |
| Kozak | GCCGCCACCATG (SEQ ID NO: 529) |
| Sequence | |
| Dead Seq212 | AAGAAGAAGCACCAGAGCGCCGCCGAGAAGAGGCAAGTGAAGAAGCT |
| | CAAGAATCAAGAGAAGGCCCAGAAGTACGCTAGCGAGCCTTCCCCCCT |
| | CCAGAGCGATACAGCTGGCGTGGAATGCTCCCAGAAAAAGACAGTCGT |
| | CAGCCACATTGCCAGCTCCAAGACACTGGCCAAGGCTATGGGACTCAA |
| | ATCCACACTGGTCATGGGCGACAAGCTGGTCATCACCAGCTTTGCTGCT |
| | AGCAAGGCTGTCGGAGGCGCTGGCTACAAAAGCGCTAACATTGAAAAA |
| | ATCACAGATCTGCAAGGAAGGGTCATTGAGGAGCACGAAAGGATGTTT |
| | AGCGCCGATGTCGGAGAGAAAAATATCGAACTGAGCAAGAATGACTGC |
| | CACACCAACGTCAACAACCCCGTGGTGACCAACATCGGAAAGGATTAC |
| | ATCGGACTGAAATCTAGGCTGGAGCAAGAGTTTTTCGGCAAGACATTC |
| | GAGAATGACAATCTGCATGTGCAGCTGGCCTACAATATCCTCGACATCA |
| | AGAAAATTCTGGGAACCTATGTGAACAATATCATTTATATCTTCTACAA |
| | TCTGAATAGGGCTGGCACCGGCAGAGATGAGAGGATGTATGACGACCT |
| | CATCGGCACACTGTACGCTTACAAACCCATGGAGGCTCAACAGACCTAT |
| | CTGCTCAAAGGCGACAAGGATATGAGGAGGTTTGAGGAGGTGAAACAG |
| | CTGCTGCAAAACACCTCCGCTTACTATGTGTATTACGGCACACTGTTCG |
| | AGAAGGTGAAGGCTAAGAGCAAGAAGGAACAGAGGGCTAAGGAGGCC |
| | GAAATCGACGCTTGTACCGCCCATAACTACGATGTGCTGAGACTGCTGT |
| | CCCTCATGagGCAGCTGTGCATGgcCTCCGTCGCTGGAACAGCCTTTAAG |
| | CTGGCTGAGTCCGCTCTGTTCAACATTGAGGATGTGCTCAGCGCCGATC |
| | TGAAGGAAATCCTCGATGAAGCCTTCTCCGGCGCCGTGAACAAGCTCA |
| | ATGACGGATTCGTGCAGCACTCCGGCAACAATCTGTACGTGCTCCAGCA |
| | GCTGTACCCTAATGAGACCATCGAGAGAATCGCCGAGAAGTACTACAG |
| | ACTCACCGTGAGGAAGGAGGATCTGAACATGGGAGTCAACATTAAAAA |
| | GCTGAGGGAGCTGATCGTGGGCCAATACTTTCCCGAGGTCCTCGACAA |
| | AGAATACGACCTCTCCAAGAATGGAGACAGCGTGGTGACATACAGAAG |
| | CAAGATTTATACCGTGATGAATTACATTCTGCTGTATTACCTCGAGGAC |
| | CACGACTCCAGCAGAGAAAGCATGGTCGAAGCTCTGAGACAAAACAGA |
| | GAGGGCGATGAAGGCAAGGAGGAGATCTATAGACAGTTTGCCAAGAA |
| | GGTGTGGAACGGCGTGTCCGGACTGTTTGGCGTGTGTCTGAACCTCTTC |
| | AAGACCGAAAAGAGAAACAAGTTTAGGAGCAAAGTCGCCCTCCCCGAT |
| | GTGTCCGGCGCTGCCTATATGCTCTCCTCCGAGAACATCGACTACTTTG |
| | TCAAGATGCTCTTCTTTGTGTGTAAGTTTCTGGATGGCAAAGAAATCAA |
| | CGAGCTGCTGTGCGCTCTGATCAACAAATTTGATAATATTGCCGATATT |
| | CTGGATGCTGCCGCTCAATGTGGCTCCTCCGTCTGGTTCGTGGACAGCT |
| | ATAGGTTCTTCGAGAGATCTAGGAGGATTAGCGCCCAGATTAGAATCGT |
| | GAAGAACATCGCTTCCAAGGATTTTAAGAAATCCAAGAAGGATTCCGA |
| | TGAGAGCTACCCCGAGCAGCTGTATCTGGATGCTCTGGCTCTGCTCGGA |
| | GACGTCATCTCCAAGTACAAGCAGAATAGAGATGGCAGCGTCGTCATC |
| | GATGACCAAGGCAATGCCGTGCTGACAGAGCAATACAAGAGGTTTAGA |
| | TATGAATTTTTCGAGGAGATCAAGAGGGACGAAAGCGGCGGCATCAAG |
| | TACAAGAAGTCCGGAAAACCCGAGTACAACCATCAGAGAAGGAATTTT |
| | ATTCTGAATAATGTGCTGAAAAGCAAATGGTTTTTCTATGTGGTGAAGT |
| | ACAATAGGCCCAGCAGCTGCAGAGAACTGATGAAGAATAAGGAAATTC |
| | TGAGGTTCGTGCTGAGAGACATCCCCGACTCCCAAGTGAGAAGATACTT |
| | TAAGGCCGTCCAAGGAGAGGAAGCTTACGCTAGCGCCGAAGCTATGAG |
| | GACAAGACTGGTCGACGCTCTGTCCCAATTTAGCGTCACAGCTTGTCTG |
| | GATGAAGTGGGCGGCATGACAGACAAGGAATTCGCCTCCCAGAGGGCC |
| | GTCGATAGCAAAGAAAAACTGAGAGCCATCATCAGACTGTATCTGACA |
| | GTCGCCTATCTGATTACCAAGAGCATGGTGAAGGTGAATACAAGGTTTA |
| | GCATTGCCTTTAGCGTGCTGGAGAGGGACTACTATCTGCTCATTGACGG |
| | CAAGAAGAAATCCAGCGACTACACCGGAGAGGATATGCTGGCTCTGAC |
| | CAGAAAATTTGTGGGCGAAGATGCTGGACTGTATAGAGAGTGGAAAGA |
| | GAAGAACGCTGAAGCCAAGGACAAATATTTTGACAAGGCCGAAAGGA |
| | AGAAGGTGCTGAGACAGAACGATAAGATGATCAGAAAGATGCACTTCA |
| | CACCCCACTCCCTCAATTACGTCCAAAAGAATCTCGAAAGCGTCCAGAG |
| | CAACGGACTGGCCGCCGTCATCAAGGAATATagAAATGCCGTCGCTcaCC |
| | TCAATATCATCAATAGACTGGACGAGTACATTGGCTCCGCTAGGGCTGA |
| | TAGCTACTACTCTCTGTACTGTTACTGCCTCCAAATGTATCTGAGCAAG |
| | AACTTCAGCGTGGGCTACCTCATCAACGTGCAAAAGCAGCTGGAGGAG |
| | CACCACACCTACATGAAGGATCTCATGTGGCTGCTCAACATCCCCTTCG |
| | CTTACAACCTCGCCAGATACAAAAATCTGTCCAACGAAAAACTCTTTTA |
| | CGACGAGGAAGCCGCCGCCGAAAAGGCTGACAAGGCTGAGAACGAGA |
| | GAGGCGAA (SEQ ID NO: 602) |
| Linker | GGAAGC |
| SV-40 NLS | CCCAAGAAGAAAAGGAAGGTC (SEQ ID NO: 532) |
| Linker | GAGGAC |
| HA Tag | TACCCCTACGATGTGCCCGACTACGCC (SEQ ID NO: 608) |
| WPRE3 | GATAATCAACCTCTGGATTACAAAATTTGTGAAAGATTGACTGGTATTC |
| | TTAACTATGTTGCTCCTTTTACGCTATGTGGATACGCTGCTTTAATGCCT |
| | TTGTATCATGCTATTGCTTCCCGTATGGCTTTCATTTTCTCCTCCTTGTAT |
| | AAATCCTGGTTAGTTCTTGCCACGGCGGAACTCATCGCCGCCTGCCTTG |
| | CCCGCTGCTGGACAGGGGCTCGGCTGTTGGGCACTGACAATTCCGTGG |
| | (SEQ ID NO: 609) |
| SV-40 poly A | AACTTGTTTATTGCAGCTTATAATGGTTACAAATAAAGCAATAGCATCA |
| | CAAATTTCACAAATAAAGCATTTTTTTCACTGCATTCTAGTTGTGGTTTG |
| | TCCAAACTCATCAATGTATCTTA (SEQ ID NO: 533) |
| 3′ITR | Aggaacccctagtgatggagttggccactccctctctgcgcgctcgctcgctcactgaggccggggaccaaaggtc |
| | gcccgacgcccgggctttgcccgggcggcctcagtgagcgagcgagcgcgcagctgcctgcagg (SEQ ID |
| | NO: 598) |

In some embodiments, an AAV vector comprising a nucleic acid encoding a CAG-targeting Cas13d composition comprises from 5′ to 3′: a sequence encoding a 5′ ITR (a first ITR), a sequence encoding an human U6 promoter, a dCas13d seq212 direct repeat, a sequence encoding a CAG guide 3 spacer sequence, a sequence encoding an EFS promoter, a sequence encoding a kozak sequence, a sequence encoding a dCas13d seq212 protein, a sequence encoding a linker sequence, a sequence encoding an SV-40 NLS, a sequence encoding a linker sequence, a sequence encoding an HA tag, a sequence encoding a WPRE, a sequence encoding an SV-40 polyA, and a 3′ ITR (a second ITR). In some embodiments, the CAG-targeting Cas13d composition is arranged as depicted in Table K.

TABLE K
Vector encoding a CAG-repeat targeting dCas13d fusion

| Plasmid | |
|---|---|
| Element | Nucleic Acid Sequences |
| 5′ ITR | Cctgcaggcagctgcgcgctcgctcgctcactgaggccgcccgggcgtcgggcgacctttggtcgcccggcctcag |
| | tgagcgagcgagcgcgcagagagggagtggccaactccatcactaggggttcct (SEQ ID NO: 597) |
| Human U6 | Gagggcctatttcccatgattccttcatatttgcatatacgatacaaggctgttagagagataattggaattaatttgactgt |
| promoter | aaacacaaagatattagtacaaaatacgtgacgtagaaagtaataatttcttgggtagtttgcagttttaaaattatgttttaa |
| | aatggactatcatatgcttaccgtaacttgaaagtatttcgatttcttggctttatatatcttgtggaaaggacgaaacacc |
| | (SEQ ID NO: 519) |
| Seq212 direct | Tagccctgcagtaaggcagggttctaagac (SEQ ID NO: 596) |
| repeat (DR) | |
| Spacer (CAG | Ctgctgctgctgctgctgctgctgct (SEQ ID NO: 459) |
| guide 3) | |
| EFS promoter | Taggtcttgaaaggagtgggaattggctccggtgcccgtcagtgggcagagcgcacatcgcccacagtccccgaga |
| | agttggggggaggggtcggcaattgatccggtgcctagagaaggtggcgcggggtaaactgggaaagtgatgtcgt |
| | gtactggctccgcctttttcccgaggggggggagaaccgtatataagtgcagtagtcgccgtgaacgttctttttcgcaa |
| | cgggtttgccgccagaacacagg (SEQ ID NO: 520) |
| Kozak Sequence | GCCGCCACCATG (SEQ ID NO: 529) |
| Dead Seq212 | AAGAAGAAGCACCAGAGCGCCGCCGAGAAGAGGCAAGTGAAGAAGCT |
| | CAAGAATCAAGAGAAGGCCCAGAAGTACGCTAGCGAGCCTTCCCCCCT |
| | CCAGAGCGATACAGCTGGCGTGGAATGCTCCCAGAAAAAGACAGTCGT |
| | CAGCCACATTGCCAGCTCCAAGACACTGGCCAAGGCTATGGGACTCAA |
| | ATCCACACTGGTCATGGGCGACAAGCTGGTCATCACCAGCTTTGCTGCT |
| | AGCAAGGCTGTCGGAGGCGCTGGCTACAAAAGCGCTAACATTGAAAAA |
| | ATCACAGATCTGCAAGGAAGGGTCATTGAGGAGCACGAAAGGATGTTT |
| | AGCGCCGATGTCGGAGAGAAAAATATCGAACTGAGCAAGAATGACTGC |
| | CACACCAACGTCAACAACCCCGTGGTGACCAACATCGGAAAGGATTAC |
| | ATCGGACTGAAATCTAGGCTGGAGCAAGAGTTTTTCGGCAAGACATTC |
| | GAGAATGACAATCTGCATGTGCAGCTGGCCTACAATATCCTCGACATCA |
| | AGAAAATTCTGGGAACCTATGTGAACAATATCATTTATATCTTCTACAA |
| | TCTGAATAGGGCTGGCACCGGCAGAGATGAGAGGATGTATGACGACCT |
| | CATCGGCACACTGTACGCTTACAAACCCATGGAGGCTCAACAGACCTAT |
| | CTGCTCAAAGGCGACAAGGATATGAGGAGGTTTGAGGAGGTGAAACAG |
| | CTGCTGCAAAACACCTCCGCTTACTATGTGTATTACGGCACACTGTTCG |
| | AGAAGGTGAAGGCTAAGAGCAAGAAGGAACAGAGGGCTAAGGAGGCC |
| | GAAATCGACGCTTGTACCGCCCATAACTACGATGTGCTGAGACTGCTGT |
| | CCCTCATGgcGCAGCTGTGCATGcaCTCCGTCGCTGGAACAGCCTTTAAG |
| | CTGGCTGAGTCCGCTCTGTTCAACATTGAGGATGTGCTCAGCGCCGATC |
| | TGAAGGAAATCCTCGATGAAGCCTTCTCCGGCGCCGTGAACAAGCTCA |
| | ATGACGGATTCGTGCAGCACTCCGGCAACAATCTGTACGTGCTCCAGCA |
| | GCTGTACCCTAATGAGACCATCGAGAGAATCGCCGAGAAGTACTACAG |
| | ACTCACCGTGAGGAAGGAGGATCTGAACATGGGAGTCAACATTAAAAA |
| | GCTGAGGGAGCTGATCGTGGGCCAATACTTTCCCGAGGTCCTCGACAA |
| | AGAATACGACCTCTCCAAGAATGGAGACAGCGTGGTGACATACAGAAG |
| | CAAGATTTATACCGTGATGAATTACATTCTGCTGTATTACCTCGAGGAC |
| | CACGACTCCAGCAGAGAAAGCATGGTCGAAGCTCTGAGACAAAACAGA |
| | GAGGGCGATGAAGGCAAGGAGGAGATCTATAGACAGTTTGCCAAGAA |
| | GGTGTGGAACGGCGTGTCCGGACTGTTTGGCGTGTGTCTGAACCTCTTC |
| | AAGACCGAAAAGAGAAACAAGTTTAGGAGCAAAGTCGCCCTCCCCGAT |
| | GTGTCCGGCGCTGCCTATATGCTCTCCTCCGAGAACATCGACTACTTTG |
| | TCAAGATGCTCTTCTTTGTGTGTAAGTTTCTGGATGGCAAAGAAATCAA |
| | CGAGCTGCTGTGCGCTCTGATCAACAAATTTGATAATATTGCCGATATT |
| | CTGGATGCTGCCGCTCAATGTGGCTCCTCCGTCTGGTTCGTGGACAGCT |
| | ATAGGTTCTTCGAGAGATCTAGGAGGATTAGCGCCCAGATTAGAATCGT |
| | GAAGAACATCGCTTCCAAGGATTTTAAGAAATCCAAGAAGGATTCCGA |
| | TGAGAGCTACCCCGAGCAGCTGTATCTGGATGCTCTGGCTCTGCTCGGA |
| | GACGTCATCTCCAAGTACAAGCAGAATAGAGATGGCAGCGTCGTCATC |
| | GATGACCAAGGCAATGCCGTGCTGACAGAGCAATACAAGAGGTTTAGA |
| | TATGAATTTTTCGAGGAGATCAAGAGGGACGAAAGCGGCGGCATCAAG |
| | TACAAGAAGTCCGGAAAACCCGAGTACAACCATCAGAGAAGGAATTTT |
| | ATTCTGAATAATGTGCTGAAAAGCAAATGGTTTTTCTATGTGGTGAAGT |
| | ACAATAGGCCCAGCAGCTGCAGAGAACTGATGAAGAATAAGGAAATTC |
| | TGAGGTTCGTGCTGAGAGACATCCCCGACTCCCAAGTGAGAAGATACTT |
| | TAAGGCCGTCCAAGGAGAGGAAGCTTACGCTAGCGCCGAAGCTATGAG |
| | GACAAGACTGGTCGACGCTCTGTCCCAATTTAGCGTCACAGCTTGTCTG |
| | GATGAAGTGGGCGGCATGACAGACAAGGAATTCGCCTCCCAGAGGGCC |
| | GTCGATAGCAAAGAAAAACTGAGAGCCATCATCAGACTGTATCTGACA |
| | GTCGCCTATCTGATTACCAAGAGCATGGTGAAGGTGAATACAAGGTTTA |
| | GCATTGCCTTTAGCGTGCTGGAGAGGGACTACTATCTGCTCATTGACGG |
| | CAAGAAGAAATCCAGCGACTACACCGGAGAGGATATGCTGGCTCTGAC |
| | CAGAAAATTTGTGGGCGAAGATGCTGGACTGTATAGAGAGTGGAAAGA |
| | GAAGAACGCTGAAGCCAAGGACAAATATTTTGACAAGGCCGAAAGGA |
| | AGAAGGTGCTGAGACAGAACGATAAGATGATCAGAAAGATGCACTTCA |
| | CACCCCACTCCCTCAATTACGTCCAAAAGAATCTCGAAAGCGTCCAGAG |
| | CAACGGACTGGCCGCCGTCATCAAGGAATATagAAATGCCGTCGCTcaCC |
| | TCAATATCATCAATAGACTGGACGAGTACATTGGCTCCGCTAGGGCTGA |
| | TAGCTACTACTCTCTGTACTGTTACTGCCTCCAAATGTATCTGAGCAAG |
| | AACTTCAGCGTGGGCTACCTCATCAACGTGCAAAAGCAGCTGGAGGAG |
| | CACCACACCTACATGAAGGATCTCATGTGGCTGCTCAACATCCCCTTCG |
| | CTTACAACCTCGCCAGATACAAAAATCTGTCCAACGAAAAACTCTTTTA |
| | CGACGAGGAAGCCGCCGCCGAAAAGGCTGACAAGGCTGAGAACGAGA |
| | GAGGCGAA (SEQ ID NO: 603) |
| Linker | GGAAGC |
| SV-40 NLS | CCCAAGAAGAAAAGGAAGGTC (SEQ ID NO: 532) |
| Linker | GAGGAC |
| HA Tag | TACCCCTACGATGTGCCCGACTACGCC (SEQ ID NO: 608) |
| WPRE3 | GATAATCAACCTCTGGATTACAAAATTTGTGAAAGATTGACTGGTATTC |
| | TTAACTATGTTGCTCCTTTTACGCTATGTGGATACGCTGCTTTAATGCCT |
| | TTGTATCATGCTATTGCTTCCCGTATGGCTTTCATTTTCTCCTCCTTGTAT |
| | AAATCCTGGTTAGTTCTTGCCACGGCGGAACTCATCGCCGCCTGCCTTG |
| | CCCGCTGCTGGACAGGGGCTCGGCTGTTGGGCACTGACAATTCCGTGG |
| | (SEQ ID NO: 609) |
| SV-40 poly A | AACTTGTTTATTGCAGCTTATAATGGTTACAAATAAAGCAATAGCATCA |
| | CAAATTTCACAAATAAAGCATTTTTTTCACTGCATTCTAGTTGTGGTTTG |
| | TCCAAACTCATCAATGTATCTTA (SEQ ID NO: 533) |
| 3′ITR | Aggaacccctagtgatggagttggccactccctctctgcgcgctcgctcgctcactgaggccgggcgaccaaaggtc |
| | gcccgacgcccgggctttgcccgggcggcctcagtgagcgagcgagcgcgcagctgcctgcagg (SEQ ID |
| | NO: 598) |

In some embodiments, an AAV vector comprising a nucleic acid encoding a CAG-targeting Cas13d composition comprises from 5′ to 3′: a sequence encoding a 5′ ITR (a first ITR), a sequence encoding an human U6 promoter, a dCas13d seq212 direct repeat, a sequence encoding a CAG guide 3 spacer sequence, a sequence encoding an EFS promoter, a sequence encoding a kozak sequence, a sequence encoding a dCas13d seq212 protein, a sequence encoding a linker sequence, a sequence encoding an SV-40 NLS, a sequence encoding a linker sequence, a sequence encoding an HA tag, a sequence encoding a WPRE, a sequence encoding an SV-40 polyA, and a 3′ ITR (a second ITR). In some embodiments, the CAG-targeting Cas13d composition is arranged as depicted in Table L.

TABLE L
Vector encoding a CAG-repeat targeting dCas13d fusion

| Plasmid | |
|---|---|
| Element | Nucleic Acid Sequences |
| 5′ ITR | Cctgcaggcagctgcgcgctcgctcgctcactgaggccgcccgggcgtcgggcgacctttggtcgcccggcctcag |
| | tgagcgagcgagcgcgcagagagggagtggccaactccatcactaggggttcct (SEQ ID NO: 597) |
| Human U6 | Gagggcctatttcccatgattccttcatatttgcatatacgatacaaggctgttagagagataattggaattaatttgactgt |
| promoter | aaacacaaagatattagtacaaaatacgtgacgtagaaagtaataatttcttgggtagtttgcagttttaaaattatgttttaa |
| | aatggactatcatatgcttaccgtaacttgaaagtatttcgatttcttggctttatatatcttgtggaaaggacgaaacacc |
| | (SEQ ID NO: 519) |
| Seq212 direct | Tagccctgcagtaaggcagggttctaagac (SEQ ID NO: 596) |
| repeat (DR) | |
| Spacer (CAG | Ctgctgctgctgctgctgctgctgct (SEQ ID NO: 459) |
| guide 3) | |
| EFS promoter | Taggtcttgaaaggagtgggaattggctccggtgcccgtcagtgggcagagcgcacatogcccacagtccccgaga |
| | agttggggggaggggtcggcaattgatccggtgcctagagaaggtggcgcggggtaaactgggaaagtgatgtcgt |
| | gtactggctccgcctttttcccgagggtgggggagaaccgtatataagtgcagtagtcgccgtgaacgttctttttcgcaa |
| | cgggtttgccgccagaacacagg (SEQ ID NO: 520) |
| Kozak Sequence | GCCGCCACCATG (SEQ ID NO: 529) |
| Dead Seq212 | AAGAAGAAGCACCAGAGCGCCGCCGAGAAGAGGCAAGTGAAGAAGCT |
| | CAAGAATCAAGAGAAGGCCCAGAAGTACGCTAGCGAGCCTTCCCCCCT |
| | CCAGAGCGATACAGCTGGCGTGGAATGCTCCCAGAAAAAGACAGTCGT |
| | CAGCCACATTGCCAGCTCCAAGACACTGGCCAAGGCTATGGGACTCAA |
| | ATCCACACTGGTCATGGGCGACAAGCTGGTCATCACCAGCTTTGCTGCT |
| | AGCAAGGCTGTCGGAGGCGCTGGCTACAAAAGCGCTAACATTGAAAAA |
| | ATCACAGATCTGCAAGGAAGGGTCATTGAGGAGCACGAAAGGATGTTT |
| | AGCGCCGATGTCGGAGAGAAAAATATCGAACTGAGCAAGAATGACTGC |
| | CACACCAACGTCAACAACCCCGTGGTGACCAACATCGGAAAGGATTAC |
| | ATCGGACTGAAATCTAGGCTGGAGCAAGAGTTTTTCGGCAAGACATTC |
| | GAGAATGACAATCTGCATGTGCAGCTGGCCTACAATATCCTCGACATCA |
| | AGAAAATTCTGGGAACCTATGTGAACAATATCATTTATATCTTCTACAA |
| | TCTGAATAGGGCTGGCACCGGCAGAGATGAGAGGATGTATGACGACCT |
| | CATCGGCACACTGTACGCTTACAAACCCATGGAGGCTCAACAGACCTAT |
| | CTGCTCAAAGGCGACAAGGATATGAGGAGGTTTGAGGAGGTGAAACAG |
| | CTGCTGCAAAACACCTCCGCTTACTATGTGTATTACGGCACACTGTTCG |
| | AGAAGGTGAAGGCTAAGAGCAAGAAGGAACAGAGGGCTAAGGAGGCC |
| | GAAATCGACGCTTGTACCGCCCATAACTACGATGTGCTGAGACTGCTGT |
| | CCCTCATGAGGCAGCTGTGCATGCACTCCGTCGCTGGAACAGCCTTTAA |
| | GCTGGCTGAGTCCGCTCTGTTCAACATTGAGGATGTGCTCAGCGCCGAT |
| | CTGAAGGAAATCCTCGATGAAGCCTTCTCCGGCGCCGTGAACAAGCTC |
| | AATGACGGATTCGTGCAGCACTCCGGCAACAATCTGTACGTGCTCCAGC |
| | AGCTGTACCCTAATGAGACCATCGAGAGAATCGCCGAGAAGTACTACA |
| | GACTCACCGTGAGGAAGGAGGATCTGAACATGGGAGTCAACATTAAAA |
| | AGCTGAGGGAGCTGATCGTGGGCCAATACTTTCCCGAGGTCCTCGACA |
| | AAGAATACGACCTCTCCAAGAATGGAGACAGCGTGGTGACATACAGAA |
| | GCAAGATTTATACCGTGATGAATTACATTCTGCTGTATTACCTCGAGGA |
| | CCACGACTCCAGCAGAGAAAGCATGGTCGAAGCTCTGAGACAAAACAG |
| | AGAGGGCGATGAAGGCAAGGAGGAGATCTATAGACAGTTTGCCAAGA |
| | AGGTGTGGAACGGCGTGTCCGGACTGTTTGGCGTGTGTCTGAACCTCTT |
| | CAAGACCGAAAAGAGAAACAAGTTTAGGAGCAAAGTCGCCCTCCCCGA |
| | TGTGTCCGGCGCTGCCTATATGCTCTCCTCCGAGAACATCGACTACTTT |
| | GTCAAGATGCTCTTCTTTGTGTGTAAGTTTCTGGATGGCAAAGAAATCA |
| | ACGAGCTGCTGTGCGCTCTGATCAACAAATTTGATAATATTGCCGATAT |
| | TCTGGATGCTGCCGCTCAATGTGGCTCCTCCGTCTGGTTCGTGGACAGC |
| | TATAGGTTCTTCGAGAGATCTAGGAGGATTAGCGCCCAGATTAGAATCG |
| | TGAAGAACATCGCTTCCAAGGATTTTAAGAAATCCAAGAAGGATTCCG |
| | ATGAGAGCTACCCCGAGCAGCTGTATCTGGATGCTCTGGCTCTGCTCGG |
| | AGACGTCATCTCCAAGTACAAGCAGAATAGAGATGGCAGCGTCGTCAT |
| | CGATGACCAAGGCAATGCCGTGCTGACAGAGCAATACAAGAGGTTTAG |
| | ATATGAATTTTTCGAGGAGATCAAGAGGGACGAAAGCGGCGGCATCAA |
| | GTACAAGAAGTCCGGAAAACCCGAGTACAACCATCAGAGAAGGAATTT |
| | TATTCTGAATAATGTGCTGAAAAGCAAATGGTTTTTCTATGTGGTGAAG |
| | TACAATAGGCCCAGCAGCTGCAGAGAACTGATGAAGAATAAGGAAATT |
| | CTGAGGTTCGTGCTGAGAGACATCCCCGACTCCCAAGTGAGAAGATAC |
| | TTTAAGGCCGTCCAAGGAGAGGAAGCTTACGCTAGCGCCGAAGCTATG |
| | AGGACAAGACTGGTCGACGCTCTGTCCCAATTTAGCGTCACAGCTTGTC |
| | TGGATGAAGTGGGCGGCATGACAGACAAGGAATTCGCCTCCCAGAGGG |
| | CCGTCGATAGCAAAGAAAAACTGAGAGCCATCATCAGACTGTATCTGA |
| | CAGTCGCCTATCTGATTACCAAGAGCATGGTGAAGGTGAATACAAGGT |
| | TTAGCATTGCCTTTAGCGTGCTGGAGAGGGACTACTATCTGCTCATTGA |
| | CGGCAAGAAGAAATCCAGCGACTACACCGGAGAGGATATGCTGGCTCT |
| | GACCAGAAAATTTGTGGGCGAAGATGCTGGACTGTATAGAGAGTGGAA |
| | AGAGAAGAACGCTGAAGCCAAGGACAAATATTTTGACAAGGCCGAAA |
| | GGAAGAAGGTGCTGAGACAGAACGATAAGATGATCAGAAAGATGCAC |
| | TTCACACCCCACTCCCTCAATTACGTCCAAAAGAATCTCGAAAGCGTCC |
| | AGAGCAACGGACTGGCCGCCGTCATCAAGGAATATAGAAATGCCGTCG |
| | CTCACCTCAATATCATCAATAGACTGGACGAGTACATTGGCTCCGCTAG |
| | GGCTGATAGCTACTACTCTCTGTACTGTTACTGCCTCCAAATGTATCTGA |
| | GCAAGAACTTCAGCGTGGGCTACCTCATCAACGTGCAAAAGCAGCTGG |
| | AGGAGCACCACACCTACATGAAGGATCTCATGTGGCTGCTCAACATCCC |
| | CTTCGCTTACAACCTCGCCAGATACgcAAATCTGTCCAACGAAAAACTCT |
| | TTTACGACGAGGAAGCCGCCGCCGAAAAGGCTGACAAGGCTGAGAACG |
| | AGAGAGGCGAA (SEQ ID NO: 604) |
| Linker | GGAAGC |
| SV-40 NLS | CCCAAGAAGAAAAGGAAGGTC (SEQ ID NO: 532) |
| Linker | GAGGAC |
| HA Tag | TACCCCTACGATGTGCCCGACTACGCC (SEQ ID NO: 608) |
| WPRE3 | GATAATCAACCTCTGGATTACAAAATTTGTGAAAGATTGACTGGTATTC |
| | TTAACTATGTTGCTCCTTTTACGCTATGTGGATACGCTGCTTTAATGCCT |
| | TTGTATCATGCTATTGCTTCCCGTATGGCTTTCATTTTCTCCTCCTTGTAT |
| | AAATCCTGGTTAGTTCTTGCCACGGCGGAACTCATCGCCGCCTGCCTTG |
| | CCCGCTGCTGGACAGGGGCTCGGCTGTTGGGCACTGACAATTCCGTGG |
| | (SEQ ID NO: 609) |
| SV-40 poly A | AACTTGTTTATTGCAGCTTATAATGGTTACAAATAAAGCAATAGCATCA |
| | CAAATTTCACAAATAAAGCATTTTTTTCACTGCATTCTAGTTGTGGTTTG |
| | TCCAAACTCATCAATGTATCTTA (SEQ ID NO: 533) |
| 3′ITR | Aggaacccctagtgatggagttggccactccctctctgcgcgctcgctcgctcactgaggccgggcgaccaaaggtc |
| | gcccgacgcccgggctttgcccgggcggcctcagtgagcgagcgagcgcgcagctgcctgcagg (SEQ ID |
| | NO: 598) |

In some embodiments, an AAV vector comprising a nucleic acid encoding a CA-targeting Cas13d composition comprises from 5′ to 3′: a sequence encoding a 5′ ITR (a first ITR), a sequence encoding an human U6 promoter, a dCas13d seq212 direct repeat, a sequence encoding a CAG guide 3 spacer sequence, a sequence encoding an EFS promoter, a sequence encoding a kozak sequence, a sequence encoding an SV-40 NLS, a sequence encoding a linker, a sequence encoding a dCas13d seq212 protein, a sequence encoding a linker sequence, a sequence encoding an E17 endonuclease, a sequence encoding a linker sequence, a sequence encoding a myc tag, a sequence encoding a WPRE, a sequence encoding an SV-40 polyA, and a 3′ ITR (a second ITR). In some embodiments, the CAG-targeting Cas13d composition is arranged as depicted in Table M. In some embodiments, the vector set forth in Table M is referred to as A01545.

TABLE M
Vector A01545 encoding a CAG-repeat targeting dCas13d fusion

| Plasmid | |
|---|---|
| Element | Nucleic Acid Sequences |
| 5′ ITR | Cctgcaggcagctgcgcgctcgctcgctcactgaggccgcccgggcgtcgggcgacctttggtcgcccggcctcag |
| | tgagcgagcgagcgcgcagagagggagtggccaactccatcactaggggttcct (SEQ ID NO: 597) |
| Human U6 | Gagggcctatttcccatgattccttcatatttgcatatacgatacaaggctgttagagagataattggaattaatttgactgt |
| promoter | aaacacaaagatattagtacaaaatacgtgacgtagaaagtaataatttcttgggtagtttgcagttttaaaattatgttttaa |
| | aatggactatcatatgcttaccgtaacttgaaagtatttcgatttcttggctttatatatcttgtggaaaggacgaaacacc |
| | (SEQ ID NO: 519) |
| Seq212 direct | Tagccctgcagtaaggcagggttctaagac (SEQ ID NO: 596) |
| repeat (DR) | |
| Spacer (CAG | Ctgctgctgctgctgctgctgctgct (SEQ ID NO: 459) |
| guide 3) | |
| EFS promoter | Taggtcttgaaaggagtgggaattggctccggtgcccgtcagtgggcagagcgcacatogcccacagtccccgaga |
| | agttggggggaggggtcggcaattgatccggtgcctagagaaggtggcgcggggtaaactgggaaagtgatgtcgt |
| | gtactggctccgcctttttcccgaggggggggagaaccgtatataagtgcagtagtcgccgtgaacgttctttttcgcaa |
| | cgggtttgccgccagaacacagg (SEQ ID NO: 520) |
| Kozak Sequence | GCCGCCACCATGG (SEQ ID NO: 529) |
| SV40 NLS | CCCAAGAAGAAAAGGAAGGTC (SEQ ID NO: 532) |
| Linker | ggaGGATCT |
| Dead Seq212 | AAGAAGAAGCACCAGAGCGCCGCCGAGAAGAGGCAAGTGAAGAAGCT |
| | CAAGAATCAAGAGAAGGCCCAGAAGTACGCTAGCGAGCCTTCCCCCCT |
| | CCAGAGCGATACAGCTGGCGTGGAATGCTCCCAGAAAAAGACAGTCGT |
| | CAGCCACATTGCCAGCTCCAAGACACTGGCCAAGGCTATGGGACTCAA |
| | ATCCACACTGGTCATGGGCGACAAGCTGGTCATCACCAGCTTTGCTGCT |
| | AGCAAGGCTGTCGGAGGCGCTGGCTACAAAAGCGCTAACATTGAAAAA |
| | ATCACAGATCTGCAAGGAAGGGTCATTGAGGAGCACGAAAGGATGTTT |
| | AGCGCCGATGTCGGAGAGAAAAATATCGAACTGAGCAAGAATGACTGC |
| | CACACCAACGTCAACAACCCCGTGGTGACCAACATCGGAAAGGATTAC |
| | ATCGGACTGAAATCTAGGCTGGAGCAAGAGTTTTTCGGCAAGACATTC |
| | GAGAATGACAATCTGCATGTGCAGCTGGCCTACAATATCCTCGACATCA |
| | AGAAAATTCTGGGAACCTATGTGAACAATATCATTTATATCTTCTACAA |
| | TCTGAATAGGGCTGGCACCGGCAGAGATGAGAGGATGTATGACGACCT |
| | CATCGGCACACTGTACGCTTACAAACCCATGGAGGCTCAACAGACCTAT |
| | CTGCTCAAAGGCGACAAGGATATGAGGAGGTTTGAGGAGGTGAAACAG |
| | CTGCTGCAAAACACCTCCGCTTACTATGTGTATTACGGCACACTGTTCG |
| | AGAAGGTGAAGGCTAAGAGCAAGAAGGAACAGAGGGCTAAGGAGGCC |
| | GAAATCGACGCTTGTACCGCCCATAACTACGATGTGCTGAGACTGCTGT |
| | CCCTCATGAGGCAGCTGTGCATGCACTCCGTCGCTGGAACAGCCTTTAA |
| | GCTGGCTGAGTCCGCTCTGTTCAACATTGAGGATGTGCTCAGCGCCGAT |
| | CTGAAGGAAATCCTCGATGAAGCCTTCTCCGGCGCCGTGAACAAGCTC |
| | AATGACGGATTCGTGCAGCACTCCGGCAACAATCTGTACGTGCTCCAGC |
| | AGCTGTACCCTAATGAGACCATCGAGAGAATCGCCGAGAAGTACTACA |
| | GACTCACCGTGAGGAAGGAGGATCTGAACATGGGAGTCAACATTAAAA |
| | AGCTGAGGGAGCTGATCGTGGGCCAATACTTTCCCGAGGTCCTCGACA |
| | AAGAATACGACCTCTCCAAGAATGGAGACAGCGTGGTGACATACAGAA |
| | GCAAGATTTATACCGTGATGAATTACATTCTGCTGTATTACCTCGAGGA |
| | CCACGACTCCAGCAGAGAAAGCATGGTCGAAGCTCTGAGACAAAACAG |
| | AGAGGGCGATGAAGGCAAGGAGGAGATCTATAGACAGTTTGCCAAGA |
| | AGGTGTGGAACGGCGTGTCCGGACTGTTTGGCGTGTGTCTGAACCTCTT |
| | CAAGACCGAAAAGAGAAACAAGTTTAGGAGCAAAGTCGCCCTCCCCGA |
| | TGTGTCCGGCGCTGCCTATATGCTCTCCTCCGAGAACATCGACTACTTT |
| | GTCAAGATGCTCTTCTTTGTGTGTAAGTTTCTGGATGGCAAAGAAATCA |
| | ACGAGCTGCTGTGCGCTCTGATCAACAAATTTGATAATATTGCCGATAT |
| | TCTGGATGCTGCCGCTCAATGTGGCTCCTCCGTCTGGTTCGTGGACAGC |
| | TATAGGTTCTTCGAGAGATCTAGGAGGATTAGCGCCCAGATTAGAATCG |
| | TGAAGAACATCGCTTCCAAGGATTTTAAGAAATCCAAGAAGGATTCCG |
| | ATGAGAGCTACCCCGAGCAGCTGTATCTGGATGCTCTGGCTCTGCTCGG |
| | AGACGTCATCTCCAAGTACAAGCAGAATAGAGATGGCAGCGTCGTCAT |
| | CGATGACCAAGGCAATGCCGTGCTGACAGAGCAATACAAGAGGTTTAG |
| | ATATGAATTTTTCGAGGAGATCAAGAGGGACGAAAGCGGCGGCATCAA |
| | GTACAAGAAGTCCGGAAAACCCGAGTACAACCATCAGAGAAGGAATTT |
| | TATTCTGAATAATGTGCTGAAAAGCAAATGGTTTTTCTATGTGGTGAAG |
| | TACAATAGGCCCAGCAGCTGCAGAGAACTGATGAAGAATAAGGAAATT |
| | CTGAGGTTCGTGCTGAGAGACATCCCCGACTCCCAAGTGAGAAGATAC |
| | TTTAAGGCCGTCCAAGGAGAGGAAGCTTACGCTAGCGCCGAAGCTATG |
| | AGGACAAGACTGGTCGACGCTCTGTCCCAATTTAGCGTCACAGCTTGTC |
| | TGGATGAAGTGGGCGGCATGACAGACAAGGAATTCGCCTCCCAGAGGG |
| | CCGTCGATAGCAAAGAAAAACTGAGAGCCATCATCAGACTGTATCTGA |
| | CAGTCGCCTATCTGATTACCAAGAGCATGGTGAAGGTGAATACAAGGT |
| | TTAGCATTGCCTTTAGCGTGCTGGAGAGGGACTACTATCTGCTCATTGA |
| | CGGCAAGAAGAAATCCAGCGACTACACCGGAGAGGATATGCTGGCTCT |
| | GACCAGAAAATTTGTGGGCGAAGATGCTGGACTGTATAGAGAGTGGAA |
| | AGAGAAGAACGCTGAAGCCAAGGACAAATATTTTGACAAGGCCGAAA |
| | GGAAGAAGGTGCTGAGACAGAACGATAAGATGATCAGAAAGATGCAC |
| | TTCACACCCCACTCCCTCAATTACGTCCAAAAGAATCTCGAAAGCGTCC |
| | AGAGCAACGGACTGGCCGCCGTCATCAAGGAATATAGAAATGCCGTCG |
| | CTgcCCTCAATATCATCAATAGACTGGACGAGTACATTGGCTCCGCTAG |
| | GGCTGATAGCTACTACTCTCTGTACTGTTACTGCCTCCAAATGTATCTGA |
| | GCAAGAACTTCAGCGTGGGCTACCTCATCAACGTGCAAAAGCAGCTGG |
| | AGGAGCACCACACCTACATGAAGGATCTCATGTGGCTGCTCAACATCCC |
| | CTTCGCTTACAACCTCGCCAGATACAAAAATCTGTCCAACGAAAAACTC |
| | TTTTACGACGAGGAAGCCGCCGCCGAAAAGGCTGACAAGGCTGAGAAC |
| | GAGAGAGGCGAA (SEQ ID NO: 605) |
| Linker | GGTGGAGGCggtAGCGGAGGtGGCGGAAGTGGCGGAGGAGGTAGT (SEQ |
| | ID NO: 612) |
| E17 | Ggtggtggcacccctaaggctcccaacctggagcctccactcccagaagaggaaaaggagggcagcgacctgaga |
| | ccagtggtcatcgatgggagcaacgtggccatgagccatgggaacaaggaggtgttctcctgccggggcatcctgct |
| | ggcagtgaactggtttctggagcggggccacacagacatcacagtgtttgtgccatcctggaggaaggagcagcctc |
| | ggcccgacgtgcccatcacagaccagcacatcctgcgggaactggagaagaagaagatcctggtgttcacaccatca |
| | cgacgcgtgggtggcaagcgggtggtgtgctatgacgacagattcattgtgaagctggcctacgagtctgacgggatc |
| | gtggtttccaacgacacataccgtgacctccaaggcgagcggcaggagtggaagcgcttcatcgaggagcggctgct |
| | catgtactccttcgtcaatgacaagtttatgccccctgatgacccactgggccggcacgggcccagcctggacaacttc |
| | ctgcgtaagaagccactcactttggag (SEQ ID NO: 611) |
| Linker | GGCGGAtct |
| Myc Tag | GAGCAgAAACTGATTAGcGAAGAgGATCTC (SEQ ID NO: 610) |
| WPRE3 | GATAATCAACCTCTGGATTACAAAATTTGTGAAAGATTGACTGGTATTC |
| | TTAACTATGTTGCTCCTTTTACGCTATGTGGATACGCTGCTTTAATGCCT |
| | TTGTATCATGCTATTGCTTCCCGTATGGCTTTCATTTTCTCCTCCTTGTAT |
| | AAATCCTGGTTAGTTCTTGCCACGGCGGAACTCATCGCCGCCTGCCTTG |
| | CCCGCTGCTGGACAGGGGCTCGGCTGTTGGGCACTGACAATTCCGTGG |
| | (SEQ ID NO: 609) |
| SV-40 poly A | AACTTGTTTATTGCAGCTTATAATGGTTACAAATAAAGCAATAGCATCA |
| | CAAATTTCACAAATAAAGCATTTTTTTCACTGCATTCTAGTTGTGGTTTG |
| | TCCAAACTCATCAATGTATCTTA (SEQ ID NO: 533) |
| 3′ITR | Aggaacccctagtgatggagttggccactccctctctgcgcgctcgctcgctcactgaggccgggcgaccaaaggtc |
| | gcccgacgcccgggctttgcccgggcggcctcagtgagcgagcgagcgcgcagctgcctgcagg (SEQ ID |
| | NO: 598) |

In some embodiments, an AAV vector comprising a nucleic acid encoding a CAG-targeting Cas13d composition comprises from 5′ to 3′: a sequence encoding a 5′ ITR (a first ITR), a sequence encoding an human U6 promoter, a dCas13d seq22 direct repeat, a sequence encoding a CAG guide 3 spacer sequence, a sequence encoding an EFS promoter, a sequence encoding a kozak sequence, a sequence encoding an SV-40 NLS, a sequence encoding a linker, a sequence encoding a dCas13d seq212 protein, a sequence encoding a linker sequence, a sequence encoding an E17 endonuclease, a sequence encoding a linker sequence, a sequence encoding a myc tag, a sequence encoding a WPRE, a sequence encoding an SV-40 polyA, and a 3′ ITR (a second ITR). In some embodiments, the CAG-targeting Cas3d composition is arranged as depicted in Table N. In some embodiments, the vector set forth in Table N is referred to as A01553.

TABLE N
Vector A01553 encoding a CAG-repeat targeting dCas13d fusion

| Plasmid | |
|---|---|
| Element | Nucleic Acid Sequences |
| 5′ ITR | Cctgcaggcagctgcgcgctcgctcgctcactgaggccgcccgggcgtcgggcgacctttggtcgcccggcctcag |
| | tgagcgagcgagcgcgcagagagggagtggccaactccatcactaggggttcct (SEQ ID NO: 597) |
| Human U6 | Gagggcctatttcccatgattccttcatatttgcatatacgatacaaggctgttagagagataattggaattaatttgactgt |
| promoter | aaacacaaagatattagtacaaaatacgtgacgtagaaagtaataatttcttgggtagtttgcagttttaaaattatgttttaa |
| | aatggactatcatatgcttaccgtaacttgaaagtatttcgatttcttggctttatatatcttgtggaaaggacgaaacacc |
| | (SEQ ID NO: 519) |
| Seq212 direct | Tagccctgcagtaaggcagggttctaagac (SEQ ID NO: 596) |
| repeat (DR) | |
| Spacer (CAG | Ctgctgctgctgctgctgctgctgct (SEQ ID NO: 459) |
| guide 3) | |
| EFS promoter | Taggtcttgaaaggagtgggaattggctccggtgcccgtcagtgggcagagcgcacatcgcccacagtccccgaga |
| | agttggggggaggggtcggcaattgatccggtgcctagagaaggtggcgcggggtaaactgggaaagtgatgtcgt |
| | gtactggctccgcctttttcccgagggtgggggagaaccgtatataagtgcagtagtcgccgtgaacgttctttttcgcaa |
| | cgggtttgccgccagaacacagg (SEQ ID NO: 520) |
| Kozak Sequence | GCCGCCACCATGG (SEQ ID NO: 529) |
| SV40 NLS | CCCAAGAAGAAAAGGAAGGTC (SEQ ID NO: 532) |
| Linker | ggaGGATCT |
| Dead Seq212 | AAGAAGAAGCACCAGAGCGCCGCCGAGAAGAGGCAAGTGAAGAAGCT |
| | CAAGAATCAAGAGAAGGCCCAGAAGTACGCTAGCGAGCCTTCCCCCCT |
| | CCAGAGCGATACAGCTGGCGTGGAATGCTCCCAGAAAAAGACAGTCGT |
| | CAGCCACATTGCCAGCTCCAAGACACTGGCCAAGGCTATGGGACTCAA |
| | ATCCACACTGGTCATGGGCGACAAGCTGGTCATCACCAGCTTTGCTGCT |
| | AGCAAGGCTGTCGGAGGCGCTGGCTACAAAAGCGCTAACATTGAAAAA |
| | ATCACAGATCTGCAAGGAAGGGTCATTGAGGAGCACGAAAGGATGTTT |
| | AGCGCCGATGTCGGAGAGAAAAATATCGAACTGAGCAAGAATGACTGC |
| | CACACCAACGTCAACAACCCCGTGGTGACCAACATCGGAAAGGATTAC |
| | ATCGGACTGAAATCTAGGCTGGAGCAAGAGTTTTTCGGCAAGACATTC |
| | GAGAATGACAATCTGCATGTGCAGCTGGCCTACAATATCCTCGACATCA |
| | AGAAAATTCTGGGAACCTATGTGAACAATATCATTTATATCTTCTACAA |
| | TCTGAATAGGGCTGGCACCGGCAGAGATGAGAGGATGTATGACGACCT |
| | CATCGGCACACTGTACGCTTACAAACCCATGGAGGCTCAACAGACCTAT |
| | CTGCTCAAAGGCGACAAGGATATGAGGAGGTTTGAGGAGGTGAAACAG |
| | CTGCTGCAAAACACCTCCGCTTACTATGTGTATTACGGCACACTGTTCG |
| | AGAAGGTGAAGGCTAAGAGCAAGAAGGAACAGAGGGCTAAGGAGGCC |
| | GAAATCGACGCTTGTACCGCCCATAACTACGATGTGCTGAGACTGCTGT |
| | CCCTCATGgcGCAGCTGTGCATGgcCTCCGTCGCTGGAACAGCCTTTAAG |
| | CTGGCTGAGTCCGCTCTGTTCAACATTGAGGATGTGCTCAGCGCCGATC |
| | TGAAGGAAATCCTCGATGAAGCCTTCTCCGGCGCCGTGAACAAGCTCA |
| | ATGACGGATTCGTGCAGCACTCCGGCAACAATCTGTACGTGCTCCAGCA |
| | GCTGTACCCTAATGAGACCATCGAGAGAATCGCCGAGAAGTACTACAG |
| | ACTCACCGTGAGGAAGGAGGATCTGAACATGGGAGTCAACATTAAAAA |
| | GCTGAGGGAGCTGATCGTGGGCCAATACTTTCCCGAGGTCCTCGACAA |
| | AGAATACGACCTCTCCAAGAATGGAGACAGCGTGGTGACATACAGAAG |
| | CAAGATTTATACCGTGATGAATTACATTCTGCTGTATTACCTCGAGGAC |
| | CACGACTCCAGCAGAGAAAGCATGGTCGAAGCTCTGAGACAAAACAGA |
| | GAGGGCGATGAAGGCAAGGAGGAGATCTATAGACAGTTTGCCAAGAA |
| | GGTGTGGAACGGCGTGTCCGGACTGTTTGGCGTGTGTCTGAACCTCTTC |
| | AAGACCGAAAAGAGAAACAAGTTTAGGAGCAAAGTCGCCCTCCCCGAT |
| | GTGTCCGGCGCTGCCTATATGCTCTCCTCCGAGAACATCGACTACTTTG |
| | TCAAGATGCTCTTCTTTGTGTGTAAGTTTCTGGATGGCAAAGAAATCAA |
| | CGAGCTGCTGTGCGCTCTGATCAACAAATTTGATAATATTGCCGATATT |
| | CTGGATGCTGCCGCTCAATGTGGCTCCTCCGTCTGGTTCGTGGACAGCT |
| | ATAGGTTCTTCGAGAGATCTAGGAGGATTAGCGCCCAGATTAGAATCGT |
| | GAAGAACATCGCTTCCAAGGATTTTAAGAAATCCAAGAAGGATTCCGA |
| | TGAGAGCTACCCCGAGCAGCTGTATCTGGATGCTCTGGCTCTGCTCGGA |
| | GACGTCATCTCCAAGTACAAGCAGAATAGAGATGGCAGCGTCGTCATC |
| | GATGACCAAGGCAATGCCGTGCTGACAGAGCAATACAAGAGGTTTAGA |
| | TATGAATTTTTCGAGGAGATCAAGAGGGACGAAAGCGGCGGCATCAAG |
| | TACAAGAAGTCCGGAAAACCCGAGTACAACCATCAGAGAAGGAATTTT |
| | ATTCTGAATAATGTGCTGAAAAGCAAATGGTTTTTCTATGTGGTGAAGT |
| | ACAATAGGCCCAGCAGCTGCAGAGAACTGATGAAGAATAAGGAAATTC |
| | TGAGGTTCGTGCTGAGAGACATCCCCGACTCCCAAGTGAGAAGATACTT |
| | TAAGGCCGTCCAAGGAGAGGAAGCTTACGCTAGCGCCGAAGCTATGAG |
| | GACAAGACTGGTCGACGCTCTGTCCCAATTTAGCGTCACAGCTTGTCTG |
| | GATGAAGTGGGCGGCATGACAGACAAGGAATTCGCCTCCCAGAGGGCC |
| | GTCGATAGCAAAGAAAAACTGAGAGCCATCATCAGACTGTATCTGACA |
| | GTCGCCTATCTGATTACCAAGAGCATGGTGAAGGTGAATACAAGGTTTA |
| | GCATTGCCTTTAGCGTGCTGGAGAGGGACTACTATCTGCTCATTGACGG |
| | CAAGAAGAAATCCAGCGACTACACCGGAGAGGATATGCTGGCTCTGAC |
| | CAGAAAATTTGTGGGCGAAGATGCTGGACTGTATAGAGAGTGGAAAGA |
| | GAAGAACGCTGAAGCCAAGGACAAATATTTTGACAAGGCCGAAAGGA |
| | AGAAGGTGCTGAGACAGAACGATAAGATGATCAGAAAGATGCACTTCA |
| | CACCCCACTCCCTCAATTACGTCCAAAAGAATCTCGAAAGCGTCCAGAG |
| | CAACGGACTGGCCGCCGTCATCAAGGAATATgcAAATGCCGTCGCTgcCC |
| | TCAATATCATCAATAGACTGGACGAGTACATTGGCTCCGCTAGGGCTGA |
| | TAGCTACTACTCTCTGTACTGTTACTGCCTCCAAATGTATCTGAGCAAG |
| | AACTTCAGCGTGGGCTACCTCATCAACGTGCAAAAGCAGCTGGAGGAG |
| | CACCACACCTACATGAAGGATCTCATGTGGCTGCTCAACATCCCCTTCG |
| | CTTACAACCTCGCCAGATACAAAAATCTGTCCAACGAAAAACTCTTTTA |
| | CGACGAGGAAGCCGCCGCCGAAAAGGCTGACAAGGCTGAGAACGAGA |
| | GAGGCGAA (SEQ ID NO: 606) |
| Linker | GGTGGAGGCggtAGCGGAGGtGGCGGAAGTGGCGGAGGAGGTAGT (SEQ |
| | ID NO: 612) |
| E17 | Ggtggtggcacccctaaggctcccaacctggagcctccactcccagaagaggaaaaggagggcagcgacctgaga |
| | ccagtggtcatcgatgggagcaacgtggccatgagccatgggaacaaggaggtgttctcctgccggggcatcctgct |
| | ggcagtgaactggtttctggagcggggccacacagacatcacagtgtttgtgccatcctggaggaaggagcagcctc |
| | ggcccgacgtgcccatcacagaccagcacatcctgcgggaactggagaagaagaagatcctggtgttcacaccatca |
| | cgacgcgtgggtggcaagcgggtggtgtgctatgacgacagattcattgtgaagctggcctacgagtctgacgggatc |
| | gtggtttccaacgacacataccgtgacctccaaggcgagcggcaggagtggaagcgcttcatcgaggagcggctgct |
| | catgtactccttcgtcaatgacaagtttatgccccctgatgacccactgggccggcacgggcccagcctggacaacttc |
| | ctgcgtaagaagccactcactttggag (SEQ ID NO: 611) |
| Linker | GGCGGAtct |
| Myc Tag | GAGCAgAAACTGATTAGcGAAGAgGATCTC (SEQ ID NO: 610) |
| WPRE3 | GATAATCAACCTCTGGATTACAAAATTTGTGAAAGATTGACTGGTATTC |
| | TTAACTATGTTGCTCCTTTTACGCTATGTGGATACGCTGCTTTAATGCCT |
| | TTGTATCATGCTATTGCTTCCCGTATGGCTTTCATTTTCTCCTCCTTGTAT |
| | AAATCCTGGTTAGTTCTTGCCACGGCGGAACTCATCGCCGCCTGCCTTG |
| | CCCGCTGCTGGACAGGGGCTCGGCTGTTGGGCACTGACAATTCCGTGG |
| | (SEQ ID NO: 609) |
| SV-40 poly A | AACTTGTTTATTGCAGCTTATAATGGTTACAAATAAAGCAATAGCATCA |
| | CAAATTTCACAAATAAAGCATTTTTTTCACTGCATTCTAGTTGTGGTTTG |
| | TCCAAACTCATCAATGTATCTTA (SEQ ID NO: 533) |
| 3′ITR | Aggaacccctagtgatggagttggccactccctctctgcgcgctcgctcgctcactgaggccgggcgaccaaaggtc |
| | gcccgacgcccgggctttgcccgggcggcctcagtgagcgagcgagcgcgcagctgcctgcagg (SEQ ID |
| | NO: 598) |

In some embodiments, an AAV vector comprising a nucleic acid encoding a CAG-targeting Cas13d composition comprises from 5′ to 3′: a sequence encoding a 5′ ITR (a first ITR), a sequence encoding an human U6 promoter, a dCas13d seq212 direct repeat, a sequence encoding a CAG guide 3 spacer sequence, a sequence encoding an EFS promoter, a sequence encoding a kozak sequence, a sequence encoding an E17 endonuclease, a sequence encoding a linker sequence, a sequence encoding a dCas13d seq212 protein, a sequence encoding a linker sequence, a sequence encoding an SV-40 NLS, a sequence encoding a linker, a sequence encoding an HAtag, a sequence encoding a WPRE, a sequence encoding an SV-40 polyA, and a 3′ ITR (a second ITR). In some embodiments, the CAG-targeting Cas13d composition is arranged as depicted in Table 0.

TABLE O
Vector encoding a CAG-repeat targeting dCas13d fusion

| Plasmid | |
|---|---|
| Element | Nucleic Acid Sequences |
| 5′ ITR | Cctgcaggcagctgcgcgctcgctcgctcactgaggccgcccgggcgtcgggcgacctttggtcgcccggcctcag |
| | tgagcgagcgagcgcgcagagagggagtggccaactccatcactaggggttcct (SEQ ID NO: 597) |
| Human U6 | Gagggcctatttcccatgattccttcatatttgcatatacgatacaaggctgttagagagataattggaattaatttgactgt |
| promoter | aaacacaaagatattagtacaaaatacgtgacgtagaaagtaataatttcttgggtagtttgcagttttaaaattatgttttaa |
| | aatggactatcatatgcttaccgtaacttgaaagtatttcgatttcttggctttatatatottgtggaaaggacgaaacacc |
| | (SEQ ID NO: 519) |
| Seq212 direct | Tagccctgcagtaaggcagggttctaagac (SEQ ID NO: 596) |
| repeat (DR) | |
| Spacer (CAG | Ctgctgctgctgctgctgctgctgct (SEQ ID NO: 459) |
| guide 3) | |
| EFS promoter | Taggtcttgaaaggagtgggaattggctccggtgcccgtcagtgggcagagcgcacatcgcccacagtccccgaga |
| | agttggggggaggggtcggcaattgatccggtgcctagagaaggtggcgcggggtaaactgggaaagtgatgtcgt |
| | gtactggctccgcctttttcccgaggggggggagaaccgtatataagtgcagtagtcgccgtgaacgttctttttcgcaa |
| | cgggtttgccgccagaacacagg (SEQ ID NO: 520) |
| Kozak Sequence | GCCGCCACCATG (SEQ ID NO: 529) |
| E17 | Ggtggtggcacccctaaggctcccaacctggagcctccactcccagaagaggaaaaggagggcagcgacctgaga |
| | ccagtggtcatcgatgggagcaacgtggccatgagccatgggaacaaggaggtgttctcctgccggggcatcctgct |
| | ggcagtgaactggtttctggagcggggccacacagacatcacagtgtttgtgccatcctggaggaaggagcagcctc |
| | ggcccgacgtgcccatcacagaccagcacatcctgcgggaactggagaagaagaagatcctggtgttcacaccatca |
| | cgacgcgtgggtggcaagcgggtggtgtgctatgacgacagattcattgtgaagctggcctacgagtctgacgggatc |
| | gtggtttccaacgacacataccgtgacctccaaggcgagcggcaggagtggaagcgcttcatcgaggagcggctgct |
| | catgtactccttcgtcaatgacaagtttatgccccctgatgacccactgggccggcacgggcccagcctggacaacttc |
| | ctgcgtaagaagccactcactttggag (SEQ ID NO: 611) |
| Linker | GGTGGAGGCggtAGCGGAGGtGGCGGAAGTGGCGGAGGAGGTAGT (SEQ |
| | ID NO: 612) |
| Dead Seq212 | AAGAAGAAGCACCAGAGCGCCGCCGAGAAGAGGCAAGTGAAGAAGCT |
| | CAAGAATCAAGAGAAGGCCCAGAAGTACGCTAGCGAGCCTTCCCCCCT |
| | CCAGAGCGATACAGCTGGCGTGGAATGCTCCCAGAAAAAGACAGTCGT |
| | CAGCCACATTGCCAGCTCCAAGACACTGGCCAAGGCTATGGGACTCAA |
| | ATCCACACTGGTCATGGGCGACAAGCTGGTCATCACCAGCTTTGCTGCT |
| | AGCAAGGCTGTCGGAGGCGCTGGCTACAAAAGCGCTAACATTGAAAAA |
| | ATCACAGATCTGCAAGGAAGGGTCATTGAGGAGCACGAAAGGATGTTT |
| | AGCGCCGATGTCGGAGAGAAAAATATCGAACTGAGCAAGAATGACTGC |
| | CACACCAACGTCAACAACCCCGTGGTGACCAACATCGGAAAGGATTAC |
| | ATCGGACTGAAATCTAGGCTGGAGCAAGAGTTTTTCGGCAAGACATTC |
| | GAGAATGACAATCTGCATGTGCAGCTGGCCTACAATATCCTCGACATCA |
| | AGAAAATTCTGGGAACCTATGTGAACAATATCATTTATATCTTCTACAA |
| | TCTGAATAGGGCTGGCACCGGCAGAGATGAGAGGATGTATGACGACCT |
| | CATCGGCACACTGTACGCTTACAAACCCATGGAGGCTCAACAGACCTAT |
| | CTGCTCAAAGGCGACAAGGATATGAGGAGGTTTGAGGAGGTGAAACAG |
| | CTGCTGCAAAACACCTCCGCTTACTATGTGTATTACGGCACACTGTTCG |
| | AGAAGGTGAAGGCTAAGAGCAAGAAGGAACAGAGGGCTAAGGAGGCC |
| | GAAATCGACGCTTGTACCGCCCATAACTACGATGTGCTGAGACTGCTGT |
| | CCCTCATGgcGCAGCTGTGCATGgcCTCCGTCGCTGGAACAGCCTTTAAG |
| | CTGGCTGAGTCCGCTCTGTTCAACATTGAGGATGTGCTCAGCGCCGATC |
| | TGAAGGAAATCCTCGATGAAGCCTTCTCCGGCGCCGTGAACAAGCTCA |
| | ATGACGGATTCGTGCAGCACTCCGGCAACAATCTGTACGTGCTCCAGCA |
| | GCTGTACCCTAATGAGACCATCGAGAGAATCGCCGAGAAGTACTACAG |
| | ACTCACCGTGAGGAAGGAGGATCTGAACATGGGAGTCAACATTAAAAA |
| | GCTGAGGGAGCTGATCGTGGGCCAATACTTTCCCGAGGTCCTCGACAA |
| | AGAATACGACCTCTCCAAGAATGGAGACAGCGTGGTGACATACAGAAG |
| | CAAGATTTATACCGTGATGAATTACATTCTGCTGTATTACCTCGAGGAC |
| | CACGACTCCAGCAGAGAAAGCATGGTCGAAGCTCTGAGACAAAACAGA |
| | GAGGGCGATGAAGGCAAGGAGGAGATCTATAGACAGTTTGCCAAGAA |
| | GGTGTGGAACGGCGTGTCCGGACTGTTTGGCGTGTGTCTGAACCTCTTC |
| | AAGACCGAAAAGAGAAACAAGTTTAGGAGCAAAGTCGCCCTCCCCGAT |
| | GTGTCCGGCGCTGCCTATATGCTCTCCTCCGAGAACATCGACTACTTTG |
| | TCAAGATGCTCTTCTTTGTGTGTAAGTTTCTGGATGGCAAAGAAATCAA |
| | CGAGCTGCTGTGCGCTCTGATCAACAAATTTGATAATATTGCCGATATT |
| | CTGGATGCTGCCGCTCAATGTGGCTCCTCCGTCTGGTTCGTGGACAGCT |
| | ATAGGTTCTTCGAGAGATCTAGGAGGATTAGCGCCCAGATTAGAATCGT |
| | GAAGAACATCGCTTCCAAGGATTTTAAGAAATCCAAGAAGGATTCCGA |
| | TGAGAGCTACCCCGAGCAGCTGTATCTGGATGCTCTGGCTCTGCTCGGA |
| | GACGTCATCTCCAAGTACAAGCAGAATAGAGATGGCAGCGTCGTCATC |
| | GATGACCAAGGCAATGCCGTGCTGACAGAGCAATACAAGAGGTTTAGA |
| | TATGAATTTTTCGAGGAGATCAAGAGGGACGAAAGCGGCGGCATCAAG |
| | TACAAGAAGTCCGGAAAACCCGAGTACAACCATCAGAGAAGGAATTTT |
| | ATTCTGAATAATGTGCTGAAAAGCAAATGGTTTTTCTATGTGGTGAAGT |
| | ACAATAGGCCCAGCAGCTGCAGAGAACTGATGAAGAATAAGGAAATTC |
| | TGAGGTTCGTGCTGAGAGACATCCCCGACTCCCAAGTGAGAAGATACTT |
| | TAAGGCCGTCCAAGGAGAGGAAGCTTACGCTAGCGCCGAAGCTATGAG |
| | GACAAGACTGGTCGACGCTCTGTCCCAATTTAGCGTCACAGCTTGTCTG |
| | GATGAAGTGGGCGGCATGACAGACAAGGAATTCGCCTCCCAGAGGGCC |
| | GTCGATAGCAAAGAAAAACTGAGAGCCATCATCAGACTGTATCTGACA |
| | GTCGCCTATCTGATTACCAAGAGCATGGTGAAGGTGAATACAAGGTTTA |
| | GCATTGCCTTTAGCGTGCTGGAGAGGGACTACTATCTGCTCATTGACGG |
| | CAAGAAGAAATCCAGCGACTACACCGGAGAGGATATGCTGGCTCTGAC |
| | CAGAAAATTTGTGGGCGAAGATGCTGGACTGTATAGAGAGTGGAAAGA |
| | GAAGAACGCTGAAGCCAAGGACAAATATTTTGACAAGGCCGAAAGGA |
| | AGAAGGTGCTGAGACAGAACGATAAGATGATCAGAAAGATGCACTTCA |
| | CACCCCACTCCCTCAATTACGTCCAAAAGAATCTCGAAAGCGTCCAGAG |
| | CAACGGACTGGCCGCCGTCATCAAGGAATATgcAAATGCCGTCGCTgcCC |
| | TCAATATCATCAATAGACTGGACGAGTACATTGGCTCCGCTAGGGCTGA |
| | TAGCTACTACTCTCTGTACTGTTACTGCCTCCAAATGTATCTGAGCAAG |
| | AACTTCAGCGTGGGCTACCTCATCAACGTGCAAAAGCAGCTGGAGGAG |
| | CACCACACCTACATGAAGGATCTCATGTGGCTGCTCAACATCCCCTTCG |
| | CTTACAACCTCGCCAGATACAAAAATCTGTCCAACGAAAAACTCTTTTA |
| | CGACGAGGAAGCCGCCGCCGAAAAGGCTGACAAGGCTGAGAACGAGA |
| | GAGGCGAA (SEQ ID NO: 607) |
| Linker | GGAAGC |
| SV40 NLS | CCCAAGAAGAAAAGGAAGGTC (SEQ ID NO: 532) |
| Linker | GAGGAC |
| HA Tag | TACCCCTACGATGTGCCCGACTACGCC (SEQ ID NO: 608) |
| WPRE3 | GATAATCAACCTCTGGATTACAAAATTTGTGAAAGATTGACTGGTATTC |
| | TTAACTATGTTGCTCCTTTTACGCTATGTGGATACGCTGCTTTAATGCCT |
| | TTGTATCATGCTATTGCTTCCCGTATGGCTTTCATTTTCTCCTCCTTGTAT |
| | AAATCCTGGTTAGTTCTTGCCACGGCGGAACTCATCGCCGCCTGCCTTG |
| | CCCGCTGCTGGACAGGGGCTCGGCTGTTGGGCACTGACAATTCCGTGG |
| | (SEQ ID NO: 609) |
| SV-40 poly A | AACTTGTTTATTGCAGCTTATAATGGTTACAAATAAAGCAATAGCATCA |
| | CAAATTTCACAAATAAAGCATTTTTTTCACTGCATTCTAGTTGTGGTTTG |
| | TCCAAACTCATCAATGTATCTTA (SEQ ID NO: 533) |
| 3′ITR | Aggaacccctagtgatggagttggccactccctctctgcgcgctcgctcgctcactgaggccgggcgaccaaaggtc |
| | gcccgacgcccgggctttgcccgggcggcctcagtgagcgagcgagcgcgcagctgcctgcagg (SEQ ID |
| | NO: 598) |

### CAG-Targeting Cas13d PUF AAV Vectors

In some embodiments of the compositions of the disclosure, CAG-targeting PUF compositions are packaged as AAV vectors. In some embodiments, CAG-targeting PUF compositions packaged as AAV vectors are set forth in SEQ ID NOs 518, 528, 534, 536, and 539.

In some embodiments, an AAV vector comprising a nucleic acid encoding a CAG-repeat targeting PUF comprises from 5′ to 3′: a sequence encoding a 5′ ITR (a first ITR), a sequence encoding an EFS/UBB promoter, a sequence encoding a kozak sequence, a sequence encoding an 8PUF protein, a sequence encoding a linker, a sequence encoding a nuclease (E17), a sequence encoding a WPRE element, a sequence encoding an SV40 polyA sequence, and a 3′ ITR (a second ITR). In some embodiments, the CAG-targeting Cas13d composition is arranged as depicted in Table P. In some embodiments, the vector set forth in Table P is referred to as A01383.

TABLE P
Vector A01383 encoding a CAG-repeat targeting PUF-E17 fusion

| Plasmid | |
|---|---|
| Element | DNA Sequence |
| 5′ ITR | CCTGCAGGCAGCTGCGCGCTCGCTCGCTCACTGAGGCCGCCCGGGCGT |
| | CGGGCGACCTTTGGTCGCCCGGCCTCAGTGAGCGAGCGAGCGCGCAGA |
| | GAGGGAGTGGCCAACTCCATCACTAGGGGTTCCT (SEQ ID NO: 597) |
| EFS/UBB | GGGCAGAGCGCACATCGCCCACAGTCCCCGAGAAGTTGGGGGGAGGGGTCGG |
| Promoter | CAATTGAaCCGGTGCCTAGAGAAGGTGGCGCGGGGTAAACTGGGAAAGTGAT |
| | GTCGTGTACTGGCTCCGCCTTTTTCCCGAGGGTGGGGGAGAACCGTATATAAG |
| | TGCAGTAGTCGCCGTGAACGTTCTTTTTCGCAACGGGTTTGCCGCCAGAACAC |
| | AGaattccagGTAAGTCCCGCAGCCGTAACGACCTTGGGGGGGTGTGAGATTCTCA |
| | TTCTAATTTTGAAGAATATTAGGTGTAAAAGCAAGAAATACAATGATCCTGAG |
| | GTGACACGCTTATGTTTTACTTTTAAACTAG (SEQ ID NO: 613) |
| Kozak | Gccgccaccatg (SEQ ID NO: 529) |
| Sequence | |
| 8PUF | gGCCGCAGCCGCCTTTTGGAAGATTTTCGAAACAACCGGTACCCCAATT |
| | TACAACTGCGGGAGATTGCCGGACATATAATGGAATTTTCCCAAGACC |
| | AGCATGGGTCCAGATTCATTCGCCTGAAACTGGAGCGTGCCACACCAG |
| | CTGAGCGCCAGCTTGTCTTCAATGAAATCCTCCAGGCTGCCTACCAACT |
| | CATGGTGGATGTGTTTGGTAGTTACGTCATTGAAAAGTTCTTTGAATTT |
| | GGCAGTCTTGAACAGAAGCTGGCTTTGGCAGAACGGATTCGAGGTCAC |
| | GTCCTGTCATTGGCACTACAGATGTATGGCTGTCGTGTTATCCAGAAAG |
| | CTCTTGAGTTTATTCCTTCAGACCAGCAGAATGAGATGGTTCGGGAACT |
| | AGATGGCCATGTCTTGAAGTGTGTGAAAGATCAGAATGGCAGTTACGT |
| | GGTTCGCAAATGCATTGAATGTGTACAGCCCCAGTCTTTGCAATTTATC |
| | ATCGATGCGTTTAAGGGACAGGTATTTGCCTTATCCACACATCCTTATG |
| | GCTCCCGAGTGATTGAGAGAATCCTGGAGCACTGTCTCCCTGACCAGA |
| | CACTCCCTATTTTAGAGGAGCTTCACCAGCACACAGAGCAGCTTGTAC |
| | AGGATCAATATGGATGTTATGTAATCCAGCATGTACTGGAGCACGGTC |
| | GTCCTGAGGATAAAAGCAAAATTGTAGCAGAAATCCGAGGCAATGTAC |
| | TTGTATTGAGTCAGCACAAATTTGCAAGCTATGTTGTGCGCAAGTGTGT |
| | TACTCACGCCTCACGTACGGAGCGCGCTGTGCTCATCGATGAGGTGTG |
| | CACCATGAACGACGGTCCCCACAGTGCCTTATACACCATGATGAAGGA |
| | CCAGTATGCCAGCTACGTGGTCGAGAAGATGATTGACGTGGCGGAGCC |
| | AGGCCAGCGGAAGATCGTCATGCATAAGATCCGACCCCACATCGCAAC |
| | TCTTCGTAAGTACACCTATGGCAAGCACATTCTGGCCAAGCTGGAGAA |
| | GTACTACATGAAGAACGGTGTTGACTTAGGC (SEQ ID NO: 614) |
| Linker | GTGGATACTGCCAATGGCAGC (SEQ ID NO: 615) |
| E17 | Ggtggtggcacccctaaggctcccaacctggagcctccactcccagaagaggaaaaggagggcagcgacctgag |
| | accagtggtcatcgatgggagcaacgtggccatgagccatgggaacaaggaggtcttctcctgccggggcatcctg |
| | ctggcagtgaactggtttctggagcggggccacacagacatcacagtgtttgtgccatcctggaggaaggagcagcc |
| | tcggcccgacgtgcccatcacagaccagcacatcctgcgggaactggagaagaagaagatcctggtgttcacacca |
| | tcacgacgcgtgggggcaagcgggtggtgtgctatgacgacagattcattgtgaagctggcctacgagtctgacgg |
| | gatcgtggtttccaacgacacataccgtgacctccaaggcgagcggcaggagtggaagcgcttcatcgaggagcgg |
| | ctgctcatgtactccttcgtcaatgacaagtttatgccccctgatgacccactgggccggcacgggcccagcctggac |
| | aacttcctgcgtaagaagccactcactttggag (SEQ ID NO: 616) |
| WPRE | Aatcaacctctggattacaaaatttgtgaaagattgactggtattcttaactatgttgctccttttacgctatgtggatacgc |
| | tgctttaatgcctttgtatcatgctattgcttcccgtatggctttcattttctcctccttgtataaatcctggttgctgtctctttat |
| | gaggagttgtggcccgttgtcaggcaacgtggcgtggtgtgcactgtgtttgctgacgcaacccccactggttggggc |
| | attgccaccacctgtcagctcctttccgggactttcgctttccccctccctattgccacggcggaactcatcgccgcctg |
| | ccttgcccgctgctggacaggggctcggctgttgggcactgacaattccgtggtgttgtcggggaaatcatcgtccttt |
| | ccttggctgctcgcctAtgttgccacctggattctgcgcgggacgtccttctgctacgtcccttcggccctcaatccagc |
| | ggaccttccttcccgcggcctgctgccggctctgcggcctcttccgcgtcttcgccttcgccctcagacgagtcggatc |
| | tccctttgggccgcctccccgc (SEQ ID NO: 617) |
| SV40 | AACTTGTTTATTGCAGCTTATAATGGTTACAAATAAAGCAATAGCATCA |
| poly A | CAAATTTCACAAATAAAGCATTTTTTTCACTGCATTCTAGTTGTGGTTT |
| | GTCCAAACTCATCAATGTATCTTA (SEQ ID NO: 533) |
| 3′ ITR | AGGAACCCCTAGTGATGGAGTTGGCCACTCCCTCTCTGCGCGCTCGCTC |
| | GCTCACTGAGGCCGGGCGACCAAAGGTCGCCCGACGCCCGGGCTTTGC |
| | CCGGGCGGCCTCAGTGAGCGAGCGAGCGCGCAGCTGCCTGCAGG (SEQ |
| | ID NO: 598) |

In some embodiments, an AAV vector comprising a nucleic acid encoding a CAG-repeat targeting PUF comprises from 5′ to 3′: a sequence encoding a 5′ ITR (a first ITR), a sequence encoding an EFS/UBB promoter, a sequence encoding a kozak sequence, a sequence encoding an 8PUF protein, a sequence encoding a linker, a sequence encoding a myc tag, a sequence encoding a WPRE element, a sequence encoding an SV40 polyA sequence, and a 3′ ITR (a second ITR). In some embodiments, the CAG-targeting Cas13d composition is arranged as depicted in Table Q. In some embodiments, the vector set forth in Table Q is referred to as A01684. In some embodiments, vector A01684 is suitable for blocking.

TABLE Q
Vector A01684 encoding a CAG-repeat targeting PUF for blocking

| | |
|---|---|
| 5′ ITR | CCTGCAGGCAGCTGCGCGCTCGCTCGCTCACTGAGGCCGCCCGGGCGT |
| | CGGGCGACCTTTGGTCGCCCGGCCTCAGTGAGCGAGCGAGCGCGCAGA |
| | GAGGGAGTGGCCAACTCCATCACTAGGGGTTCCT (SEQ ID NO: 597) |
| EFS/UBB | GGGCAGAGCGCACATCGCCCACAGTCCCCGAGAAGTTGGGGGGAGGGGTCGG |
| Promoter | CAATTGAaCCGGTGCCTAGAGAAGGTGGCGCGGGGTAAACTGGGAAAGTGAT |
| | GTCGTGTACTGGCTCCGCCTTTTTCCCGAGGGTGGGGGAGAACCGTATATAAG |
| | TGCAGTAGTCGCCGTGAACGTTCTTTTTCGCAACGGGTTTGCCGCCAGAACAC |
| | AGaattccagGTAAGTCCCGCAGCCGTAACGACCTTGGGGGGGTGTGAGATTCTCA |
| | TTCTAATTTTGAAGAATATTAGGTGTAAAAGCAAGAAATACAATGATCCTGAG |
| | GTGACACGCTTATGTTTTACTTTTAAACTAG (SEQ ID NO: 613) |
| Kozak | Gccgccaccatg (SEQ ID NO: 529) |
| Sequence | |
| 8PUF | gGCCGCAGCCGCCTTTTGGAAGATTTTCGAAACAACCGGTACCCCAATT |
| | TACAACTGCGGGAGATTGCCGGACATATAATGGAATTTTCCCAAGACC |
| | AGCATGGGTCCAGATTCATTCGCCTGAAACTGGAGCGTGCCACACCAG |
| | CTGAGCGCCAGCTTGTCTTCAATGAAATCCTCCAGGCTGCCTACCAACT |
| | CATGGTGGATGTGTTTGGTAGTTACGTCATTGAAAAGTTCTTTGAATTT |
| | GGCAGTCTTGAACAGAAGCTGGCTTTGGCAGAACGGATTCGAGGTCAC |
| | GTCCTGTCATTGGCACTACAGATGTATGGCTGTCGTGTTATCCAGAAAG |
| | CTCTTGAGTTTATTCCTTCAGACCAGCAGAATGAGATGGTTCGGGAACT |
| | AGATGGCCATGTCTTGAAGTGTGTGAAAGATCAGAATGGCAGTTACGT |
| | GGTTCGCAAATGCATTGAATGTGTACAGCCCCAGTCTTTGCAATTTATC |
| | ATCGATGCGTTTAAGGGACAGGTATTTGCCTTATCCACACATCCTTATG |
| | GCTCCCGAGTGATTGAGAGAATCCTGGAGCACTGTCTCCCTGACCAGA |
| | CACTCCCTATTTTAGAGGAGCTTCACCAGCACACAGAGCAGCTTGTAC |
| | AGGATCAATATGGATGTTATGTAATCCAGCATGTACTGGAGCACGGTC |
| | GTCCTGAGGATAAAAGCAAAATTGTAGCAGAAATCCGAGGCAATGTAC |
| | TTGTATTGAGTCAGCACAAATTTGCAAGCTATGTTGTGCGCAAGTGTGT |
| | TACTCACGCCTCACGTACGGAGCGCGCTGTGCTCATCGATGAGGTGTG |
| | CACCATGAACGACGGTCCCCACAGTGCCTTATACACCATGATGAAGGA |
| | CCAGTATGCCAGCTACGTGGTCGAGAAGATGATTGACGTGGCGGAGCC |
| | AGGCCAGCGGAAGATCGTCATGCATAAGATCCGACCCCACATCGCAAC |
| | TCTTCGTAAGTACACCTATGGCAAGCACATTCTGGCCAAGCTGGAGAA |
| | GTACTACATGAAGAACGGTGTTGACTTAGGC (SEQ ID NO: 619) |
| Linker | GGCGGAAGT (SEQ ID NO: 618) |
| Myc tag | GAGCAAAAACTGATTAGTGAAGAAGATCTC (SEQ ID NO: 620) |
| WPRE | Aatcaacctctggattacaaaatttgtgaaagattgactggtattcttaactatgttgctccttttacgctatgtggatacgc |
| | tgctttaatgcctttgtatcatgctattgcttcccgtatggctttcattttctcctccttgtataaatcctggttgctgtctctttat |
| | gaggagttgtggcccgttgtcaggcaacgtggcgtggtgtgcactgtgtttgctgacgcaacccccactggttggggc |
| | attgccaccacctgtcagctcctttccgggactttcgctttccccctccctattgccacggcggaactcatcgccgcctg |
| | ccttgcccgctgctggacaggggctcggctgttgggcactgacaattccgtggtgttgtcggggaaatcatcgtccttt |
| | ccttggctgctcgcctAtgttgccacctggattctgcgcgggacgtccttctgctacgtcccttcggccctcaatccagc |
| | ggaccttccttcccgcggcctgctgccggctctgcggcctcttccgcgtcttcgccttcgccctcagacgagtcggatc |
| | tccctttgggccgcctccccgc (SEQ ID NO: 617) |
| SV40 | AACTTGTTTATTGCAGCTTATAATGGTTACAAATAAAGCAATAGCATCA |
| poly A | CAAATTTCACAAATAAAGCATTTTTTTCACTGCATTCTAGTTGTGGTTT |
| | GTCCAAACTCATCAATGTATCTTA (SEQ ID NO: 533) |
| 3′ ITR | AGGAACCCCTAGTGATGGAGTTGGCCACTCCCTCTCTGCGCGCTCGCTC |
| | GCTCACTGAGGCCGGGCGACCAAAGGTCGCCCGACGCCCGGGCTTTGC |
| | CCGGGCGGCCTCAGTGAGCGAGCGAGCGCGCAGCTGCCTGCAGG (SEQ |
| | ID NO: 598) |

In some embodiments, an AAV vector comprising a nucleic acid encoding a CAG-repeat targeting PUF comprises from 5′ to 3′: a sequence encoding a 5′ ITR (a first ITR), a sequence encoding an EFS/UBB promoter, a sequence encoding a kozak sequence, a sequence encoding an 8PUF protein, a sequence encoding a WPRE element, a sequence encoding an SV40 polyA sequence, and a 3′ ITR (a second ITR). In some embodiments, the CAG-targeting Cas13d composition is arranged as depicted in Table R. In some embodiments, the vector set forth in Table R is referred to as A01683.

TABLE R
Vector A01683 encoding a CAG-repeat targeting PUF for blocking

| Plasmid | |
|---|---|
| Element | DNA Sequence |
| 5′ ITR | CCTGCAGGCAGCTGCGCGCTCGCTCGCTCACTGAGGCCGCCCGGGCGT |
| | CGGGCGACCTTTGGTCGCCCGGCCTCAGTGAGCGAGCGAGCGCGCAGA |
| | GAGGGAGTGGCCAACTCCATCACTAGGGGTTCCT (SEQ ID NO: 597) |
| EFS/UBB | GGGCAGAGCGCACATCGCCCACAGTCCCCGAGAAGTTGGGGGGAGGGGTCGG |
| Promoter | CAATTGAaCCGGTGCCTAGAGAAGGTGGCGCGGGGTAAACTGGGAAAGTGAT |
| | GTCGTGTACTGGCTCCGCCTTTTTCCCGAGGGTGGGGGAGAACCGTATATAAG |
| | TGCAGTAGTCGCCGTGAACGTTCTTTTTCGCAACGGGTTTGCCGCCAGAACAC |
| | AGaattccagGTAAGTCCCGCAGCCGTAACGACCTTGGGGGGGTGTGAGATTCTCA |
| | TTCTAATTTTGAAGAATATTAGGTGTAAAAGCAAGAAATACAATGATCCTGAG |
| | GTGACACGCTTATGTTTTACTTTTAAACTAG (SEQ ID NO: 613) |
| Kozak | Gccgccaccatg (SEQ ID NO: 529) |
| Sequence | |
| 8PUF | gGCCGCAGCCGCCTTTTGGAAGATTTTCGAAACAACCGGTACCCCAATT |
| | TACAACTGCGGGAGATTGCCGGACATATAATGGAATTTTCCCAAGACC |
| | AGCATGGGTCCAGATTCATTCGCCTGAAACTGGAGCGTGCCACACCAG |
| | CTGAGCGCCAGCTTGTCTTCAATGAAATCCTCCAGGCTGCCTACCAACT |
| | CATGGTGGATGTGTTTGGTAGTTACGTCATTGAAAAGTTCTTTGAATTT |
| | GGCAGTCTTGAACAGAAGCTGGCTTTGGCAGAACGGATTCGAGGTCAC |
| | GTCCTGTCATTGGCACTACAGATGTATGGCTGTCGTGTTATCCAGAAAG |
| | CTCTTGAGTTTATTCCTTCAGACCAGCAGAATGAGATGGTTCGGGAACT |
| | AGATGGCCATGTCTTGAAGTGTGTGAAAGATCAGAATGGCAGTTACGT |
| | GGTTCGCAAATGCATTGAATGTGTACAGCCCCAGTCTTTGCAATTTATC |
| | ATCGATGCGTTTAAGGGACAGGTATTTGCCTTATCCACACATCCTTATG |
| | GCTCCCGAGTGATTGAGAGAATCCTGGAGCACTGTCTCCCTGACCAGA |
| | CACTCCCTATTTTAGAGGAGCTTCACCAGCACACAGAGCAGCTTGTAC |
| | AGGATCAATATGGATGTTATGTAATCCAGCATGTACTGGAGCACGGTC |
| | GTCCTGAGGATAAAAGCAAAATTGTAGCAGAAATCCGAGGCAATGTAC |
| | TTGTATTGAGTCAGCACAAATTTGCAAGCTATGTTGTGCGCAAGTGTGT |
| | TACTCACGCCTCACGTACGGAGCGCGCTGTGCTCATCGATGAGGTGTG |
| | CACCATGAACGACGGTCCCCACAGTGCCTTATACACCATGATGAAGGA |
| | CCAGTATGCCAGCTACGTGGTCGAGAAGATGATTGACGTGGCGGAGCC |
| | AGGCCAGCGGAAGATCGTCATGCATAAGATCCGACCCCACATCGCAAC |
| | TCTTCGTAAGTACACCTATGGCAAGCACATTCTGGCCAAGCTGGAGAA |
| | GTACTACATGAAGAACGGTGTTGACTTAGGC (SEQ ID NO: 621) |
| WPRE | Aatcaacctctggattacaaaatttgtgaaagattgactggtattcttaactatgttgctccttttacgctatgtggatacgc |
| | tgctttaatgcctttgtatcatgctattgcttcccgtatggctttcattttctcctccttgtataaatcctggttgctgtctctttat |
| | gaggagttgtggcccgttgtcaggcaacgtggcgtggtgtgcactgtgtttgctgacgcaacccccactggttggggc |
| | attgccaccacctgtcagctcctttccgggactttcgctttccccctccctattgccacggcggaactcatcgccgcctg |
| | ccttgcccgctgctggacaggggctcggctgttgggcactgacaattccgtggtgttgtcggggaaatcatcgtccttt |
| | ccttggctgctcgcctAtgttgccacctggattctgcgcgggacgtccttctgctacgtcccttcggccctcaatccagc |
| | ggaccttccttcccgcggcctgctgccggctctgcggcctcttccgcgtcttcgccttcgccctcagacgagtcggatc |
| | tccctttgggccgcctccccgc (SEQ ID NO: 617) |
| SV40 | AACTTGTTTATTGCAGCTTATAATGGTTACAAATAAAGCAATAGCATCA |
| poly A | CAAATTTCACAAATAAAGCATTTTTTTCACTGCATTCTAGTTGTGGTTT |
| | GTCCAAACTCATCAATGTATCTTA (SEQ ID NO: 533) |
| 3′ ITR | AGGAACCCCTAGTGATGGAGTTGGCCACTCCCTCTCTGCGCGCTCGCTC |
| | GCTCACTGAGGCCGGGCGACCAAAGGTCGCCCGACGCCCGGGCTTTGC |
| | CCGGGCGGCCTCAGTGAGCGAGCGAGCGCGCAGCTGCCTGCAGG (SEQ |
| | ID NO: 598) |

In some embodiments, an AAV vector comprising a nucleic acid encoding a CAG-repeat targeting PUF comprises from 5′ to 3′: a sequence encoding a 5′ ITR (a first ITR), a sequence encoding an EFS/UBB promoter, a sequence encoding a kozak sequence, a sequence encoding an 8PUF protein, a linker sequence, a PIN endonuclease, a linker sequence, a myc tag, a sequence encoding a WPRE element, a sequence encoding an SV40 polyA sequence, and a 3′ ITR (a second ITR). In some embodiments, the CAG-targeting Cas13d composition is arranged as depicted in Table S1 and S2. A nucleic acid sequence encoding Vector A02249 comprises SEQ ID NO: 624. A nucleic acid sequence encoding Vector A02250 comprises SEQ ID NO: 625.

TABLE S1
Vector A02250 encoding a CAG-repeat targeting PUF fused to a PIN
endonuclease

| Plasmid | |
|---|---|
| Element | DNA Sequence |
| 5′ ITR | CCTGCAGGCAGCTGCGCGCTCGCTCGCTCACTGAGGCCGCCCGG |
| | GCGTCGGGCGACCTTTGGTCGCCCGGCCTCAGTGAGCGAGCGAG |
| | CGCGCAGAGAGGGAGTGGCCAACTCCATCACTAGGGGTTCCT |
| | (SEQ ID NO: 597) |
| EFS/UBB | GGGCAGAGCGCACATCGCCCACAGTCCCCGAGAAGTTGGGGGGAGGG |
| Promoter | GTCGGCAATTGAaCCGGTGCCTAGAGAAGGTGGCGCGGGGTAAACTG |
| | GGAAAGTGATGTCGTGTACTGGCTCCGCCTTTTTCCCGAGGGTGGGGG |
| | AGAACCGTATATAAGTGCAGTAGTCGCCGTGAACGTTCTTTTTCGCAA |
| | CGGGTTTGCCGCCAGAACACAGaattccagGTAAGTCCCGCAGCCGTAACG |
| | ACCTTGGGGGGGTGTGAGATTCTCATTCTAATTTTGAAGAATATTAGG |
| | TGTAAAAGCAAGAAATACAATGATCCTGAGGTGACACGCTTATGTTTT |
| | ACTTTTAAACTAGGT (SEQ ID NO: 613) |
| Kozak | Gccgccaccatg (SEQ ID NO: 529) |
| Sequence | |
| 8PUF | gGCCGCAGCCGCCTTTTGGAAGATTTTCGAAACAACCGGTACCC |
| | CAATTTACAACTGCGGGAGATTGCCGGACATATAATGGAATTTT |
| | CCCAAGACCAGCATGGGTCCAGATTCATTCGCCTGAAACTGGAG |
| | CGTGCCACACCAGCTGAGCGCCAGCTTGTCTTCAATGAAATCCT |
| | CCAGGCTGCCTACCAACTCATGGTGGATGTGTTTGGTAGTTACG |
| | TCATTGAAAAGTTCTTTGAATTTGGCAGTCTTGAACAGAAGCTG |
| | GCTTTGGCAGAACGGATTCGAGGTCACGTCCTGTCATTGGCACT |
| | ACAGATGTATGGCTGTCGTGTTATCCAGAAAGCTCTTGAGTTTA |
| | TTCCTTCAGACCAGCAGAATGAGATGGTTCGGGAACTAGATGGC |
| | CATGTCTTGAAGTGTGTGAAAGATCAGAATGGCAGTTACGTGGT |
| | TCGCAAATGCATTGAATGTGTACAGCCCCAGTCTTTGCAATTTA |
| | TCATCGATGCGTTTAAGGGACAGGTATTTGCCTTATCCACACAT |
| | CCTTATGGCTCCCGAGTGATTGAGAGAATCCTGGAGCACTGTCT |
| | CCCTGACCAGACACTCCCTATTTTAGAGGAGCTTCACCAGCACA |
| | CAGAGCAGCTTGTACAGGATCAATATGGATGTTATGTAATCCAG |
| | CATGTACTGGAGCACGGTCGTCCTGAGGATAAAAGCAAAATTGT |
| | AGCAGAAATCCGAGGCAATGTACTTGTATTGAGTCAGCACAAAT |
| | TTGCAAGCTATGTTGTGCGCAAGTGTGTTACTCACGCCTCACGT |
| | ACGGAGCGCGCTGTGCTCATCGATGAGGTGTGCACCATGAACG |
| | ACGGTCCCCACAGTGCCTTATACACCATGATGAAGGACCAGTAT |
| | GCCAGCTACGTGGTCGAGAAGATGATTGACGTGGCGGAGCCAG |
| | GCCAGCGGAAGATCGTCATGCATAAGATCCGACCCCACATCGC |
| | AACTCTTCGTAAGTACACCTATGGCAAGCACATTCTGGCCAAGC |
| | TGGAGAAGTACTACATGAAGAACGGTGTTGACTTAGGC(SEQ ID |
| | NO: 614) |
| Linker | GTGGATACTGCCAATGGCAGC (SEQ ID NO: 615) |
| PIN | CAGATGGAGCTCGAAATCAGGCCGCTGTTCCTCGTGCCGGACAC |
| | TAATGGTTTTATAGATCACTTGGCGTCCTTGGCTAGACTTCTGGA |
| | AAGCCGAAAGTATATATTGGTAGTGCCGTTGATTGTAATTAACG |
| | AATTGGATGGGTTGGCGAAAGGACAAGAGACTGATCACAGAGC |
| | AGGAGGCTACGCGAGGGTCGTCCAAGAGAAGGCGCGAAAAAGC |
| | ATCGAGTTCCTGGAGCAGCGATTTGAGAGCAGGGACTCATGCCT |
| | GAGAGCCCTCACGTCCCGGGGGAACGAGCTGGAGTCCATCGCTT |
| | TCCGAAGTGAAGACATTACGGGCCAACTTGGGAATAATGATGA |
| | CCTCATCTTGTCCTGCTGCCTGCACTACTGCAAGGACAAGGCTA |
| | AGGACTTCATGCCTGCCTCCAAGGAGGAGCCTATCCGATTGTTG |
| | AGGGAAGTAGTACTTTTGACGGACGACCGCAACCTCCGGGTAA |
| | AGGCGCTGACTCGAAATGTCCCAGTAAGGGATATACCGGCGTTC |
| | CTTACATGGGCTCAAGTAGGG (SEQ ID NO: 623) |
| Linker | GGCGGAtct |
| Myc tag | GAGCAgAAACTGATTAGcGAAGAgGATCTC (SEQ ID NO: 610) |
| WPRE | Aatcaacctctggattacaaaatttgtgaaagattgactggtattcttaactatgttgctccttttacgctatgtg |
| | gatacgctgctttaatgcctttgtatcatgctattgcttcccgtatggctttcattttctcctccttgtataaatcctg |
| | gttgctgtctctttatgaggagttgtggcccgttgtcaggcaacgtggcgtggtgtgcactgtgtttgctgacg |
| | caacccccactggttggggcattgccaccacctgtcagctcctttccgggactttcgetttccccctccctatt |
| | gccacggcggaactcatcgccgcctgccttgcccgctgctggacaggggctcggctgttgggcactgac |
| | aattccgtggtgttgtcggggaaatcatcgtcctttccttggctgctcgcctAtgttgccacctggattctgcg |
| | cgggacgtccttctgctacgtcccttcggccctcaatccagcggaccttccttcccgcggcctgctgccgg |
| | ctctgcggcctcttccgcgtcttcgccttcgccctcagacgagtcggatctccctttgggccgcctccccgc |
| | (SEQ ID NO: 617) |
| SV40 poly A | AACTTGTTTATTGCAGCTTATAATGGTTACAAATAAAGCAATAG |
| | CATCACAAATTTCACAAATAAAGCATTTTTTTCACTGCATTCTAG |
| | TTGTGGTTTGTCCAAACTCATCAATGTATCTTA (SEQ ID NO: 533) |
| 3′ ITR | AGGAACCCCTAGTGATGGAGTTGGCCACTCCCTCTCTGCGCGCT |
| | CGCTCGCTCACTGAGGCCGGGCGACCAAAGGTCGCCCGACGCC |
| | CGGGCTTTGCCCGGGCGGCCTCAGTGAGCGAGCGAGCGCGCAG |
| | CTGCCTGCAGG (SEQ ID NO: 598) |

TABLE S2
CAG-repeat targeting PUF fused
to a PIN endonuclease

| | | | | Amino |
|---|---|---|---|---|
| | Pro- | | | Acid |
| | tein | | Target | Sequence |
| Construct | Type | Elements | Sequence | of PUF |
| A02250 | 8PUF | N-terminal | GCAGCAGC | PUF |
| | | PUF; linker | (SEQ | SEQ |
| | | between PUF | ID | ID |
| | | and PIN | NO: | NO: |
| | | endonuclease | 476 | 549 |
| | | (VDTANGS); | | |
| | | C-terminal | | |
| | | PIN | | |
| | | Myc tag | | |

In some embodiments, an AAV vector comprising a nucleic acid encoding a CAG-repeat targeting PUF comprises from 5′ to 3′: a sequence encoding a 5′ ITR (a first ITR), a sequence encoding an EFS/UBB promoter, a sequence encoding a kozak sequence, a sequence encoding an 8PUF protein, a linker sequence, a PIN endonuclease, a sequence encoding a WPRE element, a sequence encoding a polyA sequence, and a 3′ ITR (a second ITR). In some embodiments, the CAG-targeting Cas13d composition is arranged as depicted in Table S3 and S4.

TABLE S3
Vector A02249 encoding a CAG-repeat
targeting PUF fused to a PIN
endonuclease

| Plasmid | |
|---|---|
| Element | DNA Sequence |
| 5′ ITR | CCTGCAGGCAGCTGCGCGCTCGCTC |
| | GCTCACTGAGGCCGCCCGGGCGTCG |
| | GGCGACCTTTGGTCGCCCGGCCTCA |
| | GTGAGCGAGCGAGCGCGCAGAGAGG |
| | GAGTGGCCAACTCCATCACTAGGGG |
| | TTCCT |
| | (SEQ ID NO: 597) |
| EFS/UBB | GGGCAGAGCGCACATCGCCCACAGT |
| Promoter | CCCCGAGAAGTTGGGGGGAGGGGTC |
| | GGCAATTGAaCCGGTGCCTAGAGAA |
| | GGTGGCGCGGGGTAAACTGGGAAAG |
| | TGATGTCGTGTACTGGCTCCGCCTT |
| | TTTCCCGAGGGTGGGGGAGAACCGT |
| | ATATAAGTGCAGTAGTCGCCGTGAA |
| | CGTTCTTTTTCGCAACGGGTTTGCC |
| | GCCAGAACACAGaattccagGTAAG |
| | TCCCGCAGCCGTAACGACCTTGGGG |
| | GGGTGTGAGATTCTCATTCTAATTT |
| | TGAAGAATATTAGGTGTAAAAGCAA |
| | GAAATACAATGATCCTGAGGTGACA |
| | CGCTTATGTTTTACTTTTAAACTAG |
| | GT |
| | (SEQ ID NO: 613) |
| Kozak | Gccgccaccatg |
| Sequence | (SEQ ID NO: 529) |
| 8PUF | gGCCGCAGCCGCCTTTTGGAAGATT |
| | TTCGAAACAACCGGTACCCCAATTT |
| | ACAACTGCGGGAGATTGCCGGACAT |
| | ATAATGGAATTTTCCCAAGACCAGC |
| | ATGGGTCCAGATTCATTCGCCTGAA |
| | ACTGGAGCGTGCCACACCAGCTGAG |
| | CGCCAGCTTGTCTTCAATGAAATCC |
| | TCCAGGCTGCCTACCAACTCATGGT |
| | GGATGTGTTTGGTAGTTACGTCATT |
| | GAAAAGTTCTTTGAATTTGGCAGTC |
| | TTGAACAGAAGCTGGCTTTGGCAGA |
| | ACGGATTCGAGGTCACGTCCTGTCA |
| | TTGGCACTACAGATGTATGGCTGTC |
| | GTGTTATCCAGAAAGCTCTTGAGTT |
| | TATTCCTTCAGACCAGCAGAATGAG |
| | ATGGTTCGGGAACTAGATGGCCATG |
| | TCTTGAAGTGTGTGAAAGATCAGAA |
| | TGGCAGTTACGTGGTTCGCAAATGC |
| | ATTGAATGTGTACAGCCCCAGTCTT |
| | TGCAATTTATCATCGATGCGTTTAA |
| | GGGACAGGTATTTGCCTTATCCACA |
| | CATCCTTATGGCTCCCGAGTGATTG |
| | AGAGAATCCTGGAGCACTGTCTCCC |
| | TGACCAGACACTCCCTATTTTAGAG |
| | GAGCTTCACCAGCACACAGAGCAGC |
| | TTGTACAGGATCAATATGGATGTTA |
| | TGTAATCCAGCATGTACTGGAGCAC |
| | GGTCGTCCTGAGGATAAAAGCAAAA |
| | TTGTAGCAGAAATCCGAGGCAATGT |
| | ACTTGTATTGAGTCAGCACAAATTT |
| | GCAAGCTATGTTGTGCGCAAGTGTG |
| | TTACTCACGCCTCACGTACGGAGCG |
| | CGCTGTGCTCATCGATGAGGTGTGC |
| | ACCATGAACGACGGTCCCCACAGTG |
| | CCTTATACACCATGATGAAGGACCA |
| | GTATGCCAGCTACGTGGTCGAGAAG |
| | ATGATTGACGTGGCGGAGCCAGGCC |
| | AGCGGAAGATCGTCATGCATAAGAT |
| | CCGACCCCACATCGCAACTCTTCGT |
| | AAGTACACCTATGGCAAGCACATTC |
| | TGGCCAAGCTGGAGAAGTACTACAT |
| | GAAGAACGGTGTTGACTTAGGC |
| | (SEQ ID NO: 614) |
| Linker | GTGGATACTGCCAATGGCAGC |
| | (SEQ ID NO: 615) |
| PIN | CAGATGGAGCTCGAAATCAGGCCGC |
| | TGTTCCTCGTGCCGGACACTAATGG |
| | TTTTATAGATCACTTGGCGTCCTTG |
| | GCTAGACTTCTGGAAAGCCGAAAGT |
| | ATATATTGGTAGTGCCGTTGATTGT |
| | AATTAACGAATTGGATGGGTTGGCG |
| | AAAGGACAAGAGACTGATCACAGAG |
| | CAGGAGGCTACGCGAGGGTCGTCCA |
| | AGAGAAGGCGCGAAAAAGCATCGAG |
| | TTCCTGGAGCAGCGATTTGAGAGCA |
| | GGGACTCATGCCTGAGAGCCCTCAC |
| | GTCCCGGGGGAACGAGCTGGAGTCC |
| | ATCGCTTTCCGAAGTGAAGACATTA |
| | CGGGCCAACTTGGGAATAATGATGA |
| | CCTCATCTTGTCCTGCTGCCTGCAC |
| | TACTGCAAGGACAAGGCTAAGGACT |
| | TCATGCCTGCCTCCAAGGAGGAGCC |
| | TATCCGATTGTTGAGGGAAGTAGTA |
| | CTTTTGACGGACGACCGCAACCTCC |
| | GGGTAAAGGCGCTGACTCGAAATGT |
| | CCCAGTAAGGGATATACCGGCGTTC |
| | CTTACATGGGCTCAAGTAGGG |
| | (SEQ ID NO: 623) |
| WPRE | aatcaacctctggattacaaaattt |
| | gtgaaagattgactggtattcttaa |
| | ctatgttgctccttttacgctatgt |
| | ggatacgctgctttaatgcctttgt |
| | atcatgctattgcttcccgtatggc |
| | tttcattttctcctccttgtataaa |
| | tcctggttgctgtctctttatgagg |
| | agttgtggcccgttgtcaggcaacg |
| | tggcgtggtgtgcactgtgtttgct |
| | gacgcaacccccactggttggggca |
| | ttgccaccacctgtcagctcctttc |
| | cgggactttcgetttccccctccct |
| | attgccacggcggaactcatcgccg |
| | cctgccttgcccgctgctggacagg |
| | ggctcggctgttgggcactgacaat |
| | tccgtggtgttgtcggggaaatcat |
| | cgtcctttccttggctgctcgcctA |
| | tgttgccacctggattctgcgcggg |
| | acgtccttctgctacgtcccttcgg |
| | ccctcaatccagcggaccttccttc |
| | ccgcggcctgctgccggctctgcgg |
| | cctcttccgcgtcttcgcettegcc |
| | ctcagacgagtcggatctccctttg |
| | ggccgcctccccgc |
| | (SEQ ID NO: 617) |
| SV40 poly A | AACTTGTTTATTGCAGCTTATAATG |
| | GTTACAAATAAAGCAATAGCATCAC |
| | AAATTTCACAAATAAAGCATTTTTT |
| | TCACTGCATTCTAGTTGTGGTTTGT |
| | CCAAACTCATCAATGTATCTTA |
| | (SEQ ID NO: 533) |
| 3′ ITR | AGGAACCCCTAGTGATGGAGTTGGC |
| | CACTCCCTCTCTGCGCGCTCGCTCG |
| | CTCACTGAGGCCGGGCGACCAAAGG |
| | TCGCCCGACGCCCGGGCTTTGCCCG |
| | GGCGGCCTCAGTGAGCGAGCGAGCG |
| | CGCAGCTGCCTGCAGG |
| | (SEQ ID NO: 598) |

TABLE S4
CAG-repeat targeting PUF fused
to a PIN endonuclease

| | Pro- | | Target | Amino Acid |
|---|---|---|---|---|
| | tein | | Se- | Sequence |
| Construct | Type | Elements | quence | of PUF |
| A02249 | 8PUF | N-terminal | GCAGC | PUF |
| | | PUF; linker | AGC | SEQ ID |
| | | between PUF | | NO: 549 |
| | | and PIN | | |
| | | endonuclease | | |
| | | (VDTANGS); | | |
| | | C-terminal PIN | | |

In some embodiments, nucleic acid sequences encoding CAG-targeting Cas13d proteins of the disclosure are codon optimized nucleic acid sequences. In some embodiments, the codon optimized sequence encoding a CAG-targeting Cas13d protein exhibits at least 5%, at least 10%, at least 20%, at least 30%, at least 50%, at least 75%, at least 100%, at least 200%, at least 300%, at least 500%, or at least 1000% increased translation in a human subject relative to a wild-type or non-codon optimized nucleic acid sequence.

In some aspects, a codon optimized nucleic acid sequence encoding a CAG-targeting Cas13d protein such as those put forth in SEQ ID NOs: 518, 528, 534, 536, and 539 exhibits increased stability. In some aspects, a codon optimized nucleic acid sequence encoding a CAG-targeting Cas13d protein exhibits increased stability through increased resistance to hydrolysis. In some embodiments, the codon optimized sequence encoding a CAG-targeting Cas13d protein exhibits at least 5%, at least 10%, at least 20%, at least 30%, at least 50%, at least 75%, at least 100%, at least 200%, at least 300%, at least 500%, or at least 1000% increased stability relative to a wild-type or non-codon optimized nucleic acid sequence. In some embodiments, the codon optimized sequence encoding a CAG-targeting Cas13d protein exhibits at least 5%, at least 10%, at least 20%, at least 30%, at least 50%, at least 75%, at least 100%, at least 200%, at least 300%, at least 500%, or at least 1000% increased resistance to hydrolysis in a human subject relative to a wild-type or non-codon optimized nucleic acid sequence.

In some aspects, a codon optimized nucleic acid sequence encoding a CAG-targeting Cas13d protein such as those put forth in SEQ ID NOs: 518, 528, 534, 536, and 539, can comprise no donor splice sites. In some aspects, a codon optimized nucleic acid sequence encoding a CAG-targeting Cas13d protein can comprise no more than about one, or about two, or about three, or about four, or about five, or about six, or about seven, or about eight, or about nine, or about ten donor splice sites. In some aspects, a codon optimized nucleic acid sequence encoding a CAG-targeting Cas13d protein comprises at least one, or at least two, or at least three, or at least four, or at least five, or at least six, or at least seven, or at least eight, or at least nine, or at least ten fewer donor splice sites as compared to a non-codon optimized nucleic acid sequence encoding the CAG-targeting Cas13d protein.

Without wishing to be bound by theory, the removal of donor splice sites in the codon optimized nucleic acid sequence can unexpectedly and unpredictably increase expression of the CAG-targeting Cas13d protein in vivo, as cryptic splicing is prevented. Moreover, cryptic splicing may vary between different subjects, meaning that the expression level of the CAG-targeting Cas13d protein comprising donor splice sites may unpredictably vary between different subjects. Such unpredictability is unacceptable in the context of human therapy. Accordingly, the codon optimized nucleic acid sequences put forth in SEQ ID NOs: 518, 528, 534, 536, and 539, which lacks donor splice sites, unexpectedly and surprisingly allows for increased expression of the CAG-targeting Cas13d protein in human subjects and regularizes expression of the CAG-targeting Cas13d protein across different human subjects.

In some aspects, a codon optimized nucleic acid sequence encoding a CAG-targeting Cas13d protein, such as those put forth in SEQ ID NOs: 518, 528, 534, 536, and 539, can have a GC content that differs from the GC content of the non-codon optimized nucleic acid sequence encoding the CAG-targeting Cas13d protein. In some aspects, the GC content of a codon optimized nucleic acid sequence encoding a CAG-targeting Cas13d protein is more evenly distributed across the entire nucleic acid sequence, as compared to the non-codon optimized nucleic acid sequence encoding the CAG-targeting Cas13d protein.

Without wishing to be bound by theory, by more evenly distributing the GC content across the entire nucleic acid sequence, the codon optimized nucleic acid sequence exhibits a more uniform melting temperature (“Tm”) across the length of the transcript. The uniformity of melting temperature results unexpectedly in increased expression of the codon optimized nucleic acid in a human subject, as transcription and/or translation of the nucleic acid sequence occurs with less stalling of the polymerase and/or ribosome.

In some aspects, a codon optimized nucleic acid sequence encoding a CAG-targeting Cas13d protein, such as those put forth in SEQ ID NOs: 518, 528, 534, 536, and 539, can have fewer repressive microRNA target binding sites as compared to the non-codon optimized nucleic acid sequence encoding the CAG-targeting Cas13d protein. In some aspects, a codon optimized nucleic acid sequence encoding a CAG-targeting Cas13d protein can have at least one, or at least two, or at least three, or at least four, or at least five, or at least six, or at least seven, or at least eight, or at least nine, or at least ten, or at least ten fewer repressive microRNA target binding sites as compared to the non-codon optimized nucleic acid sequence the CAG-targeting Cas13d protein.

Without wishing to be bound by theory, by having fewer repressive microRNA target binding sites, the codon optimized nucleic acid sequence encoding a CAG-targeting Cas13d protein unexpectedly exhibits increased expression in a human subject.

### Fusion Proteins

In some embodiments of the compositions and methods of the disclosure, the composition comprises a sequence encoding a target RNA-binding fusion protein comprising (a) a sequence encoding a first RNA-binding polypeptide or portion thereof; and optionally (b) a sequence encoding a second RNA-binding polypeptide, wherein the first RNA-binding polypeptide binds a target RNA, and wherein the second RNA-binding polypeptide comprises RNA-nuclease activity.

In some embodiments, a target RNA-binding fusion protein is an RNA-guided target RNA-binding fusion protein. RNA-guided target RNA-binding fusion proteins comprise at least one RNA-binding polypeptide which corresponds to a gRNA which guides the RNA-binding polypeptide to target RNA. RNA-guided target RNA-binding fusion proteins include without limitation, RNA-binding polypeptides which are CRISPR/Cas-based RNA-binding polypeptides or portions thereof.

### Signal Sequences

In some embodiments, a target RNA-binding fusion protein of the disclosure comprises a signal sequence. In some embodiments, a target RNA-binding fusion protein comprises one or more signal sequences. In some embodiments, the signal sequence(s) is a nuclear localization sequence (NLS), nuclear export signal (NES) or a combination thereof. In some embodiments, the tag sequence comprises a nuclear localization sequence (NLS). In some embodiments, the NLS sequence comprises a sequence listed in table 8. In some embodiments, the NLS signal sequence is a human NLS. In some embodiments, the human NLS signal sequence is a human pRB-NLS or a human pRB-NLS (extended version).

TABLE 8
Nuclear Localization Sequences
of the disclosure

| | | SEQ |
|---|---|---|
| | Amino acid | ID |
| Name | Sequence | NO: |
| SV40-NLS | PKKKRKV | 437 |
| human H2B-NLS | GKKRKRSRK | 438 |
| yeast H2B-NLS | GKKRSKV | 439 |
| human p53-NLS | KRALPNNTSSSPQPKKKP | 440 |
| human-cmyc-NLS | PAAKRVKLD | 441 |
| human pRB-NLS | KRSAEGSNPPKPLKKLR | 442 |
| human Nucleoplasmin- | KRPAATKKAGQAKKKK | 443 |
| NLS | LDK | |
| Human pRB-NLS | DRVLKRSAEGSNPPKP | 543 |
| (extended version) | LKKLR | |

In some embodiments, the signal sequence comprises one or more NES sequences. In some embodiments, the one or more NES sequence comprises a sequence listed in Table 9.

TABLE 9
Nuclear Export Sequences of the disclosure

| | | SEQ |
|---|---|---|
| | Amino acid | ID |
| Name | Sequence | NO: |
| HIV REV NES | LPPLERLTLD | 544 |
| Human PKI NES | LALKLAGLDI | 545 |

In some embodiments, a target RNA-binding fusion protein of the disclosure comprises a tag sequence. In some embodiments, the tag sequence is a FLAG tag.

In some embodiments, the FLAG tag sequence is DYKDDDDK (SEQ ID NO: 436).

Linker Sequences

In some embodiments, a target RNA-binding fusion protein comprises a linker sequence. In some embodiments, the linker sequence may comprise or consist of 1, 2, 3, 4, 5, 6, 7, 8, 9, 10, 15, 20, 25, 30, 35, 40, 45, 50 or any number of amino acids in between. In some embodiments, the linker sequence comprises a linker sequence listed in Table 10.

TABLE 10
Linker Sequences of the disclosure

| Linker Sequence (amino acid) | SEQ ID NO: |
|---|---|
| GGS | 410 |
| VDTANGS | 411 |
| VDTGNGS | 412 |
| SGSETPGTSESATPES | 413 |
| GGGGSGGGGS | 414 |
| GGGGGGGGSGGGGS | 415 |
| GGGGSGGGGSGGGGSGG | 416 |
| GGS | |
| EAAAKEAAAK | 417 |
| EAAAKEAAAKEAAAK | 418 |
| EAAAKEAAAKEAAAKEAA | 419 |
| AK | |
| APAPAPAP | 420 |
| APAPAPAPAPAP | 421 |
| APAPAPAPAPAPAPAPAP | 422 |
| GGGGSEAAAK | 423 |
| EAAAKGGGGS | 424 |
| GGGGSGGGGSEAAAKEAA | 425 |
| AK | |
| EAAAKEAAAKGGGGSGGG | 426 |
| GS | |
| RQTSPDPCPQLPLVPR | 427 |
| VDTGNWF | 428 |
| VDTANGSVDTGNGS | 429 |
| ARNVEERLCL | 430 |
| AIELNPSNA | 431 |
| ICGSRNL | 432 |
| VLATDMSKH | 434 |
| FLRELPEP | 435 |
| LIPKDQYYC | 436 |
| AEAAAKEAAAKA | 628 |
| AEAAAKEAAAKEAAAKA | 629 |
| AEAAAKEAAAKEAAAKEA | 630 |
| AAKA | |
| YVEFEGEQGVDEGGVSGG | 631 |
| GS | |
| GSRNLDFQALEETTEYDGG | 632 |
| Y | |
| ASSTSPVEISEWLDQKLTKS | 633 |
| DRPEL | |
| VNQCRRQSEDSTFYLG | 634 |
| AVSPLLLTTTNSSEGLSMG | 635 |
| NY | |
| LDEAYPGKKLLPDDPYEK | 636 |
| ACQ | |
| SAAAATPAVRTVPQYKYA | 637 |
| AGVRNPQQHLNAQPQVTM | |
| QQPAVHVQGQEPL | |
| GGGGSEAAAKGGGGS | 638 |
| GGGGSEAAAKGGGGSEAA | 639 |
| AKGGGGS | |
| GGGGSSGSETGGTSESATG | 640 |
| ESGGGGS | |
| SGSETPGTSESATPES | 641 |

Promoter Sequences

In aspects, CAG targeting compositions of the disclosure comprise a promoter sequence. In some embodiments, any promoter disclosed herein can be substituted for any of the other promoters recited in the RNA-targeting constructs disclosed herein. In some aspects, CAG targeting compositions comprise a truncated CAG (tCAG) promoter (SEQ ID NO: 385). In some aspects, CAG targeting compositions comprise a short EF1-alpha (EFS) promoter (SEQ ID NO: 520). In some aspects, CAG targeting compositions comprise an EFS-UBB promoter set forth in SEQ ID NO: 613. In some aspects, CAG targeting compositions comprise a human synapsin promoter set forth in SEQ ID NO: 627. In some embodiments, promoter sequences of the disclosure comprise a human EF1-alpha core promoter (SEQ ID NO: 642). In some embodiments, promoter sequences of the disclosure comprise a modified UBB intron (SEQ ID NO: 643). In some embodiments, promoter sequences of the disclosure comprise a modified CMV enhancer sequence (SEQ ID NO: 644). In some embodiments, promoter sequences of the disclosure comprise an eCMV-EFS-UBB promoter sequence (SEQ ID NO: 645).

In some embodiments, expression control by a promoter is constitutive or ubiquitous. Non-limiting exemplary promoters include a Pol III promoter such as, e.g., U6 and H1 promoters and/or a Pol II promoter e.g., SV40, CMV (optionally including the CMV enhancer), RSV (Rous Sarcoma Virus LTR promoter (optionally including RSV enhancer), CBA (hybrid CMV enhancer/chicken β-actin), CAG (hybrid CMV enhancer fused to chicken β-actin), truncated CAG, Cbh (hybrid CBA), EF-1a (human elongation factor alpha-1) or EFS (short intron-less EF-1 alpha), PGK (phosphoglycerol kinase), CEF (chicken embryo fibroblasts), UBC (ubiquitin C), GUSB (lysosomal enzyme beta-glucuronidase), UCOE (ubiquitous chromatin opening element), hAAT (alpha-1 antitrypsin), TBG (thyroxine binding globulin), Desmin (full-length or truncated), MCK (muscle creatine kinase), C5-12 (synthetic muscle promoter), CK8e (creatin kinase 8), NSE (neuron-specific enolase), Synapsin, Synapsin-1 (SYN-1), opsin, PDGF (platelet-derived growth factor), PDGF-A, MecP2 (methyl CpG-binding protein 2), CaMKII (Calcium/Calmodulin-dependent protein kinase II), mGluR2 (metabotropic glutamate receptor 2), NFL (neurofilament light), NFH (neurofilament heavy), nβ2, PPE (rat preproenkephalin), ENK (preproenkephalin), Preproenkephalin-neurofilament chimeric promoter, EAAT2 (glutamate transporter), GFAP (glial fibrillary acidic protein), MBP (myelin basic protein), human rhodopsin kinase promoter (hGRKi), β-actin promoter, dihydrofolate reductase promoter, MHCK7 (hybrid promoter of enhancer/promoter regions of muscle creatine kinase and alpha myosin heavy-chain genes) and combinations thereof. An “enhancer” is a region of DNA that can be bound by activating proteins to increase the likelihood or frequency of transcription. Non-limiting exemplary enhancers and posttranscriptional regulatory elements include the CMV enhancer, MCK enhancer, R-U5′ segment in LTR of HTLV-1, SV40 enhancer, the intron sequence between exons 2 and 3 of rabbit β-globin, and WPRE. In some embodiments an intron is used to enhance promoter activity such as a UBB intron. In some embodiments, the UBB intron is used with an EFS promoter. In some embodiments, enhancer sequences can be added in the 5′ or 3′ UTR. In some embodiments, a 5′ enhancer can be Hsp70 as set forth in SEQ ID NO: 657:

TABLE 11
8PUF protein according to SEQ ID NO: 444

| | RNA | | SEQ |
|---|---|---|---|
| PUF | Recogni- | | ID |
| Module | tion | Amino Acid Sequence | NO: |
| PUF R1′ | — | GRSRLLEDFRNNRYPNLQ | 495 |
| | | LREIAG | |
| PUF R1 | G | HIMEFSQDQHGSRFIELK | 498 |
| | | LERATPAERQLVFNEILQ | |
| PUF R2 | A | AAYQLMVDVFGCYVIQKF | 490 |
| | | FEFGSLEQKLALAERIRG | |
| PUF R3 | C | HVLSLALQMYGSYVIRKA | 508 |
| | | LEFIPSDQQNEMVRELDG | |
| PUF R4 | G | HVLKCVKDQNGSYVVEKC | 504 |
| | | IECVQPQSLQFIIDAFKG | |
| PUF R5 | A | QVFALSTHPYGCRVIQRI | 512 |
| | | LEHCLPDQTLPILEELHQ | |
| PUF R6 | C | HTEQLVQDQYGSYVIRHV | 502 |
| | | LEHGRPEDKSKIVAEIRG | |
| PUF R7 | G | NVLVLSQHKFASNVVEKC | 510 |
| | | VTHASRTERAVLIDEVCT | |
| | | MNDGPHS | |
| PUF R8 | A | ALYTMMKDQYACYVVQKM | 493 |
| | | IDVAEPGQRKIVMHKIRP | |
| PUF R8′ | — | HIATLRKYTYGKHILAKL | 496 |
| | | EKYYMKNGVDLG | |

Non-Guided RNA-Binding Fusion Proteins

In some embodiments, a target RNA-binding fusion protein is not an RNA-guided target RNA-binding fusion protein

and as such comprises at least one RNA-binding polypeptide which is capable of binding a target RNA without a corresponding gRNA sequence. Such non-guided RNA-binding polypeptides include, without limitation, at least one RNA-binding protein or RNA-binding portion thereof which is a PUF (Pumilio and FBF homology family) protein. This type RNA-binding polypeptide can be used instead of a gRNA-guided RNA binding protein such as CRISPR/Cas. The unique RNA recognition mode of PUF proteins (named for Drosophila pumilio and C. elegans fem-3 binding factor) that are involved in mediating mRNA stability and translation are well known in the art. The PUF domain of human Pumilio1, also known in the art, binds tightly to cognate RNA sequences and its specificity can be modified. It contains eight PUF modules that recognize eight consecutive RNA bases with each module recognizing a single base. Since two amino acid side chains in each module recognize the Watson-Crick edge of the corresponding base and determine the specificity of that module, a PUF protein can be designed to specifically bind most 8 to 16-nt RNA. Wang et al., Nat Methods. 2009; 6(11): 825-830. See also WO2012/068627 which is incorporated by reference herein in its entirety.

The modular nature of the PUF-RNA interaction has been used to rationally engineer the binding specificity of PUF domains (Cheong, C. G. & Hall, T. M. (2006) PNAS 103: 13635-13639; Wang, X. et al (2002) Cell 110: 501-512). However, only the successful design of PUF proteins with modules that recognize adenine, guanine or uracil have been reported prior to the teachings of WO2012/06827 supra. While the wild-type PumHD does not bind cytosine (C), molecular engineering has shown that some of the Pum units can be mutated to bind C with good yield and specificity. See e.g., Dong, S. et al. Specific and modular binding code for cytosine recognition in Pumilio/FBF (PUF) RNA-binding domains, The Journal of biological chemistry 286, 26732-26742 (2011). Accordingly, PumHD is a modified version of the WT Pumilio protein that exhibits programmable binding to arbitrary 8-base sequences of RNA. Each of the eight units of PumHD can bind to all four RNA bases, and the RNA bases flanking the target sequence do not affect binding. See also the following for art-recognized RNA-binding rules of PUF design: Filipovska A, Razif M F, Nyg\dot{a}rd K K, & Rackham O. A universal code for RNA recognition by PUF proteins. Nature chemical biology, 7(7), 425-427 (2011); Filipovska A, & Rackham O. Modular recognition of nucleic acids by PUF, TALE and PPR proteins. Molecular BioSystems, 8(3), 699-708 (2012); Abil Z, Denard C A, & Zhao H. Modular assembly of designer PUF proteins for specific post-transcriptional regulation of endogenous RNA. Journal of biological engineering, 8(1), 7 (2014); Zhao Y, Mao M, Zhang W, Wang J, Li H, Yang Y, Wang Z, & Wu J. Expanding RNA binding specificity and affinity of engineered PUF domains. Nucleic Acids Research, 46(9), 4771-4782 (2018); Shinoda K, Tsuji S. Futaki S, & Imanishi M, Nested PUF Proteins: Extending Target RNA Elements for Gene Regulation. ChemBioChem, 19(2), 171-176 (2018); Koh Y Y, Wang Y, Qiu C, Opperman L, Gross L, Tanaka Hall T M, & Wickens M. Stacking Interactions in PUF-RNA Complexes. RNA, 17(4), 718-727 (2011).

As such, it is well known in the art that human PUM1 (1186 amino acids) contains an RNA-binding domain (RBD) in the C-terminus of the protein (also known as Pumilio homology domain PUM-HD amino acid 828-amino acid 1175) and that PUFs are based on the RBD of human PUM1. There are 8 structural repeat modules of 36 amino acids (except module 7 which has 43 amino acids) for RNA binding and flanking N- and C-terminal regions important for protein structure and stability. Within each repeat module, amino acids 12, 13, and 16 are important for RNA binding with 12 and 16 responsible for RNA base recognition. Amino acid 13 stacks with RNA bases and can be modified to tune specificity and affinity. Alternatively, the PUF design may maintain amino acid 13 as human PUM1's native residue. In some embodiments of the PUF(CAG) or PUMBY(CAG) compositions disclosed herein, amino acid 13 (for stacking) will be engineered with an H and in other embodiments, will be engineered with a Y. In some embodiments, stacking residues may be modified to improve binding and specificity. Recognition occurs in reverse orientation as N- to C-terminal PUF recognizes 3′ to 5′ RNA. Accordingly, PUF engineering of 8 modules (8PUF), as known in the art, mimics a human protein. An exemplary 8-mer RNA recognition (8PUF) would be designed as follows: R1′-R1-R2-R3-R4-R5-R6-R7-R8-R8′. In one embodiment, an 8PUF is used as the RBD. In another embodiment, a variation of the 8PUF design is used to create a 14-mer RNA recognition (14PUF) RBD, 15-mer RNA recognition (15PUF) RBD, or a 16-mer RNA recognition (16PUF) RBD. In another embodiment, the PUF can be engineered to comprise a 4-mer, 5-mer, 6-mer, 7-mer, 8-mer, 9-mer, 10-mer, 11-mer, 12-mer, 13-mer, 14-mer, 15-mer, 16-mer, 24-mer, 30-mer, 36-mer, or any number of modules between. Shinoda et al., 2018; Criscuolo et al., 2020. Repeats 1-8 of wild type human PUM1 are provided herewith at SEQ ID NOS: 462-469, respectively. The nucleic acid sequence encoding the PUF domain from human PUM1 is SEQ ID NO: 470 and the amino acid sequence of the PUF domain from human PUM1 amino acids 828-1176 is SEQ ID NO: 471. See also U.S. Pat. No. 9,580,714 which is incorporated herein in its entirety.

In some embodiments of the non-guided RNA-binding fusion proteins of the disclosure, the fusion protein comprises at least one RNA-binding protein or RNA-binding portion thereof which is a PUMBY (Pumilio-based assembly) protein. RNA-binding protein PumHD, which has been widely used in native and modified form for targeting RNA, has been engineered into a protein architecture designed to yield a set of four canonical protein modules, each of which targets one RNA base. These modules (i.e., Pumby, for Pumilio-based assembly) are concatenated in chains of varying composition and length, to bind desired target RNAs. In essence, PUMBY is a more simple and modular form of PumHD, in which a single protein unit of PumHD is concatenated into arrays of arbitrary size and binding sequence specificity. The specificity of such Pumby-RNA interactions is high, with undetectable binding of a Pumby chain to RNA sequences that bear three or more mismatches from the target sequence. Katarzyna et al., PNAS, 2016; 113(19): E2579-E2588. See also US 2016/0238593 which is incorporated by reference herein in its entirety.

In some embodiments of the compositions of the disclosure, the first RNA binding protein comprises a Pumilio and FBF (PUF) protein. In some embodiments, the first RNA binding protein comprises a Pumilio-based assembly (PUMBY) protein. In some embodiments, the PUF or PUMBY RNA-binding proteins are fused with a nuclease domain such as E17.

In some embodiments of the compositions of the disclosure, at least one of the RNA-binding proteins or RNA-binding portions thereof is a PPR protein. PPR proteins (proteins with pentatricopeptide repeat (PPR) motifs derived from plants) are nuclear-encoded and exclusively controlled at the RNA level organelles (chloroplasts and mitochondria), cutting, translation, splicing, RNA editing, genes specifically acting on RNA stability. PPR proteins are typically a motif of 35 amino acids and have a structure in which a PPR motif is about 10 contiguous amino acids. The combination of PPR motifs can be used for sequence-selective binding to RNA. PPR proteins are often comprised of PPR motifs of about 10 repeat domains. PPR domains or RNA-binding domains may be configured to be catalytically inactive. WO 2013/058404 incorporated herein by reference in its entirety.

In some embodiments, the fusion protein disclosed herein comprises a linker between the at least two RNA-binding polypeptides. In some embodiments, the linker is a peptide linker. In one embodiment, the linker is VDTANGS (SEQ ID NO: 411). In some embodiments, the peptide linker comprises one or more repeats of the tri-peptide GGS. In other embodiments, the linker is a non-peptide linker. In some embodiments, the non-peptide linker comprises polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacryl amide, polyacrylate, polycyanoacrylates, lipid polymers, chitins, hyaluronic acid, heparin, or an alkyl linker.

In some embodiments, the at least one RNA-binding protein does not require multimerization for RNA-binding activity. In some embodiments, the at least one RNA-binding protein is not a monomer of a multimer complex. In some embodiments, a multimer protein complex does not comprise the RNA binding protein. In some embodiments, the at least one of RNA-binding protein selectively binds to a target sequence within the RNA molecule. In some embodiments, the at least one RNA-binding protein does not comprise an affinity for a second sequence within the RNA molecule. In some embodiments, the at least one RNA-binding protein does not comprise a high affinity for or selectively bind a second sequence within the RNA molecule. In some embodiments, the at least one RNA-binding protein comprises between 2 and 1300 amino acids, inclusive of the endpoints.

In some embodiments, the at least one RNA-binding protein of the fusion proteins disclosed herein further comprises a sequence encoding a nuclear localization signal (NLS). In some embodiments, a nuclear localization signal (NLS) is positioned at the N-terminus of the RNA binding protein. In some embodiments, the at least one RNA-binding protein comprises an NLS at a C-terminus of the protein. In some embodiments, the at least one RNA-binding protein further comprises a first sequence encoding a first NLS and a second sequence encoding a second NLS. In some embodiments, the first NLS or the second NLS is positioned at the N-terminus of the RNA-binding protein. In some embodiments, the at least one RNA-binding protein comprises the first NLS or the second NLS at a C-terminus of the protein. In some embodiments, the at least one RNA-binding protein further comprises an NES (nuclear export signal) or other peptide tag or secretory signal. In one embodiment, the tag is a FLAG tag.

In some embodiments, a fusion protein disclosed herein comprises the at least one RNA-binding protein as a first RNA-binding protein together with a second RNA-binding protein comprising or consisting of a nuclease domain.

In some embodiments, the second RNA-binding polypeptide is operably configured to the first RNA-binding polypeptide at the C-terminus of the first RNA-binding polypeptide. In some embodiments, the second RNA-binding polypeptide is operably configured to the first RNA-binding polypeptide at the N-terminus of the first RNA-binding polypeptide. In one embodiment, an exemplary fusion protein is a PUF or PUMBY-based first RNA-binding protein fused to a second RNA-binding protein which is a zinc-finger endonuclease known as ZC3H12A or truncation of it is shown in SEQ ID NO: 358 (also termed E17).

An exemplary 8-mer RNA recognition (8PUF) targeting AGCAGCAG (SEQ ID NO: 472) comprises the amino acid sequence: GRSRLLEDFRNNRYPNLQLREIAGHIMEFSQDQHGSRFIELKLERATPAERQLVFNEIL QAAYQLMVDVFGCYVIQKFFEFGSLEQKLALAERIRGHVLSLALQMYGSYVIRKAL EFIPSDQQNEMVRELDGHVLKCVKDQNGSYVVEKCIECVQPQSLQFIIDAFKGQVFA LSTHPYGCRVIQRILEHCLPDQTLPILEELHQHTEQLVQDQYGSYVIRHVLEHGRPED KSKIVAEIRGNVLVLSQHKFASNVVEKCVTHASRTERAVLIDEVCTMNDGPHSALYT MMKDQYACYVVQKMIDVAEPGQRKIVMHKIRPHIATLRKYTYGKHILAKLEKYYM KNGVDLG (SEQ ID NO: 444). In some aspects, SEQ ID NO: 444 comprises an architecture proceeding from the N-terminus to the C-terminus according to: R1′-R1-R2-R3-R4-R5-R6-R7-R8-R8′. In some aspects, SEQ ID NO: 444 is comprised of the sequences detailed in Table 11.

TABLE 11
8PUF protein according to SEQ ID NO: 444

| | RNA | | SEQ |
|---|---|---|---|
| PUF | Recogni- | | ID |
| Module | tion | Amino Acid Sequence | NO: |
| PUF R1′ | — | GRSRLLEDFRNNRYPNLQ | 495 |
| | | LREIAG | |
| PUF R1 | G | HIMEFSQDQHGSRFIELK | 498 |
| | | LERATPAERQLVFNEILQ | |
| PUF R2 | A | AAYQLMVDVFGCYVIQKF | 490 |
| | | FEFGSLEQKLALAERIRG | |
| PUF R3 | C | HVLSLALQMYGSYVIRKA | 508 |
| | | LEFIPSDQQNEMVRELDG | |
| PUF R4 | G | HVLKCVKDQNGSYVVEKC | 504 |
| | | IECVQPQSLQFIIDAFKG | |
| PUF R5 | A | QVFALSTHPYGCRVIQRI | 512 |
| | | LEHCLPDQTLPILEELHQ | |
| PUF R6 | C | HTEQLVQDQYGSYVIRHV | 502 |
| | | LEHGRPEDKSKIVAEIRG | |
| PUF R7 | G | NVLVLSQHKFASNVVEKC | 510 |
| | | VTHASRTERAVLIDEVCT | |
| | | MNDGPHS | |
| PUF R8 | A | ALYTMMKDQYACYVVQKM | 493 |
| | | IDVAEPGQRKIVMHKIRP | |
| PUF R8′ | — | HIATLRKYTYGKHILAKL | 496 |
| | | EKYYMKNGVDLG | |

An exemplary 8-mer RNA recognition (8PUF) targeting GCAGCAGC (SEQ ID NO: 476) comprises the amino acid sequence: GRSRLLEDFRNNRYPNLQLREIAGHIMEFSQDQHGSYFIRLKLERATPAERQLVFNEI LQAAYQLMVDVFGSNVIEKFFEFGSLEQKLALAERIRGHVLSLALQMYGCRVIQKAL EFIPSDQQNEMVRELDGHVLKCVKDQNGSYVVRKCIECVQPQSLQFIIDAFKGQVFA LSTHPYGSNVIERILEHCLPDQTLPILEELHQHTEQLVQDQYGCRVIQHVLEHGRPED KSKIVAEIRGNVLVLSQHKFASYVVRKCVTHASRTERAVLIDEVCTMNDGPHSALYT MMKDQYASNVVEKMIDVAEPGQRKIVMHKIRPHIATLRKYTYGKHILAKLEKYYM KNGVDLG (SEQ ID NO: 656). In some aspects, SEQ ID NO: 656 comprises an architecture proceeding from the N-terminus to the C-terminus according to: R1′-R1-2-R3-R4-R5-R6-R7-R8-R8′.

In some aspects, PUF proteins of the disclosure can be modified for improved stacking. Possible mutations for improved stacking are listed in Table T. In some embodiments, PUF modules RI, R2, R3, R4, R5, R6, R7, R8, 1′, and 8′ can be combined in any number and in any order for PUF proteins of the disclosure.

TABLE T
Stacking mutations for PUF proteins

| | RNA | | SEQ | Possible |
|---|---|---|---|---|
| Plasmid | Recogni- | | ID | stacking |
| Element | tion | Amino Acid Sequence | NO: | amino acid |
| PUF 1′ | * | GRSRLLEDFRNNRYPNLQLREIAG | | |
| PUF R1 | A* | HIMEFSQDQHGSRFIQLKLERATPAERQLVFNEILQ | 497 | R, Y |
| PUF R1 | G | HIMEFSQDQHGSRFIELKLERATPAERQLVFNEILQ | 498 | R, N, F |
| PUF R1 | U | HIMEFSQDQHGNRFIQLKLERATPAERQLVFNEILQ | 646 | R, Y, H, F |
| PUF R1 | C | HIMEFSQDQHGSRFIRLKLERATPAERQLVFNEILQ | 499 | R, Y, F |
| PUF R2 | A | AAYQLMVDVFGCYVIQKFFEFGSLEQKLALAERIRG | 490 | Y, R |
| PUF R2 | G | AAYQLMVDVFGSYVIEKFFEFGSLEQKLALAERIRG | 491 | Y, N, F |
| PUF R2 | U* | AAYQLMVDVFGNYVIQKFFEFGSLEQKLALAERIRG | 647 | Y, H, F |
| PUF R2 | C | AAYQLMVDVFGSYVIRKFFEFGSLEQKLALAERIRG | 492 | Y, F |
| PUF R3 | A* | HVLSLALQMYGCRVIQKALEFIPSDQQNEMVRELDG | 506 | R, Y, F |
| PUF R3 | G | HVLSLALQMYGSRVIEKALEFIPSDQQNEMVRELDG | 507 | R, N, F |
| PUF R3 | U | HVLSLALQMYGNRVIQKALEFIPSDQQNEMVRELDG | 648 | R, Y, H, F |
| PUF R3 | C | HVLSLALQMYGSRVIRKALEFIPSDQQNEMVRELDG | 508 | R, Y, F |
| PUF R4 | A | HVLKCVKDQNGCHVVQKCIECVQPQSLQFIIDAFKG | 503 | H, R, Y |
| PUF R4 | G | HVLKCVKDQNGSHVVEKCIECVQPQSLQFIIDAFKG | 504 | H, N, F |
| PUF R4 | U* | HVLKCVKDQNGNHVVQKCIECVQPQSLQFIIDAFKG | 649 | H, Y, F |
| PUF R4 | C | HVLKCVKDQNGSHVVRKCIECVQPQSLQFIIDAFKG | 505 | H, Y, F |
| PUF R5 | A* | QVFALSTHPYGCRVIQRILEHCLPDQTLPILEELHQ | 512 | R, Y |
| PUF R5 | G | QVFALSTHPYGSRVIERILEHCLPDQTLPILEELHQ | 513 | R, N, F |
| PUF R5 | U | QVFALSTHPYGNRVIQRILEHCLPDQTLPILEELHQ | 650 | R, Y, H, F |
| PUF R5 | C | QVFALSTHPYGSRVIRRILEHCLPDQTLPILEELHQ | 514 | R, Y, F |
| PUF R6 | A | HTEQLVQDQYGCYVIQHVLEHGRPEDKSKIVAEIRG | 500 | Y, R |
| PUF R6 | G | HTEQLVQDQYGSYVIEHVLEHGRPEDKSKIVAEIRG | 501 | Y, N, F |
| PUF R6 | U* | HTEQLVQDQYGNYVIQHVLEHGRPEDKSKIVAEIRG | 651 | Y, H, F |
| PUF R6 | C | HTEQLVQDQYGSYVIRHVLEHGRPEDKSKIVAEIRG | 502 | Y, F |
| PUF R7 | A | NVLVLSQHKFACNVVQKCVTHASRTERAVLIDEVCTMNDGPHS | 509 | N, R, Y |
| PUF R7 | G* | NVLVLSQHKFASNVVEKCVTHASRTERAVLIDEVCTMNDGPHS | 510 | N, F |
| PUF R7 | U | NVLVLSQHKFANNVVQKCVTHASRTERAVLIDEVCTMNDGPHS | 652 | N, Y, H, F |
| PUF R7 | C | NVLVLSQHKFASNVVRKCVTHASRTERAVLIDEVCTMNDGPHS | 511 | N, Y, F |
| PUF R8 | A | ALYTMMKDQYACYVVQKMIDVAEPGQRKIVMHKIRP | 493 | Y, R |
| PUF R8 | G | ALYTMMKDQYASYVVEKMIDVAEPGQRKIVMHKIRP | 489 | Y, N, F |
| PUF R8 | U* | ALYTMMKDQYANYVVQKMIDVAEPGQRKIVMHKIRP | 653 | Y, H, F |
| PUF R8 | C | ALYTMMKDQYASYVVRKMIDVAEPGQRKIVMHKIRP | 494 | Y, F |
| 8′ | * | HIATLRKYTYGKHILAKLEKYYMKNGVDLG | 496 | |

An exemplary 14-mer RNA recognition (14PUF) targeting AGCAGCAGCAGCAG (SEQ ID NO: 473) comprises the amino acid sequence: GRSRLLEDFRNNRYPNLQLREIAGHIMEFSQDQHGSRFIELKLERATPAERQLVFNEILQAAY QLMVDVFGCYVIQKFFEFGSLEQKLALAERIRGHVLSLALQMYGSYVIRKALEFIPSDQQNE MVRELDGHVLKCVKDQNGSYVVEKCIECVQPQSLQFIIDAFKGQVFALSTHPYGCRVIQRILE HCLPDQTLPILEELHQHIMEFSQDQHGSRFIRLKLERATPAERQLVFNEILQAAYQLMVDVFG SYVIEKFFEFGSLEQKLALAERIRGHVLSLALQMYGCRVIQKALEFIPSDQQNEMVRELDGHV LKCVKDQNGSYVVRKCIECVQPQSLQFIIDAFKGQVFALSTHPYGSRVIERILEHCLPDQTLPI LEELHQHTEQLVQDQYGCYVIQHVLEHGRPEDKSKIVAEIRGHTEQLVQDQYGSYVIRHVLE HGRPEDKSKIVAEIRGNVLVLSQHKFASNVVEKCVTHASRTERAVLIDEVCTMNDGPHSALY TMMKDQYACYVVQKMIDVAEPGQRKIVMHKIRPHIATLRKYTYGKHILAKLEKYYMKNGV DLG (SEQ ID NO: 445). In some aspects, SEQ ID NO: 445 comprises an architecture proceeding from the N-terminus to the C-terminus according to: R1′-R1-R2-R3-R4-R5-R1-R2-R3-R4-R5-R6-R6-R7-R8-R8′. In some aspects, SEQ ID NO: 445 is comprised of the sequences detailed in Table 12.

TABLE 12
14PUF protein according to SEQ ID NO: 445

| | RNA | | SEQ |
|---|---|---|---|
| PUF | Recogni- | | ID |
| Module | tion | Amino Acid Sequence | NO |
| PUF R1′ | — | GRSRLLEDFRNNRYPNLQ | 495 |
| | | LREIAG | |
| PUF R1 | G | HIMEFSQDQHGSRFIELK | 498 |
| | | LERATPAERQLVFNEILQ | |
| PUF R2 | A | AAYQLMVDVFGCYVIQKF | 490 |
| | | FEFGSLEQKLALAERIR | |
| | | G | |
| PUF R3 | C | HVLSLALQMYGSYVIRKA | 508 |
| | | LEFIPSDQQNEMVRELD | |
| | | G | |
| PUF R4 | G | HVLKCVKDQNGSYVVEKC | 504 |
| | | IECVQPQSLQFIIDAFKG | |
| PUF R5 | A | QVFALSTHPYGCRVIQRI | 512 |
| | | LEHCLPDQTLPILEELHQ | |
| PUF R1 | C | HIMEFSQDQHGSRFIRLK | 499 |
| | | LERATPAERQLVFNEILQ | |
| PUF R2 | G | AAYQLMVDVFGSYVIEKF | 491 |
| | | FEFGSLEQKLALAERIRG | |
| PUF R3 | A | HVLSLALQMYGCRVIQKA | 506 |
| | | LEFIPSDQQNEMVRELD | |
| | | G | |
| PUF R4 | C | HVLKCVKDQNGSYVVRKC | 505 |
| | | IECVQPQSLQFIIDAFKG | |
| PUF R5 | G | QVFALSTHPYGSR VIER | 513 |
| | | ILEHCLPDQTLPILEELH | |
| | | Q | |
| PUF R6 | A | HTEQLVQDQYGCYVIQHV | 500 |
| | | LEHGRPEDKSKIVAEIRG | |
| PUF R6 | C | HTEQLVQDQYGSYVIRHV | 502 |
| | | LEHGRPEDKSKIVAEIRG | |
| PUF R7 | G | NVLVLSQHKFASNVVEKC | 510 |
| | | VTHASRTERAVLIDEVC | |
| | | TMNDGPHS | |
| PUF R8 | A | ALYTMMKDQYACYVVQKM | 493 |
| | | IDVAEPGQRKIVMHKI | |
| | | RP | |
| PUF R8′ | — | HIATLRKYTYGKHILAKL | 496 |
| | | EKYYMKNGVDLG | |

An exemplary 14-mer RNA recognition (14PUF) targeting AGCAGCAGCAGCAG (SEQ ID NO: 473) comprises the amino acid sequence: GRSRLLEDFRNNRYPNLQLREIAGHIMEFSQDQHGSRFIELKLERATPAERQLVFNEIL QAAYQLMVDVFGCYVIQKFFEFGSLEQKLALAERIRGHVLSLALQMYGSYVIRKAL EFIPSDQQNEMVRELDGHVLKCVKDQNGSYVVEKCIECVQPQSLQFIIDAFKGQVFA LSTHPYGCRVIQRILEHCLPDQTLPILEELHQHTEQLVQDQYGSYVIRHVLEHGRPED KSKIVAEIRGHIMEFSQDQHGSRFIELKLERATPAERQLVFNEILQAAYQLMVDVFGC YVIQKFFEFGSLEQKLALAERIRGHVLSLALQMYGSYVIRKALEFIPSDQQNEMVREL DGHVLKCVKDQNGSYVVEKCIECVQPQSLQFIIDAFKGQVFALSTHPYGCRVIQRILE HCLPDQTLPILEELHQHTEQLVQDQYGSYVIRHVLEHGRPEDKSKIVAEIRGNVLVLS QHKFASNVVEKCVTHASRTERAVLIDEVCTMNDGPHSALYTMMKDQYACYVVQK MIDVAEPGQRKIVMHKIRPHIATLRKYTYGKHILAKLEKYYMKNGVDLG (SEQ ID NO: 446). In some aspects, SEQ ID NO: 446 comprises an architecture proceeding from the N-terminus to the C-terminus according to: R1′-R1-R2-R3-R4-R5-R6-R1-R2-R3-R4-R5-R6-R7-R8-R8′. In some aspects, SEQ ID NO: 446 is comprised of the sequences detailed in Table 13.

TABLE 13
14PUF protein according to SEQ ID NO: 446

| | RNA | | SEQ |
|---|---|---|---|
| PUF | Recogni- | | ID |
| Module | tion | Amino Acid Sequence | NO |
| PUF R1′ | — | GRSRLLEDFRNNRYPNLQ | 495 |
| | | LREIAG | |
| PUF R1 | G | HIMEFSQDQHGSRFIELK | 498 |
| | | LERATPAERQLVFNEILQ | |
| PUF R2 | A | AAYQLMVDVFGCYVIQKF | 490 |
| | | FEFGSLEQKLALAERIRG | |
| PUF R3 | C | HVLSLALQMYGSYVIRKA | 508 |
| | | LEFIPSDQQNEMVRELDG | |
| PUF R4 | G | HVLKCVKDQNGSYVVEKC | 504 |
| | | IECVQPQSLQFIIDAFKG | |
| PUF R5 | A | QVFALSTHPYGCRVIQRI | 512 |
| | | LEHCLPDQTLPILEELHQ | |
| PUF R6 | C | HTEQLVQDQYGSYVIRHV | 502 |
| | | LEHGRPEDKSKIVAEIRG | |
| PUF R1 | G | HIMEFSQDQHGSRFIELK | 498 |
| | | LERATPAERQLVFNEILQ | |
| PUF R2 | A | AAYQLMVDVFGCYVIQKF | 490 |
| | | FEFGSLEQKLALAERIRG | |
| PUF R3 | C | HVLSLALQMYGSYVIRKA | 508 |
| | | LEFIPSDQQNEMVRELDG | |
| PUF R4 | G | HVLKCVKDQNGSYVVEKC | 504 |
| | | IECVQPQSLQFIIDAFKG | |
| PUF R5 | A | QVFALSTHPYGCRVIQRI | 512 |
| | | LEHCLPDQTLPILEELHQ | |
| PUF R6 | C | HTEQLVQDQYGSYVIRHV | 502 |
| | | LEHGRPEDKSKIVAEIRG | |
| PUF R7 | G | NVLVLSQHKFASNVVEKC | 510 |
| | | VTHASRTERAVLIDEVCT | |
| | | MNDGPHS | |
| PUF R8 | A | ALYTMMKDQYACYVVQKM | 493 |
| | | IDVAEPGQRKIVMHKIRP | |
| PUF R8′ | — | HIATLRKYTYGKHILAKL | 496 |
| | | EKYYMKNGVDLG | |

An exemplary 15-mer RNA recognition 15PUF targeting AGCAGCAGCAGCAGC (SEQ ID NO: 474) comprises the amino acid sequence: GRSRLLEDFRNNRYPNLQLREIAGHIMEFSQDQHGSRFIRLKLERATPAERQLVFNEIL QAAYQLMVDVFGSYVIEKFFEFGSLEQKLALAERIRGHVLSLALQMYGCRVIQKALE FIPSDQQNEMVRELDGHVLKCVKDQNGSYVVRKCIECVQPQSLQFIIDAFKGQVFAL STHPYGSRVIERILEHCLPDQTLPILEELHQHIMEFSQDQHGSRFIQLKLERATPAERQL VFNEILQAAYQLMVDVFGSYVIRKFFEFGSLEQKLALAERIRGHVLSLALQMYGSRV IEKALEFIPSDQQNEMVRELDGHVLKCVKDQNGCHVVQKCIECVQPQSLQFIIDAFK GQVFALSTHPYGSRVIRRILEHCLPDQTLPILEELHQHTEQLVQDQYGSYVIEHVLEH GRPEDKSKIVAEIRGNVLVLSQHKFACNVVQKCVTHASRTERAVLIDEVCTMNDGP HSHTEQLVQDQYGSYVIRHVLEHGRPEDKSKIVAEIRGNVLVLSQHKFASNVVEKCV THASRTERAVLIDEVCTMNDGPHSALYTMMKDQYACYVVQKMIDVAEPGQRKIVM HKIRPHIATLRKYTYGKHILAKLEKYYMKNGVDLG (SEQ ID NO: 447). In some aspects, SEQ ID NO: 447 comprises an architecture proceeding from the N-terminus to the C-terminus according to: R1′-R1-R2-R3-R4-R5-R1-R2-R3-R4-R5-R6-R7-R6-R7-R8-R8′. In some aspects, SEQ ID NO: 447 is comprised of the sequences detailed in Table 14.

TABLE 14
15PUF protein according to SEQ ID NO: 447

| | RNA | | SEQ |
|---|---|---|---|
| PUF | Recog- | | ID |
| Module | nition | Amino Acid Sequence | NO |
| PUF R1′ | — | GRSRLLEDFRNNRYPNLQ | 495 |
| | | LREIAG | |
| PUF R1 | C | HIMEFSQDQHGSRFIRLK | 499 |
| | | LERATPAERQLVFNEILQ | |
| PUF R2 | G | AAYQLMVDVFGSYVIEKF | 491 |
| | | FEFGSLEQKLALAERIRG | |
| PUF R3 | A | HVLSLALQMYGCRVIQKA | 506 |
| | | LEFIPSDQQNEMVRELDG | |
| PUF R4 | C | HVLKCVKDQNGSYVVRKC | 505 |
| | | IECVQPQSLQFIIDAFKG | |
| PUF R5 | G | QVFALSTHPYGSRVIERI | 513 |
| | | LEHCLPDQTLPILEELHQ | |
| PUF R1 | A | HIMEFSQDQHGSRFIQLK | 497 |
| | | LERATPAERQLVFNEILQ | |
| PUF R2 | C | AAYQLMVDVFGSYVIRKF | 492 |
| | | FEFGSLEQKLALAERIRG | |
| PUF R3 | G | HVLSLALQMYGSRVIEKA | 507 |
| | | LEFIPSDQQNEMVRELDG | |
| PUF R4 | A | HVLKCVKDQNGCHVVQKC | 503 |
| | | IECVQPQSLQFIIDAFKG | |
| PUF R5 | C | QVFALSTHPYGSRVIRRI | 514 |
| | | LEHCLPDQTLPILEELHQ | |
| PUF R6 | G | HTEQLVQDQYGSYVIEHV | 51 |
| | | LEHGRPEDKSKIVAEIRG | |
| PUF R7 | A | NVLVLSQHKFACNVVQKC | 509 |
| | | VTHASRTERAVLIDEVCT | |
| | | MNDGPHS | |
| PUF R6 | C | HTEQLVQDQYGSYVIRHV | 502 |
| | | LEHGRPEDKSKIVAEIRG | |
| PUF R7 | G | NVLVLSQHKFASNVVEKC | 510 |
| | | VTHASRTERAVLIDEVCT | |
| | | MNDGPHS | |
| PUF R8 | A | ALYTMMKDQYACYVVQKM | 493 |
| | | IDVAEPGQRKIVMHKIRP | |
| PUF R8′ | — | HIATLRKYTYGKHILAKL | 496 |
| | | EKYYMKNGVDLG | |

An exemplary 15-mer RNA recognition (15PUF) targeting AGCAGCAGCAGCAGC (SEQ ID NO: 474) comprises the amino acid sequence: GRSRLLEDFRNNRYPNLQLREIAGHIMEFSQDQHGSRFIRLKLERATPAERQLVFNEIL QAAYQLMVDVFGSYVIEKFFEFGSLEQKLALAERIRGHVLSLALQMYGCRVIQKALE FIPSDQQNEMVRELDGHVLKCVKDQNGSYVVRKCIECVQPQSLQFIIDAFKGQVFAL STHPYGSRVIERILEHCLPDQTLPILEELHQHTEQLVQDQYGCYVIQHVLEHGRPEDK SKIVAEIRGHIMEFSQDQHGSRFIRLKLERATPAERQLVFNEILQAAYQLMVDVFGSY VIEKFFEFGSLEQKLALAERIRGHVLSLALQMYGCRVIQKALEFIPSDQQNEMVRELD GHVLKCVKDQNGSYVVRKCIECVQPQSLQFIIDAFKGQVFALSTHPYGSRVIERILEH CLPDQTLPILEELHQHTEQLVQDQYGCYVIQHVLEHGRPEDKSKIVAEIRGNVLVLSQ HKFASYVVRKCVTHASRTERAVLIDEVCTMNDGPHSNVLVLSQHKFASNVVEKCVT HASRTERAVLIDEVCTMNDGPHSALYTMMKDQYACYVVQKMIDVAEPGQRKIVMH KIRPHIATLRKYTYGKHILAKLEKYYMKNGVDLG (SEQ ID NO: 448). In some aspects, SEQ ID NO: 448 comprises an architecture proceeding from the N-terminus to the C-terminus according to: R1′-R1-R2-R3-R4-R5-R6-R1-R2-R3-R4-R5-R6-R7-R7-R8-R8′. In some aspects, SEQ ID NO: 448 is comprised of the sequences detailed in Table 15.

TABLE 15
15PUF protein according to SEQ ID NO: 448

| | RNA | | SEQ |
|---|---|---|---|
| PUF | Recog- | | ID |
| Module | nition | Amino Acid Sequence | NO |
| PUF R1′ | — | GRSRLLEDFRNNRYPNLQ | 495 |
| | | LREIAG | |
| PUF R1 | C | HIMEFSQDQHGSRFIRLK | 499 |
| | | LERATPAERQLVFNEILQ | |
| PUF R2 | G | AAYQLMVDVFGSYVIEKF | 491 |
| | | FEFGSLEQKLALAERIRG | |
| PUF R3 | A | HVLSLALQMYGCRVIQKA | 506 |
| | | LEFIPSDQQNEMVRELDG | |
| PUF R4 | C | HVLKCVKDQNGSYVVRKC | 505 |
| | | IECVQPQSLQFIIDAFKG | |
| PUF R5 | G | QVFALSTHPYGSRVIERI | 513 |
| | | LEHCLPDQTLPILEELHQ | |
| PUF R6 | A | HTEQLVQDQYGCYVIQHV | 500 |
| | | LEHGRPEDKSKIVAEIRG | |
| PUF R1 | C | HIMEFSQDQHGSRFIRLK | 499 |
| | | LERATPAERQLVFNEILQ | |
| PUF R2 | G | AAYQLMVDVFGSYVIEKF | 491 |
| | | FEFGSLEQKLALAERIRG | |
| PUF R3 | A | HVLSLALQMYGCRVIQKA | 506 |
| | | LEFIPSDQQNEMVRELDG | |
| PUF R4 | C | HVLKCVKDQNGSYVVRKC | 505 |
| | | IECVQPQSLQFIIDAFKG | |
| PUF R5 | G | QVFALSTHPYGSR VIER | 513 |
| | | ILEHCLPDQTLPILEELH | |
| | | Q | |
| PUF R6 | A | HTEQLVQDQYGCYVIQHV | 50 |
| | | LEHGRPEDKSKIVAEIRG | |
| PUF R7 | C | NVLVLSQHKFASYVVRKC | 511 |
| | | VTHASRTERAVLIDEVCT | |
| | | MN | |
| | | DGPHS | |
| PUF R7 | G | NVLVLSQHKFASNVVEKC | 510 |
| | | VTHASRTERAVLIDEVCT | |
| | | MN | |
| | | DGPHS | |
| PUF R8 | A | ALYTMMKDQYACYVVQKM | 493 |
| | | IDVAEPGQRKIVMHKIRP | |
| PUF R8′ | — | HIATLRKYTYGKHILAKL | 496 |
| | | EKYYMKNGVDLG | |

An exemplary 15-mer RNA recognition (5U) targeting AGCAGCAGCAGCAGC (SEQ ID NO: 474) comprises the amino acid sequence: GRSRLLEDFRNNRYPNLQLREIAGHIMEFSQDQHGSRFIRLKLERATPAERQLVFNEIL QAAYQLMVDVFGSYVIEKFFEFGSLEQKLALAERIRGHVLSLALQMYGCRVIQKALE FIPSDQQNEMVRELDGHVLKCVKDQNGSYVVRKCIECVQPQSLQFIIDAFKGQVFAL STHPYGSRVIERILEHCLPDQTLPILEELHQHTEQLVQDQYGCYVIQHVLEHGRPEDK SKIVAEIRGNVLVLSQHKFASYVVRKCVTHASRTERAVLIDEVCTMNDGPHSHIMEF SQDQHGSRFIELKLERATPAERQLVFNEILQAAYQLMVDVFGCYVIQKFFEFGSLEQK LALAERIRGHVLSLALQMYGSYVIRKALEFIPSDQQNEMVRELDGHVLKCVKDQNG SYVVEKCIECVQPQSLQFIIDAFKGQVFALSTHPYGCRVIQRILEHCLPDQTLPILEELH QHTEQLVQDQYGSYVIRHVLEHGRPEDKSKIVAEIRGNVLVLSQHKFASNVVEKCVT HASRTERAVLIDEVCTMNDGPHSALYTMMKDQYACYVVQKMIDVAEPGQRKIVMH KIRPHIATLRKYTYGKHILAKLEKYYMKNGVDLG (SEQ ID NO: 461). In some aspects, SEQ ID NO: 461 comprises an architecture proceeding from the N-terminus to the C-terminus according to: R1′-R1-R2-R3-R4-R5-R6-R7-R1-R2-R3-R4-R5-R6-R7-R8-R8′. In some aspects, SEQ ID NO: 461 is comprised of the sequences detailed in Table 16.

TABLE 16
15PUF protein according to SEQ ID NO: 461

| | RNA | | SEQ |
|---|---|---|---|
| PUF | Recog- | | ID |
| Module | nition | Amino Acid Sequence | NO |
| PUF R1′ | — | GRSRLLEDFRNNRYPNLQ | 495 |
| | | LREIAG | |
| PUF R1 | C | HIMEFSQDQHGSRFIRLK | 499 |
| | | LERATPAERQLVFNEILQ | |
| PUF R2 | G | AAYQLMVDVFGSYVIEKF | 491 |
| | | FEFGSLEQKLALAERIRG | |
| PUF R3 | A | HVLSLALQMYGCRVIQKA | 506 |
| | | LEFIPSDQQNEMVRELDG | |
| PUF R4 | C | HVLKCVKDQNGSYVVRKC | 505 |
| | | IECVQPQSLQFIIDAFKG | |
| PUF R5 | G | QVFALSTHPYGSRVIERI | 513 |
| | | LEHCLPDQTLPILEELHQ | |
| PUF R6 | A | HTEQLVQDQYGCYVIQHV | 500 |
| | | LEHGRPEDKSKIVAEIRG | |
| PUF R7 | C | NVLVLSQHKFASYVVRKC | 511 |
| | | VTHASRTERAVLIDEVCT | |
| | | MNDGPHS | |
| PUF R1 | G | HIMEFSQDQHGSRFIELK | 498 |
| | | LERATPAERQLVFNEILQ | |
| PUF R2 | A | AAYQLMVDVFGCYVIQKF | 490 |
| | | FEFGSLEQKLALAERIRG | |
| PUF R3 | C | HVLSLALQMYGSYVIRKA | 508 |
| | | LEFIPSDQQNEMVRELDG | |
| PUF R4 | G | HVLKCVKDQNGSYVVEKC | 504 |
| | | IECVQPQSLQFIIDAFKG | |
| PUF R5 | A | QVFALSTHPYGCRVIQRI | 512 |
| | | LEHCLPDQTLPILEELHQ | |
| PUF R6 | C | HTEQLVQDQYGSYVIRHV | 502 |
| | | LEHGRPEDKSKIVAEIRG | |
| PUF R7 | G | NVLVLSQHKFASNVVEKC | 510 |
| | | VTHASRTERAVLIDEVCT | |
| | | MNDGPHS | |
| PUF R8 | A | ALYTMMKDQYACYVVQKM | 493 |
| | | IDVAEPGQRKIVMHKIRP | |
| PUF R8′ | — | HIATLRKYTYGKHILAKL | 496 |
| | | EKYYMKNGVDLG | |

An exemplary 16-mer RNA recognition (16PUF) targeting AGCAGCAGCAGCAGCA (SEQ ID NO: 475) comprises the amino acid sequence: GRSRLLEDFRNNRYPNLQLREIAGHIMEFSQDQHGSRFIQLKLERATPAERQLVFNEI LQAAYQLMVDVFGSYVIRKFFEFGSLEQKLALAERIRGHVLSLALQMYGSRVIEKAL EFIPSDQQNEMVRELDGHVLKCVKDQNGCHVVQKCIECVQPQSLQFIIDAFKGQVFA LSTHPYGSRVIRRILEHCLPDQTLPILEELHQHIMEFSQDQHGSRFIELKLERATPAERQ LVFNEILQAAYQLMVDVFGCYVIQKFFEFGSLEQKLALAERIRGHVLSLALQMYGSY VIRKALEFIPSDQQNEMVRELDGHVLKCVKDQNGSYVVEKCIECVQPQSLQFIIDAFK GQVFALSTHPYGCRVIQRILEHCLPDQTLPILEELHQHTEQLVQDQYGSYVIRHVLEH GRPEDKSKIVAEIRGNVLVLSQHKFASNVVEKCVTHASRTERAVLIDEVCTMNDGPH SALYTMMKDQYACYVVQKMIDVAEPGQRKIVMHKIRPHTEQLVQDQYGSYVIRHV LEHGRPEDKSKIVAEIRGNVLVLSQHKFASNVVEKCVTHASRTERAVLIDEVCTMND GPHSALYTMMKDQYACYVVQKMIDVAEPGQRKIVMHKIRPHIATLRKYTYGKHILA KLEKYYMKNGVDLG (SEQ ID NO: 449). In some aspects, SEQ ID NO: 449 comprises an architecture proceeding from the N-terminus to the C-terminus according to: R1′-R1-R2-R3-R4-R5-R1-R2-R3-R4-R5-R6-R7-R8-R6-R7-R8-R8′. In some aspects, SEQ ID NO: 449 is comprised of the sequences detailed in Table 17.

TABLE 17
16PUF protein according to SEQ ID NO: 449

| | RNA | | SEQ |
|---|---|---|---|
| PUF | Recog | | ID |
| Module | nition | Amino Acid Sequence | NO |
| PUF | — | GRSRLLEDFRNNRYPNLQLREIAG | 495 |
| R1′ | | | |
| PUF | A | HIMEFSQDQHGSRFIQLKLERAT | 497 |
| R1 | | PAERQLVFNEILQ | |
| PUF R2 | C | AAYQLMVDVFGSYVIRKFFEFGS | 492 |
| | | LEQKLALAERIRG | |
| PUF R3 | G | HVLSLALQMYGSRVIEKALEFIP | 507 |
| | | SDQQNEMVRELDG | |
| PUF R4 | A | HVLKCVKDQNGCHVVQKCIECVQ | 503 |
| | | PQSLQFIIDAFKG | |
| PUF R5 | C | QVFALSTHPYGSRVIRRILEHCL | 514 |
| | | PDQTLPILEELHQ | |
| PUF | G | HIMEFSQDQHGSRFIELKLERAT | 498 |
| R1 | | PAERQLVFNEILQ | |
| PUF R2 | A | AAYQLMVDVFGCYVIQKFFEFGS | 490 |
| | | LEQKLALAERIRG | |
| PUF R3 | C | HVLSLALQMYGSYVIRKALEFIP | 508 |
| | | SDQQNEMVRELDG | |
| PUF R4 | G | HVLKCVKDQNGSYVVEKCIECVQ | 54 |
| | | PQSLQFIIDAFKG | |
| PUF R5 | A | QVFALSTHPYGCRVIQRILEHCL | 512 |
| | | PDQTLPILEELHQ | |
| PUF R6 | C | HTEQLVQDQYGSYVIRHVLEHGR | 502 |
| | | PEDKSKIVAEIRG | |
| PUF R7 | G | NVLVLSQHKFASNVVEKCVTHASR | 510 |
| | | TERAVLIDEVCTMNDGPHS | |
| PUF R8 | A | ALYTMMKDQYACYVVQKMIDVAEP | 493 |
| | | GQRKIVMHKIRP | |
| PUF R6 | C | HTEQLVQDQYGSYVIRHVLEHGR | 502 |
| | | PEDKSKIVAEIRG | |
| PUF R7 | G | NVLVLSQHKFASNVVEKCVTHAS | 510 |
| | | RTERAVLIDEVCTMNDGPHS | |
| PUF R8 | A | ALYTMMKDQYACYVVQKMIDVAE | 493 |
| | | PGQRKIVMHKIRP | |
| PUF | — | HIATLRKYTYGKHILAKLEKYYMK | 496 |
| R8′ | | NGVDLG | |

An exemplary 16-mer RNA recognition (16PUF) targeting AGCAGCAGCAGCAGCA (SEQ ID NO: 475) comprises the amino acid sequence: GRSRLLEDFRNNRYPNLQLREIAGHIMEFSQDQHGSRFIQLKLERATPAERQLVFNEI LQAAYQLMVDVFGSYVIRKFFEFGSLEQKLALAERIRGHVLSLALQMYGSRVIEKAL EFIPSDQQNEMVRELDGHVLKCVKDQNGCHVVQKCIECVQPQSLQFIIDAFKGQVFA LSTHPYGSRVIRRILEHCLPDQTLPILEELHQHTEQLVQDQYGSYVIEHVLEHGRPED KSKIVAEIRGHIMEFSQDQHGSRFIQLKLERATPAERQLVFNEILQAAYQLMVDVFGS YVIRKFFEFGSLEQKLALAERIRGHVLSLALQMYGSRVIEKALEFIPSDQQNEMVREL DGHVLKCVKDQNGCHVVQKCIECVQPQSLQFIIDAFKGQVFALSTHPYGSRVIRRILE HCLPDQTLPILEELHQHTEQLVQDQYGSYVIEHVLEHGRPEDKSKIVAEIRGNVLVLS QHKFACNVVQKCVTHASRTERAVLIDEVCTMNDGPHSALYTMMKDQYASYVVRK MIDVAEPGQRKIVMHKIRPNVLVLSQHKFASNVVEKCVTHASRTERAVLIDEVCTM NDGPHSALYTMMKDQYACYVVQKMIDVAEPGQRKIVMHKIRPHIATLRKYTYGKH ILAKLEKYYMKNGVDLG (SEQ ID NO: 450). In some aspects, SEQ ID NO: 450 comprises an architecture proceeding from the N-terminus to the C-terminus according to: R1′-R1-R2-R3-R4-R5-R6-R1-R2-R3-R4-R5-R6-R7-R8-R7-R8-R8′. In some aspects, SEQ ID NO: 450 is comprised of the sequences detailed in Table 18.

TABLE 18
16PUF protein according to SEQ ID NO: 450

| | RNA | | SEQ |
|---|---|---|---|
| PUF | Recog | | ID |
| Module | nition | Amino Acid Sequence | NO |
| PUF R1′ | — | GRSRLLEDFRNNRYPNLQLREIAG | 495 |
| PUF R1 | A | HIMEFSQDQHGSRFIQLKLERATPAERQLVFNEILQ | 497 |
| PUF R2 | C | AAYQLMVDVFGSYVIRKFFEFGSLEQKLALAERIRG | 492 |
| PUF R3 | G | HVLSLALQMYGSRVIEKALEFIPSDQQNEMVRELDG | 507 |
| PUF R4 | A | HVLKCVKDQNGCHVVQKCIECVQPQSLQFIIDAFKG | 503 |
| PUF R5 | C | QVFALSTHPYGSRVIRRILEHCLPDQTLPILEELHQ | 514 |
| PUF R6 | G | HTEQLVQDQYGSYVIEHVLEHGRPEDKSKIVAEIRG | 501 |
| PUF R1 | A | HIMEFSQDQHGSRFIQLKLERATPAERQLVFNEILQ | 497 |
| PUF R2 | C | AAYQLMVDVFGSYVIRKFFEFGSLEQKLALAERIRG | 492 |
| PUF R3 | G | HVLSLALQMYGSRVIEKALEFIPSDQQNEMVRELDG | 507 |
| PUF R4 | A | HVLKCVKDQNGCHVVQKCIECVQPQSLQFIIDAFKG | 503 |
| PUF R5 | C | QVFALSTHPYGSRVIRRILEHCLPDQTLPILEELHQ | 514 |
| PUF R6 | G | HTEQLVQDQYGSYVIEHVLEHGRPEDKSKIVAEIRG | 501 |
| PUF R7 | A | NVLVLSQHKFACNVVQKCVTHASRTERAVLIDEVCT | 509 |
| | | MNDGPHS | |
| PUF R8 | C | ALYTMMKDQYASYVVRKMIDVAEPGQRKIVMHKIRP | 494 |
| PUF R7 | G | NVLVLSQHKFASNVVEKCVTHASRTERAVLIDEVCT | 51 |
| | | MNDGPHS | |
| PUF R8 | A | ALYTMMKDQYACYVVQKMIDVAEPGQRKIVMHKIRP | 493 |
| PUF R8′ | — | HIATLRKYTYGKHILAKLEKYYMKNGVDLG | 496 |

An exemplary 16-mer RNA recognition (16PUF) targeting AGCAGCAGCAGCAGCA (SEQ ID NO: 475) comprises the amino acid sequence: GRSRLLEDFRNNRYPNLQLREIAGHIMEFSQDQHGSRFIQLKLERATPAERQLVFNEI LQAAYQLMVDVFGSYVIRKFFEFGSLEQKLALAERIRGHVLSLALQMYGSRVIEKAL EFIPSDQQNEMVRELDGHVLKCVKDQNGCHVVQKCIECVQPQSLQFIIDAFKGQVFA LSTHPYGSRVIRRILEHCLPDQTLPILEELHQHTEQLVQDQYGSYVIEHVLEHGRPED KSKIVAEIRGNVLVLSQHKFACNVVQKCVTHASRTERAVLIDEVCTMNDGPHSALY TMMKDQYASYVVRKMIDVAEPGQRKIVMHKIRPHIMEFSQDQHGSRFIELKLERATP AERQLVFNEILQAAYQLMVDVFGCYVIQKFFEFGSLEQKLALAERIRGHVLSLALQM YGSYVIRKALEFIPSDQQNEMVRELDGHVLKCVKDQNGSYVVEKCIECVQPQSLQFII DAFKGQVFALSTHPYGCRVIQRILEHCLPDQTLPILEELHQHTEQLVQDQYGSYVIRH VLEHGRPEDKSKIVAEIRGNVLVLSQHKFASNVVEKCVTHASRTERAVLIDEVCTMN DGPHSALYTMMKDQYACYVVQKMIDVAEPGQRKIVMHKIRPHIATLRKYTYGKHIL AKLEKYYMKNGVDLG (SEQ ID NO: 451). In some aspects, SEQ ID NO: 451 comprises an architecture proceeding from the N-terminus to the C-terminus according to: R1′-R1-R2-R3-R4-R5-R6-R7-R8-R1-R2-R3-R4-R5-R6-R7-R8-R8′. In some aspects, SEQ ID NO: 451 is comprised of the sequences detailed in Table 19.

TABLE 19
16PUF protein according to SEQ ID NO: 451

| | RNA | | SEQ |
|---|---|---|---|
| PUF | Recog | | ID |
| Module | nition | Amino Acid Sequence | NO |
| PUF R1′ | — | GRSRLLEDFRNNRYPNLQLREIAG | 495 |
| PUF R1 | A | HIMEFSQDQHGSRFIQLKLERATPAERQLVFNEILQ | 497 |
| PUF R2 | C | AAYQLMVDVFGSYVIRKFFEFGSLEQKLALAERIRG | 492 |
| PUF R3 | G | HVLSLALQMYGSRVIEKALEFIPSDQQNEMVRELDG | 507 |
| PUF R4 | A | HVLKCVKDQNGCHVVQKCIECVQPQSLQFIIDAFKG | 503 |
| PUF R5 | C | QVFALSTHPYGSRVIRRILEHCLPDQTLPILEELHQ | 514 |
| PUF R6 | G | HTEQLVQDQYGSYVIEHVLEHGRPEDKSKIVAEIRG | 501 |
| PUF R7 | A | NVLVLSQHKFACNVVQKCVTHASRTERAVLIDEVCT | 509 |
| | | MNDGPHS | |
| PUF R8 | C | ALYTMMKDQYASYVVRKMIDVAEPGQRKIVMHKIRP | 494 |
| PUF R1 | G | HIMEFSQDQHGSRFIELKLERATPAERQLVFNEILQ | 498 |
| PUF R2 | A | AAYQLMVDVFGCYVIQKFFEFGSLEQKLALAERIRG | 490 |
| PUF R3 | C | HVLSLALQMYGSYVIRKALEFIPSDQQNEMVRELDG | 508 |
| PUF R4 | G | HVLKCVKDQNGSYVVEKCIECVQPQSLQFIIDAFKG | 504 |
| PUF R5 | A | QVFALSTHPYGCRVIQRILEHCLPDQTLPILEELHQ | 512 |
| PUF R6 | C | HTEQLVQDQYGSYVIRHVLEHGRPEDKSKIVAEIRG | 52 |
| PUF R7 | G | NVLVLSQHKFASNVVEKCVTHASRTERAVLIDEVCT | 510 |
| | | MNDGPHS | |
| PUF R8 | A | ALYTMMKDQYACYVVQKMIDVAEPGQRKIVMHKIRP | 493 |
| PUF R8′ | — | HIATLRKYTYGKHILAKLEKYYMKNGVDLG | 496 |

An exemplary 8-mer RNA recognition (8PUF) targeting CAGCAGCA (SEQ ID NO: 453) comprises the amino acid sequence: GRSRLLEDFRNNRYPNLQLREIAGHIMEFSQDQHGSRFIQLKLERATPAERQLVFNEI LQAAYQLMVDVFGSYVIRKFFEFGSLEQKLALAERIRGHVLSLALQMYGSRVIEKAL EFIPSDQQNEMVRELDGHVLKCVKDQNGCYVVQKCIECVQPQSLQFIIDAFKGQVFA LSTHPYGSRVIRRILEHCLPDQTLPILEELHQHTEQLVQDQYGSYVIEHVLEHGRPED KSKIVAEIRGNVLVLSQHKFACNVVQKCVTHASRTERAVLIDEVCTMNDGPHSALY TMMKDQYASYVVRKMIDVAEPGQRKIVMHKIRPHIATLRKYTYGKHILAKLEKYY MKNGVDLG (SEQ ID NO: 480). In some aspects, SEQ ID NO: 480 comprises an architecture proceeding from the N-terminus to the C-terminus according to: R1′-R1-R2-R3-R4-R5-R6-R7-R8-R8′. In some aspects, SEQ ID NO: 480 is comprised of the sequences detailed in Table 20.

TABLE 20
8PUF protein according to SEQ ID NO: 480

| | SEQ | RNA | SEQ | | SEQ |
|---|---|---|---|---|---|
| PUF | ID | Recog | ID | | ID |
| Module | NO | nition | NO | Amino Acid Sequence | NO |
| PUF R1′ | 495 | | 495 | GRSRLLEDFRNNRYPNLQ | 495 |
| | | | | LREIAG | |
| PUF R1 | 497 | A | 497 | HIMEFSQDQHGSRFIQLK | 497 |
| | | | | LERATPAERQLVFNEILQ | |
| PUF R2 | 492 | C | 492 | AAYQLMVDVFGSYVIRKF | 492 |
| | | | | FEFGSLEQKLALAERIRG | |
| PUF R3 | 507 | G | 507 | HVLSLALQMYGSRVIEKA | 507 |
| | | | | LEFIPSDQQNEMVRELDG | |
| PUF R4 | 503 | A | 503 | HVLKCVKDQNGCYVVQKC | 503 |
| | | | | IECVQPQSLQFIIDAFKG | |
| PUF R5 | 514 | C | 514 | QVFALSTHPYGSRVIRRI | 514 |
| | | | | LEHCLPDQTLPILEELHQ | |
| PUF R6 | 501 | G | 501 | HTEQLVQDQYGSYVIEHV | 501 |
| | | | | LEHGRPEDKSKIVAEIRG | |
| PUF R7 | 509 | A | 509 | NVLVLSQHKFACNVVQKC | 509 |
| | | | | VTHASRTERAVLIDEVCT | |
| | | | | MNDGPHS | |
| PUF R8 | 494 | C | 494 | ALYTMMKDQYASYVVRKM | 494 |
| | | | | IDVAEPGQRKIVMHKIRP | |
| PUF R8′ | 496 | | 496 | HIATLRKYTYGKHILAKL | 496 |
| | | | | EKYYMKNGVDLG | |

An exemplary 14-mer RNA recognition (14PUF) targeting CAGCAGCAGCAGCA (SEQ ID NO: 454) comprises the amino acid sequence: GRSRLLEDFRNNRYPNLQLREIAGHIMEFSQDQHGSRFIQLKLERATPAERQLVFNEI LQAAYQLMVDVFGSYVIRKFFEFGSLEQKLALAERIRGHVLSLALQMYGSRVIEKAL EFIPSDQQNEMVRELDGHVLKCVKDQNGCHVVQKCIECVQPQSLQFIIDAFKGQVFA LSTHPYGSRVIRRILEHCLPDQTLPILEELHQHIMEFSQDQHGSRFIELKLERATPAERQ LVFNEILQAAYQLMVDVFGCYVIQKFFEFGSLEQKLALAERIRGHVLSLALQMYGSY VIRKALEFIPSDQQNEMVRELDGHVLKCVKDQNGSYVVEKCIECVQPQSLQFIIDAFK GQVFALSTHPYGCRVIQRILEHCLPDQTLPILEELHQHTEQLVQDQYGSYVIRHVLEH GRPEDKSKIVAEIRGHTEQLVQDQYGSYVIEHVLEHGRPEDKSKIVAEIRGNVLVLSQ HKFACNVVQKCVTHASRTERAVLIDEVCTMNDGPHSALYTMMKDQYASYVVRKMI DVAEPGQRKIVMHKIRPHIATLRKYTYGKHILAKLEKYYMKNGVDLG (SEQ ID NO: 481). In some aspects, SEQ ID NO: 481 comprises an architecture proceeding from the N-terminus to the C-terminus according to: R1′-R-R2-R3-R4-R5-R1-R2-R3-R4-R5-R6-R6-R7-R8-R8′. In some aspects, SEQ ID NO: 481 is comprised of the sequences detailed in Table 21.

TABLE 21
14PUF protein according to SEQ ID NO: 481

| | SEQ | RNA | SEQ | | SEQ |
|---|---|---|---|---|---|
| PUF | ID | Recog | ID | | ID |
| Module | NO | nition | NO | Amino Acid Sequence | NO |
| PUF R1′ | 495 | | 495 | GRSRLLEDFRNNRYPNLQ | 495 |
| | | | | LREIAG | |
| PUF R1 | 497 | A | 497 | HIMEFSQDQHGSRFIQLK | 497 |
| | | | | LERATPAERQLVFNEILQ | |
| PUF R2 | 492 | C | 492 | AAYQLMVDVFGSYVIRKF | 492 |
| | | | | FEFGSLEQKLALAERIRG | |
| PUF R3 | 507 | G | 507 | HVLSLALQMYGSRVIEKA | 507 |
| | | | | LEFIPSDQQNEMVRELDG | |
| PUF R4 | 503 | A | 503 | HVLKCVKDQNGCHVVQKC | 503 |
| | | | | IECVQPQSLQFIIDAFKG | |
| PUF R5 | 514 | C | 514 | QVFALSTHPYGSRVIRRI | 514 |
| | | | | LEHCLPDQTLPILEELHQ | |
| PUF R1 | 498 | G | 498 | HIMEFSQDQHGSRFIELK | 498 |
| | | | | LERATPAERQLVFNEILQ | |
| PUF R2 | 490 | A | 490 | AAYQLMVDVFGCYVIQKF | 490 |
| | | | | FEFGSLEQKLALAERIRG | |
| PUF R3 | 508 | C | 508 | HVLSLALQMYGSYVIRKA | 508 |
| | | | | LEFIPSDQQNEMVRELDG | |
| PUF R4 | 504 | G | 504 | HVLKCVKDQNGSYVVEKC | 504 |
| | | | | IECVQPQSLQFIIDAFKG | |
| PUF R5 | 512 | A | 512 | QVFALSTHPYGCRVIQRI | 512 |
| | | | | LEHCLPDQTLPILEELHQ | |
| PUF R6 | 502 | C | 502 | HTEQLVQDQYGSYVIRHV | 502 |
| | | | | LEHGRPEDKSKIVAEIRG | |
| PUF R6 | 501 | G | 501 | HTEQLVQDQYGSYVIEHV | 501 |
| | | | | LEHGRPEDKSKIVAEIRG | |
| PUF R7 | 509 | A | 509 | NVLVLSQHKFACNVVQKC | 509 |
| | | | | VTHASRTERAVLIDEVCT | |
| | | | | MNDGPHS | |
| PUF R8 | 494 | C | 494 | ALYTMMKDQYASYVVRKM | 494 |
| | | | | IDVAEPGQRKIVMHKIRP | |
| PUF R8′ | 496 | | 496 | HIATLRKYTYGKHILAKL | 496 |
| | | | | EKYYMKNGVDLG | |

An exemplary 14-mer RNA recognition (14PUF) targeting CAGCAGCAGCAGCA (SEQ ID NO: 454) comprises the amino acid sequence: GRSRLLEDFRNNRYPNLQLREIAGHIMEFSQDQHGSRFIQLKLERATPAERQLVFNEILQAAY QLMVDVFGSYVIRKFFEFGSLEQKLALAERIRGHVLSLALQMYGSRVIEKALEFIPSDQQNEM VRELDGHVLKCVKDQNGCHVVQKCIECVQPQSLQFIIDAFKGQVFALSTHPYGSRVIRRILEH CLPDQTLPILEELHQHTEQLVQDQYGSYVIEHVLEHGRPEDKSKIVAEIRGHIMEFSQDQHGS RFIQLKLERATPAERQLVFNEILQAAYQLMVDVFGSYVIRKFFEFGSLEQKLALAERIRGHVL SLALQMYGSRVIEKALEFIPSDQQNEMVRELDGHVLKCVKDQNGCHVVQKCIECVQPQSLQ FIIDAFKGQVFALSTHPYGSRVIRRILEHCLPDQTLPILEELHQHTEQLVQDQYGSYVIEHVLE HGRPEDKSKIVAEIRGNVLVLSQHKFACNVVQKCVTHASRTERAVLIDEVCTMNDGPHSAL YTMMKDQYASYVVRKMIDVAEPGQRKIVMHKIRPHIATLRKYTYGKHILAKLEKYYMKNG VDLG (SEQ ID NO: 482). In some aspects, SEQ ID NO: 482 comprises an architecture proceeding from the N-terminus to the C-terminus according to: R1′-R1-R2-R3-R4-R5-R6-R1-R2-R3-R4-R5-R6-R7-R8-R8′. In some aspects, SEQ ID NO: 482 is comprised of the sequences detailed in Table 22.

TABLE 22
14PUF protein according to SEQ ID NO: 482

| PUF | SEQ | RNA | SEQ | | SEQ |
|---|---|---|---|---|---|
| | ID | Recog | ID | | ID |
| Module | NO | nition | NO | Amino Acid Sequence | NO |
| PUF R1′ | 495 | | 495 | GRSRLLEDFRNNRYPNLQ | 495 |
| | | | | LREIAG | |
| PUF R1 | 497 | A | 497 | HIMEFSQDQHGSRFIQLK | 497 |
| | | | | LERATPAERQLVFNEILQ | |
| PUF R2 | 492 | C | 492 | AAYQLMVDVFGSYVIRKF | 492 |
| | | | | FEFGSLEQKLALAERIRG | |
| PUF R3 | 507 | G | 507 | HVLSLALQMYGSRVIEKA | 507 |
| | | | | LEFIPSDQQNEMVRELDG | |
| PUF R4 | 503 | A | 503 | HVLKCVKDQNGCHVVQKC | 503 |
| | | | | IECVQPQSLQFIIDAFKG | |
| PUF R5 | 514 | C | 514 | QVFALSTHPYGSRVIRRI | 514 |
| | | | | LEHCLPDQTLPILEELHQ | |
| PUF R6 | 501 | G | 501 | HTEQLVQDQYGSYVIEHV | 501 |
| | | | | LEHGRPEDKSKIVAEIRG | |
| PUF R1 | 497 | A | 497 | HIMEFSQDQHGSRFIQLK | 497 |
| | | | | LERATPAERQLVFNEILQ | |
| PUF R2 | 492 | C | 492 | AAYQLMVDVFGSYVIRKF | 492 |
| | | | | FEFGSLEQKLALAERIRG | |
| PUF R3 | 507 | G | 507 | HVLSLALQMYGSRVIEKA | 507 |
| | | | | LEFIPSDQQNEMVRELDG | |
| PUF R4 | 503 | A | 503 | HVLKCVKDQNGCHVVQKC | 503 |
| | | | | IECVQPQSLQFIIDAFKG | |
| PUF R5 | 514 | C | 514 | QVFALSTHPYGSR VIRR | 514 |
| | | | | ILEHCLPDQTLPILEELH | |
| | | | | Q | |
| PUF R6 | 501 | G | 501 | HTEQLVQDQYGSYVIEH | 501 |
| | | | | VLEHGRPEDKSKIVAEIR | |
| | | | | G | |
| PUF R7 | 509 | A | 509 | NVLVLSQHKFACNVVQKC | 509 |
| | | | | VTHASRTERAVLIDEVCT | |
| | | | | MND | |
| | | | | GPHS | |
| PUF R8 | 494 | C | 494 | ALYTMMKDQYASYVVRKM | 494 |
| | | | | IDVAEPGQRKIVMHKIRP | |
| PUF R8′ | 496 | | 496 | HIATLRKYTYGKHILAKL | 496 |
| | | | | EKYYMKNGVDLG | |

An exemplary 15-mer RNA recognition (15PUF) targeting CAGCAGCAGCAGCAG (SEQ ID NO: 455) comprises the amino acid sequence: GRSRLLEDFRNNRYPNLQLREIAGHIMEFSQDQHGSRFIELKLERATPAERQLVFNEILQAAY QLMVDVFGCYVIQKFFEFGSLEQKLALAERIRGHVLSLALQMYGSYVIRKALEFIPSDQQNE MVRELDGHVLKCVKDQNGSYVVEKCIECVQPQSLQFIIDAFKGQVFALSTHPYGCRVIQRILE HCLPDQTLPILEELHQHIMEFSQDQHGSRFIRLKLERATPAERQLVFNEILQAAYQLMVDVFG SYVIEKFFEFGSLEQKLALAERIRGHVLSLALQMYGCRVIQKALEFIPSDQQNEMVRELDGHV LKCVKDQNGSYVVRKCIECVQPQSLQFIIDAFKGQVFALSTHPYGSRVIERILEHCLPDQTLPI LEELHQHTEQLVQDQYGCYVIQHVLEHGRPEDKSKIVAEIRGNVLVLSQHKFASYVVRKCVT HASRTERAVLIDEVCTMNDGPHSHTEQLVQDQYGSYVIEHVLEHGRPEDKSKIVAEIRGNVL VLSQHKFACNVVQKCVTHASRTERAVLIDEVCTMNDGPHSALYTMMKDQYASYVVRKMID VAEPGQRKIVMHKIRPHIATLRKYTYGKHILAKLEKYYMKNGVDLG (SEQ ID NO: 483). In some aspects, SEQ ID NO: 483 comprises an architecture proceeding from the N-terminus to the C-terminus according to: R1′-R1-R2-R3-R4-R5-R1-R2-R3-R4-R5-R6-R7-R6-R7-R8-R8′. In some aspects, SEQ ID NO: 483 is comprised of the sequences detailed in Table 23.

TABLE 23
15PUF protein according to SEQ ID NO: 483

| | SEQ | RNA | SEQ | | SEQ |
|---|---|---|---|---|---|
| PUF | ID | Recog | ID | | ID |
| Module | NO | nition | NO | Amino Acid Sequence | NO |
| PUF R1′ | 495 | | 495 | GRSRLLEDFRNNRYPNLQ | 495 |
| | | | | LREIAG | |
| R1 | 498 | G | 498 | HIMEFSQDQHGSRFIELK | 498 |
| | | | | LERATPAERQLVFNEILQ | |
| R2 | 490 | A | 490 | AAYQLMVDVFGCYVIQKF | 490 |
| | | | | FEFGSLEQKLALAERIRG | |
| R3 | 508 | C | 508 | HVLSLALQMYGSYVIRKA | 508 |
| | | | | LEFIPSDQQNEMVRELDG | |
| R4 | 504 | G | 504 | HVLKCVKDQNGSYVVEKC | 504 |
| | | | | IECVQPQSLQFIIDAFKG | |
| R5 | 512 | A | 512 | QVFALSTHPYGCRVIQRI | 512 |
| | | | | LEHCLPDQTLPILEELHQ | |
| RI | 499 | C | 499 | HIMEFSQDQHGSRFIRLK | 499 |
| | | | | LERATPAERQLVFNEILQ | |
| R2 | 491 | G | 491 | AAYQLMVDVFGSYVIEKF | 491 |
| | | | | FEFGSLEQKLALAERIRG | |
| R3 | 506 | A | 506 | HVLSLALQMYGCRVIQKA | 506 |
| | | | | LEFIPSDQQNEMVRELDG | |
| R4 | 505 | C | 505 | HVLKCVKDQNGSYVVRKC | 505 |
| | | | | IECVQPQSLQFIIDAFKG | |
| R5 | 513 | G | 513 | QVFALSTHPYGSRVIERI | 513 |
| | | | | LEHCLPDQTLPILEELHQ | |
| R6 | 500 | A | 500 | HTEQLVQDQYGCYVIQHV | 500 |
| | | | | LEHGRPEDKSKIVAEIRG | |
| R7 | 511 | C | 511 | NVLVLSQHKFASYVVRKC | 511 |
| | | | | VTHASRTERAVLIDEVCT | |
| | | | | MNDGPHS | |
| R6 | 501 | G | 501 | HTEQLVQDQYGSYVIEHV | 501 |
| | | | | LEHGRPEDKSKIVAEIRG | |
| R7 | 509 | A | 509 | NVLVLSQHKFACNVVQKC | 509 |
| | | | | VTHASRTERAVLIDEVCT | |
| | | | | MNDGPHS | |
| R8 | 494 | C | 494 | ALYTMMKDQYASYVVRKM | 494 |
| | | | | IDVAEPGQRKIVMHKIRP | |
| PUF R8′ | 496 | | 496 | HIATLRKYTYGKHILAKL | 496 |
| | | | | EKYYMKNGVDLG | |

An exemplary 15-mer RNA recognition (15PUF) targeting CAGCAGCAGCAGCAG (SEQ ID NO: 455) comprises the amino acid sequence: GRSRLLEDFRNNRYPNLQLREIAGHIMEFSQDQHGSRFIELKLERATPAERQLVFNEILQAAY QLMVDVFGCYVIQKFFEFGSLEQKLALAERIRGHVLSLALQMYGSYVIRKALEFIPSDQQNE MVRELDGHVLKCVKDQNGSYVVEKCIECVQPQSLQFIIDAFKGQVFALSTHPYGCRVIQRILE HCLPDQTLPILEELHQHTEQLVQDQYGSYVIRHVLEHGRPEDKSKIVAEIRGHIMEFSQDQHG SRFIELKLERATPAERQLVFNEILQAAYQLMVDVFGCYVIQKFFEFGSLEQKLALAERIRGHV LSLALQMYGSYVIRKALEFIPSDQQNEMVRELDGHVLKCVKDQNGSYVVEKCIECVQPQSL QFIIDAFKGQVFALSTHPYGCRVIQRILEHCLPDQTLPILEELHQHTEQLVQDQYGSYVIRHVL EHGRPEDKSKIVAEIRGNVLVLSQHKFASNVVEKCVTHASRTERAVLIDEVCTMNDGPHSNV LVLSQHKFACNVVQKCVTHASRTERAVLIDEVCTMNDGPHSALYTMMKDQYASYVVRKMI DVAEPGQRKIVMHKIRPHIATLRKYTYGKHILAKLEKYYMKNGVDLG (SEQ ID NO: 484). In some aspects, SEQ ID NO: 484 comprises an architecture proceeding from the N-terminus to the C-terminus according to: R1′-R1-R2-R3-R4-R5-R6-R1-R2-R3-R4-R5-R6-R7-R7-R8-R8′. In some aspects, SEQ ID NO: 484 is comprised of the sequences detailed in Table 24.

TABLE 24
15PUF protein according to SEQ ID NO: 484

| | RNA | | SEQ |
|---|---|---|---|
| PUF | Recog | | ID |
| Module | nition | Amino Acid Sequence | NO |
| PUF R1′ | | HIMEFSQDQHGSRFIELKLERATPAERQLVFNEILQ | 498 |
| R1 | G | AAYQLMVDVFGCYVIQKFFEFGSLEQKLALAERIRG | 498 |
| R2 | A | HVLSLALQMYGSYVIRKALEFIPSDQQNEMVRELDG | 490 |
| R3 | C | HVLKCVKDQNGSYVVEKCIECVQPQSLQFIIDAFKG | 508 |
| R4 | G | QVFALSTHPYGCRVIQRILEHCLPDQTLPILEELHQ | 504 |
| R5 | A | HTEQLVQDQYGSYVIRHVLEHGRPEDKSKIVAEIRG | 512 |
| R6 | C | HIMEFSQDQHGSRFIELKLERATPAERQLVFNEILQ | 498 |
| R1 | G | AAYQLMVDVFGCYVIQKFFEFGSLEQKLALAERIRG | 498 |
| R2 | A | HVLSLALQMYGSYVIRKALEFIPSDQQNEMVRELDG | 490 |
| R3 | C | HVLKCVKDQNGSYVVEKCIECVQPQSLQFIIDAFKG | 508 |
| R4 | G | QVFALSTHPYGCRVIQRILEHCLPDQTLPILEELHQ | 504 |
| R5 | A | HTEQLVQDQYGSYVIRHVLEHGRPEDKSKIVAEIRG | 512 |
| R6 | C | NVLVLSQHKFASNVVEKCVTHASRTERAVLIDEVCT | 502 |
| | | MNDGPHS | |
| R7 | G | NVLVLSQHKFACNVVQKCVTHASRTERAVLIDEVCT | 510 |
| | | MNDGPHS | |
| R7 | A | ALYTMMKDQYASYVVRKMIDVAEPGQRKIVMHKIRP | 509 |
| R8 | C | HIATLRKYTYGKHILAKLEKYYMKNGVDLG | 496 |
| PUF R8′ | | HIMEFSQDQHGSRFIELKLERATPAERQLVFNEILQ | 498 |

An exemplary 15-mer RNA recognition (15PUF) targeting CAGCAGCAGCAGCAG (SEQ ID NO: 455) comprises the amino acid sequence: GRSRLLEDFRNNRYPNLQLREIAGHIMEFSQDQHGSRFIELKLERATPAERQLVFNEILQAAY QLMVDVFGCYVIQKFFEFGSLEQKLALAERIRGHVLSLALQMYGSYVIRKALEFIPSDQQNE MVRELDGHVLKCVKDQNGSYVVEKCIECVQPQSLQFIIDAFKGQVFALSTHPYGCRVIQRILE HCLPDQTLPILEELHQHTEQLVQDQYGSYVIRHVLEHGRPEDKSKIVAEIRGNVLVLSQHKFA SNVVEKCVTHASRTERAVLIDEVCTMNDGPHSHIMEFSQDQHGSRFIQLKLERATPAERQLV FNEILQAAYQLMVDVFGSYVIRKFFEFGSLEQKLALAERIRGHVLSLALQMYGSRVIEKALEF IPSDQQNEMVRELDGHVLKCVKDQNGCHVVQKCIECVQPQSLQFIIDAFKGQVFALSTHPYG SRVIRRILEHCLPDQTLPILEELHQHTEQLVQDQYGSYVIEHVLEHGRPEDKSKIVAEIRGNVL VLSQHKFACNVVQKCVTHASRTERAVLIDEVCTMNDGPHSALYTMMKDQYASYVVRKMID VAEPGQRKIVMHKIRPHIATLRKYTYGKHILAKLEKYYMKNGVDLG (SEQ ID NO: 485). In some aspects, SEQ ID NO: 485 comprises an architecture proceeding from the N-terminus to the C-terminus according to: R1′-R1-R2-R3-R4-R5-R6-R7-R1-R2-R3-R4-R5-R6-R7-R8-R8′. In some aspects, SEQ ID NO: 485 is comprised of the sequences detailed in Table 25.

TABLE 25
15PUF protein according to SEQ ID NO: 485

| PUF | RNA | | SEQ |
|---|---|---|---|
| Module | Recognition | Amino Acid Sequence | ID NO |
| PUF R1′ | | GRSRLLEDFRNNRYPNLQ | 495 |
| | | LREIAG | |
| R1 | G | HIMEFSQDQHGSRFIELK | 498 |
| | | LERATPAERQLVFNEILQ | |
| R2 | A | AAYQLMVDVFGCYVIQKF | 490 |
| | | FEFGSLEQKLALAERIRG | |
| R3 | C | HVLSLALQMYGSYVIRKA | 508 |
| | | LEFIPSDQQNEMVRELDG | |
| R4 | G | HVLKCVKDQNGSYVVEKC | 504 |
| | | IECVQPQSLQFIIDAFKG | |
| R5 | A | QVFALSTHPYGCRVIQRI | 512 |
| | | LEHCLPDQTLPILEELHQ | |
| R6 | C | HTEQLVQDQYGSYVIRHV | 502 |
| | | LEHGRPEDKSKIVAEIRG | |
| R7 | G | NVLVLSQHKFASNVVEKC | 510 |
| | | VTHASRTERAVLIDEVCT | |
| | | MNDGPHS | |
| R1 | A | HIMEFSQDQHGSRFIQLK | 497 |
| | | LERATPAERQLVFNEILQ | |
| R2 | C | AAYQLMVDVFGSYVIRKF | 492 |
| | | FEFGSLEQKLALAERIRG | |
| R3 | G | HVLSLALQMYGSRVIEKA | 507 |
| | | LEFIPSDQQNEMVRELDG | |
| R4 | A | HVLKCVKDQNGCHVVQKC | 503 |
| | | IECVQPQSLQFIIDAFKG | |
| R5 | C | QVFALSTHPYGSRVIRRI | 514 |
| | | LEHCLPDQTLPILEELHQ | |
| R6 | G | HTEQLVQDQYGSYVIEHV | 501 |
| | | LEHGRPEDKSKIVAEIRG | |
| R7 | A | NVLVLSQHKFACNVVQKC | 509 |
| | | VTHASRTERAVLIDEVCT | |
| | | MNDGPHS | |
| R& | C | ALYTMMKDQYASYVVRKM | 494 |
| | | IDVAEPGQRKIVMHKIRP | |
| PUF R8′ | | HIATLRKYTYGKHILAKL | 496 |
| | | EKYYMKNGVDLG | |

An exemplary 16-mer RNA recognition (16PUF) targeting CAGCAGCAGCAGCAGC (SEQ ID NO: 456) comprises the amino acid sequence: GRSRLLEDFRNNRYPNLQLREIAGHIMEFSQDQHGSRFIRLKLERATPAERQLVFNEILQAAY QLMVDVFGSYVIEKFFEFGSLEQKLALAERIRGHVLSLALQMYGCRVIQKALEFIPSDQQNE MVRELDGHVLKCVKDQNGSYVVRKCIECVQPQSLQFIIDAFKGQVFALSTHPYGSRVIERILE HCLPDQTLPILEELHQHIMEFSQDQHGSRFIQLKLERATPAERQLVFNEILQAAYQLMVDVFG SYVIRKFFEFGSLEQKLALAERIRGHVLSLALQMYGSRVIEKALEFIPSDQQNEMVRELDGHV LKCVKDQNGCHVVQKCIECVQPQSLQFIIDAFKGQVFALSTHPYGSRVIRRILEHCLPDQTLPI LEELHQHTEQLVQDQYGSYVIEHVLEHGRPEDKSKIVAEIRGNVLVLSQHKFACNVVQKCVT HASRTERAVLIDEVCTMNDGPHSALYTMMKDQYASYVVRKMIDVAEPGQRKIVMHKIRPH TEQLVQDQYGSYVIEHVLEHGRPEDKSKIVAEIRGNVLVLSQHKFACNVVQKCVTHASRTER AVLIDEVCTMNDGPHSALYTMMKDQYASYVVRKMIDVAEPGQRKIVMHKIRPHIATLRKYT YGKHILAKLEKYYMKNGVDLG (SEQ ID NO: 486). In some aspects, SEQ ID NO: 486 comprises an architecture proceeding from the N-terminus to the C-terminus according to: R1′-R1-R2-R3-R4-R5-R1-R2-R3-R4-R5-R6-R7-R8-R6-R7-R8-R8′. In some aspects, SEQ ID NO: 486 is comprised of the sequences detailed in Table 26.

TABLE 26
16PUF protein according to SEQ ID NO: 486

| PUF | RNA | | SEQ |
|---|---|---|---|
| Module | Recognition | Amino Acid Sequence | ID NO |
| PUF R1′ | | GRSRLLEDFRNNRYPNLQ | 495 |
| | | LREIAG | |
| R1 | C | HIMEFSQDQHGSRFIRLK | 499 |
| | | LERATPAERQLVFNEILQ | |
| R2 | G | AAYQLMVDVFGSYVIEKF | 491 |
| | | FEFGSLEQKLALAERIRG | |
| R3 | A | HVLSLALQMYGCRVIQKA | 506 |
| | | LEFIPSDQQNEMVRELDG | |
| R4 | C | HVLKCVKDQNGSYVVRKC | 505 |
| | | IECVQPQSLQFIIDAFKG | |
| R5 | G | QVFALSTHPYGSRVIERI | 513 |
| | | LEHCLPDQTLPILEELHQ | |
| R1 | A | HIMEFSQDQHGSRFIQLK | 497 |
| | | LERATPAERQLVFNEILQ | |
| R2 | C | AAYQLMVDVFGSYVIRKF | 492 |
| | | FEFGSLEQKLALAERIRG | |
| R3 | G | HVLSLALQMYGSRVIEKA | 507 |
| | | LEFIPSDQQNEMVRELDG | |
| R4 | A | HVLKCVKDQNGCHVVQKC | 503 |
| | | IECVQPQSLQFIIDAFKG | |
| R5 | C | QVFALSTHPYGSRVIRRI | 514 |
| | | LEHCLPDQTLPILEELHQ | |
| R6 | G | HTEQLVQDQYGSYVIEHV | 51 |
| | | LEHGRPEDKSKIVAEIRG | |
| R7 | A | NVLVLSQHKFACNVVQKC | 509 |
| | | VTHASRTERAVLIDEVCT | |
| | | MNDGPHS | |
| R8 | C | ALYTMMKDQYASYVVRKM | 494 |
| | | IDVAEPGQRKIVMHKIRP | |
| R6 | G | HTEQLVQDQYGSYVIEHV | 501 |
| | | LEHGRPEDKSKIVAEIRG | |
| R7 | A | NVLVLSQHKFACNVVQKC | 509 |
| | | VTHASRTERAVLIDEVCT | |
| | | MNDGPHS | |
| R8 | C | ALYTMMKDQYASYVVRKM | 494 |
| | | IDVAEPGQRKIVMHKIRP | |
| PUF R8′ | | HIATLRKYTYGKHILAKL | 496 |
| | | EKYYMKNGVDLG | |

An exemplary 16-mer RNA recognition (16PUF) targeting CAGCAGCAGCAGCAGC (SEQ ID NO: 456) comprises the amino acid sequence: GRSRLLEDFRNNRYPNLQLREIAGHIMEFSQDQHGSRFIRLKLERATPAERQLVFNEIL QAAYQLMVDVFGSYVIEKFFEFGSLEQKLALAERIRGHVLSLALQMYGCRVIQKALE FIPSDQQNEMVRELDGHVLKCVKDQNGSYVVRKCIECVQPQSLQFIIDAFKGQVFAL STHPYGSRVIERILEHCLPDQTLPILEELHQHTEQLVQDQYGCYVIQHVLEHGRPEDK SKIVAEIRGHIMEFSQDQHGSRFIRLKLERATPAERQLVFNEILQAAYQLMVDVFGSY VIEKFFEFGSLEQKLALAERIRGHVLSLALQMYGCRVIQKALEFIPSDQQNEMVRELD GHVLKCVKDQNGSYVVRKCIECVQPQSLQFIIDAFKGQVFALSTHPYGSRVIERILEH CLPDQTLPILEELHQHTEQLVQDQYGCYVIQHVLEHGRPEDKSKIVAEIRGNVLVLSQ HKFASYVVRKCVTHASRTERAVLIDEVCTMNDGPHSALYTMMKDQYASYVVEKMI DVAEPGQRKIVMHKIRPNVLVLSQHKFACNVVQKCVTHASRTERAVLIDEVCTMND GPHSALYTMMKDQYASYVVRKMIDVAEPGQRKIVMHKIRPHIATLRKYTYGKHILA KLEKYYMKNGVDLG (SEQ ID NO: 487). In some aspects, SEQ ID NO: 487 comprises an architecture proceeding from the N-terminus to the C-terminus according to: R1′-R1-R2-R3-R4-R5-R6-R1-R2-R3-R4-R5-R6-R7-R8-R7-R8-R8′. In some aspects, SEQ ID NO: 487 is comprised of the sequences detailed in Table 27.

TABLE 27
16PUF protein according to SEQ ID NO: 487

| PUF | RNA | | SEQ |
|---|---|---|---|
| Module | Recognition | Amino Acid Sequence | ID NO |
| PUF R1′ | | GRSRLLEDFRNNRYPNLQ | 495 |
| | | LREIAG | |
| R1 | C | HIMEFSQDQHGSRFIRLK | 499 |
| | | LERATPAERQLVFNEILQ | |
| R2 | G | AAYQLMVDVFGSYVIEKF | 491 |
| | | FEFGSLEQKLALAERIRG | |
| R3 | A | HVLSLALQMYGCRVIQKA | 506 |
| | | LEFIPSDQQNEMVRELDG | |
| R4 | C | HVLKCVKDQNGSYVVRKC | 55 |
| | | IECVQPQSLQFIIDAFKG | |
| R5 | G | QVFALSTHPYGSRVIERI | 513 |
| | | LEHCLPDQTLPILEELHQ | |
| R6 | A | HTEQLVQDQYGCYVIQHV | 500 |
| | | LEHGRPEDKSKIVAEIRG | |
| R1 | C | HIMEFSQDQHGSRFIRLK | 499 |
| | | LERATPAERQLVFNEILQ | |
| R2 | G | AAYQLMVDVFGSYVIEKF | 491 |
| | | FEFGSLEQKLALAERIRG | |
| R3 | A | HVLSLALQMYGCRVIQKA | 506 |
| | | LEFIPSDQQNEMVRELDG | |
| R4 | C | HVLKCVKDQNGSYVVRKC | 505 |
| | | IECVQPQSLQFIIDAFKG | |
| R5 | G | QVFALSTHPYGSRVIERI | 513 |
| | | LEHCLPDQTLPILEELHQ | |
| R6 | A | HTEQLVQDQYGCYVIQHV | 500 |
| | | LEHGRPEDKSKIVAEIRG | |
| R7 | C | NVLVLSQHKFASYVVRKC | 511 |
| | | VTHASRTERAVLIDEVCT | |
| | | MNDGPHS | |
| R8 | G | ALYTMMKDQYASYVVEKM | 489 |
| | | IDVAEPGQRKIVMHKIRP | |
| R7 | A | NVLVLSQHKFACNVVQKC | 509 |
| | | VTHASRTERAVLIDEVCT | |
| | | MNDGPHS | |
| R8 | C | ALYTMMKDQYASYVVRKM | 494 |
| | | IDVAEPGQRKIVMHKIRP | |
| PUF R8′ | | HIATLRKYTYGKHILAKL | 496 |
| | | EKYYMKNGVDLG | |

An exemplary 16-mer RNA recognition 16PUF targeting CAGCAGCAGCAGCAGC (SEQ ID NO: 456) comprises the amino acid sequence: GRSRLLEDFRNNRYPNLQLREIAGHIMEFSQDQHGSRFIRLKLERATPAERQLVFNEILQAAY QLMVDVFGSYVIEKFFEFGSLEQKLALAERIRGHVLSLALQMYGCRVIQKALEFIPSDQQNE MVRELDGHVLKCVKDQNGSYVVRKCIECVQPQSLQFIIDAFKGQVFALSTHPYGSRVIERILE HCLPDQTLPILEELHQHTEQLVQDQYGCYVIQHVLEHGRPEDKSKIVAEIRGNVLVLSQHKFA SYVVRKCVTHASRTERAVLIDEVCTMNDGPHSALYTMMKDQYASYVVEKMIDVAEPGQRK IVMHKIRPHIMEFSQDQHGSRFIQLKLERATPAERQLVFNEILQAAYQLMVDVFGSYVIRKFF EFGSLEQKLALAERIRGHVLSLALQMYGSRVIEKALEFIPSDQQNEMVRELDGHVLKCVKDQ NGCHVVQKCIECVQPQSLQFIIDAFKGQVFALSTHPYGSRVIRRILEHCLPDQTLPILEELHQH TEQLVQDQYGSYVIEHVLEHGRPEDKSKIVAEIRGNVLVLSQHKFACNVVQKCVTHASRTER AVLIDEVCTMNDGPHSALYTMMKDQYASYVVRKMIDVAEPGQRKIVMHKIRPHIATLRKYT YGKHILAKLEKYYMKNGVDLG (SEQ ID NO: 488). In some aspects, SEQ ID NO: 488 comprises an architecture proceeding from the N-terminus to the C-terminus according to: R1′-R1-R2-R3-R4-R5-R6-R7-R8-R1-R2-R3-R4-R5-R6-R7-R8-R8′. In some aspects, SEQ ID NO: 488 is comprised of the sequences detailed in Table 28.

TABLE 28
16PUF protein according to SEQ ID NO: 488

| PUF | RNA | | SEQ |
|---|---|---|---|
| Module | Recognition | Amino Acid Sequence | ID NO |
| PUF R1′ | | HIMEFSQDQHGSRFIRLK | 499 |
| | | LERATPAERQLVFNEILQ | |
| R1 | C | AAYQLMVDVFGSYVIEKF | 491 |
| | | FEFGSLEQKLALAERIRG | |
| R2 | G | HVLSLALQMYGCRVIQKA | 507 |
| | | LEFIPSDQQNEMVRELDG | |
| R3 | A | HVLKCVKDQNGSYVVRKC | 503 |
| | | IECVQPQSLQFIIDAFKG | |
| | C | QVFALSTHPYGSRVIERI | 514 |
| R4 | | LEHCLPDQTLPILEELHQ | |
| R5 | G | HTEQLVQDQYGCYVIQHV | 501 |
| | | LEHGRPEDKSKIVAEIRG | |
| R6 | A | NVLVLSQHKFASYVVRKC | 509 |
| | | VTHASRTERAVLIDEVCT | |
| | | MNDGPHS | |
| R7 | C | ALYTMMKDQYASYVVEKM | 494 |
| | | IDVAEPGQRKIVMHKIRP | |
| R8 | G | HIMEFSQDQHGSRFIQLK | 498 |
| | | LERATPAERQLVFNEILQ | |
| R1 | A | AAYQLMVDVFGSYVIRKF | 491 |
| | | FEFGSLEQKLALAERIRG | |
| R2 | C | HVLSLALQMYGSRVIEKA | 507 |
| | | LEFIPSDQQNEMVRELDG | |
| R3 | G | HVLKCVKDQNGCHVVQKC | 503 |
| | | IECVQPQSLQFIIDAFKG | |
| R4 | A | QVFALSTHPYGSRVIRRI | 514 |
| | | LEHCLPDQTLPILEELHQ | |
| R5 | C | HTEQLVQDQYGSYVIEHV | 501 |
| | | LEHGRPEDKSKIVAEIRG | |
| R6 | G | NVLVLSQHKFACNVVQKC | 509 |
| | | VTHASRTERAVLIDEVCT | |
| | | MNDGPHS | |
| R7 | A | ALYTMMKDQYASYVVRKM | 494 |
| | | IDVAEPGQRKIVMHKIRP | |
| R8 | C | HIATLRKYTYGKHILAKL | 496 |
| | | EKYYMKNGVDLG | |
| PUF R8′ | | HIMEFSQDQHGSRFIRLK | 499 |
| | | LERATPAERQLVFNEILQ | |

An exemplary 8-mer RNA recognition (8PUF) targeting GCAGCAGC (SEQ ID NO: 476) comprises the amino acid sequence: GRS RLLEDFRNNRYPNLQLREIAGHIMEFSQDQHGSRFIRLKLERATPAERQLVFNEIL QAAYQLMVDVFGSYVIEKFFEFGSLEQKLALAERIRGHVLSLALQMYGCRVIQKALE FIPSDQQNEMVRELDGHVLKCVKDQNGSYVVRKCIECVQPQSLQFIIDAFKGQVFAL STHPYGSRVIERILEHCLPDQTLPILEELHQHTEQLVQDQYGCYVIQHVLEHGRPEDK SKIVAEIRGNVLVLSQHKFASYVVRKCVTHASRTERAVLIDEVCTMNDGPHSALYT MMKDQYASYVVEKMIDVAEPGQRKIVMHKIRPHIATLRKYTYGKHILAKLEKYYM KNGVDLG (SEQ ID NO: 549). In some aspects, SEQ ID NO: 549 comprises an architecture proceeding from the N-terminus to the C-terminus according to: R1′-R1-R2-R3-R4-R5-R6-R7-R8-R8′. In some aspects, SEQ ID NO: 549 is comprised of the sequences detailed in Table 29.

TABLE 29
8PUF protein according to SEQ ID NO: 549

| PUF | RNA | | SEQ |
|---|---|---|---|
| Module | Recognition | Amino Acid Sequence | ID NO |
| PUF R1′ | | GRSRLLEDFRNNRYPNLQ | 495 |
| | | LREIAG | |
| R1 | C | HIMEFSQDQHGSRFIRLK | 499 |
| | | LERATPAERQLVFNEILQ | |
| R2 | G | AAYQLMVDVFGSYVIEKF | 491 |
| | | FEFGSLEQKLALAERIRG | |
| R3 | A | HVLSLALQMYGCRVIQKA | 506 |
| | | LEFIPSDQQNEMVRELDG | |
| R4 | C | HVLKCVKDQNGSYVVRKC | 505 |
| | | IECVQPQSLQFIIDAFKG | |
| R5 | G | QVFALSTHPYGSRVIERI | 513 |
| | | LEHCLPDQTLPILEELHQ | |
| R6 | A | HTEQLVQDQYGCYVIQHV | 500 |
| | | LEHGRPEDKSKIVAEIRG | |
| R7 | C | NVLVLSQHKFASYVVRKC | 511 |
| | | VTHASRTERAVLIDEVCT | |
| | | MNDGPHS | |
| R8 | G | ALYTMMKDQYASYVVEKM | 489 |
| | | IDVAEPGQRKIVMHKIRP | |
| PUF R8′ | | HIATLRKYTYGKHILAKL | 496 |
| | | EKYYMKNGVDLG | |

An exemplary 14-mer RNA recognition (14PUF) targeting GCAGCAGCAGCAGC (SEQ ID NO: 477) comprises the amino acid sequence: GRSRLLEDFRNNRYPNLQLREIAGHIMEFSQDQHGSRFIRLKLERATPAERQLVFNEIL QAAYQLMVDVFGSYVIEKFFEFGSLEQKLALAERIRGHVLSLALQMYGCRVIQKALE FIPSDQQNEMVRELDGHVLKCVKDQNGSYVVRKCIECVQPQSLQFIIDAFKGQVFAL STHPYGSRVIERILEHCLPDQTLPILEELHQHTEQLVQDQYGCYVIQHVLEHGRPEDK SKIVAEIRGNVLVLSQHKFASYVVRKCVTHASRTERAVLIDEVCTMNDGPHSALYT MMKDQYASYVVEKMIDVAEPGQRKIVMHKIRPHIATLRKYTYGKHILAKLEKYYM KNGVDLG (SEQ ID NO: 550). In some aspects, SEQ ID NO: 550 comprises an architecture proceeding from the N-terminus to the C-terminus according to: R1′-R1-R2-R3-R4-R5-R1-R2-R3-R4-R5-R6-R6-R7-R8-R8′. In some aspects, SEQ ID NO: 550 is comprised of the sequences detailed in Table 30.

TABLE 30
14PUF protein according to SEQ ID NO: 550

| PUF | RNA | | SEQ |
|---|---|---|---|
| Module | Recognition | Amino Acid Sequence | ID NO |
| PUF R1′ | | GRSRLLEDFRNNRYPNLQ | 495 |
| | | LREIAG | |
| R1 | C | HIMEFSQDQHGSRFIRLK | 499 |
| | | LERATPAERQLVFNEILQ | |
| R2 | G | AAYQLMVDVFGSYVIEKF | 491 |
| | | FEFGSLEQKLALAERIRG | |
| R3 | A | HVLSLALQMYGCRVIQKA | 506 |
| | | LEFIPSDQQNEMVRELDG | |
| R4 | C | HVLKCVKDQNGSHVVRKC | 505 |
| | | IECVQPQSLQFIIDAFKG | |
| R5 | G | QVFALSTHPYGSRVIERI | 513 |
| | | LEHCLPDQTLPILEELHQ | |
| R1 | A | HIMEFSQDQHGSRFIQLK | 497 |
| | | LERATPAERQLVFNEILQ | |
| R2 | C | AAYQLMVDVFGSYVIRKF | 492 |
| | | FEFGSLEQKLALAERIRG | |
| R3 | G | HVLSLALQMYGSRVIEKA | 507 |
| | | LEFIPSDQQNEMVRELDG | |
| R4 | A | HVLKCVKDQNGCHVVQKC | 503 |
| | | IECVQPQSLQFIIDAFKG | |
| R5 | C | QVFALSTHPYGSRVIRRI | 514 |
| | | LEHCLPDQTLPILEELHQ | |
| R6 | G | HTEQLVQDQYGSYVIEHV | 501 |
| | | LEHGRPEDKSKIVAEIRG | |
| R6 | A | HTEQLVQDQYGCYVIQHV | 500 |
| | | LEHGRPEDKSKIVAEIRG | |
| R7 | C | NVLVLSQHKFASYVVRKC | 511 |
| | | VTHASRTERAVLIDEVCT | |
| | | MNDGPHS | |
| R8 | G | ALYTMMKDQYASYVVEKM | 489 |
| | | IDVAEPGQRKIVMHKIRP | |
| PUF R8′ | | HIATLRKYTYGKHILAKL | 496 |
| | | EKYYMKNGVDLG | |

An exemplary 14-mer RNA recognition (14PUF) targeting GCAGCAGCAGCAGC (SEQ ID NO: 477) comprises the amino acid sequence: GRSRLLEDFRNNRYPNLQLREIAGHIMEFSQDQHGSRFIRLKLERATPAERQLVFNEILQAAY QLMVDVFGSYVIEKFFEFGSLEQKLALAERIRGHVLSLALQMYGCRVIQKALEFIPSDQQNE MVRELDGHVLKCVKDQNGSHVVRKCIECVQPQSLQFIIDAFKGQVFALSTHPYGSRVIERILE HCLPDQTLPILEELHQHTEQLVQDQYGCYVIQHVLEHGRPEDKSKIVAEIRGHIMEFSQDQHG SRFIRLKLERATPAERQLVFNEILQAAYQLMVDVFGSYVIEKFFEFGSLEQKLALAERIRGHVL SLALQMYGCRVIQKALEFIPSDQQNEMVRELDGHVLKCVKDQNGSHVVRKCIECVQPQSLQ FIIDAFKGQVFALSTHPYGSRVIERILEHCLPDQTLPILEELHQHTEQLVQDQYGCYVIQHVLE HGRPEDKSKIVAEIRGNVLVLSQHKFASYVVRKCVTHASRTERAVLIDEVCTMNDGPHSALY TMMKDQYASYVVEKMIDVAEPGQRKIVMHKIRPHIATLRKYTYGKHILAKLEKYYMKNGV DLG (SEQ ID NO: 551). In some aspects, SEQ ID NO: 551 comprises an architecture proceeding from the N-terminus to the C-terminus according to: R1′-R1-R2-R3-R4-R5-R6-R1-R2-R3-R4-R5-R6-R7-R8-R8′. In some aspects, SEQ ID NO: 551 is comprised of the sequences detailed in Table 31.

TABLE 31
14PUF protein according to SEQ ID NO: 551

| PUF | RNA | | SEQ |
|---|---|---|---|
| Module | Recognition | Amino Acid Sequence | ID NO |
| PUF R1′ | | GRSRLLEDFRNNRYPNLQ | 495 |
| | | LREIAG | |
| R1 | C | HIMEFSQDQHGSRFIRLK | 499 |
| | | LERATPAERQLVFNEILQ | |
| R2 | G | AAYQLMVDVFGSYVIEKF | 491 |
| | | FEFGSLEQKLALAERIRG | |
| R3 | A | HVLSLALQMYGCRVIQKA | 506 |
| | | LEFIPSDQQNEMVRELDG | |
| R4 | C | HVLKCVKDQNGSHVVRKC | 505 |
| | | IECVQPQSLQFIIDAFKG | |
| R5 | G | QVFALSTHPYGSRVIERI | 513 |
| | | LEHCLPDQTLPILEELHQ | |
| R6 | A | HTEQLVQDQYGCYVIQHV | 500 |
| | | LEHGRPEDKSKIVAEIRG | |
| R1 | C | HIMEFSQDQHGSRFIRLK | 499 |
| | | LERATPAERQLVFNEILQ | |
| R2 | G | AAYQLMVDVFGSYVIEKF | 491 |
| | | FEFGSLEQKLALAERIRG | |
| R3 | A | HVLSLALQMYGCRVIQKA | 506 |
| | | LEFIPSDQQNEMVRELDG | |
| R4 | C | HVLKCVKDQNGSHVVRKC | 505 |
| | | IECVQPQSLQFIIDAFKG | |
| R5 | G | QVFALSTHPYGSRVIERI | 513 |
| | | LEHCLPDQTLPILEELHQ | |
| R6 | A | HTEQLVQDQYGCYVIQHV | 500 |
| | | LEHGRPEDKSKIVAEIRG | |
| R7 | C | NVLVLSQHKFASYVVRKC | 511 |
| | | VTHASRTERAVLIDEVCT | |
| | | MNDGPHS | |
| R8 | G | ALYTMMKDQYASYVVEKM | 489 |
| | | IDVAEPGQRKIVMHKIRP | |
| PUF R8′ | | HIATLRKYTYGKHILAKL | 496 |
| | | EKYYMKNGVDLG | |

An exemplary 15-mer RNA recognition (15PUF) targeting GCAGCAGCAGCAGCA (SEQ ID NO: 478) comprises the amino acid sequence: GRSRLLEDFRNNRYPNLQLREIAGHIMEFSQDQHGSRFIQLKLERATPAERQLVFNEI LQAAYQLMVDVFGSYVIRKFFEFGSLEQKLALAERIRGHVLSLALQMYGSRVIEKAL EFIPSDQQNEMVRELDGHVLKCVKDQNGCHVVQKCIECVQPQSLQFIIDAFKGQVFA LSTHPYGSRVIRRILEHCLPDQTLPILEELHQHIMEFSQDQHGSRFIELKLERATPAERQ LVFNEILQAAYQLMVDVFGCYVIQKFFEFGSLEQKLALAERIRGHVLSLALQMYGSY VIRKALEFIPSDQQNEMVRELDGHVLKCVKDQNGSYVVEKCIECVQPQSLQFIIDAFK GQVFALSTHPYGCRVIQRILEHCLPDQTLPILEELHQHTEQLVQDQYGSYVIRHVLEH GRPEDKSKIVAEIRGNVLVLSQHKFASNVVEKCVTHASRTERAVLIDEVCTMNDGPH SHTEQLVQDQYGCYVIQHVLEHGRPEDKSKIVAEIRGNVLVLSQHKFASYVVRKCV THASRTERAVLIDEVCTMNDGPHSALYTMMKDQYASYVVEKMIDVAEPGQRKIVM HKIRPHIATLRKYTYGKHILAKLEKYYMKNGVDLG (SEQ ID NO: 552). In some aspects, SEQ ID NO: 552 comprises an architecture proceeding from the N-terminus to the C-terminus according to: R1′-R1-R2-R3-R4-R5-R1-R2-R3-R4-R5-R6-R7-R6-R7-R8-R8′. In some aspects, SEQ ID NO: 552 is comprised of the sequences detailed in Table 32.

TABLE 32
15PUF protein according to SEQ ID NO: 552

| PUF | RNA | | SEQ |
|---|---|---|---|
| Module | Recognition | Amino Acid Sequence | ID NO |
| PUF R1′ | — | GRSRLLEDFRNNRYPNLQLREIAG | 495 |
| R1 | A | HIMEFSQDQHGSRFIQLKLERATPAERQLVFNEILQ | 497 |
| R2 | C | AAYQLMVDVFGSYVIRKFFEFGSLEQKLALAERIRG | 492 |
| R3 | G | HVLSLALQMYGSRVIEKALEFIPSDQQNEMVRELDG | 507 |
| R4 | A | HVLKCVKDQNGCHVVQKCIECVQPQSLQFIIDAFKG | 503 |
| R5 | C | QVFALSTHPYGSRVIRRILEHCLPDQTLPILEELHQ | 514 |
| RI | G | HIMEFSQDQHGSRFIELKLERATPAERQLVFNEILQ | 498 |
| R2 | A | AAYQLMVDVFGCYVIQKFFEFGSLEQKLALAERIRG | 490 |
| R3 | C | HVLSLALQMYGSYVIRKALEFIPSDQQNEMVRELDG | 508 |
| R4 | G | HVLKCVKDQNGSYVVEKCIECVQPQSLQFIIDAFKG | 504 |
| R5 | A | QVFALSTHPYGCRVIQRILEHCLPDQTLPILEELHQ | 512 |
| R6 | C | HTEQLVQDQYGSYVIRHVLEHGRPEDKSKIVAEIRG | 502 |
| R7 | G | NVLVLSQHKFASNVVEKCVTHASRTERAVLIDEVCTMNDG | 510 |
| | | PHS | |
| R6 | A | HTEQLVQDQYGCYVIQHVLEHGRPEDKSKIVAEIRG | 500 |
| R7 | C | NVLVLSQHKFASYVVRKCVTHASRTERAVLIDEVCTMNDG | 511 |
| | | PHS | |
| R8 | G | ALYTMMKDQYASYVVEKMIDVAEPGQRKIVMHKIRP | 489 |
| PUF R8′ | — | HIATLRKYTYGKHILAKLEKYYMKNGVDLG | 496 |

An exemplary 15-mer RNA recognition (15PUF) targeting GCAGCAGCAGCAGCA (SEQ ID NO: 478) comprises the amino acid sequence: GRSRLLEDFRNNRYPNLQLREIAGHIMEFSQDQHGSRFIQLKLERATPAERQLVFNEI LQAAYQLMVDVFGSYVIRKFFEFGSLEQKLALAERIRGHVLSLALQMYGSRVIEKAL EFIPSDQQNEMVRELDGHVLKCVKDQNGCHVVQKCIECVQPQSLQFIIDAFKGQVFA LSTHPYGSRVIRRILEHCLPDQTLPILEELHQHTEQLVQDQYGSYVIEHVLEHGRPED KSKIVAEIRGHIMEFSQDQHGSRFIQLKLERATPAERQLVFNEILQAAYQLMVDVFGS YVIRKFFEFGSLEQKLALAERIRGHVLSLALQMYGSRVIEKALEFIPSDQQNEMVREL DGHVLKCVKDQNGCHVVQKCIECVQPQSLQFIIDAFKGQVFALSTHPYGSRVIRRILE HCLPDQTLPILEELHQHTEQLVQDQYGSYVIEHVLEHGRPEDKSKIVAEIRGNVLVLS QHKFACNVVQKCVTHASRTERAVLIDEVCTMNDGPHSNVLVLSQHKFASYVVRKC VTHASRTERAVLIDEVCTMNDGPHSALYTMMKDQYASYVVEKMIDVAEPGQRKIV MHKIRPHIATLRKYTYGKHILAKLEKYYMKNGVDLG (SEQ ID NO: 553). In some aspects, SEQ ID NO: 553 comprises an architecture proceeding from the N-terminus to the C-terminus according to: R1′-R1-R2-R3-R4-R5-R6-R1-R2-R3-R4-R5-R6-R7-R7-R8-R8′. In some aspects, SEQ ID NO: 553 is comprised of the sequences detailed in Table 33.

TABLE 33
15PUF protein according to SEQ ID NO: 553

| PUF | RNA | | SEQ |
|---|---|---|---|
| Module | Recognition | Amino Acid Sequence | ID NO |
| PUF R1′ | - | GRSRLLEDFRNNRYPNLQLREIAG | 495 |
| R1 | A | HIMEFSQDQHGSRFIQLKLERATPAERQLVFNEILQ | 497 |
| R2 | C | AAYQLMVDVFGSYVIRKFFEFGSLEQKLALAERIRG | 492 |
| R3 | G | HVLSLALQMYGSRVIEKALEFIPSDQQNEMVRELDG | 507 |
| R4 | A | HVLKCVKDQNGCHVVQKCIECVQPQSLQFIIDAFKG | 503 |
| R5 | C | QVFALSTHPYGSRVIRRILEHCLPDQTLPILEELHQ | 514 |
| R6 | G | HTEQLVQDQYGSYVIEHVLEHGRPEDKSKIVAEIRG | 501 |
| R1 | A | HIMEFSQDQHGSRFIQLKLERATPAERQLVFNEILQ | 497 |
| R2 | C | AAYQLMVDVFGSYVIRKFFEFGSLEQKLALAERIRG | 492 |
| R3 | G | HVLSLALQMYGSRVIEKALEFIPSDQQNEMVRELDG | 507 |
| R4 | A | HVLKCVKDQNGCHVVQKCIECVQPQSLQFIIDAFKG | 503 |
| R5 | C | QVFALSTHPYGSRVIRRILEHCLPDQTLPILEELHQ | 514 |
| R6 | G | HTEQLVQDQYGSYVIEHVLEHGRPEDKSKIVAEIRG | 501 |
| R7 | A | NVLVLSQHKFACNVVQKCVTHASRTERAVLIDEVCTMND | 509 |
| | | GPHS | |
| R7 | C | NVLVLSQHKFASYVVRKCVTHASRTERAVLIDEVCTMNDG | 511 |
| | | PHS | |
| R8 | G | ALYTMMKDQYASYVVEKMIDVAEPGQRKIVMHKIRP | 489 |
| PUF R8′ | — | HIATLRKYTYGKHILAKLEKYYMKNGVDLG | 496 |

An exemplary 15-mer RNA recognition targeting GCAGCAGCAGCAGCA (SEQ ID NO: 478) comprises the amino acid sequence: GRSRLLEDFRNNRYPNLQLREIAGHIMEFSQDQHGSRFIQLKLERATPAERQLVFNEI LQAAYQLMVDVFGSYVIRKFFEFGSLEQKLALAERIRGHVLSLALQMYGSRVIEKAL EFIPSDQQNEMVRELDGHVLKCVKDQNGCHVVQKCIECVQPQSLQFIIDAFKGQVFA LSTHPYGSRVIRRILEHCLPDQTLPILEELHQHTEQLVQDQYGSYVIEHVLEHGRPED KSKIVAEIRGNVLVLSQHKFACNVVQKCVTHASRTERAVLIDEVCTMNDGPHSHIME FSQDQHGSRFIRLKLERATPAERQLVFNEILQAAYQLMVDVFGSYVIEKFFEFGSLEQ KLALAERIRGHVLSLALQMYGCRVIQKALEFIPSDQQNEMVRELDGHVLKCVKDQN GSHVVRKCIECVQPQSLQFIIDAFKGQVFALSTHPYGSRVIERILEHCLPDQTLPILEEL HQHTEQLVQDQYGCYVIQHVLEHGRPEDKSKIVAEIRGNVLVLSQHKFASYVVRKC VTHASRTERAVLIDEVCTMNDGPHSALYTMMKDQYASYVVEKMIDVAEPGQRKIV MHKIRPHIATLRKYTYGKHILAKLEKYYMKNGVDLG (SEQ ID NO: 554). In some aspects, SEQ ID NO: 554 comprises an architecture proceeding from the N-terminus to the C-terminus according to: R1′-R1-R2-R3-R4-R5-R6-R7-R1-R2-R3-R4-R5-R6-R7-R8-R8′. In some aspects, SEQ ID NO: 554 is comprised of the sequences detailed in Table 34.

TABLE 34
15PUF protein according to SEQ ID NO: 554

| PUF | RNA | | SEQ |
|---|---|---|---|
| Module | Recognition | Amino Acid Sequence | ID NO |
| PUF R1′ | — | GRSRLLEDFRNNRYPNLQLREIAG | 495 |
| R1 | A | HIMEFSQDQHGSRFIQLKLERATPAERQLVFNEILQ | 497 |
| R2 | C | AAYQLMVDVFGSYVIRKFFEFGSLEQKLALAERIRG | 492 |
| R3 | G | HVLSLALQMYGSRVIEKALEFIPSDQQNEMVRELDG | 507 |
| R4 | A | HVLKCVKDQNGCHVVQKCIECVQPQSLQFIIDAFKG | 503 |
| R5 | C | QVFALSTHPYGSRVIRRILEHCLPDQTLPILEELHQ | 514 |
| R6 | G | HTEQLVQDQYGSYVIEHVLEHGRPEDKSKIVAEIRG | 501 |
| R7 | A | NVLVLSQHKFACNVVQKCVTHASRTERAVLIDEVCTMND | 509 |
| | | GPHS | |
| R1 | C | HIMEFSQDQHGSRFIRLKLERATPAERQLVFNEILQ | 499 |
| R2 | G | AAYQLMVDVFGSYVIEKFFEFGSLEQKLALAERIRG | 491 |
| R3 | A | HVLSLALQMYGCRVIQKALEFIPSDQQNEMVRELDG | 506 |
| R4 | C | HVLKCVKDQNGSHVVRKCIECVQPQSLQFIIDAFKG | 505 |
| R5 | G | QVFALSTHPYGSRVIERILEHCLPDQTLPILEELHQ | 513 |
| R6 | A | HTEQLVQDQYGCYVIQHVLEHGRPEDKSKIVAEIRG | 500 |
| R7 | C | NVLVLSQHKFASYVVRKCVTHASRTERAVLIDEVCTMNDG | 511 |
| | | PHS | |
| R8 | G | ALYTMMKDQYASYVVEKMIDVAEPGQRKIVMHKIRP | 489 |
| PUF R8′ | — | HIATLRKYTYGKHILAKLEKYYMKNGVDLG | 496 |

An exemplary 16-mer RNA recognition (16PUF) targeting GCAGCAGCAGCAGCAG (SEQ ID NO: 479) comprises the amino acid sequence: GRSRLLEDFRNNRYPNLQLREIAGHIMEFSQDQHGSRFIELKLERATPAERQLVFNEIL QAAYQLMVDVFGCYVIQKFFEFGSLEQKLALAERIRGHVLSLALQMYGSYVIRKAL EFIPSDQQNEMVRELDGHVLKCVKDQNGSYVVEKCIECVQPQSLQFIIDAFKGQVFA LSTHPYGCRVIQRILEHCLPDQTLPILEELHQHIMEFSQDQHGSRFIRLKLERATPAER QLVFNEILQAAYQLMVDVFGSYVIEKFFEFGSLEQKLALAERIRGHVLSLALQMYGC RVIQKALEFIPSDQQNEMVRELDGHVLKCVKDQNGSHVVRKCIECVQPQSLQFIIDA FKGQVFALSTHPYGSRVIERILEHCLPDQTLPILEELHQHTEQLVQDQYGCYVIQHVL EHGRPEDKSKIVAEIRGNVLVLSQHKFASYVVRKCVTHASRTERAVLIDEVCTMNDG PHSALYTMMKDQYASYVVEKMIDVAEPGQRKIVMHKIRPHTEQLVQDQYGCYVIQ HVLEHGRPEDKSKIVAEIRGNVLVLSQHKFASYVVRKCVTHASRTERAVLIDEVCTM NDGPHSALYTMMKDQYASYVVEKMIDVAEPGQRKIVMHKIRPHIATLRKYTYGKHI LAKLEKYYMKNGVDLG (SEQ ID NO: 555). In some aspects, SEQ ID NO: 555 comprises an architecture proceeding from the N-terminus to the C-terminus according to: R1′-R1-R2-R3-R4-R5-R1-R2-R3-R4-R5-R6-R7-R8-R6-R7-R8-R8′. In some aspects, SEQ ID NO: 555 is comprised of the sequences detailed in Table 35.

TABLE 35
16PUF protein according to SEQ ID NO: 555

| PUF | RNA | | SEQ |
|---|---|---|---|
| Module | Recognition | Amino Acid Sequence | ID NO |
| PUF R1′ | — | GRSRLLEDFRNNRYPNLQLREIAG | 495 |
| R1 | G | HIMEFSQDQHGSRFIELKLERATPAERQLVFNEILQ | 498 |
| R2 | A | AAYQLMVDVFGCYVIQKFFEFGSLEQKLALAERIRG | 490 |
| R3 | C | HVLSLALQMYGSYVIRKALEFIPSDQQNEMVRELDG | 508 |
| R4 | G | HVLKCVKDQNGSYVVEKCIECVQPQSLQFIIDAFKG | 504 |
| R5 | A | QVFALSTHPYGCRVIQRILEHCLPDQTLPILEELHQ | 512 |
| R1 | C | HIMEFSQDQHGSRFIRLKLERATPAERQLVFNEILQ | 499 |
| R2 | G | AAYQLMVDVFGSYVIEKFFEFGSLEQKLALAERIRG | 491 |
| R3 | A | HVLSLALQMYGCRVIQKALEFIPSDQQNEMVRELDG | 506 |
| R4 | C | HVLKCVKDQNGSHVVRKCIECVQPQSLQFIIDAFKG | 505 |
| R5 | G | QVFALSTHPYGSRVIERILEHCLPDQTLPILEELHQ | 513 |
| R6 | A | HTEQLVQDQYGCYVIQHVLEHGRPEDKSKIVAEIRG | 50 |
| R7 | C | NVLVLSQHKFASYVVRKCVTHASRTERAVLIDEVCTMNDG | 511 |
| | | PHS | |
| R8 | G | ALYTMMKDQYASYVVEKMIDVAEPGQRKIVMHKIRP | 489 |
| R6 | A | HTEQLVQDQYGCYVIQHVLEHGRPEDKSKIVAEIRG | 500 |
| R7 | C | NVLVLSQHKFASYVVRKCVTHASRTERAVLIDEVCTMNDG | 511 |
| | | PHS | |
| R8 | G | ALYTMMKDQYASYVVEKMIDVAEPGQRKIVMHKIRP | 489 |
| PUF R8′ | — | HIATLRKYTYGKHILAKLEKYYMKNGVDLG | 496 |

An exemplary 16-mer RNA recognition (16PUF) targeting GCAGCAGCAGCAGCAG (SEQ ID NO: 479) comprises the amino acid sequence: GRSRLLEDFRNNRYPNLQLREIAGHIMEFSQDQHGSRFIELKLERATPAERQLVFNEIL QAAYQLMVDVFGCYVIQKFFEFGSLEQKLALAERIRGHVLSLALQMYGSYVIRKAL EFIPSDQQNEMVRELDGHVLKCVKDQNGSYVVEKCIECVQPQSLQFIIDAFKGQVFA LSTHPYGCRVIQRILEHCLPDQTLPILEELHQHTEQLVQDQYGSYVIRHVLEHGRPED KSKIVAEIRGHIMEFSQDQHGSRFIELKLERATPAERQLVFNEILQAAYQLMVDVFGC YVIQKFFEFGSLEQKLALAERIRGHVLSLALQMYGSYVIRKALEFIPSDQQNEMVREL DGHVLKCVKDQNGSYVVEKCIECVQPQSLQFIIDAFKGQVFALSTHPYGCRVIQRILE HCLPDQTLPILEELHQHTEQLVQDQYGSYVIRHVLEHGRPEDKSKIVAEIRGNVLVLS QHKFASNVVEKCVTHASRTERAVLIDEVCTMNDGPHSALYTMMKDQYACYVVQK MIDVAEPGQRKIVMHKIRPNVLVLSQHKFASYVVRKCVTHASRTERAVLIDEVCTM NDGPHSALYTMMKDQYASYVVEKMIDVAEPGQRKIVMHKIRPHIATLRKYTYGKHI LAKLEKYYMKNGVDLG (SEQ ID NO: 556). In some aspects, SEQ ID NO: 556 comprises an architecture proceeding from the N-terminus to the C-terminus according to: R1′-R1-R2-R3-R4-R5-R6-R1-R2-R3-R4-R5-R6-R7-R8-R7-R8-R8′. In some aspects, SEQ ID NO: 556 is comprised of the sequences detailed in Table 36.

TABLE 36
16PUF protein according to SEQ ID NO: 556

| PUF | RNA | | SEQ |
|---|---|---|---|
| Module | Recognition | Amino Acid Sequence | ID NO |
| PUF R1′ | — | GRSRLLEDFRNNRYPNLQLREIAG | 495 |
| R1 | G | HIMEFSQDQHGSRFIELKLERATPAERQLVFNEILQ | 498 |
| R2 | A | AAYQLMVDVFGCYVIQKFFEFGSLEQKLALAERIRG | 490 |
| R3 | C | HVLSLALQMYGSYVIRKALEFIPSDQQNEMVRELDG | 508 |
| R4 | G | HVLKCVKDQNGSYVVEKCIECVQPQSLQFIIDAFKG | 504 |
| R5 | A | QVFALSTHPYGCRVIQRILEHCLPDQTLPILEELHQ | 512 |
| R6 | C | HTEQLVQDQYGSYVIRHVLEHGRPEDKSKIVAEIRG | 502 |
| R1 | G | HIMEFSQDQHGSRFIELKLERATPAERQLVFNEILQ | 498 |
| R2 | A | AAYQLMVDVFGCYVIQKFFEFGSLEQKLALAERIRG | 490 |
| R3 | C | HVLSLALQMYGSYVIRKALEFIPSDQQNEMVRELDG | 508 |
| R4 | G | HVLKCVKDQNGSYVVEKCIECVQPQSLQFIIDAFKG | 504 |
| R5 | A | QVFALSTHPYGCRVIQRILEHCLPDQTLPILEELHQ | 512 |
| R6 | C | HTEQLVQDQYGSYVIRHVLEHGRPEDKSKIVAEIRG | 502 |
| R7 | G | NVLVLSQHKFASNVVEKCVTHASRTERAVLIDEVCTMNDG | 510 |
| | | PHS | |
| R8 | A | ALYTMMKDQYACYVVQKMIDVAEPGQRKIVMHKIRP | 493 |
| R7 | C | NVLVLSQHKFASYVVRKCVTHASRTERAVLIDEVCTMNDG | 511 |
| | | PHS | |
| R8 | G | ALYTMMKDQYASYVVEKMIDVAEPGQRKIVMHKIRP | 489 |
| PUF R8′ | — | HIATLRKYTYGKHILAKLEKYYMKNGVDLG | 496 |

An exemplary 16-mer RNA recognition 16PUF targeting GCAGCAGCAGCAGCAG (SEQ ID NO: 479) comprises the amino acid sequence: GRSRLLEDFRNNRYPNLQLREIAGHIMEFSQDQHGSRFIELKLERATPAERQLVFNEIL QAAYQLMVDVFGCYVIQKFFEFGSLEQKLALAERIRGHVLSLALQMYGSYVIRKAL EFIPSDQQNEMVRELDGHVLKCVKDQNGSYVVEKCIECVQPQSLQFIIDAFKGQVFA LSTHPYGCRVIQRILEHCLPDQTLPILEELHQHTEQLVQDQYGSYVIRHVLEHGRPED KSKIVAEIRGNVLVLSQHKFASNVVEKCVTHASRTERAVLIDEVCTMNDGPHSALYT MMKDQYACYVVQKMIDVAEPGQRKIVMHKIRPHIMEFSQDQHGSRFIRLKLERATP AERQLVFNEILQAAYQLMVDVFGSYVIEKFFEFGSLEQKLALAERIRGHVLSLALQM YGCRVIQKALEFIPSDQQNEMVRELDGHVLKCVKDQNGSHVVRKCIECVQPQSLQF IIDAFKGQVFALSTHPYGSRVIERILEHCLPDQTLPILEELHQHTEQLVQDQYGCYVIQ HVLEHGRPEDKSKIVAEIRGNVLVLSQHKFASYVVRKCVTHASRTERAVLIDEVCTM NDGPHSALYTMMKDQYASYVVEKMIDVAEPGQRKIVMHKIRPHIATLRKYTYGKHI LAKLEKYYMKNGVDLG (SEQ ID NO: 557). In some aspects, SEQ ID NO: 557 comprises an architecture proceeding from the N-terminus to the C-terminus according to: R1′-R1-R2-R3-R4-R5-R6-R7-R8-R1-R2-R3-R4-R5-R6-R7-R8-R8′. In some aspects, SEQ ID NO: 557 is comprised of the sequences detailed in Table 37.

TABLE 37
16PUF protein according to SEQ ID NO: 557

| PUF | RNA | | SEQ |
|---|---|---|---|
| Module | Recognition | Amino Acid Sequence | ID NO |
| PUF R1′ | — | GRSRLLEDFRNNRYPNLQLREIAG | 495 |
| R1 | G | HIMEFSQDQHGSRFIELKLERATPAERQLVFNEILQ | 498 |
| R2 | A | AAYQLMVDVFGCYVIQKFFEFGSLEQKLALAERIRG | 490 |
| R3 | C | HVLSLALQMYGSYVIRKALEFIPSDQQNEMVRELDG | 508 |
| R4 | G | HVLKCVKDQNGSYVVEKCIECVQPQSLQFIIDAFKG | 504 |
| R5 | A | QVFALSTHPYGCRVIQRILEHCLPDQTLPILEELHQ | 512 |
| R6 | C | HTEQLVQDQYGSYVIRHVLEHGRPEDKSKIVAEIRG | 502 |
| R7 | G | NVLVLSQHKFASNVVEKCVTHASRTERAVLIDEVCTMNDG | 510 |
| | | PHS | |
| R8 | A | ALYTMMKDQYACYVVQKMIDVAEPGQRKIVMHKIRP | 493 |
| R1 | C | HIMEFSQDQHGSRFIRLKLERATPAERQLVFNEILQ | 499 |
| R2 | G | AAYQLMVDVFGSYVIEKFFEFGSLEQKLALAERIRG | 491 |
| R3 | A | HVLSLALQMYGCRVIQKALEFIPSDQQNEMVRELDG | 506 |
| R4 | C | HVLKCVKDQNGSHVVRKCIECVQPQSLQFIIDAFKG | 505 |
| R5 | G | QVFALSTHPYGSRVIERILEHCLPDQTLPILEELHQ | 513 |
| R6 | A | HTEQLVQDQYGCYVIQHVLEHGRPEDKSKIVAEIRG | 500 |
| R7 | C | NVLVLSQHKFASYVVRKCVTHASRTERAVLIDEVCTMNDG | 511 |
| | | PHS | |
| R8 | G | ALYTMMKDQYASYVVEKMIDVAEPGQRKIVMHKIRP | 489 |
| PUF R8′ | — | HIATLRKYTYGKHILAKLEKYYMKNGVDLG | 496 |

An exemplary 8-mer RNA recognition (8PUFtargeting GCAGCAGC (SEQ ID NO: 476 corn rises the amino acid sequence:

(SEQ ID NO: 568)
GRSRLLEDFRNNRYPNLQLREIAGHIMEFSQDQHGSRFIRLKLERATPA
ERQLVFNEILQAAYQLMVDVFGSYVIEKFFEFGSLEQKLALAERIRGHV
LSLALQMYGCRVIQKALEFIPSDQQNEMVRELDGHVLKCVKDQNGSHVV
RKCIECVQPQSLQFIIDAFKGQVFALSTHPYGSRVIERILEHCLPDQTL
PILEELHQHTEQLVQDQYGCYVIQHVLEHGRPEDKSKIVAEIRGNVLVL
SQHKFASYVVRKCVTHASRTERAVLIDEVCTMNDGPHSALYTMMKDQYA
SYVVEKMIDVAEPGQRKIVMHKIRPHIATLRKYTYGKHILAKLEKYYMK
NGVDLG.

An exemplary 14-mer RNA recognition (14PUF) targeting GCAGCAGCAGCAGC (SEQ ID NO: 477) comprises the amino acid sequence:

(SEQ ID NO: 569)
GRSRLLEDFRNNRYPNLQLREIAGHIMEFSQDQHGSRFIRLKLERATPA
ERQLVFNEILQAAYQLMVDVFGSYVIEKFFEFGSLEQKLALAERIRGHV
LSLALQMYGCRVIQKALEFIPSDQQNEMVRELDGHVLKCVKDQNGSYVV
RKCIECVQPQSLQFIIDAFKGQVFALSTHPYGSRVIERILEHCLPDQTL
PILEELHQHIMEFSQDQHGSRFIQLKLERATPAERQLVFNEILQAAYQL
MVDVFGSYVIRKFFEFGSLEQKLALAERIRGHVLSLALQMYGSRVIEKA
LEFIPSDQQNEMVRELDGHVLKCVKDQNGCHVVQKCIECVQPQSLQFII
DAFKGQVFALSTHPYGSRVIRRILEHCLPDQTLPILEELHQHTEQLVQD
QYGSYVIEHVLEHGRPEDKSKIVAEIRGHTEQLVQDQYGCYVIQHVLEH
GRPEDKSKIVAEIRGNVLVLSQHKFASYVVRKCVTHASRTERAVLIDEV
CTMNDGPHSALYTMMKDQYASYVVEKMIDVAEPGQRKIVMHKIRPHIAT
LRKYTYGKHILAKLEKYYMKNGVDLG.

An exemplary 14-mer RNA recognition (14PUF) targeting GCAGCAGCAGCAGC (SEQ ID NO: 477) comprises the amino acid sequence:

(SEQ ID NO: 570)
GRSRLLEDFRNNRYPNLQLREIAGHIMEFSQDQHGSRFIRLKLERATPA
ERQLVFNEILQAAYQLMVDVFGSYVIEKFFEFGSLEQKLALAERIRGHV
LSLALQMYGCRVIQKALEFIPSDQQNEMVRELDGHVLKCVKDQNGSYVV
RKCIECVQPQSLQFIIDAFKGQVFALSTHPYGSRVIERILEHCLPDQTL
PILEELHQHTEQLVQDQYGCYVIQHVLEHGRPEDKSKIVAEIRGHIMEF
SQDQHGSRFIRLKLERATPAERQLVFNEILQAAYQLMVDVFGSYVIEKF
FEFGSLEQKLALAERIRGHVLSLALQMYGCRVIQKALEFIPSDQQNEMV
RELDGHVLKCVKDQNGSYVVRKCIECVQPQSLQFIIDAFKGQVFALSTH
PYGSRVIERILEHCLPDQTLPILEELHQHTEQLVQDQYGCYVIQHVLEH
GRPEDKSKIVAEIRGNVLVLSQHKFASYVVRKCVTHASRTERAVLIDEV
CTMNDGPHSALYTMMKDQYASYVVEKMIDVAEPGQRKIVMHKIRPHIAT
LRKYTYGKHILAKLEKYYMKNGVDLG.

An exemplary 15-mer RNA recognition (15PUF) targeting GCAGCAGCAGCAGCA (SEQ ID NO: 478) comprises the amino acid sequence:

(SEQ ID NO: 571)
GRSRLLEDFRNNRYPNLQLREIAGHIMEFSQDQHGSRFIQLKLERATPA
ERQLVFNEILQAAYQLMVDVFGSYVIRKFFEFGSLEQKLALAERIRGHV
LSLALQMYGSRVIEKALEFIPSDQQNEMVRELDGHVLKCVKDQNGCHVV
QKCIECVQPQSLQFIIDAFKGQVFALSTHPYGSRVIRRILEHCLPDQTL
PILEELHQHTEQLVQDQYGSYVIEHVLEHGRPEDKSKIVAEIRGNVLVL
SQHKFACNVVQKCVTHASRTERAVLIDEVCTMNDGPHSHIMEFSQDQHG
SRFIRLKLERATPAERQLVFNEILQAAYQLMVDVFGSYVIEKFFEFGSL
EQKLALAERIRGHVLSLALQMYGCRVIQKALEFIPSDQQNEMVRELDGH
VLKCVKDQNGSYVVRKCIECVQPQSLQFIIDAFKGQVFALSTHPYGSRV
IERILEHCLPDQTLPILEELHQHTEQLVQDQYGCYVIQHVLEHGRPEDK
SKIVAEIRGNVLVLSQHKFASYVVRKCVTHASRTERAVLIDEVCTMNDG
PHSALYTMMKDQYASYVVEKMIDVAEPGQRKIVMHKIRPHIATLRKYTY
GKHILAKLEKYYMKNGVDLG.

An exemplary 16-mer RNA recognition (16PUF) targeting GCAGCAGCAGCAGCAG (SEQ ID NO: 479) comprises the amino acid sequence:

(SEQ ID NO: 572)
GRSRLLEDFRNNRYPNLQLREIAGHIMEFSQDQHGSRFIELKLERATPA
ERQLVFNEILQAAYQLMVDVFGCYVIQKFFEFGSLEQKLALAERIRGHV
LSLALQMYGSYVIRKALEFIPSDQQNEMVRELDGHVLKCVKDQNGSYVV
EKCIECVQPQSLQFIIDAFKGQVFALSTHPYGCRVIQRILEHCLPDQTL
PILEELHQHIMEFSQDQHGSRFIRLKLERATPAERQLVFNEILQAAYQL
MVDVFGSYVIEKFFEFGSLEQKLALAERIRGHVLSLALQMYGCRVIQKA
LEFIPSDQQNEMVRELDGHVLKCVKDQNGSYVVRKCIECVQPQSLQFII
DAFKGQVFALSTHPYGSRVIERILEHCLPDQTLPILEELHQHTEQLVQD
QYGCYVIQHVLEHGRPEDKSKIVAEIRGNVLVLSQHKFASYVVRKCVTH
ASRTERAVLIDEVCTMNDGPHSALYTMMKDQYASYVVEKMIDVAEPGQR
KIVMHKIRPHTEQLVQDQYGCYVIQHVLEHGRPEDKSKIVAEIRGNVLV
LSQHKFASYVVRKCVTHASRTERAVLIDEVCTMNDGPHSALYTMMKDQY
ASYVVEKMIDVAEPGQRKIVMHKIRPHIATLRKYTYGKHILAKLEKYYM
KNGVDLG

An exemplary 16-mer RNA recognition (16PUF) targeting GCAGCAGCAGCAGCAG (SEQ ID NO: 479) comprises the amino acid sequence:

(SEQ ID NO: 573)
GRSRLLEDFRNNRYPNLQLREIAGHIMEFSQDQHGSRFIELKLERATPA
ERQLVFNEILQAAYQLMVDVFGCYVIQKFFEFGSLEQKLALAERIRGHV
LSLALQMYGSYVIRKALEFIPSDQQNEMVRELDGHVLKCVKDQNGSYVV
EKCIECVQPQSLQFIIDAFKGQVFALSTHPYGCRVIQRILEHCLPDQTL
PILEELHQHTEQLVQDQYGSYVIRHVLEHGRPEDKSKIVAEIRGNVLVL
SQHKFASNVVEKCVTHASRTERAVLIDEVCTMNDGPHSALYTMMKDQYA
CYVVQKMIDVAEPGQRKIVMHKIRPHIMEFSQDQHGSRFIRLKLERATP
AERQLVFNEILQAAYQLMVDVFGSYVIEKFFEFGSLEQKLALAERIRGH
VLSLALQMYGCRVIQKALEFIPSDQQNEMVRELDGHVLKCVKDQNGSYV
VRKCIECVQPQSLQFIIDAFKGQVFALSTHPYGSRVIERILEHCLPDQT
LPILEELHQHTEQLVQDQYGCYVIQHVLEHGRPEDKSKIVAEIRGNVLV
LSQHKFASYVVRKCVTHASRTERAVLIDEVCTMNDGPHSALYTMMKDQY
ASYVVEKMIDVAEPGQRKIVMHKIRPHIATLRKYTYGKHILAKLEKYYM
KNGVDLG

In some embodiments, nucleic acid sequences encoding PUF proteins of the disclosure are codon optimized nucleic acid sequences. In some embodiments, the codon optimized sequence encoding a PUF protein exhibits at least 5%, at least 10%, at least 20%, at least 30%, at least 50%, at least 75%, at least 100%, at least 200%, at least 300%, at least 500%, or at least 1000% increased expression in a human subject relative to a wild-type or non-codon optimized nucleic acid sequence. In some embodiments, an 8PUF protein of the disclosure is encoded by a nucleic acid sequences comprising SEQ ID NO: 576 or 581. In some embodiments, a nucleotide sequence encoding a CAG-targeting fusion protein comprises, from 5′ to 3′: a flag tag, H2B nuclear localization sequence, an 8PUF, and an E17 nuclease is set forth in SEQ ID NO: 578. In some embodiments, a nucleotide sequence encoding a CAG-targeting fusion protein comprises, from 5′ to 3′: a H2B nuclear localization sequence, an 8PUF, an E17 nuclease, and a PKI NES is set forth in SEQ ID NO: 575. In some embodiments, a nucleotide sequence encoding a CAG-targeting fusion protein comprises, from 5′ to 3′: a H2B nuclear localization sequence, an 8PUF, and an E17 nuclease in SEQ ID NO: 577. In some embodiments, a nucleotide sequence encoding a CAG-targeting fusion protein comprises, from 5′ to 3′: an H2B nuclear localization sequence, an 8PUF, and an E17 nuclease is set forth in SEQ ID NO: 579. In some embodiments, a nucleotide sequence encoding a CAG-targeting fusion protein comprises, from 5′ to 3′: an H2B nuclear localization sequence, an 8PUF, an E17 nuclease and PKI nuclear export sequences is set forth in SEQ ID NO: 574. In some embodiments, a nucleotide sequence encoding a CAG-targeting fusion protein comprises, from 5′ to 3′: an RB NLS, an 8PUF and an E17 nuclease is set forth in SEQ ID NO: 580 or 582.

In some embodiments, nucleic acid sequences encoding PUF proteins of the disclosure are codon optimized nucleic acid sequences. In some embodiments, the codon optimized sequence encoding a PUF protein exhibits at least 5%, at least 10%, at least 20%, at least 30%, at least 50%, at least 75%, at least 100%, at least 200%, at least 300%, at least 500%, or at least 1000% increased translation in a human subject relative to a wild-type or non-codon optimized nucleic acid sequence.

In some aspects, a codon optimized nucleic acid sequence encoding a PUF protein such as those put forth in SEQ ID NOs: 574-582 exhibits increased stability. In some aspects, a codon optimized nucleic acid sequence encoding a PUF protein exhibits increased stability through increased resistance to hydrolysis. In some embodiments, the codon optimized sequence encoding a PUF protein exhibits at least 5%, at least 10%, at least 20%, at least 30%, at least 50%, at least 75%, at least 100%, at least 200%, at least 300%, at least 500%, or at least 1000% increased stability relative to a wild-type or non-codon optimized nucleic acid sequence. In some embodiments, the codon optimized sequence encoding a PUF protein exhibits at least 5%, at least 10%, at least 20%, at least 30%, at least 50%, at least 75%, at least 100%, at least 200%, at least 300%, at least 500%, or at least 1000% increased resistance to hydrolysis in a human subject relative to a wild-type or non-codon optimized nucleic acid sequence.

In some aspects, a codon optimized nucleic acid sequence encoding a PUF protein such as those put forth in SEQ ID NOs: 574-582, can comprise no donor splice sites. In some aspects, a codon optimized nucleic acid sequence encoding a PUF protein can comprise no more than about one, or about two, or about three, or about four, or about five, or about six, or about seven, or about eight, or about nine, or about ten donor splice sites. In some aspects, a codon optimized nucleic acid sequence encoding a PUF protein comprises at least one, or at least two, or at least three, or at least four, or at least five, or at least six, or at least seven, or at least eight, or at least nine, or at least ten fewer donor splice sites as compared to a non-codon optimized nucleic acid sequence encoding the PUF protein.

Without wishing to be bound by theory, the removal of donor splice sites in the codon optimized nucleic acid sequence can unexpectedly and unpredictably increase expression of the PUF protein in vivo, as cryptic splicing is prevented. Moreover, cryptic splicing may vary between different subjects, meaning that the expression level of the PUF protein comprising donor splice sites may unpredictably vary between different subjects. Such unpredictability is unacceptable in the context of human therapy. Accordingly, the codon optimized nucleic acid sequences put forth in SEQ ID NOs: 574-582, which lacks donor splice sites, unexpectedly and surprisingly allows for increased expression of the PUF protein in human subjects and regularizes expression of the PUF protein across different human subjects.

In some aspects, a codon optimized nucleic acid sequence encoding a PUF protein, such as those put forth in SEQ ID NOs: 574-582, can have a GC content that differs from the GC content of the non-codon optimized nucleic acid sequence encoding the PUF protein. In some aspects, the GC content of a codon optimized nucleic acid sequence encoding a PUF protein is more evenly distributed across the entire nucleic acid sequence, as compared to the non-codon optimized nucleic acid sequence encoding the PUF protein.

Without wishing to be bound by theory, by more evenly distributing the GC content across the entire nucleic acid sequence, the codon optimized nucleic acid sequence exhibits a more uniform melting temperature (“Tm”) across the length of the transcript. The uniformity of melting temperature results unexpectedly in increased expression of the codon optimized nucleic acid in a human subject, as transcription and/or translation of the nucleic acid sequence occurs with less stalling of the polymerase and/or ribosome.

In some aspects, a codon optimized nucleic acid sequence encoding a PUF protein, such as those put forth in SEQ ID NOs: 574-582, can have fewer repressive microRNA target binding sites as compared to the non-codon optimized nucleic acid sequence encoding the PUF protein. In some aspects, a codon optimized nucleic acid sequence encoding a PUF protein can have at least one, or at least two, or at least three, or at least four, or at least five, or at least six, or at least seven, or at least eight, or at least nine, or at least ten, or at least ten fewer repressive microRNA target binding sites as compared to the non-codon optimized nucleic acid sequence the PUF protein.

Without wishing to be bound by theory, by having fewer repressive microRNA target binding sites, the codon optimized nucleic acid sequence encoding a PUF protein unexpectedly exhibits increased expression in a human subject.

In some embodiments, an 8PUF protein can be encoded by a nucleic acid sequence comprising:

(SEQ ID NO: 452)
GGACGAAGCCGACTCTTGGAAGACTTCAGAAACAATCGGTATCCGAACC
TTCAGCTGAGAGAAATTGCTGGTCACATCATGGAATTTTCTCAAGATCA
ACATGGAAGCCGGTTTATTGAACTTAAACTCGAACGAGCCACCCCGGCC
GAAAGGCAATTGGTGTTCAATGAAATTCTTCAGGCCGCATACCAACTCA
TGGTTGATGTTTTTGGGAACTATGTTATTCAAAAGTTTTTTGAGTTCGG
GTCACTGGAGCAAAAGTTGGCATTGGCAGAGCGAATCCGGGGCCATGTT
CTGAGCCTCGCTCTCCAAATGTACGGTAGTTATGTCATTCGCAAAGCAC
TCGAGTTCATACCATCAGATCAACAGAATGAGATGGTGCGGGAGCTGGA
TGGGCATGTTTTGAAATGCGTGAAAGACCAAAACGGTAGCTACGTAGTT
GAGAAATGCATCGAATGCGTCCAACCACAGTCTCTCCAATTTATTATAG
ATGCATTTAAGGGTCAGGTTTTCGCGCTTTCTACGCACCCGTATGGGAA
CCGAGTGATTCAGAGAATCTTGGAGCACTGCCTGCCGGATCAGACACTC
CCTATCTTGGAGGAATTGCACCAGCATACCGAACAATTGGTGCAAGATC
AATACGGTTCATATGTTATTCGGCACGTTCTTGAGCATGGAAGGCCAGA
GGACAAGTCAAAGATCGTCGCTGAGATTAGAGGTAACGTATTGGTGCTC
TCACAACACAAATTTGCATCTAATGTGGTGGAGAAATGTGTTACTCATG
CTTCTAGAACGGAAAGGGCAGTTCTCATAGACGAAGTTTGCACAATGAA
TGATGGTCCTCATAGCGCACTTTATACCATGATGAAGGACCAGTATGCA
AACTATGTCGTCCAGAAAATGATCGATGTGGCGGAGCCCGGTCAACGGA
AAATCGTGATGCACAAAATCCGACCTCACATTGCTACACTCAGAAAATA
CACGTATGGAAAACATATTCTGGCTAAGCTGGAGAAATATTACATGAAG
AATGGAGTGGATCTGGGG.

An exemplary 14-mer RNA recognition (14PUMBY) targeting CAGCAGCAGCAGCA (SEQ ID NO: 454) comprises the amino acid sequence: GRSRLLEDFRNNRYPNLQLREIAGHTEQLVQDQYGCYVIQHVLEHGRPEDKSKIVAEIRGHT EQLVQDQYGSYVIRHVLEHGRPEDKSKIVAEIRGHTEQLVQDQYGSYVIEHVLEHGRPEDKS KIVAEIRGHTEQLVQDQYGCYVIQHVLEHGRPEDKSKIVAEIRGHTEQLVQDQYGSYVIRHV LEHGRPEDKSKIVAEIRGHTEQLVQDQYGSYVIEHVLEHGRPEDKSKIVAEIRGHTEQLVQDQ YGCYVIQHVLEHGRPEDKSKIVAEIRGHTEQLVQDQYGSYVIRHVLEHGRPEDKSKIVAEIRG HTEQLVQDQYGSYVIEHVLEHGRPEDKSKIVAEIRGHTEQLVQDQYGCYVIQHVLEHGRPED KSKIVAEIRGHTEQLVQDQYGSYVIRHVLEHGRPEDKSKIVAEIRGHTEQLVQDQYGSYVIEH VLEHGRPEDKSKIVAEIRGHTEQLVQDQYGCYVIQHVLEHGRPEDKSKIVAEIRGHTEQLVQ DQYGSYVIRHVLEHGRPEDKSKIVAEIRGHIATLRKYTYGKHILAKLEKYYMKNGVDLG (SEQ ID NO: 548). In some aspects, SEQ ID NO: 548 comprises an architecture proceeding from the N-terminus to the C-terminus according to: R1′-R6-R6-R6-R6-R6-R6-R6-R6-R6-R6-R6-R6-R6-R6-R8′. In some aspects, SEQ ID NO: 548 is comprised of the sequences detailed in Table 38.

TABLE 38
14Pumby protein according to SEQ ID NO: 548

| PUF | RNA | | SEQ ID |
|---|---|---|---|
| Module | Recognition | Amino Acid Sequence | NO: |
| PUF R1′ | — | GRSRLLEDFRNNRYPNLQLREIAG | 495 |
| R6 | A | HTEQLVQDQYGCYVIQHVLEHGRPEDKSKIVAEIRG | 500 |
| R6 | C | HTEQLVQDQYGSYVIRHVLEHGRPEDKSKIVAEIRG | 502 |
| R6 | G | HTEQLVQDQYGSYVIEHVLEHGRPEDKSKIVAEIRG | 501 |
| R6 | A | HTEQLVQDQYGCYVIQHVLEHGRPEDKSKIVAEIRG | 500 |
| R6 | C | HTEQLVQDQYGSYVIRHVLEHGRPEDKSKIVAEIRG | 502 |
| R6 | G | HTEQLVQDQYGSYVIEHVLEHGRPEDKSKIVAEIRG | 501 |
| R6 | A | HTEQLVQDQYGCYVIQHVLEHGRPEDKSKIVAEIRG | 500 |
| R6 | C | HTEQLVQDQYGSYVIRHVLEHGRPEDKSKIVAEIRG | 502 |
| R6 | G | HTEQLVQDQYGSYVIEHVLEHGRPEDKSKIVAEIRG | 501 |
| R6 | A | HTEQLVQDQYGCYVIQHVLEHGRPEDKSKIVAEIRG | 500 |
| R6 | C | HTEQLVQDQYGSYVIRHVLEHGRPEDKSKIVAEIRG | 502 |
| R6 | G | HTEQLVQDQYGSYVIEHVLEHGRPEDKSKIVAEIRG | 501 |
| R6 | A | HTEQLVQDQYGCYVIQHVLEHGRPEDKSKIVAEIRG | 500 |
| R6 | C | HTEQLVQDQYGSYVIRHVLEHGRPEDKSKIVAEIRG | 502 |
| PUF R8′ | — | HIATLRKYTYGKHILAKLEKYYMKNGVDLG | 496 |

An exemplary 14-mer RNA recognition (14PUMBY) targeting GCAGCAGCAGCAGC (SEQ ID NO: 477) comprises the amino acid sequence: GRSRLLEDFRNNRYPNLQLREIAGHTEQLVQDQYGSYVIRHVLEHGRPEDKSKIVAEIRGHTE QLVQDQYGSYVIEHVLEHGRPEDKSKIVAEIRGHTEQLVQDQYGCYVIQHVLEHGRPEDKSK IVAEIRGHTEQLVQDQYGSYVIRHVLEHGRPEDKSKIVAEIRGHTEQLVQDQYGSYVIEHVLE HGRPEDKSKIVAEIRGHTEQLVQDQYGCYVIQHVLEHGRPEDKSKIVAEIRGHTEQLVQDQY GSYVIRHVLEHGRPEDKSKIVAEIRGHTEQLVQDQYGSYVIEHVLEHGRPEDKSKIVAEIRGH TEQLVQDQYGCYVIQHVLEHGRPEDKSKIVAEIRGHTEQLVQDQYGSYVIRHVLEHGRPEDK SKIVAEIRGHTEQLVQDQYGSYVIEHVLEHGRPEDKSKIVAEIRGHTEQLVQDQYGCYVIQHV LEHGRPEDKSKIVAEIRGHTEQLVQDQYGSYVIRHVLEHGRPEDKSKIVAEIRGHTEQLVQDQ YGSYVIEHVLEHGRPEDKSKIVAEIRGHIATLRKYTYGKHILAKLEKYYMKNGVDLG (SEQ ID NO: 558). In some aspects, SEQ ID NO: 558 comprises an architecture proceeding from the N-terminus to the C-terminus according to: R1′-R6-R6-R6-R6-R6-R6-R6-R6-R6-R6-R6-R6-R6-R6-R8′. In some aspects, SEQ ID NO: 558 is comprised of the sequences detailed in Table 39.

TABLE 39
14Pumby protein according to SEQ ID NO: 558

| PUF | RNA | | SEQ ID |
|---|---|---|---|
| Module | Recognition | Amino Acid Sequence | NO: |
| PUF R1′ | — | GRSRLLEDFRNNRYPNLQLREIAG | 495 |
| R6 | C | HTEQLVQDQYGSYVIRHVLEHGRPEDKSKIVAEIRG | 502 |
| R6 | G | HTEQLVQDQYGSYVIEHVLEHGRPEDKSKIVAEIRG | 501 |
| R6 | A | HTEQLVQDQYGCYVIQHVLEHGRPEDKSKIVAEIRG | 500 |
| R6 | C | HTEQLVQDQYGSYVIRHVLEHGRPEDKSKIVAEIRG | 502 |
| R6 | G | HTEQLVQDQYGSYVIEHVLEHGRPEDKSKIVAEIRG | 501 |
| R6 | A | HTEQLVQDQYGCYVIQHVLEHGRPEDKSKIVAEIRG | 500 |
| R6 | C | HTEQLVQDQYGSYVIRHVLEHGRPEDKSKIVAEIRG | 502 |
| R6 | G | HTEQLVQDQYGSYVIEHVLEHGRPEDKSKIVAEIRG | 501 |
| R6 | A | HTEQLVQDQYGCYVIQHVLEHGRPEDKSKIVAEIRG | 500 |
| R6 | C | HTEQLVQDQYGSYVIRHVLEHGRPEDKSKIVAEIRG | 502 |
| R6 | G | HTEQLVQDQYGSYVIEHVLEHGRPEDKSKIVAEIRG | 501 |
| R6 | A | HTEQLVQDQYGCYVIQHVLEHGRPEDKSKIVAEIRG | 500 |
| R6 | C | HTEQLVQDQYGSYVIRHVLEHGRPEDKSKIVAEIRG | 502 |
| R6 | G | HTEQLVQDQYGSYVIEHVLEHGRPEDKSKIVAEIRG | 501 |
| PUF R8′ | — | HIATLRKYTYGKHILAKLEKYYMKNGVDLG | 496 |

An exemplary 14-mer RNA recognition (14PUMBY) targeting AGCAGCAGCAGCAG (SEQ ID NO: 473) comprises the amino acid sequence: GRSRLLEDFRNNRYPNLQLREIAGHTEQLVQDQYGSYVIEHVLEHGRPEDKSKIVAEIRGHTE QLVQDQYGCYVIQHVLEHGRPEDKSKIVAEIRGHTEQLVQDQYGSYVIRHVLEHGRPEDKSK IVAEIRGHTEQLVQDQYGSYVIEHVLEHGRPEDKSKIVAEIRGHTEQLVQDQYGCYVIQHVLE HGRPEDKSKIVAEIRGHTEQLVQDQYGSYVIRHVLEHGRPEDKSKIVAEIRGHTEQLVQDQY GSYVIEHVLEHGRPEDKSKIVAEIRGHTEQLVQDQYGCYVIQHVLEHGRPEDKSKIVAEIRGH TEQLVQDQYGSYVIRHVLEHGRPEDKSKIVAEIRGHTEQLVQDQYGSYVIEHVLEHGRPEDK SKIVAEIRGHTEQLVQDQYGCYVIQHVLEHGRPEDKSKIVAEIRGHTEQLVQDQYGSYVIRH VLEHGRPEDKSKIVAEIRGHTEQLVQDQYGSYVIEHVLEHGRPEDKSKIVAEIRGHTEQLVQD QYGCYVIQHVLEHGRPEDKSKIVAEIRGHIATLRKYTYGKHILAKLEKYYMKNGVDLG (SEQ ID NO: 547). In some aspects, SEQ ID NO: 547 comprises an architecture proceeding from the N-terminus to the C-terminus according to: R1′-R6-R6-R6-R6-R6-R6-R6-R6-R6-R6-R6-R6-R6-R6-R8′. In some aspects, SEQ ID NO: 547 is comprised of the sequences detailed in Table 40.

TABLE 40
14Pumby protein according to SEQ ID NO: 547

| PUF | RNA | | |
|---|---|---|---|
| Module | Recognition | Amino Acid Sequence | SEQ ID NO: |
| PUF R1′ | — | GRSRLLEDFRNNRYPNLQLREIAG | 495 |
| R6 | G | HTEQLVQDQYGSYVIEHVLEHGRPEDKSKIVAEIRG | 501 |
| R6 | A | HTEQLVQDQYGCYVIQHVLEHGRPEDKSKIVAEIRG | 500 |
| R6 | C | HTEQLVQDQYGSYVIRHVLEHGRPEDKSKIVAEIRG | 502 |
| R6 | G | HTEQLVQDQYGSYVIEHVLEHGRPEDKSKIVAEIRG | 501 |
| R6 | A | HTEQLVQDQYGCYVIQHVLEHGRPEDKSKIVAEIRG | 500 |
| R6 | C | HTEQLVQDQYGSYVIRHVLEHGRPEDKSKIVAEIRG | 502 |
| R6 | G | HTEQLVQDQYGSYVIEHVLEHGRPEDKSKIVAEIRG | 501 |
| R6 | A | HTEQLVQDQYGCYVIQHVLEHGRPEDKSKIVAEIRG | 500 |
| R6 | C | HTEQLVQDQYGSYVIRHVLEHGRPEDKSKIVAEIRG | 502 |
| R6 | G | HTEQLVQDQYGSYVIEHVLEHGRPEDKSKIVAEIRG | 501 |
| R6 | A | HTEQLVQDQYGCYVIQHVLEHGRPEDKSKIVAEIRG | 500 |
| R6 | C | HTEQLVQDQYGSYVIRHVLEHGRPEDKSKIVAEIRG | 502 |
| R6 | G | HTEQLVQDQYGSYVIEHVLEHGRPEDKSKIVAEIRG | 501 |
| R6 | A | HTEQLVQDQYGCYVIQHVLEHGRPEDKSKIVAEIRG | 500 |
| PUF R8′ | — | HIATLRKYTYGKHILAKLEKYYMKNGVDLG | 496 |

In some aspects, fusion proteins of the disclosure comprise a PUF according to SEQ ID NOs: 444-451, 461, 480-488, or 549-557. In some aspects, fusion proteins of the disclosure are arranged from N- to C-terminus as set forth in any one of Tables 41-49.

TABLE 41
Exemplary 8PUF targeting CAG Fusion Protein

| Plasmid | RNA | |
|---|---|---|
| Element | Recognition | Amino Acid Sequence |
| 8PUF | CAGCAGCA | GRSRLLEDFRNNRYPNLQLREIAGHI |
| Frame 1 | | MEFSQDQHGSRFIQLKLERATPAERQ |
| | | LVFNEILQAAYQLMVDVFGSYVIRKF |
| | | FEFGSLEQKLALAERIRGHVLSLALQ |
| | | MYGSRVIEKALEFIPSDQQNEMVREL |
| | | DGHVLKCVKDQNGCYVVQKCIECVQP |
| | | QSLQFIIDAFKGQVFALSTHPYGSRV |
| | | IRRILEHCLPDQTLPILEELHQHTEQ |
| | | LVQDQYGSYVIEHVLEHGRPEDKSKI |
| | | VAEIRGNVLVLSQHKFACNVVQKCVT |
| | | HASRTERAVLIDEVCTMNDGPHSALY |
| | | TMMKDQYASYVVRKMIDVAEPGQRKI |
| | | VMHKIRPHIATLRKYTYGKHILAKLE |
| | | KYYMKNGVDLG |
| | | (SEQ ID NO: 480) |
| Linker | — | VDTANGS |
| | | (SEQ ID NO: 411) |
| E17 | — | GGGTPKAPNLEPPLPEEEKEGSDLRP |
| endonuclease | | VVIDGSNVAMSHGNKEVFSCRGILLA |
| | | VNWFLERGHTDITVFVPSWRKEQPRP |
| | | DVPITDQHILRELEKKKILVFTPSRR |
| | | VGGKRVVCYDDRFIVKLAYESDGIVV |
| | | SNDTYRDLQGERQEWKRFIEERLLMY |
| | | SFVNDKFMPPDDPLGRHGPSLDNFLR |
| | | KKPLTLE |
| | | (SEQ ID NO: 358) |

TABLE 42
Exemplary 8PUF targeting CAG Fusion Protein

| Plasmid | RNA | |
|---|---|---|
| Element | Recognition | Amino Acid Sequence |
| 8PUF | GCAGCAGC | GRSRLLEDFRNNRYPNLQLREIAGHI |
| Frame 2 | | MEFSQDQHGSRFIRLKLERATPAERQ |
| | | LVFNEILQAAYQLMVDVFGSYVIEKF |
| | | FEFGSLEQKLALAERIRGHVLSLALQ |
| | | MYGCRVIQKALEFIPSDQQNEMVREL |
| | | DGHVLKCVKDQNGSYVVRKCIECVQP |
| | | QSLQFIIDAFKGQVFALSTHPYGSRV |
| | | IERILEHCLPDQTLPILEELHQHTEQ |
| | | LVQDQYGCYVIQHVLEHGRPEDKSKI |
| | | VAEIRGNVLVLSQHKFASYVVRKCVT |
| | | HASRTERAVLIDEVCTMNDGPHSALY |
| | | TMMKDQYASYVVEKMIDVAEPGQRKI |
| | | VMHKIRPHIATLRKYTYGKHILAKLE |
| | | KYYMKNGVDLG |
| | | (SEQ ID NO: 549) |
| Linker | — | VDTANGS |
| | | (SEQ ID NO: 411) |
| E17 | — | GGGTPKAPNLEPPLPEEEKEGSDLRP |
| endonuclease | | VVIDGSNVAMSHGNKEVFSCRGILLA |
| | | VNWFLERGHTDITVFVPSWRKEQPRP |
| | | DVPITDQHILRELEKKKILVFTPSRR |
| | | VGGKRVVCYDDRFIVKLAYESDGIVV |
| | | SNDTYRDLQGERQEWKRFIEERLLMY |
| | | SFVNDKFMPPDDPLGRHGPSLDNFLR |
| | | KKPLTLE |
| | | (SEQ ID NO: 358) |

TABLE 43
Exemplary 8PUF targeting CAG Fusion Protein

| | RNA | |
|---|---|---|
| Plasmid Element | Recognition | Amino Acid Sequence |
| Extra amino acids | | GSIVAVSRGM |
| between NLS and | | (SEQ ID NO: 387) |
| R1′ | | |
| 8PUF | CAGCAGCA | GRSRLLEDFRNNRYPNLQLRE |
| | | IAGHIMEFSQDQHGSRFIQLK |
| | | LERATPAERQLVFNEILQAAY |
| | | QLMVDVFGSYVIRKFFEFGSL |
| | | EQKLALAERIRGHVLSLALQM |
| | | YGSRVIEKALEFIPSDQQNEM |
| | | VRELDGHVLKCVKDQNGCYVV |
| | | QKCIECVQPQSLQFIIDAFKG |
| | | QVFALSTHPYGSRVIRRILEH |
| | | CLPDQTLPILEELHQHTEQLV |
| | | QDQYGSYVIEHVLEHGRPEDK |
| | | SKIVAEIRGNVLVLSQHKFAC |
| | | NVVQKCVTHASRTERAVLIDE |
| | | VCTMNDGPHSALYTMMKDQYA |
| | | SYVVRKMIDVAEPGQRKIVMH |
| | | KIRPHIATLRKYTYGKHILAK |
| | | LEKYYMKNGVDLG |
| | | (SEQ ID NO: 480) |
| Extra amino acids | | GRRDRMA |
| between R8′ and | | (SEQ ID NO: 386) |
| Linker | | |
| Linker | | VDTANGS |
| | | (SEQ ID NO: 411) |
| E17 | | GGGTPKAPNLEPPLPEEEKEG |
| | | SDLRPVVIDGSNVAMSHGNKE |
| | | VFSCRGILLAVNWFLERGHTD |
| | | ITVFVPSWRKEQPRPDVPITD |
| | | QHILRELEKKKILVFTPSRRV |
| | | GGKRVVCYDDRFIVKLAYESD |
| | | GIVVSNDTYRDLQGERQEWKR |
| | | FIEERLLMYSFVNDKFMPPDD |
| | | PLGRHGPSLDNFLRKKPLTLE |
| | | (SEQ ID NO: 358) |

TABLE 44
Exemplary 14PUF targeting CAG Fusion Protein

| Plasmid | RNA | |
|---|---|---|
| Element | Recognition | Amino Acid Sequence |
| human pRB-NLS | | KRSAEGSNPPKPLKKLR |
| | | (SEQ ID NO: 442) |
| 14PUF | CAGCAGCAGCA | GRSRLLEDFRNNRYPNLQLREIAGH |
| | GCA | IMEFSQDQHGSRFIQLKLERATPAE |
| | | RQLVFNEILQAAYQLMVDVFGSYVI |
| | | RKFFEFGSLEQKLALAERIRGHVLS |
| | | LALQMYGSRVIEKALEFIPSDQQNE |
| | | MVRELDGHVLKCVKDQNGCHVVQKC |
| | | IECVQPQSLQFIIDAFKGQVFALST |
| | | HPYGSRVIRRILEHCLPDQTLPILE |
| | | ELHQHIMEFSQDQHGSRFIELKLER |
| | | ATPAERQLVFNEILQAAYQLMVDVF |
| | | GCYVIQKFFEFGSLEQKLALAERIR |
| | | GHVLSLALQMYGSYVIRKALEFIPS |
| | | DQQNEMVRELDGHVLKCVKDQNGSY |
| | | VVEKCIECVQPQSLQFIIDAFKGQV |
| | | FALSTHPYGCRVIQRILEHCLPDQT |
| | | LPILEELHQHTEQLVQDQYGSYVIR |
| | | HVLEHGRPEDKSKIVAEIRGHTEQL |
| | | VQDQYGSYVIEHVLEHGRPEDKSKI |
| | | VAEIRGNVLVLSQHKFACNVVQKCV |
| | | THASRTERAVLIDEVCTMNDGPHSA |
| | | LYTMMKDQYASYVVRKMIDVAEPGQ |
| | | RKIVMHKIRPHIATLRKYTYGKHIL |
| | | AKLEKYYMKNGVDLG |
| | | (SEQ ID NO: 481) |
| Linker | | VDTANGS |
| | | (SEQ ID NO: 411) |
| E17 | | GGGTPKAPNLEPPLPEEEKEGSDLR |
| | | PVVIDGSNVAMSHGNKEVFSCRGIL |
| | | LAVNWFLERGHTDITVFVPSWRKEQ |
| | | PRPDVPITDQHILRELEKKKILVFT |
| | | PSRRVGGKRVVCYDDRFIVKLAYES |
| | | DGIVVSNDTYRDLQGERQEWKRFIE |
| | | ERLLMYSFVNDKFMPPDDPLGRHGP |
| | | SLDNFLRKKPLTLE |
| | | (SEQ ID NO: 358) |

TABLE 45
Exemplary 8PUF targeting CAG Fusion Protein

| | RNA | |
|---|---|---|
| Plasmid Element | Recognition | Amino Acid Sequence |
| H2B-NLS | | GKKRKRSRK |
| | | (SEQ ID NO: 438) |
| Extra amino | | GSIVAVSRGM |
| acids between | | (SEQ ID NO: 387) |
| NLS and R1′ | | |
| 8PUF | GCAGCAGC | GRSRLLEDFRNNRYPNLQLREIA |
| | | GHIMEFSQDQHGSRFIRLKLERA |
| | | TPAERQLVFNEILQAAYQLMVDV |
| | | FGSYVIEKFFEFGSLEQKLALAE |
| | | RIRGHVLSLALQMYGCRVIQKAL |
| | | EFIPSDQQNEMVRELDGHVLKCV |
| | | KDQNGSYVVRKCIECVQPQSLQF |
| | | IIDAFKGQVFALSTHPYGSRVIE |
| | | RILEHCLPDQTLPILEELHQHTE |
| | | QLVQDQYGCYVIQHVLEHGRPED |
| | | KSKIVAEIRGNVLVLSQHKFASY |
| | | VVRKCVTHASRTERAVLIDEVCT |
| | | MNDGPHSALYTMMKDQYASYVVE |
| | | KMIDVAEPGQRKIVMHKIRPHIA |
| | | TLRKYTYGKHILAKLEKYYMKNG |
| | | VDLG |
| | | (SEQ ID NO: 549) |
| Extra amino | | GRRDRMA |
| acids between | | (SEQ ID NO: 386) |
| R8′and Linker | | |
| Linker | | VDTANGS |
| | | (SEQ ID NO: 411) |
| E17 | | GGGTPKAPNLEPPLPEEEKEGSD |
| | | LRPVVIDGSNVAMSHGNKEVFSC |
| | | RGILLAVNWFLERGHTDITVFVP |
| | | SWRKEQPRPDVPITDQHILRELE |
| | | KKKILVFTPSRRVGGKRVVCYDD |
| | | RFIVKLAYESDGIVVSNDTYRDL |
| | | QGERQEWKRFIEERLLMYSFVND |
| | | KFMPPDDPLGRHGPSLDNFLRKK |
| | | PLTLE |
| | | (SEQ ID NO: 358) |

TABLE 46
Exemplary 8PUF targeting CAG Fusion Protein

| | RNA | |
|---|---|---|
| Plasmid Element | Recognition | Amino Acid Sequence |
| RB-NLS | | DRVLKRSAEGSNPPKPLKKLR |
| | | (SEQ ID NO: 543) |
| Linker | | GGS |
| | | (SEQ ID NO: 410) |
| Extra amino | | IVAVSRGM |
| acids between | | (SEQ ID NO: 388) |
| NLS and R1′ | | |
| 8PUF | GCAGCAGC | GRSRLLEDFRNNRYPNLQLREI |
| | | AGHIMEFSQDQHGSRFIRLKLE |
| | | RATPAERQLVFNEILQAAYQLM |
| | | VDVFGSYVIEKFFEFGSLEQKL |
| | | ALAERIRGHVLSLALQMYGCRV |
| | | IQKALEFIPSDQQNEMVRELDG |
| | | HVLKCVKDQNGSYVVRKCIECV |
| | | QPQSLQFIIDAFKGQVFALSTH |
| | | PYGSRVIERILEHCLPDQTLPI |
| | | LEELHQHTEQLVQDQYGCYVIQ |
| | | HVLEHGRPEDKSKIVAEIRGNV |
| | | LVLSQHKFASYVVRKCVTHASR |
| | | TERAVLIDEVCTMNDGPHSALY |
| | | TMMKDQYASYVVEKMIDVAEPG |
| | | QRKIVMHKIRPHIATLRKYTYG |
| | | KHILAKLEKYYMKNGVDLG |
| | | (SEQ ID NO: 549) |
| Extra amino | | GRRDRMA |
| acids between | | (SEQ ID NO: 386) |
| R8′and Linker | | |
| Linker | | VDTANGS |
| | | (SEQ ID NO: 411) |
| E17 | | GGGTPKAPNLEPPLPEEEKEGS |
| | | DLRPVVIDGSNVAMSHGNKEVF |
| | | SCRGILLAVNWFLERGHTDITV |
| | | FVPSWRKEQPRPDVPITDQHIL |
| | | RELEKKKILVFTPSRRVGGKRV |
| | | VCYDDRFIVKLAYESDGIVVSN |
| | | DTYRDLQGERQEWKRFIEERLL |
| | | MYSFVNDKFMPPDDPLGRHGPS |
| | | LDNFLRKKPLTLE |
| | | (SEQ ID NO: 358) |

TABLE 47
Exemplary 8PUF targeting CAG Fusion Protein

| Plasmid | RNA | |
|---|---|---|
| Element | Recognition | Amino Acid Sequence |
| RB-NLS | | DRVLKRSAEGSNPPKPLKKLR |
| | | (SEQ ID NO: 543) |
| Linker | | GGS |
| | | (SEQ ID NO: 410) |
| 8PUF | GCAGCAGC | GRSRLLEDFRNNRYPNLQLREIAGHIMEFS |
| | | QDQHGSRFIRLKLERATPAERQLVFNEILQ |
| | | AAYQLMVDVFGSYVIEKFFEFGSLEQKLAL |
| | | AERIRGHVLSLALQMYGCRVIQKALEFIPS |
| | | DQQNEMVRELDGHVLKCVKDQNGSHVVRKC |
| | | IECVQPQSLQFIIDAFKGQVFALSTHPYGS |
| | | RVIERILEHCLPDQTLPILEELHQHTEQLV |
| | | QDQYGCYVIQHVLEHGRPEDKSKIVAEIRG |
| | | NVLVLSQHKFASYVVRKCVTHASRTERAVL |
| | | IDEVCTMNDGPHSALYTMMKDQYASYVVEK |
| | | MIDVAEPGQRKIVMHKIRPHIATLRKYTYG |
| | | KHILAKLEKYYMKNGVDLG |
| | | (SEQ ID NO: 568) |
| Linker | | VDTANGS |
| | | (SEQ ID NO: 411) |
| E17 | | GGGTPKAPNLEPPLPEEEKEGSDLRPVVID |
| | | GSNVAMSHGNKEVFSCRGILLAVNWFLERG |
| | | HTDITVFVPSWRKEQPRPDVPITDQHILRE |
| | | LEKKKILVFTPSRRVGGKRVVCYDDRFIVK |
| | | LAYESDGIVVSNDTYRDLQGERQEWKRFIE |
| | | ERLLMYSFVNDKFMPPDDPLGRHGPSLDNF |
| | | LRKKPLTLE |
| | | (SEQ ID NO: 358) |

TABLE 48
Exemplary 8PUF targeting CAG Fusion Protein

| | RNA | |
|---|---|---|
| Plasmid Element | Recognition | Amino Acid Sequence |
| H2B-NLS | | GKKRKRSRK |
| | | (SEQ ID NO: 438) |
| Extra amino | | GSIVAVSRGM |
| acids between | | (SEQ ID NO: 387) |
| NLS and R1′ | | |
| 8PUF | GCAGCAGC | GRSRLLEDFRNNRYPNLQLREI |
| | | AGHIMEFSQDQHGSRFIRLKLE |
| | | RATPAERQLVFNEILQAAYQLM |
| | | VDVFGSYVIEKFFEFGSLEQKL |
| | | ALAERIRGHVLSLALQMYGCRV |
| | | IQKALEFIPSDQQNEMVRELDG |
| | | HVLKCVKDQNGSYVVRKCIECV |
| | | QPQSLQFIIDAFKGQVFALSTH |
| | | PYGSRVIERILEHCLPDQTLPI |
| | | LEELHQHTEQLVQDQYGCYVIQ |
| | | HVLEHGRPEDKSKIVAEIRGNV |
| | | LVLSQHKFASYVVRKCVTHASR |
| | | TERAVLIDEVCTMNDGPHSALY |
| | | TMMKDQYASYVVEKMIDVAEPG |
| | | QRKIVMHKIRPHIATLRKYTYG |
| | | KHILAKLEKYYMKNGVDLG |
| | | (SEQ ID NO: 549) |
| Extra amino | | GRRDRMA |
| acids between | | (SEQ ID NO: 386) |
| R8′and Linker | | |
| Linker | | VDTANGS |
| | | (SEQ ID NO: 411) |
| E17 | | GGGTPKAPNLEPPLPEEEKEGS |
| | | DLRPVVIDGSNVAMSHGNKEVF |
| | | SCRGILLAVNWFLERGHTDITV |
| | | FVPSWRKEQPRPDVPITDQHIL |
| | | RELEKKKILVFTPSRRVGGKRV |
| | | VCYDDRFIVKLAYESDGIVVSN |
| | | DTYRDLQGERQEWKRFIEERLL |
| | | MYSFVNDKFMPPDDPLGRHGPS |
| | | LDNFLRKKPLTLE |
| | | (SEQ ID NO: 358) |
| PKI-NES | | LALKLAGLDI |
| | | (SEQ ID NO: 545) |

TABLE 49
Exemplary 8PUF targeting CAG Fusion Protein

| | RNA | |
|---|---|---|
| Plasmid Element | Recognition | Amino Acid Sequence |
| H2B-NLS | | GKKRKRSRK (SEQ ID NO: 438) |
| Extra amino acids | | GSIVAVSRG (SEQ ID NO: 385) |
| between NLS and R1′ | | |
| 8PUF | GCAGCAGC | GRSRLLEDFRNNRYPNLQLREIAGHIMEFSQDQHGSRFIRLKLERATPAERQLVFN |
| | | EILQAAYQLMVDVFGSYVIEKFFEFGSLEQKLALAERIRGHVLSLALQMYGCRVIQ |
| | | KALEFIPSDQQNEMVRELDGHVLKCVKDQNGSYVVRKCIECVQPQSLQFIIDAFKG |
| | | QVFALSTHPYGSRVIERILEHCLPDQTLPILEELHQHTEQLVQDQYGCYVIQHVLE |
| | | HGRPEDKSKIVAEIRGNVLVLSQHKFASYVVRKCVTHASRTERAVLIDEVCTMNDG |
| | | PHSALYTMMKDQYASYVVEKMIDVAEPGQRKIVMHKIRPHIATLRKYTYGKHILAK |
| | | LEKYYMKNGVDLG (SEQ ID NO: 549) |
| Extra amino acids | | GRRDRMA (SEQ ID NO: 386) |
| between R8′ and | | |
| Linker | | |
| Linker | | VDTANGS (SEQ ID NO: 411) |
| E17 | | GGGTPKAPNLEPPLPEEEKEGSDLRPVVIDGSNVAMSHGNKEVFSCRGILLAVNWF |
| | | LERGHTDITVFVPSWRKEQPRPDVPITDQHILRELEKKKILVFTPSRRVGGKRVVC |
| | | YDDRFIVKLAYESDGIVVSNDTYRDLQGERQEWKRFIEERLLMYSFVNDKFMPPDD |
| | | PLGRHGPSLDNFLRKKPLTLE (SEQ ID NO: 358) |
| Human PKI NES | | LALKLAGLDI (SEQ ID NO: 545) |

8PUF targeting CAGf2 w/ stacking mutations (C binding mutant) w/ or w/out endonuclease

| | Protein | | | |
|---|---|---|---|---|
| Construct | Type | Elements | Sequence | Amino Acid Sequence |
| n/a | 8PUF | N-terminal | GCAGCAGC | GRSRLLEDFRNNRYPNLQLREIAGHIMEFSQ |
| | | 8PUF with or | | DQHGSFFIRLKLERATPAERQLVFNEILQAA |
| | | without C- | | YQLMVDVFGSYVIEKFFEFGSLEQKLALAER |
| | | terminal E17 | | IRGHVLSLALQMYGCRVIQKALEFIPSDQQN |
| | | with linker | | EMVRELDGHVLKCVKDQNGSFVVRKCIECVQ |
| | | between | | PQSLQFIIDAFKGQVFALSTHPYGSRVIERI |
| | | 8PUF and E17 | | LEHCLPDQTLPILEELHQHTEQLVQDQYGCY |
| | | | | VIQHVLEHGRPEDKSKIVAEIRGNVLVLSQH |
| | | | | KFASFVVRKCVTHASRTERAVLIDEVCTMND |
| | | | | GPHSALYTMMKDQYASYVVEKMIDVAEPGQR |
| | | | | KIVMHKIRPHIATLRKYTYGKHILAKLEKYY |
| | | | | MKNGVDLG (SEQ ID NO: 658) |

Amino acid sequences of transgene elements in order N-terminal to C-terminal
(for *cleaving or blocking):

| Plasmid Element | Amino Acid Sequences |
|---|---|
| 8PUF | GRSRLLEDFRNNRYPNLQLREIAGHIMEFSQDQHGSFFIRLKLERATPAERQLVFNEILQAAY |
| | QLMVDVFGSYVIEKFFEFGSLEQKLALAERIRGHVLSLALQMYGCRVIQKALEFIPSDQQNEM |
| | VRELDGHVLKCVKDQNGSFVVRKCIECVQPQSLQFIIDAFKGQVFALSTHPYGSRVIERILEH |
| | CLPDQTLPILEELHQHTEQLVQDQYGCYVIQHVLEHGRPEDKSKIVAEIRGNVLVLSQHKFAS |
| | FVVRKCVTHASRTERAVLIDEVCTMNDGPHSALYTMMKDQYASYVVEKMIDVAEPGQRKIVMH |
| | KIRPHIATLRKYTYGKHILAKLEKYYMKNGVDLG (SEQ ID NO: 658) |
| *Linker | VDTANGS (SEQ ID NO: 411) |
| *E17 | GGGTPKAPNLEPPLPEEEKEGSDLRPVVIDGSNVAMSHGNKEVFSCRGILLAVNWFLERGHTD |
| | ITVFVPSWRKEQPRPDVPITDQHILRELEKKKILVFTPSRRVGGKRVVCYDDRFIVKLAYESD |
| | GIVVSNDTYRDLQGERQEWKRFIEERLLMYSFVNDKFMPPDDPLGRHGPSLDNFLRKKPLTLE |
| | (SEQ ID NO: 358) |

### Vectors

In some embodiments of the compositions and methods of the disclosure, a vector comprises a guide RNA of the disclosure. In some embodiments, the vector comprises at least one guide RNA of the disclosure. In some embodiments, the vector comprises one or more guide RNA(s) of the disclosure. In some embodiments, the vector comprises two or more guide RNAs of the disclosure. In one embodiment, the vector comprises three guide RNAs. In one embodiment, the vector comprises four guide RNAs. In some embodiments, the vector further comprises a guided or non-guided RNA-binding protein of the disclosure. In some embodiments, the vector further comprises an RNA-binding fusion protein of the disclosure. In some embodiments, the fusion protein comprises a first RNA binding protein and a second RNA binding protein. In some embodiments, the RNA-guided RNA-binding systems comprising an RNA-binding protein and a gRNA are in a single vector. In a particular embodiment, the single vector comprises the RNA-guided RNA-binding systems which are Cas13d RNA-guided RNA-binding systems or catalytic deactivated Cas13d (dCas13d) RNA-guided RNA-binding systems. In one embodiment, the single vector comprises the Cas13d RNA-guided RNA-binding systems which are CasRx or dCasRx RNA-guided RNA-binding systems. In another embodiment, the single vector comprises a non-guided RNA-binding system comprising a PUF or PUMBY-based protein fused with a nuclease domain from ZC3H12A, such as E17 (SEQ ID NO: 358). In another embodiment, the single vector comprises a dCas13d RNA-binding system fused with a nuclease domain from ZC3H12A, such as E17 (SEQ ID NO: 359).

In some embodiments of the compositions and methods of the disclosure, a first vector comprises a guide RNA of the disclosure and a second vector comprises an RNA-binding protein or RNA-binding fusion protein of the disclosure. In some embodiments, the first vector comprises at least one guide RNA of the disclosure. In some embodiments, the first vector comprises one or more guide RNA(s) of the disclosure. In some embodiments, the first vector comprises two or more guide RNA(s) of the disclosure. In some embodiments, the fusion protein comprises a first RNA binding protein and a second RNA binding protein. In some embodiments, the first vector and the second vector are identical vectors or vector serotypes. In some embodiments, the first vector and the second vector are not identical vectors or vector serotypes. In some embodiments of the compositions and methods of the disclosure, the RNA-binding systems capable of targeting toxic CAG RNA repeats are in a single vector.

One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. In some embodiments, the vector is a lentivirus (such as an integration-deficient lentiviral vector) or adeno-associated viral (AAV) vector. Vectors are capable of autonomous replication in a host cell into which they are introduced such as e.g., bacterial vectors having a bacterial origin of replication and episomal mammalian vectors and other vectors such as, e.g., non-episomal mammalian vectors, are integrated into the genome of a host cell upon introduction into the host cell, and thereby are replicated along with the host genome.

In some embodiments, vectors such as e.g., expression vectors, are capable of directing the expression of genes to which they are operatively-linked. Common expression vectors are often in the form of plasmids. In some embodiments, recombinant expression vectors comprise a nucleic acid provided herein such as e.g., a guide RNA which can be expressed from a DNA sequence, and a nucleic acid encoding a Cas 13d protein, in a form suitable for expression of a protein in a host cell. Recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence such as e.g., in an in vitro transcription/translation system or in a host cell when the vector is introduced into the host cell. Certain embodiments of a vector depend on factors such as the choice of the host cell to be transformed, and the level of expression desired. A vector can be introduced into host cells to thereby produce transcripts, proteins, or peptides, including fusion proteins or peptides, encoded by nucleic acids as described herein such as, e.g., CRISPR transcripts, proteins, enzymes, mutant forms thereof, fusion proteins thereof, etc.

In some embodiments of the compositions and methods of the disclosure, a vector of the disclosure is a viral vector. In some embodiments, the viral vector comprises a sequence isolated or derived from a retrovirus. In some embodiments, the viral vector comprises a sequence isolated or derived from a lentivirus. In some embodiments, the viral vector comprises a sequence isolated or derived from an adenovirus. In some embodiments, the viral vector comprises a sequence isolated or derived from an adeno-associated virus (AAV). In some embodiments, the viral vector is replication incompetent. In some embodiments, the viral vector is isolated or recombinant. In some embodiments, the viral vector is self-complementary.

The term “adeno-associated virus” or “AAV” as used herein refers to a member of the class of viruses associated with this name and belonging to the genus Dependoparvovirus, family Parvoviridae. Adeno-associated virus is a single-stranded DNA virus that grows in cells in which certain functions are provided by a co-infecting helper virus. General information and reviews of AAV can be found in, for example, Carter, 1989, Handbook of Parvoviruses, Vol. 1, pp. 169-228, and Berns, 1990, Virology, pp. 1743-1764, Raven Press, (New York). It is fully expected that the same principles described in these reviews will be applicable to additional AAV serotypes characterized after the publication dates of the reviews because it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3: 1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control. Multiple serotypes of this virus are known to be suitable for gene delivery; all known serotypes can infect cells from various tissue types.

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA to generate AAV vectors. The rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

Recombinant AAV (rAAV) genomes of the invention comprise, consist essentially of, or consist of a nucleic acid molecule encoding a CAG-repeat targeting composition (such as a PUF, PUMBY, or RNA-guided protein) and one or more AAV ITRs flanking the nucleic acid molecule. Production of pseudotyped rAAV is disclosed in, for example, WO2001083692. Other types of rAAV variants, for example rAAV with capsid mutations, are also contemplated. See, e.g., Marsic et al., Molecular Therapy, 22(11): 1900-1909 (2014). The nucleotide sequences of the genomes of various AAV serotypes are known in the art.

In some embodiments of the compositions and methods of the disclosure, the viral vector comprises a sequence isolated or derived from an adeno-associated virus (AAV). In some embodiments, the viral vector comprises an inverted terminal repeat sequence or a capsid sequence that is isolated or derived from an AAV of serotype AAV1, AAV2, AAV3, AAV4, AAV5, AAV6, AAV7, AAV8, AAV9, AAV10 (AAVrh10), AAV11 or AAV12. In some embodiments, the AAV serotype is AAVrh.74. In one embodiment, the AAV vector comprises a modified capsid. In one embodiment the AAV vector is an AAV2-Tyr mutant vector. In one embodiment the AAV vector comprises a capsid with a non-tyrosine amino acid at a position that corresponds to a surface-exposed tyrosine residue in position Tyr252, Tyr272, Tyr275, Tyr281, Tyr508, Tyr612, Tyr704, Tyr720, Tyr730 or Tyr673 of wild-type AAV2. See also WO 2008/124724 incorporated herein in its entirety. In some embodiments, the AAV vector comprises an engineered capsid. AAV vectors comprising engineered capsids include without limitation, AAV2.7m8, AAV9.7m8, AAV2 2tYF, and AAV8 Y733F). In some embodiments, the viral vector is replication incompetent. In some embodiments, the viral vector is isolated or recombinant (rAAV). In some embodiments, the viral vector is self-complementary (scAAV).

In some embodiments of the compositions and methods of the disclosure, a vector of the disclosure is a non-viral vector. In some embodiments, the vector comprises or consists of a nanoparticle, a micelle, a liposome or lipoplex, a polymersome, a polyplex or a dendrimer. In some embodiments, the vector is an expression vector or recombinant expression system. As used herein, the term “recombinant expression system” refers to a genetic construct for the expression of certain genetic material formed by recombination.

In some embodiments of the compositions and methods of the disclosure, an expression vector, viral vector or non-viral vector provided herein, includes without limitation, an expression control element. An “expression control element” as used herein refers to any sequence that regulates the expression of a coding sequence, such as a gene. Exemplary expression control elements include but are not limited to promoters, enhancers, microRNAs, post-transcriptional regulatory elements, polyadenylation signal sequences, and introns. Expression control elements may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. In some embodiments, expression control by a promoter is tissue-specific. In some embodiments, expression control by a promoter is constitutive or ubiquitous. Non-limiting exemplary promoters include a Pol III promoter such as, e.g., U6 and H1 promoters and/or a Pol II promoter e.g., SV40, CMV (optionally including the CMV enhancer), RSV (Rous Sarcoma Virus LTR promoter (optionally including RSV enhancer), CBA (hybrid CMV enhancer/chicken β-actin), CAG (hybrid CMV enhancer fused to chicken β-actin), truncated CAG, Cbh (hybrid CBA), EF-1a (human elongation factor alpha-1) or EFS (short intron-less EF-1 alpha), PGK (phosphoglycerol kinase), CEF (chicken embryo fibroblasts), UBC (ubiquitinC), GUSB (lysosomal enzyme beta-glucuronidase), UCOE (ubiquitous chromatin opening element), hAAT (alpha-1 antitrypsin), TBG (thyroxine binding globulin), Desmin (full-length (SEQ ID NO: 654) or truncated (SEQ ID NO: 655)), MCK (muscle creatine kinase), C5-12 (synthetic muscle promoter), CK8e (creatin kinase 8), NSE (neuron-specific enolase), Synapsin, Synapsin-1 (SYN-1), opsin, PDGF (platelet-derived growth factor), PDGF-A, MecP2 (methyl CpG-binding protein 2), CaMKII (Calcium/Calmodulin-dependent protein kinase II), mGluR2 (metabotropic glutamate receptor 2), NFL (neurofilament light), NFH (neurofilament heavy), nβ2, PPE (rat preproenkephalin), ENK (preproenkephalin), Preproenkephalin-neurofilament chimeric promoter, EAAT2 (glutamate transporter), GFAP (glial fibrillary acidic protein), MBP (myelin basic protein), human rhodopsin kinase promoter (hGRKi), β-actin promoter, dihydrofolate reductase promoter, MHCK7 (hybrid promoter of enhancer/promoter regions of muscle creatine kinase and alpha myosin heavy-chain genes) and combinations thereof. An “enhancer” is a region of DNA that can be bound by activating proteins to increase the likelihood or frequency of transcription. Non-limiting exemplary enhancers and posttranscriptional regulatory elements include the CMV enhancer, MCK enhancer, R-U5′ segment in LTR of HTLV-1, SV40 enhancer, the intron sequence between exons 2 and 3 of rabbit β-globin, and Woodchuck Hepatitis Virus (WHP) Posttranscriptional Regulatory Element (WPRE). In some embodiments an intron is used to enhance promoter activity such as a UBB intron. In some embodiments, the UBB intron is used with an EFS promoter.

In some embodiments of the compositions and methods of the disclosure, an expression vector, viral vector or non-viral vector provided herein, includes without limitation, vector elements such as an IRES or 2A peptide sites for configuration of “multicistronic” or “polycistronic” or “bicistronic” or tricistronic” constructs, i.e., having double or triple or multiple coding areas or exons, and as such will have the capability to express from mRNA two or more proteins from a single construct. Multicistronic vectors simultaneously express two or more separate proteins from the same mRNA. The two strategies most widely used for constructing multicistronic configurations are through the use of an IRES or a 2A self-cleaving site. An “IRES” refers to an internal ribosome entry site or portion thereof of viral, prokaryotic, or eukaryotic origin which are used within polycistronic vector constructs. In some embodiments, an IRES is an RNA element that allows for translation initiation in a cap-independent manner. The term “self-cleaving peptides” or “sequences encoding self-cleaving peptides” or “2A self-cleaving site” refer to linking sequences which are used within vector constructs to incorporate sites to promote ribosomal skipping and thus to generate two polypeptides from a single promoter, such self-cleaving peptides include without limitation, T2A, and P2A peptides or other sequences encoding the self-cleaving peptides.

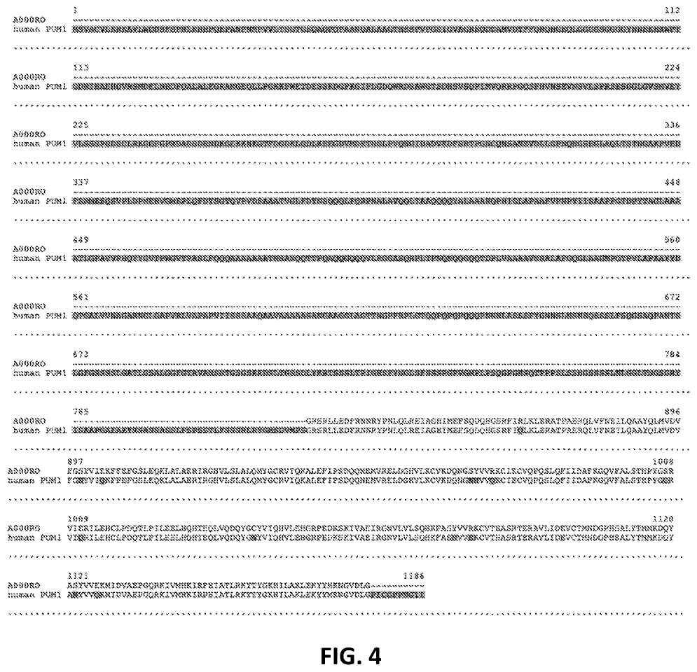

In one embodiment, exemplary vector configurations are shown in FIGS. 4A-4C. Exemplary vector configurations comprise a promoter or regulatory sequence (promoter/enhancer combination) driving the expression of the nucleic acid encoding the CAG-targeting PUF-endonuclease fusion. In another embodiment, a vector configuration comprises a promoter driving expression of the RNA-guided Cas RNase RNA-binding protein, or dCas protein fusion in operable linkage with a second promoter driving expressing of a cognate gRNA. In another embodiment, the vector configuration comprises a linker and one or more tags.

In some embodiments, the vector is a viral vector. In some embodiments, the vector is an adenoviral vector, an adeno-associated viral (AAV) vector, or a lentiviral vector. In some embodiments, the vector is a retroviral vector, an adenoviral/retroviral chimera vector, a herpes simplex viral I or II vector, a parvoviral vector, a reticuloendotheliosis viral vector, a polioviral vector, a papillomaviral vector, a vaccinia viral vector, or any hybrid or chimeric vector incorporating favorable aspects of two or more viral vectors. In some embodiments, the vector further comprises one or more expression control elements operably linked to the polynucleotide. In some embodiments, the vector further comprises one or more selectable markers. In some embodiments, the AAV vector has low toxicity. In some embodiments, the AAV vector does not incorporate into the host genome, thereby having a low probability of causing insertional mutagenesis. In some embodiments, the AAV vector can encode a range of total polynucleotides from 4.5 kb to 4.75 kb. In some embodiments, exemplary AAV vectors that may be used in any of the herein described compositions, systems, methods, and kits can include an AAV1 vector, a modified AAV1 vector, an AAV2 vector, a modified AAV2 vector, an AAV2-Tyr mutant vector, an AAV3 vector, a modified AAV3 vector, an AAV4 vector, a modified AAV4 vector, an AAV5 vector, a modified AAV5 vector, an AAV6 vector, a modified AAV6 vector, an AAV7 vector, a modified AAV7 vector, an AAV8 vector, an AAV9 vector, an AAV.rh10 vector, a modified AAV.rh10 vector, an AAVrh.74, an AAV.rh32/33 vector, a modified AAV.rh32/33 vector, an AAV.rh43 vector, a modified AAV.rh43 vector, an AAV.rh64R1 vector, and a modified AAV.rh64R1 vector, an AAV-Tyr mutant vector, and any combinations or equivalents thereof. In some embodiments, the lentiviral vector is an integrase-competent lentiviral vector (ICLV). In some embodiments, the lentiviral vector can refer to the transgene plasmid vector as well as the transgene plasmid vector in conjunction with related plasmids (e.g., a packaging plasmid, a rev expressing plasmid, an envelope plasmid) as well as a lentiviral-based particle capable of introducing exogenous nucleic acid into a cell through a viral or viral-like entry mechanism. Lentiviral vectors are well-known in the art (see, e.g., Trono D. (2002) Lentiviral vectors, New York: Spring-Verlag Berlin Heidelberg and Durand et al. (2011) Viruses 3(2):132-159 doi: 10.3390/v3020132). In some embodiments, exemplary lentiviral vectors that may be used in any of the herein described compositions, systems, methods, and kits can include a human immunodeficiency virus (HIV) 1 vector, a modified human immunodeficiency virus (HIV) 1 vector, a human immunodeficiency virus (HIV) 2 vector, a modified human immunodeficiency virus (HIV) 2 vector, a sooty mangabey simian immunodeficiency virus (SIV_{SM}) vector, a modified sooty mangabey simian immunodeficiency virus (SIV_{SM}) vector, a African green monkey simian immunodeficiency virus (SIV_{AGM}) vector, a modified African green monkey simian immunodeficiency virus (SIV_{AGM}) vector, an equine infectious anemia virus (EIAV) vector, a modified equine infectious anemia virus (EIAV) vector, a feline immunodeficiency virus (FIV) vector, a modified feline immunodeficiency virus (FIV) vector, a Visna/maedi virus (VNV/VMV) vector, a modified Visna/maedi virus (VNV/VMV) vector, a caprine arthritis-encephalitis virus (CAEV) vector, a modified caprine arthritis-encephalitis virus (CAEV) vector, a bovine immunodeficiency virus (BIV), or a modified bovine immunodeficiency virus (BIV).

### Nucleic Acids

Provided herein are the nucleic acid sequences encoding RNA-binding CAG repeat-targeting systems disclosed herein for use in gene transfer and expression techniques described herein. It should be understood, although not always explicitly stated that the sequences provided herein can be used to provide the expression product as well as substantially identical sequences that produce a protein that has the same biological properties. These “biologically equivalent” or “biologically active” or “equivalent” polypeptides are encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid sequence to the reference polypeptide when compared using sequence identity methods run under default conditions. Specific polypeptide sequences are provided as examples of particular embodiments. Modifications to the sequences to amino acids with alternate amino acids that have similar charge. Additionally, an equivalent polynucleotide is one that hybridizes under stringent conditions to the reference polynucleotide or its complement or in reference to a polypeptide, a polypeptide encoded by a polynucleotide that hybridizes to the reference encoding polynucleotide under stringent conditions or its complementary strand. Alternatively, an equivalent polypeptide or protein is one that is expressed from an equivalent polynucleotide.

The nucleic acid sequences (e.g., polynucleotide sequences) disclosed herein may be codon-optimized which is a technique well known in the art. In some embodiments disclosed herein, exemplary Cas sequences, such as e.g., a nucleic acid sequence encoding SEQ ID NO: 92 (Cas13d known as CasRx) or the nucleic acid sequence encoding SEQ ID NO: 298 (Cas13d known as CasRx), are codon optimized for expression in human cells. Codon optimization refers to the fact that different cells differ in their usage of particular codons. This codon bias corresponds to a bias in the relative abundance of particular tRNAs in the cell type. By altering the codons in the sequence to match with the relative abundance of corresponding tRNAs, it is possible to increase expression. It is also possible to decrease expression by deliberately choosing codons for which the corresponding tRNAs are known to be rare in a particular cell type. Codon usage tables are known in the art for mammalian cells, as well as for a variety of other organisms. Based on the genetic code, nucleic acid sequences coding for, e.g., a Cas protein, can be generated. In some embodiments, such a sequence is optimized for expression in a host or target cell, such as a host cell used to express the Cas protein or a cell in which the disclosed methods are practiced (such as in a mammalian cell, e.g., a human cell). Codon preferences and codon usage tables for a particular species can be used to engineer isolated nucleic acid molecules encoding a Cas protein (such as one encoding a protein having at least 80%, at least 85%, at least 90%, at least 92%, at least 95%, at least 96%, at least 97%, at least 98%, at least 99%, or 100% sequence identity to its corresponding wild-type protein) that takes advantage of the codon usage preferences of that particular species. For example, the Cas proteins disclosed herein can be designed to have codons that are preferentially used by a particular organism of interest. In one example, a Cas nucleic acid sequence is optimized for expression in human cells, such as one having at least 70%, at least 80%, at least 85%, at least 90%, at least 92%, at least 95%, at least 98%, or at least 99% sequence identity to its corresponding wild-type or originating nucleic acid sequence. In some embodiments, an isolated nucleic acid molecule encoding at least one Cas protein (which can be part of a vector) includes at least one Cas protein coding sequence that is codon optimized for expression in a eukaryotic cell, or at least one Cas protein coding sequence codon optimized for expression in a human cell. In one embodiment, such a codon optimized Cas coding sequence has at least 80%, at least 85%, at least 90%, at least 92%, at least 95%, at least 96%, at least 97%, at least 98%, at least 99%, or 100% sequence identity to its corresponding wild-type or originating sequence. In another embodiment, a eukaryotic cell codon optimized nucleic acid sequence encodes a Cas protein having at least 85%, at least 90%, at least 92%, at least 95%, at least 96%, at least 97%, at least 98%, at least 99%, or 100% sequence identity to its corresponding wild-type or originating protein. In another embodiment, a variety of clones containing functionally equivalent nucleic acids may be routinely generated, such as nucleic acids which differ in sequence but which encode the same Cas protein sequence. Silent mutations in the coding sequence result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA. Tables showing the standard genetic code can be found in various sources (see, for example, Stryer, 1988, Biochemistry, 3.sup.rd Edition, W.H. 5 Freeman and Co., NY).

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PC reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

### Cells

In some embodiments of the compositions and methods of the disclosure, a cell of the disclosure is a prokaryotic cell.

In some embodiments of the compositions and methods of the disclosure, a cell of the disclosure is a eukaryotic cell. In some embodiments, the cell is a mammalian cell. In some embodiments, the cell is a bovine, murine, feline, equine, porcine, canine, simian, or human cell. In some embodiments, the cell is a non-human mammalian cell such as a non-human primate cell.

In some embodiments, a cell of the disclosure is a somatic cell. In some embodiments, a cell of the disclosure is a germline cell. In some embodiments, a germline cell of the disclosure is not a human cell.

In some embodiments of the compositions and methods of the disclosure, a cell of the disclosure is a stem cell. In some embodiments, a cell of the disclosure is an embryonic stem cell. In some embodiments, an embryonic stem cell of the disclosure is not a human cell. In some embodiments, a cell of the disclosure is a multipotent stem cell or a pluripotent stem cell. In some embodiments, a cell of the disclosure is an adult stem cell. In some embodiments, a cell of the disclosure is an induced pluripotent stem cell (iPSC). In some embodiments, a cell of the disclosure is a hematopoietic stem cell (HSC).

In some embodiments of the compositions and methods of the disclosure, a somatic cell of the disclosure is a neuronal cell. In one embodiment, a cell or cells of a patient treated with compositions disclosed herein include, without limitation, central nervous system (neurons), peripheral nervous system (neurons), peripheral motor neurons, and/or sensory neurons. In one embodiment, a neuronal cell is a glial cell.

In some embodiments of the compositions and methods of the disclosure, a somatic cell of the disclosure is a fibroblast or an epithelial cell. In some embodiments, an epithelial cell of the disclosure forms a squamous cell epithelium, a cuboidal cell epithelium, a columnar cell epithelium, a stratified cell epithelium, a pseudostratified columnar cell epithelium or a transitional cell epithelium. In some embodiments, an epithelial cell of the disclosure forms a gland including, but not limited to, a pineal gland, a thymus gland, a pituitary gland, a thyroid gland, an adrenal gland, an apocrine gland, a holocrine gland, a merocrine gland, a serous gland, a mucous gland and a sebaceous gland. In some embodiments, an epithelial cell of the disclosure contacts an outer surface of an organ including, but not limited to, a lung, a spleen, a stomach, a pancreas, a bladder, an intestine, a kidney, a gallbladder, a liver, a larynx or a pharynx. In some embodiments, an epithelial cell of the disclosure contacts an outer surface of a blood vessel or a vein.

In some embodiments of the compositions and methods of the disclosure, a somatic cell of the disclosure is a primary cell.

In some embodiments of the compositions and methods of the disclosure, a somatic cell of the disclosure is a cultured cell.

In some embodiments of the compositions and methods of the disclosure, a somatic cell of the disclosure is in vivo, in vitro, ex vivo or in situ.

In some embodiments of the compositions and methods of the disclosure, a somatic cell of the disclosure is autologous or allogeneic.

Methods of Use

The disclosure provides a method of modifying level of expression of an RNA molecule of the disclosure or a protein encoded by the RNA molecule comprising contacting the composition of the disclosure and the RNA molecule under conditions suitable for binding of one or more of the guide RNA or the RNA-binding protein or RNA-binding fusion protein (or a portion thereof) to the RNA molecule.

The disclosure provides a method of modifying an activity of a protein encoded by an RNA molecule comprising contacting the composition of the disclosure and the RNA molecule under conditions suitable for binding of one or more of the guide RNA or the RNA-binding protein or the fusion protein (or a portion thereof) to the RNA molecule.

The disclosure provides a method of modifying level of expression of an RNA molecule of the disclosure or a protein encoded by the RNA molecule comprising contacting the composition of the disclosure and a cell comprising the RNA molecule under conditions suitable for binding of one or more of the guide RNA or the RNA-binding protein or fusion protein (or a portion thereof) to the RNA molecule. In some embodiments, the cell is in vivo, in vitro, ex vivo or in situ. In some embodiments, the composition of the disclosure comprises a vector comprising a guide RNA of the disclosure and an RNA-binding protein or fusion protein of the disclosure. In some embodiments, the vector is an AAV.

The disclosure provides a method of modifying an activity of a protein encoded by an RNA molecule comprising contacting the composition of the disclosure and a cell comprising the RNA molecule under conditions suitable for binding of one or more of the guide RNA or the RNA-binding protein or fusion protein (or a portion thereof) to the RNA molecule.

The disclosure provides a method of modifying the level of expression of an RNA molecule of the disclosure or a protein encoded by the RNA molecule comprising contacting the composition of the disclosure and the RNA molecule under conditions suitable for RNA nuclease activity wherein the RNA-binding protein or fusion protein induces a break in the RNA molecule.

The disclosure provides a method of modifying an activity of a protein encoded by an RNA molecule comprising contacting the composition of the disclosure and the RNA molecule under conditions suitable for RNA nuclease activity wherein the RNA-binding protein or fusion protein induces a break in the RNA molecule.

The disclosure provides a method of modifying a level of expression of an RNA molecule of the disclosure or a protein encoded by the RNA molecule comprising contacting the composition of the disclosure and a cell comprising the RNA molecule under conditions suitable for RNA nuclease activity wherein the RNA-binding protein or fusion protein induces a break in the RNA molecule. In some embodiments, the cell is in vivo, in vitro, ex vivo or in situ. In some embodiments, the composition comprises a vector comprising composition comprising a guide RNA of the disclosure and an RNA-binding fusion protein of the disclosure. In some embodiments, the vector is an AAV.

The disclosure provides a method of modifying an activity of a protein encoded by an RNA molecule comprising contacting the composition and a cell comprising the RNA molecule under conditions suitable for RNA nuclease activity wherein the RNA-binding protein or fusion protein induces a break in the RNA molecule. In some embodiments, the cell is in vivo, in vitro, ex vivo or in situ. In some embodiments, the composition comprises a vector comprising composition comprising a guide RNA or a single guide RNA of the disclosure and a nucleic acid sequence encoding an RNA-binding protein or fusion protein of the disclosure. In some embodiments, the vector is an AAV.

The disclosure provides a method of treating a disease or disorder comprising administering to a subject a therapeutically effective amount of a composition of the disclosure. In one embodiment, the disclosure provides a method of treating CAG repeat diseases. In another embodiment, the CAG repeat disorder is HD or SCA1. In another embodiment, the CAG repeat disorder is selected from the group consisting of HD, SCA1, SCA2, SCA3, SCA6, SCA7, SCA12, SCA17, Spinal and Bulbar Muscular Atrophy, and Denatorubral-Pallidoluysian Atrophy.

The disclosure provides a method of treating a CAG repeat diseases such as HD and SCA1 in a patient in need of such treatment comprising administering to the patient a therapeutically effective amount of a composition of the disclosure, wherein the composition comprises a vector comprising a guide RNA of the disclosure and a nucleic acid sequence encoding an RNA-binding protein or an RNA-binding protein fusion protein of the disclosure, wherein the composition modifies, reduces, destroys, knocks down or ablates a level of expression of a toxic CAG repeat RNA (compared to the level of expression of a toxic CAG repeat RNA treated with a non-targeting (NT) control or compared to no treatment). In one embodiment, the level of reduction of the target toxic CAG repeat RNA or toxic repeats encoded by the target RNA is compared to the level of reduction of the target RNA or toxic repeats encoded by the target RNA when treated with a non RNase Cas-based system (e.g., such as RCas9). In another embodiment, the level of reduction is 1-fold or greater. In another embodiment, the level of reduction is 2-fold, 3-fold, 4-fold, 5-fold, 6-fold, 7-fold, 8-fold, 9-fold or 10-fold. In another embodiment, the level of reduction is 10-fold or greater. In another embodiment, the level of reduction is between 10-fold and 20-fold. In another embodiment, the level of reduction is 11-fold, 12-fold, 13-fold, 14-fold, 15-fold, 16-fold, 17-fold, 18-fold, 19-fold, or 20-fold. In another embodiment, the gene therapy compositions disclosed herein when administered to a patient lead to 20%-100% destruction of the toxic CAG repeat RNA. In one embodiment, the % elimination of the toxic CAG repeat RNA is any of 20-99%, 25%-99%, 50%-99%, 80%-99%, 90%-99%, 95%-99%. In one embodiment, the % elimination is 90%, 91%, 92%, 93%, 94%, 95%, 96%, 97%, 98%, or 99%. In another embodiment, % elimination is complete elimination or 100% elimination of the toxic CAG repeat RNA.

In some embodiments, CAG-repeat RNA targeting compositions of the disclosure alter expression of proteins translated from CAG-repeat containing RNA (such as mRNA). In some aspects, the protein expression is reduced or eliminated. In some aspects, a CAG repeat comprising protein is mutated HTT (mHTT). In some aspects, a CAG repeat comprising protein is mutated ataxin-1 (mATXN1).

In some embodiments of the compositions and methods of the disclosure, a disease or disorder of the patient to be treated includes, without limitation, a disease or disorder related to CAG microsatellite repeat expansion expression. In some embodiments, the disease or disorder is related to CAG microsatellite repeat expansion in the HTT gene (HD) or ATXN1 gene (SCA1). In some embodiments of the compositions and methods of the disclosure, a disease or disorder of the disclosure is HD or SCA1.

In some embodiments of the methods of the disclosure, a subject of the disclosure has been diagnosed with a CAG repeat disorder. In some embodiments of the methods of the disclosure, a subject of the disclosure has been diagnosed with a CAG repeat disorder such as HD or SCA1. In some embodiments, the subject of the disclosure presents at least one sign or symptom of a CAG repeat disorder. In some embodiments, the subject of the disclosure presents at least one sign or symptom of HD. In some embodiments, the subject of the disclosure presents at least one sign or symptom of SCA1. At least one HD sign or HD symptom includes, without limitation, depression, poor coordination (with walking, speaking, swallowing), chorea, cognitive impairment (learning, lack of decisiveness, reasoning, decline in thinking abilities), and/or seizures. At least one SCA1 sign or SCA1 symptom includes, without limitation, coordination and balance issues (ataxia), speech and swallowing difficulties, muscle stiffness (spasticity), weakness in the muscles that control eye movements (nystagmus), cognitive impairment (with processing, learning, memory), sensory neuropathy, dystonia, atrophy, fasciculations, tremors, and/or chorea. In one embodiment, at least one sign or symptom of the CAG repeat disease such as HD or SCA1 is ameliorated by treatment with the compositions disclosed herein. In some embodiments, the subject has a biomarker predictive of a risk of developing a CAG repeat disease such as HD or SCA1. In some embodiments, the biomarker is a genetic mutation.

In some embodiments of the methods of the disclosure, a subject of the disclosure is female. In some embodiments of the methods of the disclosure, a subject of the disclosure is male. In some embodiments, a subject of the disclosure has two XX or XY chromosomes. In some embodiments, a subject of the disclosure has two XX or XY chromosomes and a third chromosome, either an X or a Y.

In some embodiments of the methods of the disclosure, a subject of the disclosure is a neonate, an infant, a child, an adult, a senior adult, or an elderly adult. In some embodiments of the methods of the disclosure, a subject of the disclosure is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 days old. In some embodiments of the methods of the disclosure, a subject of the disclosure is at least 1, 2, 3, 4, 5, 6, 7, 8, 9, 10, 11 or 12 months old. In some embodiments of the methods of the disclosure, a subject of the disclosure is at least 1, 2, 3, 4, 5, 6, 7, 8, 9, 10, 15, 20, 25, 30, 35, 40, 45, 50, 55, 60, 65, 70, 75, 80, 85, 90, 95, 100 or any number of years or partial years in between of age.

In some embodiments of the methods of the disclosure, a subject of the disclosure is a mammal. In some embodiments, a subject of the disclosure is a non-human mammal.

In some embodiments of the methods of the disclosure, a subject of the disclosure is a human.

In some embodiments of the methods of the disclosure, a therapeutically effective amount comprises a single dose of a composition of the disclosure. In some embodiments, a therapeutically effective amount comprises a therapeutically effective amount comprises at least one dose of a composition of the disclosure. In some embodiments, a therapeutically effective amount comprises a therapeutically effective amount comprises one or more dose(s) of a composition of the disclosure.

In some embodiments of the methods of the disclosure, a therapeutically effective amount eliminates a sign or symptom of the disease or disorder. In some embodiments, a therapeutically effective amount reduces a severity of a sign or symptom of the disease or disorder.

In some embodiments of the methods of the disclosure, a therapeutically effective amount eliminates the disease or disorder.

In some embodiments of the methods of the disclosure, a therapeutically effective amount prevents an onset of a disease or disorder. In some embodiments, a therapeutically effective amount delays the onset of a disease or disorder. In some embodiments, a therapeutically effective amount reduces the severity of a sign or symptom of the disease or disorder. In some embodiments, a therapeutically effective amount improves a prognosis for the subject.

In some embodiments of the methods of the disclosure, a composition of the disclosure is administered to the subject via intracerebral administration. In some embodiments, the composition of the disclosure is administered to the subject by an intrastriatal route. In some embodiments, the composition of the disclosure is administered to the subject by a stereotaxic injection or an infusion. In some embodiments, the composition is administered to the brain. In some embodiments of the methods of the disclosure, a composition of the disclosure is administered to the subject locally.

In some embodiments, the compositions disclosed herein are formulated as pharmaceutical compositions. Briefly, pharmaceutical compositions for use as disclosed herein may comprise a protein(s) or a polynucleotide encoding the protein(s), optionally comprised in an AAV, which is optionally also immune orthogonal, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the disclosure may be formulated for routes of administration, such as e.g., oral, enteral, topical, transdermal, intranasal, and/or inhalation; and for routes of administration via injection or infusion such as, e.g., intravenous, intramuscular, subpial, intrathecal, intraparenchymal, intrathecal, intrastriatal, subcutaneous, intradermal, intraperitoneal, intratumoral, intravenous, intraocular, and/or parenteral administration. In certain embodiments, the compositions of the present disclosure are formulated for intracerebral or intrastriatal administration.

### EXAMPLES

### Example 1: Cas13d and PUF Systems Destroy Toxic CAG Repeats

### Methods

Transfection, RNA extraction, FISH, qRT-PCR Analysis

Cleavage efficiency of CAG repeats in vitro was detected by exogenously expressing 80 CAG repeats driven by the CMV promoter and assessing knockdown of CAG-repeat containing RNA using an in house designed qRT-PCR assay and or FISH (DAPI staining and fluorescent CAG probe). Immunofluorescence using anti-polyQ antibody indicated elimination of toxic Poly-Q protein aggregates. Cas and CAG spacer systems or PUF protein linked to the endonuclease E17 proteins targeting CAG repeats were used to evaluate cleavage of CAG-repeat containing RNA. For all experiments 1 ug of the effector or effector and guide were used to transfect cells using Lipofectamine 3000 (Thermo) into CosM6 cells (according to the manufacturer's protocol) along with the 50 ng of the pCMV-CAG80 reporter plasmid. Cells were subjected to qRT-PCR or FISH for analysis. (A myc-tagged version of PUF-CAG-E17 was used and protein expression was detected by IF (immunofluorescence) using an anti-myc antibody.) Transfected cells were harvested 48 h post-transfection, and for qRT-PCR RNA was extracted using the Qiagen RNeasy kit, and qRT-PCR for the CAG repeat was performed using the Quantabio 1-step qRT-PCR kit, Biorad qPCR machine and the following primer sets: CAG Forward: CAAAGACCACGACGGAGATT (SEQ ID NO: 584) Reverse: TCAGCTTCTGCTCCAGATCC (SEQ ID NO: 585). CAG expression was normalized to GAPDH reference gene and calculated relative to no targeting control conditions.

In some aspects, a truncated CAG (tCAG) promoter (SEQ ID NO: 389) was used. In some aspects, a short EF1-alpha (EFS) promoter (SEQ ID NO: 520) was used.

For Cas13d systems, the spacers used in CAG targeting guides are as follows:

| Spacer | Spacer Sequences |
|---|---|
| CAG guide 1 | tgctgctgctgctgctgctgctgctg |
| | (SEQ ID NO: 457) |
| CAG guide 2 | gctgctgctgctgctgctgctgctgc |
| | (SEQ ID NO: 458) |
| CAG guide 3 | ctgctgctgctgctgctgctgctgct |
| | (SEQ ID NO: 459) |

For PUF targeting CAG, the construct encoding the following 8PUF(CAG) was used:

| Protein | | Target | Amino Acid |
|---|---|---|---|
| Type | Elements | Sequence | Sequence of PUF |
| 8PUF | Linker between | CAGCAGCA | GRSRLLEDFRNNRYPNLQL |
| Frame 1 | PUF and E17 | | REIAGHIMEFSQDQHGSRF |
| | endonuclease | | IQLKLERATPAERQLVFNE |
| | (VDTANGS); | | ILQAAYQLMVDVFGSYVIR |
| | C-terminal | | KFFEFGSLEQKLALAERIR |
| | E17; Some | | GHVLSLALQMYGSRVIEKA |
| | extra amino | | LEFIPSDQQNEMVRELDGH |
| | acids before | | VLKCVKDQNGCYVVQKCIE |
| | R1′ and | | CVQPQSLQFIIDAFKGQVF |
| | between R8′ | | ALSTHPYGSRVIRRILEHC |
| | and linker. | | LPDQTLPILEELHQHTEQL |
| | R4 amino acid | | VQDQYGSYVIEHVLEHGRP |
| | 13 Y instead | | EDKSKIVAEIRGNVLVLSQ |
| | of H | | HKFACNVVQKCVTHASRTE |
| | | | RAVLIDEVCTMNDGPHSAL |
| | | | YTMMKDQYASYVVRKMIDV |
| | | | AEPGQRKIVMHKIRPHIAT |
| | | | LRKYTYGKHILAKLEKYYM |
| | | | KNGVDLG |
| | | | (SEQ ID NO: 480) |
| 8PUF | N-terminal | GCAGCAGC | GRSRLLEDFRNNRYPNLQL |
| Frame 2 | PUF and E17 | | REIAGHIMEFSQDQHGSRF |
| | endonuclease | | IRLKLERATPAERQLVFNE |
| | (VDTANGS); | | ILQAAYQLMVDVFGSYVIE |
| | C-terminal E17 | | KFFEFGSLEQKLALAERIR |
| | | | GHVLSLALQMYGCRVIQKA |
| | | | LEFIPSDQQNEMVRELDGH |
| | | | VLKCVKDQNGSYVVRKCIE |
| | | | CVQPQSLQFIIDAFKGQVF |
| | | | ALSTHPYGSRVIERILEHC |
| | | | LPDQTLPILEELHQHTEQL |
| | | | VQDQYGCYVIQHVLEHGRP |
| | | | EDKSKIVAEIRGNVLVLSQ |
| | | | HKFASYVVRKCVTHASRTE |
| | | | RAVLIDEVCTMNDGPHSAL |
| | | | YTMMKDQYASYVVEKMIDV |
| | | | AEPGQRKIVMHKIRPHIAT |
| | | | LRKYTYGKHILAKLEKYYM |
| | | | KNGVDLG |
| | | | (SEQ ID NO: 549) |

### Example 2: Targeting Expanded CAG Repeats at the RNA Level for the Treatment of CAG Repeat Disease Huntington's Disease by PUF-E17

A transgene encoding CAG-targeting PUF linked to the endonuclease E17 (derived from human ZC3HT12A gene) is delivered via either an intrastriatal route via viral or nonviral approaches. The PUF targeting CAG construct for AAV-based delivery in the below art-recognized animal model for Huntington's Disease, R6/2 mouse model, is:

| Protein | | Target | Amino Acid |
|---|---|---|---|
| Type | Elements | Sequence | Sequence of PUF |
| 8PUF | Linker between | CAGCAGCA | GRSRLLEDFRNNRYPNLQL |
| Frame 1 | PUF and E17 | | REIAGHIMEFSQDQHGSRF |
| | endonuclease | | IQLKLERATPAERQLVFNE |
| | (VDTANGS); | | ILQAAYQLMVDVFGSYVIR |
| | C-terminal | | KFFEFGSLEQKLALAERIR |
| | E17; Some | | GHVLSLALQMYGSRVIEKA |
| | extra amino | | LEFIPSDQQNEMVRELDGH |
| | acids before | | VLKCVKDQNGCYVVQKCIE |
| | R1′ and | | CVQPQSLQFIIDAFKGQVF |
| | between R8′ | | ALSTHPYGSRVIRRILEHC |
| | and linker. | | LPDQTLPILEELHQHTEQL |
| | R4 amino acid | | VQDQYGSYVIEHVLEHGRP |
| | 13 Y instead | | EDKSKIVAEIRGNVLVLSQ |
| | of H | | HKFACNVVQKCVTHASRTE |
| | | | RAVLIDEVCTMNDGPHSAL |
| | | | YTMMKDQYASYVVRKMIDV |
| | | | AEPGQRKIVMHKIRPHIAT |
| | | | LRKYTYGKHILAKLEKYYM |
| | | | KNGVDLG |
| | | | (SEQ ID NO: 480) |
| 8PUF | N-terminal | GCAGCAGC | GRSRLLEDFRNNRYPNLQL |
| Frame 2 | PUF and E17 | | REIAGHIMEFSQDQHGSRF |
| | endonuclease | | IRLKLERATPAERQLVFNE |
| | (VDTANGS); | | ILQAAYQLMVDVFGSYVIE |
| | C-terminal E17 | | KFFEFGSLEQKLALAERIR |
| | | | GHVLSLALQMYGCRVIQKA |
| | | | LEFIPSDQQNEMVRELDGH |
| | | | VLKCVKDQNGSYVVRKCIE |
| | | | CVQPQSLQFIIDAFKGQVF |
| | | | ALSTHPYGSRVIERILEHC |
| | | | LPDQTLPILEELHQHTEQL |
| | | | VQDQYGCYVIQHVLEHGRP |
| | | | EDKSKIVAEIRGNVLVLSQ |
| | | | HKFASYVVRKCVTHASRTE |
| | | | RAVLIDEVCTMNDGPHSAL |
| | | | YTMMKDQYASYVVEKMIDV |
| | | | AEPGQRKIVMHKIRPHIAT |
| | | | LRKYTYGKHILAKLEKYYM |
| | | | KNGVDLG |
| | | | (SEQ ID NO: 549) |

In order to target expanded CAG repeats associated with HD, AAV vector with DNA encoding CAG-targeting PUF-E17 is delivered to via bilateral stereotaxic injection. PUF-E17 expression is driven by a promoter (FIG. 3A). In some aspects, a truncated CAG (tCAG) promoter (SEQ ID NO: 389) was used.

### Example 3: Assessment of CAG-Vectors in HD Mouse Models

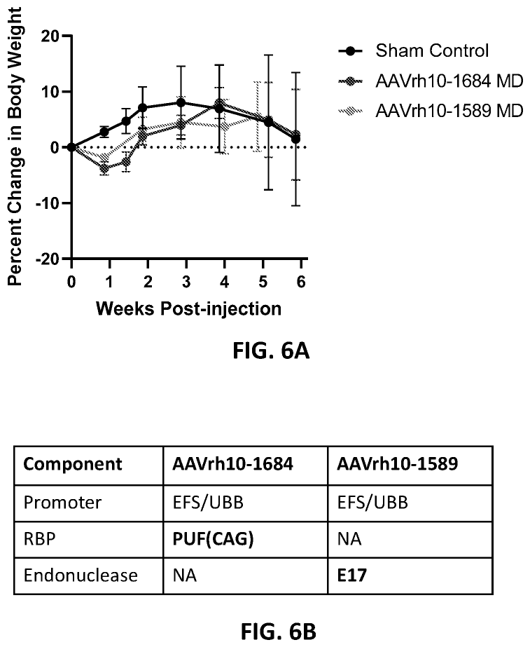

CAG-targeting PUF AAVrh10-1684 and AAVrh10-1589 (comprising the features in FIG. 6B3) were tested in a R6/2 mouse model. Body weight of the mice was evaluated in the weeks following injection.

FIG. 6A is a graph depicting percent change in body weight in mice treated with either an AAVrh10-1684 vector or AAVrh10-1589 vector at a mid-dose relative to a sham control.

FIG. 6B is a table depicting the vector composition of the AAVrh10-1684 vector and the AAVrh10-1589 vector. AAVrh10-1684 comprises an EFS/UBB promoter controlling expression of a CAG-targeted PUF protein lacking an endonuclease fusion. AAVrh1-1589 comprises an EFS/UBB promoter controlling expression of an E17 endonuclease lacking a CAG-targeting RNA binding protein.

### Example 4: Optimization of CAG-Repeat Targeting RNA Delivery in Non-Human Primates

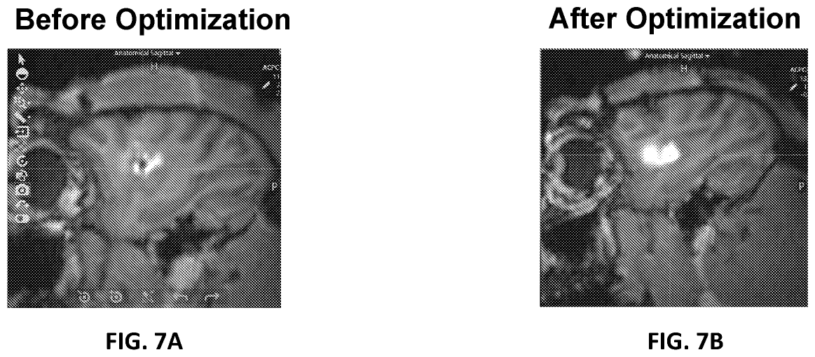

AAVrh10-1383 (LBIO-210) was evaluated to assess tolerability in different species. In a non-human primate delivery of LBIO-210 was optimized according to the following: reduced volume and flow rate; altered cannula type; identified ideal cannula placement. 08431 FIG. 7 is a series of images depicting gadoteridol expression representative of delivery of AAVrh10-1383 (LBIO-210) in non-human primates before (FIG. 7A) and after (FIG. 7i) delivery optimization.

| Surgery | Dose | Surgery | | |
|---|---|---|---|---|
| # | Level | Comments | In-life observations | Interpretation |
| 1 | High | Overfilling | Mild left leg tremor | Procedure- |
| | | of putamen | developed 5-6 days | related |
| | | likely; some | post-injection | |
| | | vector efflux | | |
| 2 | Low | Large amount of | No observations | LBIO-210 |
| | | vector efflux; Air | | well-tolerated |
| | | bubble observed | | |
| | | at injection site | | |
| 3 | High | Good targeting | No observations | LBIO-210 well- |
| | | | | tolerated so far |
| 4 | Low | Good targeting | Mild bilateral tremor | Waiting for |
| | | | developed 8 days | neuroradiologist |
| | | | post-injection | review of MRI |
| 5 | High | Good targeting | No observations | LBIO-210 well- |
| 6 | High | Good targeting | No observations | tolerated so far |
| 7 | High | Likely cortical | Left arm and left | Procedure- |
| | | damage during | leg weakness | related |
| | | injection due to | developed 3 | |
| | | cannula | days post-injection | |
| | | deflection | | |

### Example 5: CAG-Targeting RCas9 System Reduces Mutant HTT Protein with No Change in Mutant HTT RNA Levels

A CAG-repeat targeting RCas9 system was evaluated to assess the impact of HTT protein expression by targeting CAG-repeat RNA in mice.

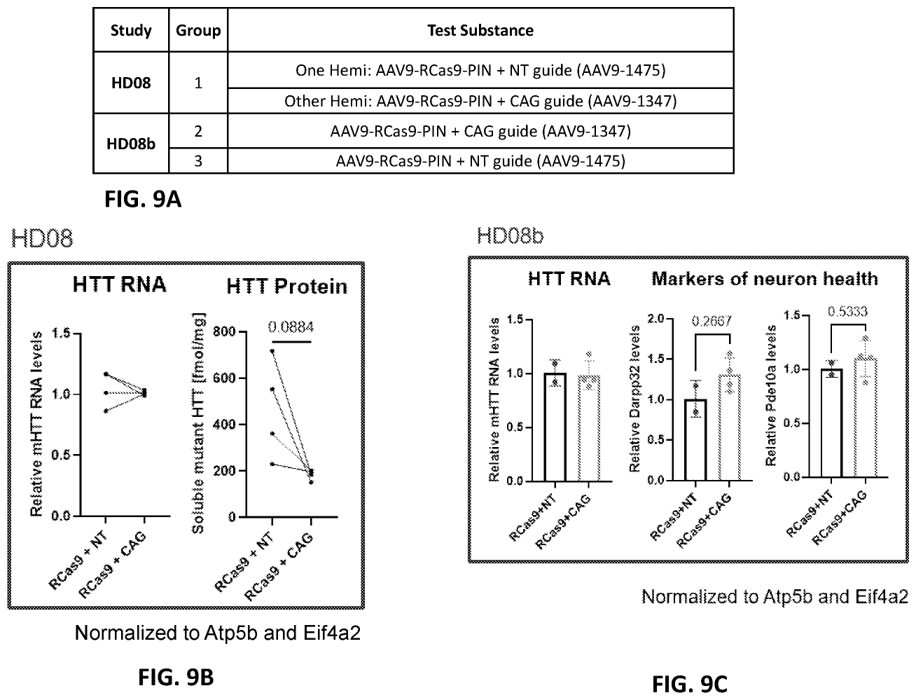

FIG. 9A is a table depicting rCas9 constructs used in FIGS. 9B and 9C. Study HD08 group 1 is divided into two halves (hemispheres): hemi 1 utilized AAV9-rCas9-PIN and a non-targeting (NT) guide RNA (AAV9-1475) while the other hemi (hemi 2) utilized AAV9-rCas9-PIN with a CAG repeat-targeting guide RNA (AAV9-1347). Study HD08b was divided into group 2 (AAV9-RCas9-PIN+CAG guide (AAV9-1347) and group 3 AAV9-RCas9-PIN+NT guide (AAV9-1475).

FIG. 9B is a series of graphs depicting relative mutant HTT (mHTT) RNA levels and protein (soluble mHTT) levels in mice following treatment with RCas9+NT or RCas9+CAG (Study HD08). *mHTT RNA levels normalized to Atp5b and Eif4a2.

FIG. 9C is a series of graphs depicting relative mutant HTT (mHTT) RNA levels in mice following treatment with RCas9+NT or RCas9+CAG and relative Darpp32 levels and relative Pdel0a levels*. (Study HD08b). *Normalized to Atp5b and Eif4a2.

No body weight loss was observed following treatment. Further, no change in mutant HTT RNA levels suggests that PIN is a weak endonuclease (FIG. 9B). However, a large reduction in soluble mutant HTT protein [3 out of 4 animals showed meaningful reductions (44-74% decrease)].

### Example 6: Establishing zQ175 P1 Cortical Neuron Cultures as an Efficacy and Safety Model

P1 cortical neurons were derived from zQ175 knock-in (zQ175 KI) allele mice has the mouse HTT exon 1 replaced with human HTT exon 1 sequences with an about 190 CAG repeat tract. These B6J.zQ175 KI mice (Jax Lab, Stock No. 027410) are useful for studying Huntington's disease pathogenesis and for the assessment of potential therapeutic interventions. Isolation and culture of P1 neurons from zQ175 mice facilitates higher-throughput assessments of gene therapy constructs in a relevant neuronal disease model.

Overall Method

Isolate P1 neurons from zQ175 mice using papain dissociation method and mature cultures for 10 days (adding AraC on day 3. Transduce cultures with viral constructs (i.e. CAG-targeting proteins of the disclosure) on day 10. Maintain cultures for 7 days post-transduction sampling supernatant and cell lysates for efficacy and safety assessments at appropriate timepoints.

Methods

Results

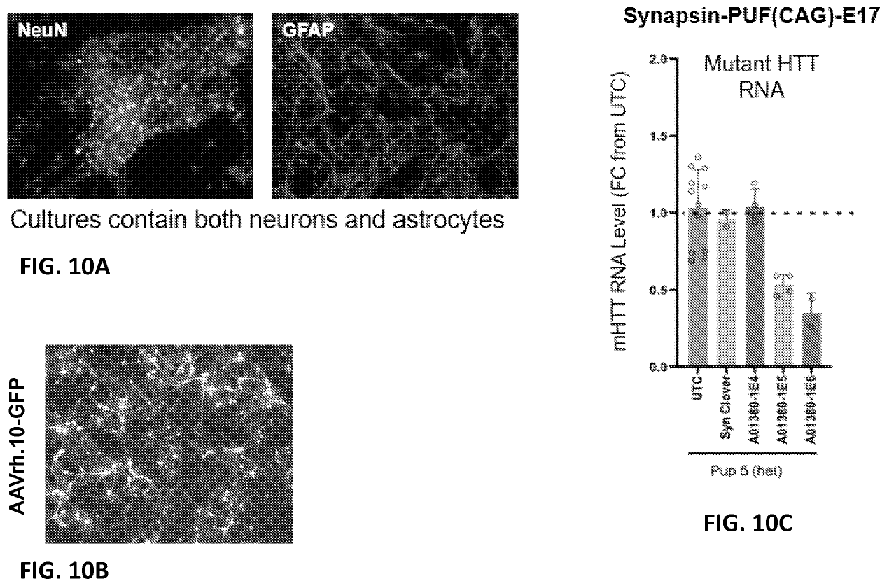

Established zQ175 P1 cortical neuron cultures contain both neurons and astrocytes as measured by fluorescent microscopy and immunohistochemical staining (FIG. 10A).

Next, cultured cells were assessed for the ability to transduce AAVrh10 vectors. AN AAVrh10 vector encoding green fluorescent protein (GFP) is readily transduced and GFP is readily expressed (FIG. 10B).

Mutant HTT (mHTT) levels were assessed following treatment of the cell culture with CAG-targeting AAV constructs of the disclosure and mHTT levels were compared to untreated control (UTC) (FIG. 10C). Vector A01380 (synapsin-PUF(CAG)-E17) comprising the neuron-specific promoter synapsin delivered at an MOI of 1E4, 1E5, and 1E6. Dose-dependent reduction in mHTT levels were observed with increasing dosage of A01380 vector (FIG. 10C).

### Example 7: HD Patient-Derived Cells Allow Evaluation of Allele Preference and Efficacy Across a Range of CAG Repeat Lengths

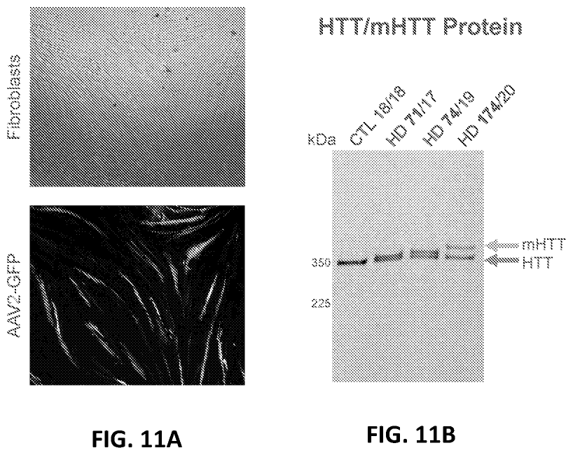

Patient-derived cells allow evaluation of allele preference and efficacy across a range of varying CAG repeat lengths. FIG. 11A is a series of images of Huntington Disease patient-derived fibroblasts. FIG. 11B is an image of a gel depicting both wild-type and mutated HTT. These fibroblasts are a useful system for testing CAG-targeting compositions of the disclosure.

### Example 8: Assessment of Cas13d CAG-Targeting Constructs in zQ175 P1 Neurons

P1 cortical neurons were derived from zQ175 knock-in (zQ175 KI) allele mice has the mouse HTT exon 1 replaced with human HTT exon 1 sequences with an about 190 CAG repeat tract. These B6J.zQ175 KI mice (Jax Lab, Stock No. 027410) are useful for studying Huntington's disease pathogenesis and for the assessment of potential therapeutic interventions. Isolation and culture of P1 neurons from zQ175 mice facilitates higher-throughput assessments of gene therapy constructs in a relevant neuronal disease model.

### Overall Method

Isolate P1 neurons from zQ175 mice using papain dissociation method and mature cultures for 10 days (adding AraC on day 3. Transduce cultures with viral constructs (i.e., CAG-targeting proteins of the disclosure) on day 10. Maintain cultures for 7 days post-transduction sampling supernatant and cell lysates for efficacy and safety assessments at appropriate timepoints.

### Methods

### In-Life:

Day 1: Cells isolated, plated, and maintained in 24-well plates as described in previous slide

Day 3: Ara-C administration begins at final concentration of 1 uM

Day 10: Perform AAV transductions at 1E5 and 1E6 MOI. Sample baseline media and cell lysates (if possible, samples permitting) prior to administering transductions

Day 13: Harvest media and cell lysates for 3 day post-transduction timepoint (if possible, samples permitting)

Day 17: Harvest media and cell lysates for 7 day post-transduction timepoint

### Endpoint Assays:

RNA prepared and qRT-PCR ran to quantitate expression levels of constructs and target transcripts.

Protein prepared for assessment of mHTT and WT HT protein levels via Meso Scale Discovery (MSD).

LDH-Glo cytotoxicity assay.

### Analysis:

Target transcript expression normalized to reference gene panel (GAPDH, EIF4A2, and ATP5B)

HKG-normalized data normalized to standard curve to account for primer-to-primer variation in efficiency.

Cytotoxicity data background subtracted and plotted as fold change from untreated control.

### Materials

AAVs: Details listed in Table U.
RNA Prep: Rneasy 96 (Qiagen, 74182)
qRT-PCR: TaqPath 1-Step Multiplex Master Mix (ThermoFisher, A28522)
Primers: HTT-FAM, mGAPDH-HEX, mEIF4A2-Cy5, and mATP5B-HEX
Cell Health: Cytotoxicity (LDH-Glo, J2380, Promega)

TABLE U
Vectors used in study and study design

| Test Articles | Cell/Animals | Dose (MOI) | Timepoints |
|---|---|---|---|
| dCas13d dSeq212-CAG- | 1. zQ175 | 1E5 and 1E6 | D7 post- |
| AAVrh10.A01553 | P1 | | transduction |
| Cas13d Seq212-CAG | Neurons | | |
| Guide | | | |
| Only-AAVrh10.A01477 | | | |
| dCas13d dSeq212-CAG- | | | |
| AAVrh10.A01479 | | | |
| PUF-CAG - | | | |
| AAVrh10.A01383 | | | |
| shRNA-CAG-AAVrh9 | | | |

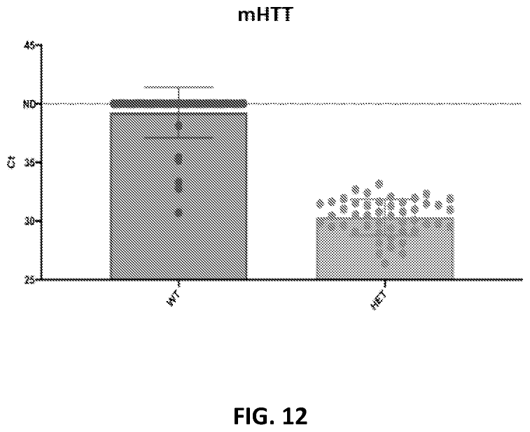

Mutant HTT (mHTT) expression was assessed in P1 neuronal cultures derived from untreated WT and HET pups as measured by qRT-PCR (FIG. 12). HET-specific expression of mHTT was demonstrated using raw Cts, whereas in 40 of 46 wildtype samples no mHTT was detected.

CAG-repeat targeting constructs of the disclosure were assessed for their ability to alter mHTT expression in P1 neuronal cultures. The P1 neuronal cultures were transduced with vectors of the disclosure including CAG-targeting PUF proteins and CAG-targeting dCas13d (Seq212) proteins for 7 days. Vectors used include those in table U Doses included 1E5 and 1E6 MOI. mHTT and WT HT expression levels were measured by qRT-PCR

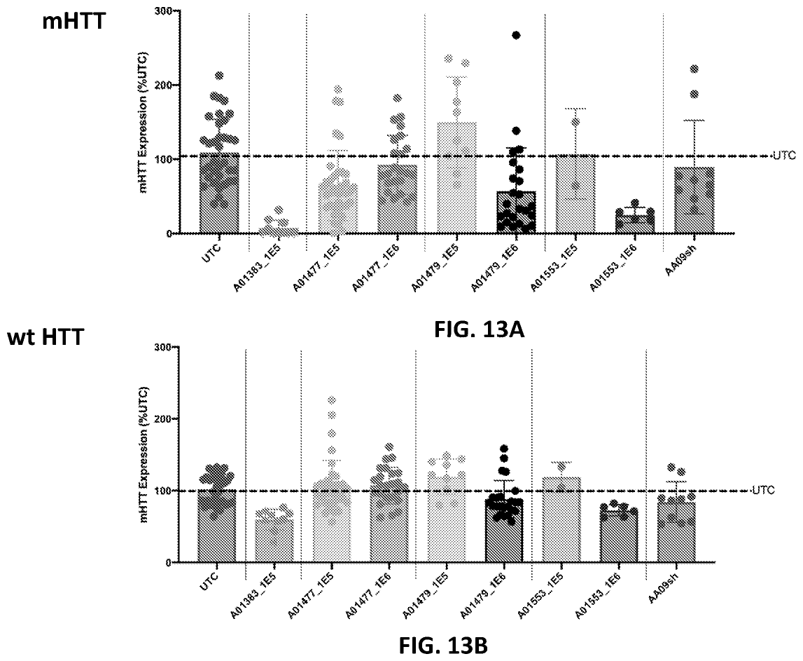

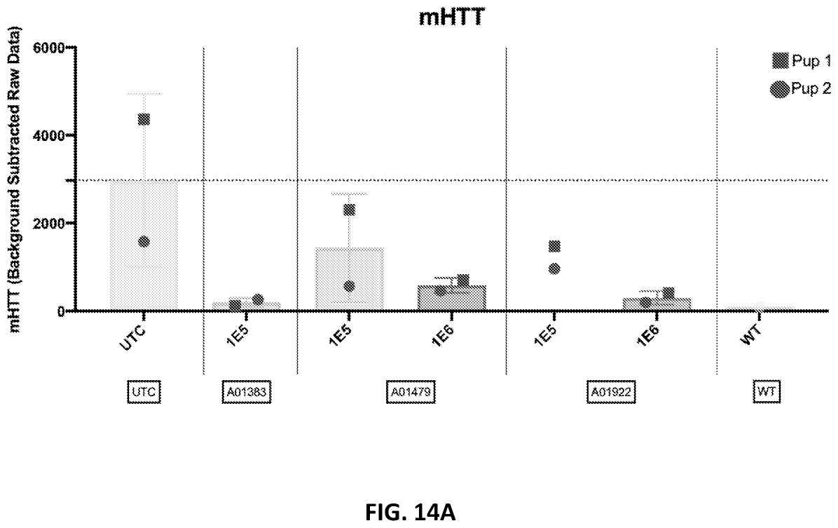

mHTT-specific knockdown (KD) was observed with CAG-targeting constructs A01383, A01479, and A01553 as assessed by increased delta Ct where increased knockdown is indicated by higher delta Ct (FIG. 13A). Wildtype HTT levels were largely unaffected (FIG. 13B).

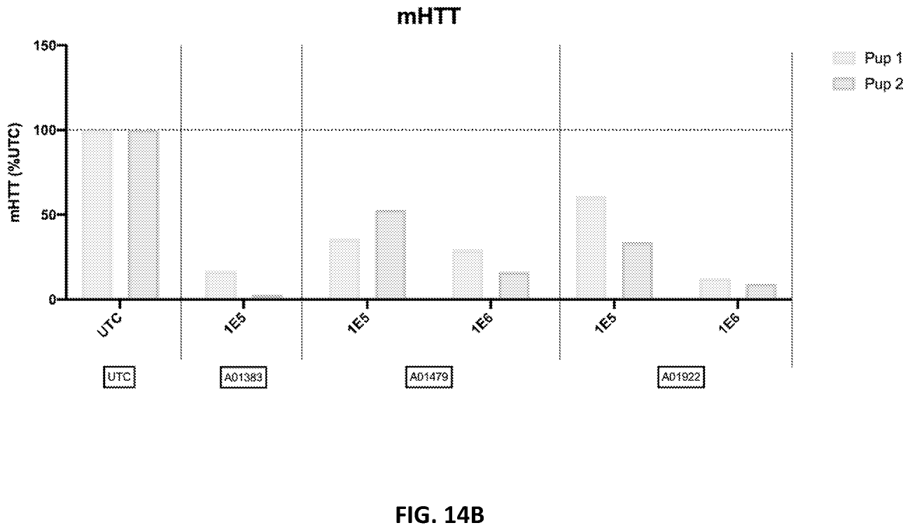

P1 neurons derived from heterozygous zQ175 mouse pups were transduced with CAG-targeting PUF and Cas1d Seq212 constructs at 1E5 and 1E6 MOI for 7 days. mHTT protein levels were measured by Meso Scale Discovery Immunoassay (MSD) (FIG. 14A and FIG. 14B). P1 neurons were prepared from zQ175 heterozygous pups using a papain dissociation method. After 10 days of maturation, neurons transduced with CAG-targeting PUF and Cas13d Seq212 constructs at 1E5 and 1E6 MOI for 7 days. Cells lysed and mHTT protein levels measured using Meso Scale Discovery Immunoassay (MSD). mHTT protein knockdown was observed with CAG-targeting constructs A01383, A01479, and A01922.

Expression of CAG-repeat targeting cas13d constructs were assessed to measure both casl3d expression and guide RNA expression in mHTT protein KD observed with CAG-targeting constructs A01383, A01479, and A01922

dCas13d (Seq212) and guide RNA expression levels were measured by qRT-PCR.

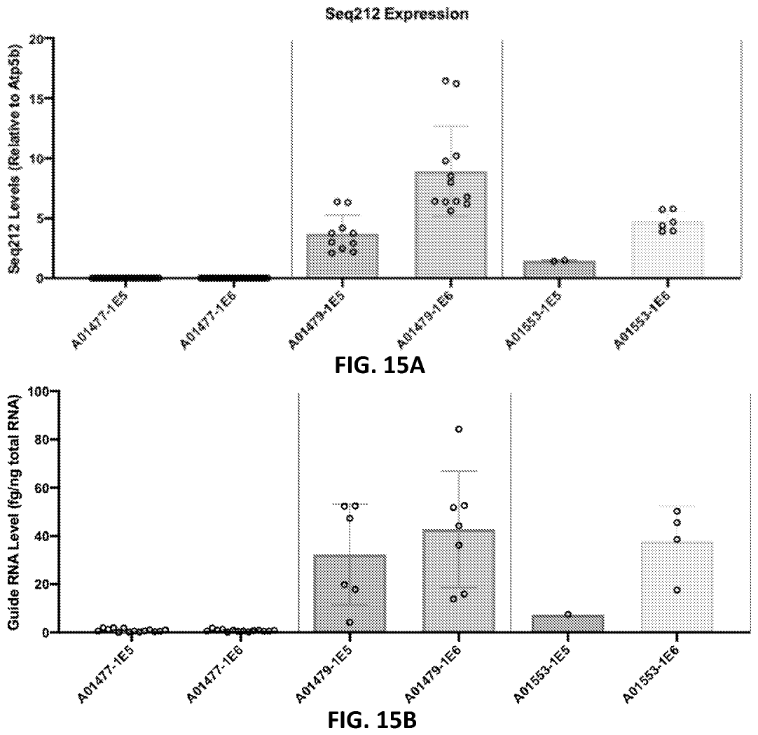

dCas13d-expressing constructs A01479 and A01553 exhibit similar levels of dCas13d expression (Higher expression=Lower delta Ct) (FIG. 15A).

Comparable dose responsive guide RNA levels was observed with dCas13d-expressing constructs A01479 and A0155 (FIG. 15B). Low guide RNA levels with “guide only” (No Seq212) construct A01477 was observed.

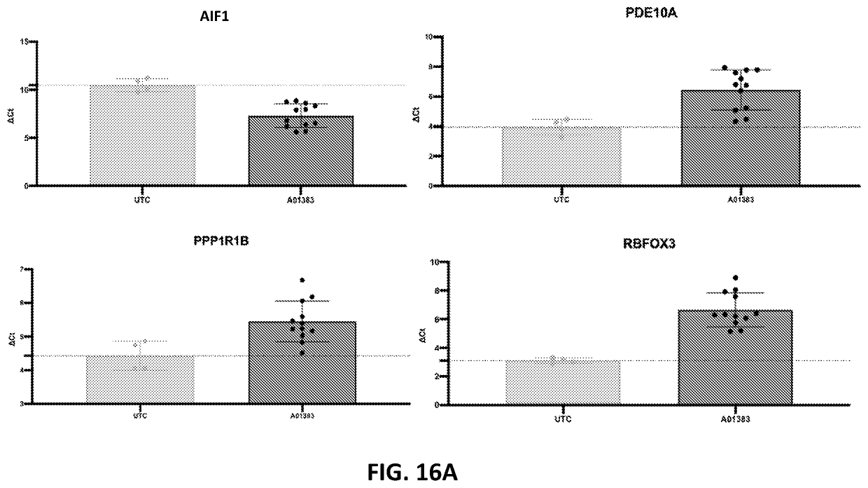

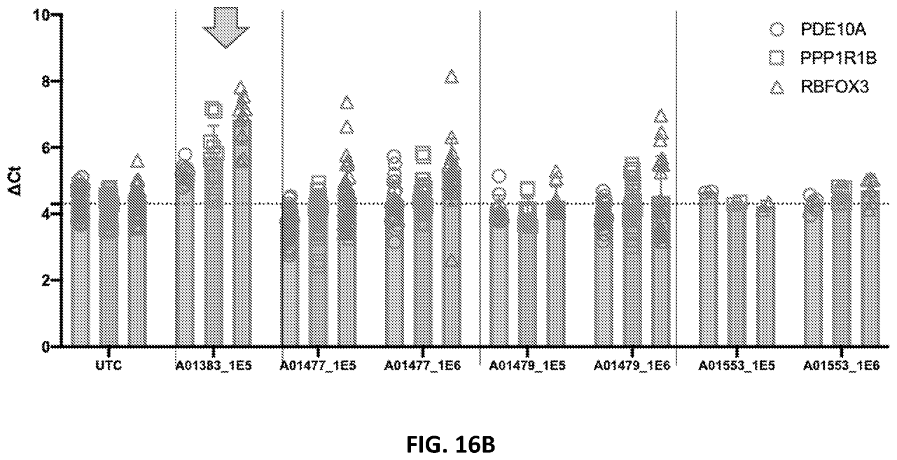

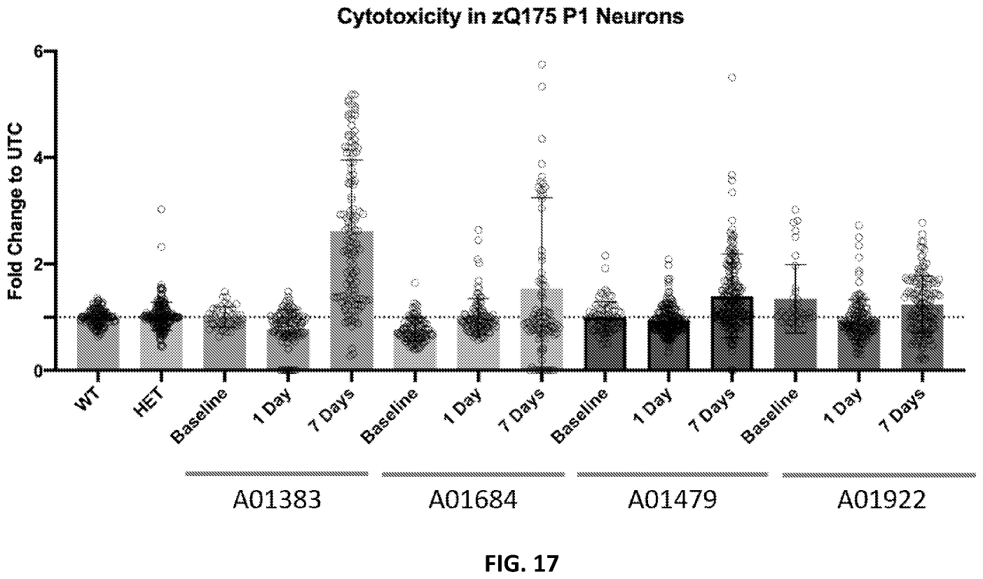

Neuronal health signatures evaluated in P1 neurons transduced with CAG-targeting PUF A01383 at 1E5 MOI for 7 days. Neuronal and microglial activation marker, AIF1, PDE10A, PPPIR1B, and RBFOX3 expression levels measured by qRT-PCR. Neuronal and microglial activation marker expression levels measured by qRT-PCR (FIG. 16A and FIG. 16B). CAG-repeat targeting PUF construct A01383-specific neuronal health signature observed (compared to dCas13d constructs). Lower expression=increased delta Ct. Stimulated expression=lowered delta Ct. Further, cytotoxicity was assessed for each vector construct. P1 neurons transduced with CAG-targeting constructs at 1E5 MOI for 7 days (FIG. 17). Cytotoxicity was assessed using LDH-Glo (Promega). A01383-enriched cytotoxicity observed (compared to dCas13d Seq212 constructs). A neuronal health gene signature was developed that can be predictive of in vivo safety.

### INCORPORATION BY REFERENCE

Every document cited herein, including any cross referenced or related patent or application is hereby incorporated herein by reference in its entirety unless expressly excluded or otherwise limited. The citation of any document is not an admission that it is prior art with respect to any invention disclosed or embodiment herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

### OTHER EMBODIMENTS

While particular embodiments of the disclosure have been illustrated and described, various other changes and modifications can be made without departing from the spirit and scope of the disclosure. The scope of the appended claims includes all such changes and modifications that are within the scope of this disclosure.

What is claimed is:

1. A composition comprising a nucleic acid sequence encoding an RNA-binding polypeptide comprising a non-guided RNA binding polypeptide or a guided RNA-binding polypeptide capable of binding a toxic target CAG repeat RNA sequence.

2. The composition of claim 1, wherein the RNA-binding polypeptide is a fusion protein.

3. The composition of claim 2, wherein the fusion protein comprises the RNA binding polypeptide fused to an endonuclease capable of cleaving the toxic CAG repeat RNA sequence.

4. The composition of any one of the preceding claims, wherein the non-guided RNA binding polypeptide is a PUF or PUMBY protein.

5. The composition of any one of the preceding claims, wherein the guided RNA-binding polypeptide is a Cas13d protein.

6. The composition of any one of the preceding claims, wherein the cas13d protein is catalytically dead.

7. The composition of any one of the preceding claims, wherein the casl3d protein comprises an amino acid sequence set forth in any one of SEQ ID NOs 587 or 590-594.

8. The composition of any one of the preceding claims, wherein the endonuclease is a nuclease domain of a ZC3H12A zinc-finger endonuclease.

9. The composition of any one of the preceding claims, wherein the PUF RNA binding protein comprises an amino acid sequence set forth in any one of SEQ ID NOs 444-451, 461, 480-488, 549-557, or 656.

10. The composition of any one of the preceding claims, wherein the PUF RNA binding protein comprises an amino acid sequence set forth in SEQ ID NO: 549 or 480.

11. The composition of any one of the preceding claims, wherein the toxic target CAG RNA repeat sequence comprises any one of the nucleic acid sequences set forth in SEQ ID NOs 453-456 or 472-479.

12. The composition of any one of the preceding claims, wherein the toxic target CAG RNA repeat sequence comprises the nucleic acid sequence set forth in any one of SEQ ID NO: 453 or 472.

13. The composition of any one of the preceding claims, wherein the CAG-targeting PUF protein is encoded by a nucleic acid sequence as set forth in SEQ ID NO: 577, 581, 614, 619, 621, or 622.

14. The composition of any one of the preceding claims, wherein the PUF or PUMBY protein is a human PUF or PUMBY protein.

15. The composition of any one of the preceding claims, wherein the PUF or PUMBY protein is linked to the ZC3H12A endonuclease by a linker sequence.

16. The composition of any one of the preceding claims, wherein the linker comprises the amino acid sequence set forth in SEQ ID NO: 411.

17. The composition of any one of the preceding claims, wherein the fusion protein comprises one or more signal sequences selected from the group consisting of a nuclear localization sequence (NLS), and a nuclear export sequence (NES).

18. The composition of any one of the preceding claims, wherein the ZC3H12A zinc finger nuclease comprises the amino acid sequence set forth in SEQ ID NO: 358 or SEQ ID NO: 359.

19. The composition of any one of the preceding claims, wherein the fusion protein comprises the amino acid sequence set forth in any one of SEQ ID NO: 460.

20. The composition of any one of the preceding claims, wherein the fusion protein is encoded by a nucleic acid sequence comprising SEQ ID NO: 574-582.

21. The composition of any one of the preceding claims, wherein the nucleic acid molecule encoding the fusion protein comprises a promoter.

22. The composition of claim 14, wherein the promoter is a tCAG promoter, EFS/UBB promoter, or synapsin promoter.

23. A vector comprising the composition of any one of the preceding claims.

24. The vector of claim 23, wherein the vector is selected from the group consisting of: adeno-associated virus (AAV), retrovirus, lentivirus, adenovirus, nanoparticle, micelle, liposome, lipoplex, polymersome, polyplex, and dendrimer.

25. The vector of claim 23, which is an AAV vector.

26. An AAV vector of any one of the preceding claims, wherein the AAV vector comprises:
a first AAV ITR sequence;
a first promoter sequence;
a polynucleotide sequence encoding for at least one CAG-repeat RNA binding polypeptide; and
a second AAV ITR sequence.

27. The AAV vector of any one of the preceding claims, wherein the CAG-repeat RNA binding polypeptide comprises a PUF or PUMBY protein.

28. The AAV vector of any one of the preceding claims, wherein the polynucleotide sequence encoding the PUF or PUMBY sequence comprises a nucleic acid sequence set forth in SEQ ID NO: 577, 581, 614, 619, 621, or 622.

29. The AAV vector of any one of the preceding claims, wherein the CAG-repeat RNA binding polypeptide comprises a Cas13d protein.

30. The AAV vector of any one of the preceding claims, wherein the polynucleotide sequence encoding the Cas13d sequence comprises a nucleic acid sequence set forth in SEQ ID NO: 587 or 590-594.

31. The AAV vector of any one of the preceding claims, wherein the first promoter sequence comprises a nucleic acid sequence set forth in SEQ ID NO: 389, 627, or 613.

32. The AAV vector of any one of the preceding claims, wherein the first AAV ITR sequence comprises a nucleic acid sequence set forth in SEQ ID NO: 597 or 598.

33. The AAV vector of any one of the preceding claims, wherein the second AAV ITR sequence comprises a nucleic acid sequence set forth in SEQ ID NO: 597 or 598.

34. The AAV vector of any one of the preceding claims, wherein the vector further comprises a second promoter sequence.

35. The AAV vector of any one of the preceding claims, wherein the second promoter controls expression of a guide RNA (gRNA) wherein the gRNA comprises (i) a DR sequence and (ii) a spacer sequence.

36. The AAV vector of any one of the preceding claims, wherein the second promoter comprises a nucleic acid sequence set forth in SEQ ID NO: 519.

37. The AAV vector of any one of the preceding claims, wherein the vector further comprises a polyA sequence.

38. The AAV vector of any one of the preceding claims, wherein the vector comprises at least one linker sequence.

39. The AAV vector of any one of the preceding claims, wherein the vector comprises at least one nuclear localization sequence.

40. The AAV vector of any one of the preceding claims, wherein the vector is encoded be a nucleic set forth in any of one of SEQ ID NO: 588, 589, 624, or 625.

41. A pharmaceutical composition comprising:
a) the AAV viral vector of any one of claims 25-40; and
b) at least one pharmaceutically acceptable excipient and/or additive.

42. An AAV viral vector comprising:
a) an AAV vector of any one of the preceding claims; and
b) an AAV capsid protein.

43. The AAV viral vector of claim 42, wherein the AAV capsid protein is an AAV1 capsid protein, an AAV2 capsid protein, an AAV4 capsid protein, an AAV5 capsid protein, an AAV6 capsid protein, an AAV7 capsid protein, an AAV8 capsid protein, an AAV9 capsid protein, an AAV10 capsid protein, an AAV 11 capsid protein, an AAV12 capsid protein, an AAV13 capsid protein, an AAVPHP.B capsid protein, an AAVrh74 capsid protein or an AAVrh.10 capsid protein.

44. The AAV viral vector of claim 43, wherein the AAV capsid protein is an AAV9 or AAVrh10 capsid protein

45. A cell comprising the vector of any one of the preceding claims.

46. A method of treating a CAG repeat disease in a mammal comprising administering a composition or AAV vector according to any one of claims 1-45 to a toxic target CAG microsatellite repeat expansion (MRE) RNA sequence in tissues of the mammal whereby the level of expression of the toxic target RNA is reduced.

47. The method of claim 46, wherein the composition or AAV vector is administered to the subject intravenously, intrathecally, intracerebrally, intraventricularly, intranasally, intratracheally, intra-aurally, intra-ocularly, or peri-ocularly, orally, rectally, transmucosally, inhalationally, transdermally, parenterally, subcutaneously, intradermally, intramuscularly, intracisternally, intranervally, intrapleurally, topically, intralymphatically, intracisternally or intranerve.

48. The method of claim 46, wherein the composition or AAV vector is administered to the subject intravenously.

49. The method of claim 46, wherein the CAG repeat disorder is Huntington's Disease (HD) or Spinocerebellar Ataxia Type 1 (SCA1)

50. The method of claim 46, wherein the reduced level of expression of the toxic target RNA thereby ameliorates symptoms of HD or SCA1 in the mammal.

51. The method of claim 46, wherein the level of expression of the toxic target RNA is reduced compared to the reduction in the level of expression of untreated toxic target CAG RNA.

52. The method of claim 46, wherein the toxic CAG repeat is a CAG³⁶ or more.

53. The method of claim 46, wherein the toxic CAG repeat is a CAG⁸⁰ repeat.

54. The method of claim 46, wherein the level of reduction is between 1-fold and 20-fold.
